SEQUENCE LISTING (127 sequences)

SEQ ID NO: 1 - protein, 5 aa, synthetic construct
  REGION 1..5: note = CD3orig HCDR1
TYAMN

SEQ ID NO: 2 - protein, 5 aa, synthetic construct
  REGION 1..5: note = CD3opt HCDR1 (P033.078) (P035.093) (P021.045)
SYAMN

SEQ ID NO: 3 - protein, 5 aa, synthetic construct
  REGION 1..5: note = CD3opt HCDR1 (P035.064) (P004.042)
NYAMN

SEQ ID NO: 4 - protein, 19 aa, synthetic construct
  REGION 1..19: note = CD3orig HCDR2 CD3opt HCDR2 (P035.093) (P021.045)
RIRSKYNNYATYYADSVKG

SEQ ID NO: 5 - protein, 19 aa, synthetic construct
  REGION 1..19: note = CD3opt HCDR2 (P033.078)
RIRSKYNEYATYYADSVKG

SEQ ID NO: 6 - protein, 19 aa, synthetic construct
  REGION 1..19: note = CD3opt HCDR2 (P035.064)
RIRSKHNGYATYYADSVKG

SEQ ID NO: 7 - protein, 19 aa, synthetic construct
  REGION 1..19: note = CD3opt HCDR2 (P004.042)
RIRTKYNEYATYYADSVKG

SEQ ID NO: 8 - protein, 14 aa, synthetic construct
  REGION 1..14: note = CD3orig HCDR3
HGNFGNSYVSWFAY

SEQ ID NO: 9 - protein, 14 aa, synthetic construct
  REGION 1..14: note = CD3opt HCDR3 (P033.078)
ASNFPSSFVSYFGY

SEQ ID NO: 10 - protein, 14 aa, synthetic construct
  REGION 1..14: note = CD3opt HCDR3 (P035.093)
ASNFPASYVSYFAY

SEQ ID NO: 11 - protein, 14 aa, synthetic construct
  REGION 1..14: note = CD3opt HCDR3 (P035.064)
ASNFPSSYVSYFGY

SEQ ID NO: 12 - protein, 14 aa, synthetic construct
  REGION 1..14: note = CD3opt HCDR3 (P021.045)
ASNFPSSYVSYFAY

SEQ ID NO: 13 - protein, 14 aa, synthetic construct
  REGION 1..14: note = CD3opt HCDR3 (P004.042)
ASNFPQSYVSYFGY

SEQ ID NO: 14 - protein, 125 aa, synthetic construct
  REGION 1..125: note = CD3orig VH
EVQLLESGGGLVQPGGSLRLSCAASGFTFSTYAMNWVRQAPGKGLEWVSRIRSKYNNYATYYADSVKGRFTISRDDSKNTLYLQMNSLRAEDTAVYYCVRHGNFGNSYVSWFAYWGQGTLVTVSS

SEQ ID NO: 15 - protein, 125 aa, synthetic construct
  REGION 1..125: note = CD3opt VH (P033.078)
EVQLLESGGGLVQPGGSLRLSCAASGFTFESYAMNWVRQAPGKGLEWVSRIRSKYNEYATYYADSVKGRFTISRDDSKNTLYLQMNSLRAEDTAVYYCVRASNFPSSFVSYFGYWGQGTLVTVSS

SEQ ID NO: 16 - protein, 125 aa, synthetic construct
  REGION 1..125: note = CD3opt VH (P035.093)
EVQLLESGGGLVQPGGSLRLSCAASGFTFSSYAMNWVRQAPGKGLEWVSRIRSKYNNYATYYADSVKGRFTISRDDSKNTLYLQMNSLRAEDTAVYYCVRASNFPASYVSYFAYWGQGTLVTVSS

SEQ ID NO: 17 - protein, 125 aa, synthetic construct
  REGION 1..125: note = CD3opt VH (P035.064)
EVQLLESGGGLVQPGGSLRLSCAASGFDFDNYAMNWVRQAPGKGLEWVSRIRSKHNGYATYYADSVKGRFTISRDDSKNTLYLQMNSLRAEDTAVYYCVRASNFPSSYVSYFGYWGQGTLVTVSS

SEQ ID NO: 18 - protein, 125 aa, synthetic construct
  REGION 1..125: note = CD3opt VH (P021.045)
EVQLLESGGGLVQPGGSLRLSCAASGFTFSSYAMNWVRQAPGKGLEWVSRIRSKYNNYATYYADSVKGRFTISRDDSKNTLYLQMNSLRAEDTAVYYCVRASNFPSSYVSYFAYWGQGTLVTVSS

SEQ ID NO: 19 - protein, 125 aa, synthetic construct
  REGION 1..125: note = CD3opt VH (P004.042)
EVQLLESGGGLVQPGGSLRLSCAASGFQFDNYAMNWVRQAPGKGLEWVSRIRTKYNEYATYYADSVKGRFTISRDDSKNTLYLQMNSLRAEDTAVYYCVRASNFPQSYVSYFGYWGQGTLVTVSS

SEQ ID NO: 20 - protein, 14 aa, synthetic construct
  REGION 1..14: note = CD3orig / CD3opt LCDR1
GSSTGAVTTSNYAN

SEQ ID NO: 21 - protein, 7 aa, synthetic construct
  REGION 1..7: note = CD3orig / CD3opt LCDR2
GTNKRAP

SEQ ID NO: 22 - protein, 9 aa, synthetic construct
  REGION 1..9: note = CD3orig / CD3opt LCDR3
ALWYSNLWV

SEQ ID NO: 23 - protein, 109 aa, synthetic construct
  REGION 1..109: note = CD3orig / CD3opt VL
QAVVTQEPSLTVSPGGTVTLTCGSSTGAVTTSNYANWVQEKPGQAFRGLIGGTNKRAPGTPARFSGSLLGGKAALTLSGAQPEDEAEYYCALWYSNLWVFGGGTKLTVL

SEQ ID NO: 24 - protein, 5 aa, synthetic construct
  REGION 1..5: note = TYRP1 HCDR1
DYFLH

SEQ ID NO: 25 - protein, 17 aa, synthetic construct
  REGION 1..17: note = TYRP1 HCDR2
WINPDNGNTVYAQKFQG

SEQ ID NO: 26 - protein, 12 aa, synthetic construct
  REGION 1..12: note = TYRP1 HCDR3
RDYTYEKAALDY

SEQ ID NO: 27 - protein, 121 aa, synthetic construct
  REGION 1..121: note = TYRP1 VH
QVQLVQSGAEVKKPGASVKVSCKASGFNIKDYFLHWVRQAPGQGLEWMGWINPDNGNTVYAQKFQGRVTMTADTSTSTVYMELSSLRSEDTAVYYCTRRDYTYEKAALDYWGQGTLVTVSS

SEQ ID NO: 28 - protein, 11 aa, synthetic construct
  REGION 1..11: note = TYRP1 LCDR1
RASGNIYNYLA

SEQ ID NO: 29 - protein, 7 aa, synthetic construct
  REGION 1..7: note = TYRP1 LCDR2
DAKTLAD

SEQ ID NO: 30 - protein, 9 aa, synthetic construct
  REGION 1..9: note = TYRP1 LCDR3
QHFWSLPFT

SEQ ID NO: 31 - protein, 107 aa, synthetic construct
  REGION 1..107: note = TYRP1 VL
DIQMTQSPSSLSASVGDRVTITCRASGNIYNYLAWYQQKPGKVPKLLIYDAKTLADGVPSRFSGSGSGTDFTLTISSLQPEDVATYYCQHFWSLPFTFGQGTKLEIK

SEQ ID NO: 32 - protein, 674 aa, synthetic construct
  REGION 1..674: note = TYRP1 VH-CH1(EE) - CD3orig/CD3opt VL-CH1 - Fc (knob, PGLALA)
QVQLVQSGAEVKKPGASVKVSCKASGFNIKDYFLHWVRQAPGQGLEWMGWINPDNGNTVYAQKFQGRVTMTADTSTSTVYMELSSLRSEDTAVYYCTRRDYTYEKAALDYWGQGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDGGGGSGGGGSQAVVTQEPSLTVSPGGTVTLTCGSSTGAVTTSNYANWVQEKPGQAFRGLIGGTNKRAPGTPARFSGSLLGGKAALTLSGAQPEDEAEYYCALWYSNLWVFGGGTKLTVLSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 33 - protein, 449 aa, synthetic construct
  REGION 1..449: note = TYRP1 VH-CH1(EE) -Fc (hole, PGLALA)
QVQLVQSGAEVKKPGASVKVSCKASGFNIKDYFLHWVRQAPGQGLEWMGWINPDNGNTVYAQKFQGRVTMTADTSTSTVYMELSSLRSEDTAVYYCTRRDYTYEKAALDYWGQGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 34 - protein, 214 aa, synthetic construct
  REGION 1..214: note = TYRP1 VL-CL(RK)
DIQMTQSPSSLSASVGDRVTITCRASGNIYNYLAWYQQKPGKVPKLLIYDAKTLADGVPSRFSGSGSGTDFTLTISSLQPEDVATYYCQHFWSLPFTFGQGTKLEIKRTVAAPSVFIFPPSDRKLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 35 - protein, 232 aa, synthetic construct
  REGION 1..232: note = CD3orig VH-CL
EVQLLESGGGLVQPGGSLRLSCAASGFTFSTYAMNWVRQAPGKGLEWVSRIRSKYNNYATYYADSVKGRFTISRDDSKNTLYLQMNSLRAEDTAVYYCVRHGNFGNSYVSWFAYWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 36 - protein, 232 aa, synthetic construct
  REGION 1..232: note = CD3opt (P033.078) VH-CL
EVQLLESGGGLVQPGGSLRLSCAASGFTFESYAMNWVRQAPGKGLEWVSRIRSKYNEYATYYADSVKGRFTISRDDSKNTLYLQMNSLRAEDTAVYYCVRASNFPSSFVSYFGYWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 37 - protein, 232 aa, synthetic construct
  REGION 1..232: note = CD3opt (P035.093) VH-CL
EVQLLESGGGLVQPGGSLRLSCAASGFTFSSYAMNWVRQAPGKGLEWVSRIRSKYNNYATYYADSVKGRFTISRDDSKNTLYLQMNSLRAEDTAVYYCVRASNFPASYVSYFAYWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 38 - protein, 232 aa, synthetic construct
  REGION 1..232: note = CD3opt (P035.064) VH-CL
EVQLLESGGGLVQPGGSLRLSCAASGFDFDNYAMNWVRQAPGKGLEWVSRIRSKHNGYATYYADSVKGRFTISRDDSKNTLYLQMNSLRAEDTAVYYCVRASNFPSSYVSYFGYWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 39 - protein, 232 aa, synthetic construct
  REGION 1..232: note = CD3opt (P021.045) VH-CL
EVQLLESGGGLVQPGGSLRLSCAASGFTFSSYAMNWVRQAPGKGLEWVSRIRSKYNNYATYYADSVKGRFTISRDDSKNTLYLQMNSLRAEDTAVYYCVRASNFPSSYVSYFAYWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 40 - protein, 232 aa, synthetic construct
  REGION 1..232: note = CD3opt (P004.042) VH-CL
EVQLLESGGGLVQPGGSLRLSCAASGFQFDNYAMNWVRQAPGKGLEWVSRIRTKYNEYATYYADSVKGRFTISRDDSKNTLYLQMNSLRAEDTAVYYCVRASNFPQSYVSYFGYWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 41 - protein, 360 aa, synthetic construct [sequence omitted]

SEQ ID NO: 42 - protein, 325 aa, synthetic construct
  REGION 1..325: note = Human CD3 delta stalk - Fc (hole) - Avi
FKIPIEELEDRVFVNCNTSITWVEGTVGTLLSDITRLDLGKRILDPRGIYRCNGTDIYKDKESTVQVHYRMCRSEQLYFQGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGKSGGLNDIFEAQKIEWHE

SEQ ID NO: 43 - protein, 351 aa, synthetic construct
  REGION 1..351: note = Cynomolgus CD3 epsilon stalk - Fc (knob) - Avi
QDGNEEMGSITQTPYQVSISGTTVILTCSQHLGSEAQWQHNGKNKEDSGDRLFLPEFSEMEQSGYYVCYPRGSNPEDASHHLYLKARVSENCVDEQLYFQGGSPKSADKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKSGGLNDIFEAQKIEWHE

SEQ ID NO: 44 - protein, 334 aa, synthetic construct
  REGION 1..334: note = Cynomolgus CD3 delta stalk - Fc (hole) - Avi
FKIPVEELEDRVFVKCNTSVTWVEGTVGTLLTNNTRLDLGKRILDPRGIYRCNGTDIYKDKESAVQVHYRMSQNCVDEQLYFQGGSPKSADKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGKSGGLNDIFEAQKIEWHE

SEQ ID NO: 45 - protein, 186 aa, Homo sapiens
QDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 46 - protein, 177 aa, Macaca fascicularis
QDGNEEMGSITQTPYQVSISGTTVILTCSQHLGSEAQWQHNGKNKEDSGDRLFLPEFSEMEQSGYYVCYPRGSNPEDASHHLYLKARVCENCMEMDVMAVATIVIVDICITLGLLLLVYYWSKNRKAKAKPVTRGAGAGGRQRGQNKERPPPVPNPDYEPIRKGQQDLYSGLNQRRI

SEQ ID NO: 47 - protein, 225 aa, synthetic construct
  REGION 1..225: note = hIgG1 Fc region
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 48 - protein, 10 aa, synthetic construct
  REGION 1..10: note = linker GGGGSGGGGS
GGGGSGGGGS

SEQ ID NO: 49 - protein, 11 aa, synthetic construct
  REGION 1..11: note = linker DGGGGSGGGGS
DGGGGSGGGGS

SEQ ID NO: 50 - protein, 107 aa, synthetic construct [sequence omitted]

SEQ ID NO: 51 - protein, 105 aa, synthetic construct
  REGION 1..105: note = Human lambda CL domain
QPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 52 - protein, 328 aa, synthetic construct
  REGION 1..328: note = Human IgG1 heavy chain constant region (CH1-CH2-CH3)
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 53 - protein, 120 aa, synthetic construct
  REGION 1..120: note = 16D5 VH
EVQLVESGGGLVKPGGSLRLSCAASGFTFSNAWMSWVRQAPGKGLEWVGRIKSKTDGGTTDYAAPVKGRFTISRDDSKNTLYLQMNSLKTEDTAVYYCTTPWEWSWYDYWGQGTLVTVSS

SEQ ID NO: 54 - protein, 5 aa, synthetic construct
  REGION 1..5: note = 16D5 CDRH1
NAWMS

SEQ ID NO: 55 - protein, 19 aa, synthetic construct
  REGION 1..19: note = 16D5 CDRH2
RIKSKTDGGTTDYAAPVKG

SEQ ID NO: 56 - protein, 9 aa, synthetic construct
  REGION 1..9: note = 16D5 CDRH3
PWEWSWYDY

SEQ ID NO: 57 - protein, 118 aa, synthetic construct
  REGION 1..118: note = ID_4.24.72 VH
QIQLVQSGPELKKPGETVKISCKASGYTVTDYSMNWVKQAPGKCLKWMGWINTETGEPRYTDDFKGRFAFSLETSASTAYLQINNLKNEDSATYFCAREGDYDVFDYWGHGTTLKVSS

SEQ ID NO: 58 - protein, 5 aa, synthetic construct
  REGION 1..5: note = ID_4.24.72 CDRH1
DYSMN

SEQ ID NO: 59 - protein, 17 aa, synthetic construct
  REGION 1..17: note = ID_4.24.72 CDRH2
WINTETGEPRYTDDFKG

SEQ ID NO: 60 - protein, 9 aa, synthetic construct
  REGION 1..9: note = ID_4.24.72 CDRH3
EGDYDVFDY

SEQ ID NO: 61 - protein, 111 aa, synthetic construct
  REGION 1..111: note = ID_4.24.72 VL
DIVLTQSPASLAVSLGQRATISCRASKSVSTSSYSYMHWYQQKPGQPPKLLIKYVSYLESGVPARFSGSGSGTDFTLNIHPVEEEDAATYYCQHSREFPYTFGCGTKLEIK

SEQ ID NO: 62 - protein, 15 aa, synthetic construct
  REGION 1..15: note = ID_4.24.72 CDRL1
RASKSVSTSSYSYMH

SEQ ID NO: 63 - protein, 7 aa, synthetic construct
  REGION 1..7: note = ID_4.24.72 CDRL2
YVSYLES

SEQ ID NO: 64 - protein, 9 aa, synthetic construct
  REGION 1..9: note = ID_4.24.72 CDRL3
QHSREFPYT

SEQ ID NO: 65 - protein, 969 aa, synthetic construct
  REGION 1..969: note = FOLR1 proTCB with protease linker and anti-CD3 P035.093 K chain
QIQLVQSGPELKKPGETVKISCKASGYTVTDYSMNWVKQAPGKCLKWMGWINTETGEPRYTDDFKGRFAFSLETSASTAYLQINNLKNEDSATYFCAREGDYDVFDYWGHGTTLKVSSGGGGSGGGGSGGGGSGGGGSDIVLTQSPASLAVSLGQRATISCRASKSVSTSSYSYMHWYQQKPGQPPKLLIKYVSYLESGVPARFSGSGSGTDFTLNIHPVEEEDAATYYCQHSREFPYTFGCGTKLEIKSGGGSGGGGSPMAKKGGGGSGGGGSGGGGSGGSEVQLLESGGGLVQPGGSLRLSCAASGFTFSSYAMNWVRQAPGKGLEWVSRIRSKYNNYATYYADSVKGRFTISRDDSKNTLYLQMNSLRAEDTAVYYCVRASNFPASYVSYFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSEVQLVESGGGLVKPGGSLRLSCAASGFTFSNAWMSWVRQAPGKGLEWVGRIKSKTDGGTTDYAAPVKGRFTISRDDSKNTLYLQMNSLKTEDTAVYYCTTPWEWSWYDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 66 - protein, 448 aa, synthetic construct
  REGION 1..448: note = FOLR1 proTCB with protease linker and anti-CD3 P035.093 H chain
EVQLVESGGGLVKPGGSLRLSCAASGFTFSNAWMSWVRQAPGKGLEWVGRIKSKTDGGTTDYAAPVKGRFTISRDDSKNTLYLQMNSLKTEDTAVYYCTTPWEWSWYDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 67 - protein, 215 aa, synthetic construct
  REGION 1..215: note = FOLR1 proTCB with protease linker and anti-CD3 P035.093 L chain
QAVVTQEPSLTVSPGGTVTLTCGSSTGAVTTSNYANWVQEKPGQAFRGLIGGTNKRAPGTPARFSGSLLGGKAALTLSGAQPEDEAEYYCALWYSNLWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 68 - protein, 33 aa, synthetic construct
  REGION 1..33: note = Matriptase linker
SGGGSGGGGSPMAKKGGGGSGGGGSGGGGSGGS

SEQ ID NO: 69 - protein, 969 aa, synthetic construct
  REGION 1..969: note = FOLR1proTCB with protease linker CD3 P035.093 K chain
QIQLVQSGPELKKPGETVKISCKASGYTVTDYSMNWVKQAPGKCLKWMGWINTETGEPRYTDDFKGRFAFSLETSASTAYLQINNLKNEDSATYFCAREGDYDVFDYWGHGTTLKVSSGGGGSGGGGSGGGGSGGGGSDIVLTQSPASLAVSLGQRATISCRASKSVSTSSYSYMHWYQQKPGQPPKLLIKYVSYLESGVPARFSGSGSGTDFTLNIHPVEEEDAATYYCQHSREFPYTFGCGTKLEIKGGGGSVHMPLGFLGPRQARVVNGGGGGSGGGGSEVQLLESGGGLVQPGGSLRLSCAASGFTFSSYAMNWVRQAPGKGLEWVSRIRSKYNNYATYYADSVKGRFTISRDDSKNTLYLQMNSLRAEDTAVYYCVRASNFPASYVSYFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSEVQLVESGGGLVKPGGSLRLSCAASGFTFSNAWMSWVRQAPGKGLEWVGRIKSKTDGGTTDYAAPVKGRFTISRDDSKNTLYLQMNSLKTEDTAVYYCTTPWEWSWYDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 70 - protein, 33 aa, synthetic construct
  REGION 1..33: note = Linker GGGGSVHMPLGFLGPRQARVVNGGGGGSGGGGS
GGGGSVHMPLGFLGPRQARVVNGGGGGSGGGGS

SEQ ID NO: 71 - protein, 969 aa, synthetic construct
  REGION 1..969: note = FOLR1proTCB with non-cleavable linker CD3 P035.093 K chain
QIQLVQSGPELKKPGETVKISCKASGYTVTDYSMNWVKQAPGKCLKWMGWINTETGEPRYTDDFKGRFAFSLETSASTAYLQINNLKNEDSATYFCAREGDYDVFDYWGHGTTLKVSSGGGGSGGGGSGGGGSGGGGSDIVLTQSPASLAVSLGQRATISCRASKSVSTSSYSYMHWYQQKPGQPPKLLIKYVSYLESGVPARFSGSGSGTDFTLNIHPVEEEDAATYYCQHSREFPYTFGCGTKLEIKGGGGSGGGGSGGGGSGGGGGGGSGGGGSGGGGSEVQLLESGGGLVQPGGSLRLSCAASGFTFSSYAMNWVRQAPGKGLEWVSRIRSKYNNYATYYADSVKGRFTISRDDSKNTLYLQMNSLRAEDTAVYYCVRASNFPASYVSYFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSEVQLVESGGGLVKPGGSLRLSCAASGFTFSNAWMSWVRQAPGKGLEWVGRIKSKTDGGTTDYAAPVKGRFTISRDDSKNTLYLQMNSLKTEDTAVYYCTTPWEWSWYDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 72 - protein, 33 aa, synthetic construct
  REGION 1..33: note = Linker GGGGSGGGGSGGGGSGGGGGGGSGGGGSGGGGS
GGGGSGGGGSGGGGSGGGGGGGSGGGGSGGGGS

SEQ ID NO: 73 - protein, 687 aa, synthetic construct
  REGION 1..687: note = FOLR1 TCB CD3 P035.093 K chain
EVQLLESGGGLVQPGGSLRLSCAASGFTFSSYAMNWVRQAPGKGLEWVSRIRSKYNNYATYYADSVKGRFTISRDDSKNTLYLQMNSLRAEDTAVYYCVRASNFPASYVSYFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSEVQLVESGGGLVKPGGSLRLSCAASGFTFSNAWMSWVRQAPGKGLEWVGRIKSKTDGGTTDYAAPVKGRFTISRDDSKNTLYLQMNSLKTEDTAVYYCTTPWEWSWYDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 74 - protein, 969 aa, synthetic construct
  REGION 1..969: note = FOLR1proTCB with matriptase linker CD3 CH2527 K chain
QIQLVQSGPELKKPGETVKISCKASGYTVTDYSMNWVKQAPGKCLKWMGWINTETGEPRYTDDFKGRFAFSLETSASTAYLQINNLKNEDSATYFCAREGDYDVFDYWGHGTTLKVSSGGGGSGGGGSGGGGSGGGGSDIVLTQSPASLAVSLGQRATISCRASKSVSTSSYSYMHWYQQKPGQPPKLLIKYVSYLESGVPARFSGSGSGTDFTLNIHPVEEEDAATYYCQHSREFPYTFGCGTKLEIKGGGGSGGGGSRQARVVNGGGGGSGGGGSGGGGSEVQLLESGGGLVQPGGSLRLSCAASGFTFSTYAMNWVRQAPGKGLEWVSRIRSKYNNYATYYADSVKGRFTISRDDSKNTLYLQMNSLRAEDTAVYYCVRHGNFGNSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSEVQLVESGGGLVKPGGSLRLSCAASGFTFSNAWMSWVRQAPGKGLEWVGRIKSKTDGGTTDYAAPVKGRFTISRDDSKNTLYLQMNSLKTEDTAVYYCTTPWEWSWYDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 75 - protein, 33 aa, synthetic construct
  REGION 1..33: note = Linker GGGGSGGGGSRQARVVNGGGGGSGGGGSGGGGS
GGGGSGGGGSRQARVVNGGGGGSGGGGSGGGGS

SEQ ID NO: 76 - protein, 969 aa, synthetic construct
  REGION 1..969: note = FOLR1proTCB with MMP2/9-matriptase linker CD3 CH2527 K chain
QIQLVQSGPELKKPGETVKISCKASGYTVTDYSMNWVKQAPGKCLKWMGWINTETGEPRYTDDFKGRFAFSLETSASTAYLQINNLKNEDSATYFCAREGDYDVFDYWGHGTTLKVSSGGGGSGGGGSGGGGSGGGGSDIVLTQSPASLAVSLGQRATISCRASKSVSTSSYSYMHWYQQKPGQPPKLLIKYVSYLESGVPARFSGSGSGTDFTLNIHPVEEEDAATYYCQHSREFPYTFGCGTKLEIKGGGGSVHMPLGFLGPRQARVVNGGGGGSGGGGSEVQLLESGGGLVQPGGSLRLSCAASGFTFSTYAMNWVRQAPGKGLEWVSRIRSKYNNYATYYADSVKGRFTISRDDSKNTLYLQMNSLRAEDTAVYYCVRHGNFGNSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSEVQLVESGGGLVKPGGSLRLSCAASGFTFSNAWMSWVRQAPGKGLEWVGRIKSKTDGGTTDYAAPVKGRFTISRDDSKNTLYLQMNSLKTEDTAVYYCTTPWEWSWYDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 77 - protein, 33 aa, synthetic construct; identical to SEQ ID NO: 70
  REGION 1..33: note = Linker GGGGSVHMPLGFLGPRQARVVNGGGGGSGGGGS

SEQ ID NO: 78 - protein, 969 aa, synthetic construct
  REGION 1..969: note = FOLR1 proTCB with non-cleavable linker CD3 CH2527 K chain
QIQLVQSGPELKKPGETVKISCKASGYTVTDYSMNWVKQAPGKCLKWMGWINTETGEPRYTDDFKGRFAFSLETSASTAYLQINNLKNEDSATYFCAREGDYDVFDYWGHGTTLKVSSGGGGSGGGGSGGGGSGGGGSDIVLTQSPASLAVSLGQRATISCRASKSVSTSSYSYMHWYQQKPGQPPKLLIKYVSYLESGVPARFSGSGSGTDFTLNIHPVEEEDAATYYCQHSREFPYTFGCGTKLEIKGGGGSGGGGSGGGGSGGGGGGGSGGGGSGGGGSEVQLLESGGGLVQPGGSLRLSCAASGFTFSTYAMNWVRQAPGKGLEWVSRIRSKYNNYATYYADSVKGRFTISRDDSKNTLYLQMNSLRAEDTAVYYCVRHGNFGNSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSEVQLVESGGGLVKPGGSLRLSCAASGFTFSNAWMSWVRQAPGKGLEWVGRIKSKTDGGTTDYAAPVKGRFTISRDDSKNTLYLQMNSLKTEDTAVYYCTTPWEWSWYDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 79 - protein, 118 aa, synthetic construct
  REGION 1..118: note = H1L1 VH
QVQLVQSGSELKKPGASVKVSCKASGYTVTDYSMNWVRQAPGQGLEWMGWINTETGEPRYTDDFKGRFVFSLDTSVSTAYLQISSLKAEDTAVYYCAREGDYDVFDYWGQGTLVTVSS

SEQ ID NO: 80 - protein, 111 aa, synthetic construct
  REGION 1..111: note = H1L1 VL
DIVMTQSPDSLAVSLGERATINCRASKSVSTSSYSYMHWYQQKPGQPPKLLIKYVSYLESGVPDRFSGSGSGTDFTLTISSLQAEDVAVYYCQHSREFPYTFGQGTKLEIK

SEQ ID NO: 81 - protein, 111 aa, synthetic construct
  REGION 1..111: note = H1L2 VL
DIVMTQSPDSLAVSLGERATINCKSSKSVSTSSYSYMHWYQQKPGQPPKLLIKYVSYLESGVPDRFSGSGSGTDFTLTISSLQAEDVAVYYCQHSREFPYTFGQGTKLEIK

SEQ ID NO: 82 - protein, 15 aa, synthetic construct
  REGION 1..15: note = H1L2 CDRL1
KSSKSVSTSSYSYMH

SEQ ID NO: 83 - protein, 118 aa, synthetic construct
  REGION 1..118: note = H2L2 VH
QVQLVQSGSELKKPGASVKVSCKASGYTVTDYSMNWVRQAPGQGLEWMGWINTETGEPRYTDDFTGRFVFSLDTSVSTAYLQISSLKAEDTAVYYCAREGDYDVFDYWGQGTLVTVSS

SEQ ID NO: 84 - protein, 17 aa, synthetic construct
  REGION 1..17: note = H2L2 CDRH2
WINTETGEPRYTDDFTG

SEQ ID NO: 85 - protein, 118 aa, synthetic construct
  REGION 1..118: note = H3L2 VH
QVQLVQSGSELKKPGASVKVSCKASGYTVTDYSMNWVRQAPGQGLEWMGWINTETGEPRYTQGFKGRFVFSLDTSVSTAYLQISSLKAEDTAVYYCAREGDYDVFDYWGQGTLVTVSS

SEQ ID NO: 86 - protein, 17 aa, synthetic construct
  REGION 1..17: note = H3L2 CDRH2
WINTETGEPRYTQGFKG

SEQ ID NO: 87 - protein, 111 aa, synthetic construct
  REGION 1..111: note = H3L3 VL
DIVMTQSPDSLAVSLGERATINCRASKSVSTSSYSYMHWYQQKPGQPPKLLIKYVSYLESGVPDRFSGSGSGTDFTLTISSLQAEDVAVYYCQQSREFPYTFGQGTKLEIK

SEQ ID NO: 88 - protein, 9 aa, synthetic construct
  REGION 1..9: note = H3L3 CDRL3
QQSREFPYT

SEQ ID NO: 89 - protein, 118 aa, synthetic construct
  REGION 1..118: note = H7L5 VH
QVQLVQSGAEVKKPGASVKVSCKASGYTVTDYSMNWVRQAPGQGLEWMGWINTETGEPRYTDDFKGRVTMTRDTSISTAYMELSRLRSDDTAVYYCAREGDYDVFDYWGQGTLVTVSS

SEQ ID NO: 90 - protein, 111 aa, synthetic construct
  REGION 1..111: note = H7L5 VL
DIVMTQSPDSLAVSLGERATINCRASKSVSTSSYSYMHWYQQKPGQPPKLLIYYVSYLESGVPDRFSGSGSGTDFTLTISSLQAEDVAVYYCQHSREFPYTFGQGTKLEIK

SEQ ID NO: 91 - protein, 249 aa, synthetic construct
  REGION 1..249: note = H1L1 scFv
QVQLVQSGSELKKPGASVKVSCKASGYTVTDYSMNWVRQAPGQCLEWMGWINTETGEPRYTDDFKGRFVFSLDTSVSTAYLQISSLKAEDTAVYYCAREGDYDVFDYWGQGTLVTVSSGGGGSGGGGSGGGGSGGGGSDIVMTQSPDSLAVSLGERATINCRASKSVSTSSYSYMHWYQQKPGQPPKLLIKYVSYLESGVPDRFSGSGSGTDFTLTISSLQAEDVAVYYCQHSREFPYTFGCGTKLEIK

SEQ ID NO: 92 - protein, 249 aa, synthetic construct
  REGION 1..249: note = H1L2 scFv
QVQLVQSGSELKKPGASVKVSCKASGYTVTDYSMNWVRQAPGQCLEWMGWINTETGEPRYTDDFKGRFVFSLDTSVSTAYLQISSLKAEDTAVYYCAREGDYDVFDYWGQGTLVTVSSGGGGSGGGGSGGGGSGGGGSDIVMTQSPDSLAVSLGERATINCKSSKSVSTSSYSYMHWYQQKPGQPPKLLIKYVSYLESGVPDRFSGSGSGTDFTLTISSLQAEDVAVYYCQHSREFPYTFGCGTKLEIK

SEQ ID NO: 93 - protein, 249 aa, synthetic construct
  REGION 1..249: note = H2L2 scFv
QVQLVQSGSELKKPGASVKVSCKASGYTVTDYSMNWVRQAPGQCLEWMGWINTETGEPRYTDDFTGRFVFSLDTSVSTAYLQISSLKAEDTAVYYCAREGDYDVFDYWGQGTLVTVSSGGGGSGGGGSGGGGSGGGGSDIVMTQSPDSLAVSLGERATINCKSSKSVSTSSYSYMHWYQQKPGQPPKLLIKYVSYLESGVPDRFSGSGSGTDFTLTISSLQAEDVAVYYCQHSREFPYTFGCGTKLEIK

SEQ ID NO: 94 - protein, 249 aa, synthetic construct
  REGION 1..249: note = H3L2 scFv
QVQLVQSGSELKKPGASVKVSCKASGYTVTDYSMNWVRQAPGQCLEWMGWINTETGEPRYTQGFKGRFVFSLDTSVSTAYLQISSLKAEDTAVYYCAREGDYDVFDYWGQGTLVTVSSGGGGSGGGGSGGGGSGGGGSDIVMTQSPDSLAVSLGERATINCKSSKSVSTSSYSYMHWYQQKPGQPPKLLIKYVSYLESGVPDRFSGSGSGTDFTLTISSLQAEDVAVYYCQHSREFPYTFGCGTKLEIK

SEQ ID NO: 95 - protein, 969 aa, synthetic construct
  REGION 1..969: note = FolR1 pro TCB P035.093 H1L1 K chain
QVQLVQSGSELKKPGASVKVSCKASGYTVTDYSMNWVRQAPGQCLEWMGWINTETGEPRYTDDFKGRFVFSLDTSVSTAYLQISSLKAEDTAVYYCAREGDYDVFDYWGQGTLVTVSSGGGGSGGGGSGGGGSGGGGSDIVMTQSPDSLAVSLGERATINCRASKSVSTSSYSYMHWYQQKPGQPPKLLIKYVSYLESGVPDRFSGSGSGTDFTLTISSLQAEDVAVYYCQHSREFPYTFGCGTKLEIKGGGGSGGGGSPMAKKGGGGSGGGGSGGGGSGGSEVQLLESGGGLVQPGGSLRLSCAASGFTFSSYAMNWVRQAPGKGLEWVSRIRSKYNNYATYYADSVKGRFTISRDDSKNTLYLQMNSLRAEDTAVYYCVRASNFPASYVSYFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSEVQLVESGGGLVKPGGSLRLSCAASGFTFSNAWMSWVRQAPGKGLEWVGRIKSKTDGGTTDYAAPVKGRFTISRDDSKNTLYLQMNSLKTEDTAVYYCTTPWEWSWYDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 96 - protein, 969 aa, synthetic construct
  REGION 1..969: note = FolR1 pro TCB P035.093 H1L2 K chain
QVQLVQSGSELKKPGASVKVSCKASGYTVTDYSMNWVRQAPGQCLEWMGWINTETGEPRYTDDFKGRFVFSLDTSVSTAYLQISSLKAEDTAVYYCAREGDYDVFDYWGQGTLVTVSSGGGGSGGGGSGGGGSGGGGSDIVMTQSPDSLAVSLGERATINCKSSKSVSTSSYSYMHWYQQKPGQPPKLLIKYVSYLESGVPDRFSGSGSGTDFTLTISSLQAEDVAVYYCQHSREFPYTFGCGTKLEIKGGGGSGGGGSPMAKKGGGGSGGGGSGGGGSGGSEVQLLESGGGLVQPGGSLRLSCAASGFTFSSYAMNWVRQAPGKGLEWVSRIRSKYNNYATYYADSVKGRFTISRDDSKNTLYLQMNSLRAEDTAVYYCVRASNFPASYVSYFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSEVQLVESGGGLVKPGGSLRLSCAASGFTFSNAWMSWVRQAPGKGLEWVGRIKSKTDGGTTDYAAPVKGRFTISRDDSKNTLYLQMNSLKTEDTAVYYCTTPWEWSWYDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 97 - protein, 969 aa, synthetic construct
  REGION 1..969: note = FolR1 pro TCB P035.093 H2L2 K chain
QVQLVQSGSELKKPGASVKVSCKASGYTVTDYSMNWVRQAPGQCLEWMGWINTETGEPRYTDDFTGRFVFSLDTSVSTAYLQISSLKAEDTAVYYCAREGDYDVFDYWGQGTLVTVSSGGGGSGGGGSGGGGSGGGGSDIVMTQSPDSLAVSLGERATINCKSSKSVSTSSYSYMHWYQQKPGQPPKLLIKYVSYLESGVPDRFSGSGSGTDFTLTISSLQAEDVAVYYCQHSREFPYTFGCGTKLEIKGGGGSGGGGSPMAKKGGGGSGGGGSGGGGSGGSEVQLLESGGGLVQPGGSLRLSCAASGFTFSSYAMNWVRQAPGKGLEWVSRIRSKYNNYATYYADSVKGRFTISRDDSKNTLYLQMNSLRAEDTAVYYCVRASNFPASYVSYFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSEVQLVESGGGLVKPGGSLRLSCAASGFTFSNAWMSWVRQAPGKGLEWVGRIKSKTDGGTTDYAAPVKGRFTISRDDSKNTLYLQMNSLKTEDTAVYYCTTPWEWSWYDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 98 - protein, 969 aa, synthetic construct
  REGION 1..969: note = FolR1 pro TCB P035.093 H3L2 K chain
QVQLVQSGSELKKPGASVKVSCKASGYTVTDYSMNWVRQAPGQCLEWMGWINTETGEPRYTQGFKGRFVFSLDTSVSTAYLQISSLKAEDTAVYYCAREGDYDVFDYWGQGTLVTVSSGGGGSGGGGSGGGGSGGGGSDIVMTQSPDSLAVSLGERATINCKSSKSVSTSSYSYMHWYQQKPGQPPKLLIKYVSYLESGVPDRFSGSGSGTDFTLTISSLQAEDVAVYYCQHSREFPYTFGCGTKLEIKGGGGSGGGGSPMAKKGGGGSGGGGSGGGGSGGSEVQLLESGGGLVQPGGSLRLSCAASGFTFSSYAMNWVRQAPGKGLEWVSRIRSKYNNYATYYADSVKGRFTISRDDSKNTLYLQMNSLRAEDTAVYYCVRASNFPASYVSYFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSEVQLVESGGGLVKPGGSLRLSCAASGFTFSNAWMSWVRQAPGKGLEWVGRIKSKTDGGTTDYAAPVKGRFTISRDDSKNTLYLQMNSLKTEDTAVYYCTTPWEWSWYDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 99 - protein, 35 aa, synthetic construct
  REGION 1..35: note = MMP Protease linker
GGGGSGGGGSGPLGLWSQGGGGSGGGGSGGGGSGG

SEQ ID NO: 100 - protein, 8 aa, synthetic construct
  REGION 1..8: note = Protease recognition site 1
RQARVVNG

SEQ ID NO: 101 - protein, 18 aa, synthetic construct
  REGION 1..18: note = Protease recognition site 2
VHMPLGFLGPGRSRGSFP

SEQ ID NO: 102 - protein, 21 aa, synthetic construct
  REGION 1..21: note = Protease recognition site 3
  REGION 9..13: note = misc_feature - Xaa can be any naturally occurring amino acid
RQARVVNGXXXXXVPLSLYSG

SEQ ID NO: 103 - protein, 16 aa, synthetic construct
  REGION 1..16: note = Protease recognition site 4
RQARVVNGVPLSLYSG

SEQ ID NO: 104 - protein, 7 aa, synthetic construct
  REGION 1..7: note = Protease recognition site 5
PLGLWSQ

SEQ ID NO: 105 - protein, 18 aa, synthetic construct
  REGION 1..18: note = Protease recognition site 6
VHMPLGFLGPRQARVVNG

SEQ ID NO: 106 - protein, 6 aa, synthetic construct
  REGION 1..6: note = Protease recognition site 7
FVGGTG

SEQ ID NO: 107 - protein, 8 aa, synthetic construct
  REGION 1..8: note = Protease recognition site 8
KKAAPVNG

SEQ ID NO: 108 - protein, 8 aa, synthetic construct
  REGION 1..8: note = Protease recognition site 9
PMAKKVNG

SEQ ID NO: 109 - protein, 8 aa, synthetic construct
  REGION 1..8: note = Protease recognition site 10
QARAKVNG

SEQ ID NO: 110 - protein, 10 aa, synthetic construct
  REGION 1..10: note = Protease recognition site 11
VHMPLGFLGP

SEQ ID NO: 111 - protein, 5 aa, synthetic construct
  REGION 1..5: note = Protease recognition site 12
QARAK

SEQ ID NO: 112 - protein, 15 aa, synthetic construct
  REGION 1..15: note = Protease recognition site 13
VHMPLGFLGPPMAKK

SEQ ID NO: 113 - protein, 5 aa, synthetic construct
  REGION 1..5: note = Protease recognition site 14
KKAAP

SEQ ID NO: 114 - protein, 5 aa, synthetic construct
  REGION 1..5: note = Protease recognition site 15
PMAKK

SEQ ID NO: 115 - protein, 33 aa, synthetic construct; identical to SEQ ID NO: 70
  REGION 1..33: note = Combined MMP9 MK062, 33 AA for CD3

SEQ ID NO: 116 - protein, 33 aa, synthetic construct
  REGION 1..33: note = Cathepsin S/B
GGGGSGGGGSGGGGSFVGGTGGGGSGGGGSGGS

SEQ ID NO: 117 - protein, 33 aa, synthetic construct
  REGION 1..33: note = KKAAPVNG
GGGGSGGGGSKKAAPVNGGGGGSGGGGSGGGGS

SEQ ID NO: 118 - protein, 33 aa, synthetic construct
  REGION 1..33: note = PMAKKVNG
GGGGSGGGGSPMAKKVNGGGGGSGGGGSGGGGS

SEQ ID NO: 119 - protein, 33 aa, synthetic construct
  REGION 1..33: note = QARAKVNG
GGGGSGGGGSQARAKVNGGGGGSGGGGSGGGGS

SEQ ID NO: 120 - protein, 33 aa, synthetic construct
  REGION 1..33: note = MMP9
GGGGSGGGGSVHMPLGFLGPGGGGSGGGGSGGS

SEQ ID NO: 121 - protein, 33 aa, synthetic construct
  REGION 1..33: note = QARAK
GGGGSGGGGSQARAKGGGGSGGGGSGGGGSGGS

SEQ ID NO: 122 - protein, 33 aa, synthetic construct
  REGION 1..33: note = MMP9-PMAKK
GGGGSVHMPLGFLGPPMAKKGGGGSGGGGSGGS

SEQ ID NO: 123 - protein, 33 aa, synthetic construct
  REGION 1..33: note = KKAAP
GGGGSGGGGSKKAAPGGGGSGGGGSGGGGSGGS

SEQ ID NO: 124 - protein, 33 aa, synthetic construct
  REGION 1..33: note = PMAKK
GGGGSGGGGSPMAKKGGGGSGGGGSGGGGSGGS

SEQ ID NO: 125 - protein, 28 aa, synthetic construct
  REGION 1..28: note = Combined NF9/Mat5 linker
GGGGSVHMPLGFLGPGRSRGSFPGGGGS

SEQ ID NO: 126 - protein, 41 aa, synthetic construct
  REGION 1..41: note = Combined MK062 MMP9
GGGGSGGGGSRQARVVNGGGGGSVPLSLYSGGGGGSGGGGS

SEQ ID NO: 127 - protein, 36 aa, synthetic construct
  REGION 1..36: note = Combined MK062 MMP9
GGGGSGGGGSRQARVVNGVPLSLYSGGGGGSGGGGS

(19) United States
(12) Patent Application Publication
(10) Pub. No.: US 2023/0287145 A1
(43) Pub. Date: Sep. 14, 2023

(54) PROTEASE-ACTIVATED T CELL BISPECIFIC ANTIBODIES

(71) Applicant: Hoffmann-La Roche Inc., Little Falls, NJ (US)

(72) Inventors: Peter BRUENKER, Hittnau (CH); Alejandro CARPY GUTIERREZ CIRLOS, München (DE); Anne FREIMOSER-GRUNDSCHOBER, Zürich (CH); Martina GEIGER, Obfelden (CH); Thomas HOFER, Zürich (CH); Christian KLEIN, Weilheim (DE); Ekkehard MOESSNER, Kreuzlingen (CH); Christiane NEUMANN, Oberweningen (CH)

(73) Assignee: Hoffmann-La Roche Inc., Little Falls, NJ (US)

(21) Appl. No.: 18/066,529

(22) Filed: Dec. 15, 2022

Related U.S. Application Data
(63) Continuation of application No. PCT/EP2021/066335, filed on Jun. 17, 2021.

(30) Foreign Application Priority Data
Jun. 19, 2020 (EP) 20181072.8

(51) Int. Cl. C07K 16/46; C12N 15/63; A61P 35/00

(52) U.S. Cl. CPC C07K 16/468; C12N 15/63; A61P 35/00; C07K 2317/31; C07K 2317/565; C07K 2317/622; C07K 2317/55; C07K 2317/71

(57) ABSTRACT

The present invention generally relates to novel protease-activatable T cell activating bispecific molecules and idiotype-specific polypeptides. The present invention also relates to polynucleotides encoding such protease-activatable T cell activating bispecific molecules and idiotype-specific polypeptides, and vectors and host cells comprising such polynucleotides. The invention further relates to methods for producing the protease-activatable T cell activating bispecific molecules and idiotype-specific polypeptides of the invention, and to methods of using these protease-activatable T cell activating bispecific molecules and idiotype-specific polypeptides in the treatment of disease.

### CROSS REFERENCE TO RELATED APPLICATIONS

This application is a continuation of International Application No. PCT/EP2021/066335, filed Jun. 17, 2021, which claims priority to European Application No. 20181072.8 filed Jun. 19, 2020, which are incorporated herein by reference in its entirety.

### SEQUENCE LISTING

This application contains a Sequence Listing which has been submitted electronically in XML format and is hereby incorporated by reference in its entirety. Said XML copy, created on Dec. 14, 2022, is named P36114-US Sequence Listing.xml and is 179,019 bytes in size.

### FIELD OF THE INVENTION

The present invention generally relates to novel protease-activatable antigen-binding molecules that comprise an anti-idiotype-binding moiety which reversibly masks antigen binding of the molecule. Specifically, the invention relates to T cell binding molecules having an anti-idiotype-binding moiety that masks the CD3-binding moiety until cleaved by a protease. This allows the CD3-binding moiety to be inaccessible or “masked” until it is in proximity to a target tissue, such as a tumor, e.g., tumor-infiltrating T cells. In addition, the present invention relates to polynucleotides encoding such protease-activated T cell binding molecules and idiotype-specific polypeptides, and vectors and host cells comprising such polynucleotides. The invention further relates to methods for producing the protease-activated T cell binding molecules of the invention, and to methods of using the same, e.g., in the treatment of disease.

### BACKGROUND

The selective destruction of an individual target cell or a specific target cell type is often desirable in a variety of clinical settings. For example, it is a primary goal of cancer therapy to specifically destroy tumor cells, while leaving healthy cells and tissues intact and undamaged.

An attractive way of achieving this is by inducing an immune response against the tumor, to make immune effector cells such as natural killer (NK) cells or cytotoxic T lymphocytes (CTLs) attack and destroy tumor cells. In this regard, bispecific antibodies designed to bind with one “arm” to a surface antigen on target cells, and with the second “arm” to an activating, invariant component of the T cell receptor (TCR) complex, have become of interest in recent years. The simultaneous binding of such an antibody to both of its targets will force a temporary interaction between target cell and T cell, causing activation of any cytotoxic T cell and subsequent lysis of the target cell. Hence, the immune response is re-directed to the target cells and is independent of peptide antigen presentation by the target cell or the specificity of the T cell as would be relevant for normal MHC-restricted activation of CTLs.

In this context it is crucial that CTLs are activated only when in close proximity to a target cell, i.e., the immunological synapse is mimicked. Particularly desirable are T cell activating bispecific molecules that do not require lymphocyte preconditioning or co-stimulation in order to elicit efficient lysis of target cells. Several bispecific antibody formats have been developed and their suitability for T cell mediated immunotherapy investigated. These include BiTE (bispecific T cell engager) molecules (Nagorsen and Bäuerle, Exp Cell Res 317, 1255-1260 (2011)), diabodies (Holliger et al., Prot Eng 9, 299-305 (1996)) and derivatives thereof, such as tandem diabodies (Kipriyanov et al., J Mol Biol 293, 41-66 (1999)), DART (dual affinity retargeting) molecules, (Moore et al., Blood 117, 4542-51 (2011)), and triomabs (Seimetz et al., Cancer Treat Rev 36, 458-467 (2010)).

The task of generating bispecific molecules suitable for treatment provides several technical challenges related to efficacy, toxicity, applicability and produceability that have to be met. In instances where the bispecific molecule targets an antigen on a target cell, e.g., a cancer cell, that is also expressed in non-target tissue, toxicity can occur. There is thus a need for efficacious T cell activating bispecific molecules that unleash full T cell activation in the presence of target cells but not in the presence of normal cells or tissue.

### SUMMARY OF THE INVENTION

The invention generally relates to T cell activating bispecific molecules that are activated selectively in the presence of a target cell.

In one aspect, provided is a protease-activatable T cell activating bispecific molecule comprising
(a) a first antigen binding moiety capable of binding to CD3, wherein the first antigen binding moiety comprises
   (i) a heavy chain variable region (VH) comprising a heavy chain complementary determining region (HCDR) 1 of SEQ ID NO: 2, a HCDR 2 of SEQ ID NO: 4, and a HCDR 3 of SEQ ID NO: 10, and
   (ii) a light chain variable region (VL) comprising a light chain complementarity 5 determining region (LCDR) 1 of SEQ ID NO: 20, a LCDR 2 of SEQ ID NO: 21 and a LCDR 3 of SEQ ID NO: 22;
(b) a second antigen binding moiety capable of binding to a target cell antigen; and
(c) a masking moiety covalently attached to the T cell bispecific binding molecule through a protease-cleavable linker, wherein the masking moiety is capable of binding to the idiotype of the first antigen binding moiety thereby reversibly concealing the first or the second antigen binding moiety.

In one aspect, the VH comprises an amino acid sequence that is at least about 95%, 96%, 97%, 98%, 99% or 100% identical to the amino acid sequence of SEQ ID NO: 16, and/or the VL comprises an amino acid sequence that is at least about 95%, 96%, 97%, 98%, 99% or 100% identical to the amino acid sequence of SEQ ID NO: 23.

In one aspect, the masking moiety is covalently attached to the first antigen binding moiety and reversibly conceals the first antigen binding moiety.

In one aspect, the masking moiety is covalently attached to the heavy chain variable region of the first antigen binding moiety.

In one aspect, the masking moiety is an anti-idiotypic scFv.

In one aspect, the second antigen binding moiety is a crossover Fab molecule wherein either the variable or the constant regions of the Fab light chain and the Fab heavy chain are exchanged.

In one aspect, the first antigen binding moiety is a conventional Fab molecule.

In one aspect, provided is the protease-activatable T cell activating bispecific molecule as herein above described, comprising not more than one antigen binding moiety capable of binding to CD3.

In one aspect, provided is the protease-activatable T cell activating bispecific molecule as herein above described, comprising, comprising a third antigen binding moiety which is a Fab molecule capable of binding to a target cell antigen.

In one aspect, the third antigen binding moiety is identical to the second antigen binding moiety.

In one aspect, the second antigen binding moiety is capable of binding to a target cell antigen selected from the group consisting of FolR1 and TYRP1.

In one aspect, the first and the second antigen binding moiety are fused to each other, optionally via a peptide linker.

In one aspect, the second antigen binding moiety is fused at the C-terminus of the Fab heavy chain to the N-terminus of the Fab heavy chain of the first antigen binding moiety.

In one aspect, the first antigen binding moiety is fused at the C-terminus of the Fab heavy chain to the N-terminus of the Fab heavy chain of the second antigen binding moiety.

In one aspect, provided is the protease-activatable T cell activating bispecific molecule as herein above described, additionally comprising an Fc domain composed of a first and a second subunit capable of stable association.

In one aspect, the Fc domain is an IgG, specifically an IgG1 or IgG4, Fc domain.

In one aspect, the Fc domain exhibits reduced binding affinity to an Fc receptor and/or reduced effector function, as compared to a native IgG1 Fc domain.

In one aspect, the masking moiety comprises a heavy chain variable region comprising:
(a) a heavy chain complementarity determining region (CDR H) 1 amino acid sequence of DYSMN (SEQ ID NO:58);
(b) a CDR H2 amino acid sequence selected from the group consisting of WINTETGEPRYTDDFKG (SEQ ID NO:59), WINTETGEPRYTDDFTG (SEQ ID NO:84) and WINTETGEPRYTQGFKG (SEQ ID NO:86);
(c) a CDR H3 amino acid sequence of EGDYDVFDY (SEQ ID NO:60); and a light chain variable region comprising:
(d) a light chain (CDR L)1 amino acid sequence selected from the group consisting of RASKSVSTSSYSYMH (SEQ ID NO:62) and KSSKSVSTSSYSYMH (SEQ ID NO:82);
(e) a CDR L2 amino acid sequence of YVSYLES (SEQ ID NO:63); and
(f) a CDR L3 amino acid sequence selected from the group consisting of QHSREFPYT (SEQ ID NO:64) and QQSREFPYT (SEQ ID NO:88).

In one aspect, the masking moiety comprises a heavy chain variable region comprising:
(a) a heavy chain complementarity determining region (CDR H) 1 amino acid sequence of DYSMN (SEQ ID NO:58);
(b) a CDR H2 amino acid sequence of WINTETGEPRYTDDFKG (SEQ ID NO:59);
(c) a CDR H3 amino acid sequence of EGDYDVFDY (SEQ ID NO:60); and a light chain variable region comprising:
(d) a light chain (CDR L)1 amino acid sequence of RASKSVSTSSYSYMH (SEQ ID NO: 62);
(e) a CDR L2 amino acid sequence of YVSYLES (SEQ ID NO:63); and
(f) a CDR L3 amino acid sequence of QHSREFPYT (SEQ ID NO:64).

In one aspect, the masking moiety comprises a heavy chain variable region comprising:
(a) a heavy chain complementarity determining region (CDR H) 1 amino acid sequence of SYGVS (SEQ ID NO:58);
(b) a CDR H2 amino acid sequence of IIWGDGSTNYHSALIS (SEQ ID NO:59);
(c) a CDR H3 amino acid sequence of GITTVVDDYYAMDY (SEQ ID NO:60); and a light chain variable region comprising:
(d) a light chain (CDR L)1 amino acid sequence of KSSKSVSTSSYSYMH (SEQ ID NO: 82);
(e) a CDR L2 amino acid sequence of AATFLAD (SEQ ID NO:63); and
(f) a CDR L3 amino acid sequence of QHYYSTPYT (SEQ ID NO:64).

In one aspect, the masking moiety comprises a heavy chain variable region comprising:
(a) a heavy chain complementarity determining region (CDR H) 1 amino acid sequence of SYGVS (SEQ ID NO:58);
(b) a CDR H2 amino acid sequence of WINTETGEPRYTDDFTG (SEQ ID NO:84);
(c) a CDR H3 amino acid sequence of GITTVVDDYYAMDY (SEQ ID NO:60); and a light chain variable region comprising:
(d) a light chain (CDR L)1 amino acid sequence of KSSKSVSTSSYSYMH (SEQ ID NO: 82);
(e) a CDR L2 amino acid sequence of AATFLAD (SEQ ID NO:63); and
(f) a CDR L3 amino acid sequence of QHYYSTPYT (SEQ ID NO:64).

In one aspect, the masking moiety comprises a heavy chain variable region comprising:
(a) a heavy chain complementarity determining region (CDR H) 1 amino acid sequence of SYGVS (SEQ ID NO:58);
(b) a CDR H2 amino acid sequence of WINTETGEPRYTQGFKG (SEQ ID NO:86);
(c) a CDR H3 amino acid sequence of GITTVVDDYYAMDY (SEQ ID NO:60); and a light chain variable region comprising:
(d) a light chain (CDR L)1 amino acid sequence of KSSKSVSTSSYSYMH (SEQ ID NO:82);
(e) a CDR L2 amino acid sequence of AATFLAD (SEQ ID NO:63); and
(f) a CDR L3 amino acid sequence of QHYYSTPYT (SEQ ID NO:64).

In one aspect, the protease cleavable linker comprises at least one protease recognition sequence.

In one aspect, the protease recognition sequence is selected from the group consisting of:
(a) RQARVVNG (SEQ ID NO:100);
(b) VHMPLGFLGPGRSRGSFP (SEQ ID NO:101);
(c) RQARVVNGXXXXXVPLSLYSG (SEQ ID NO:102);
(d) RQARVVNGVPLSLYSG (SEQ ID NO:103);
(e) PLGLWSQ (SEQ ID NO:104);
(f) VHMPLGFLGPRQARVVNG (SEQ ID NO:105);
(g) FVGGTG (SEQ ID NO:106);
(h) KKAAPVNG (SEQ ID NO:107);
(i) PMAKKVNG (SEQ ID NO:108);
(j) QARAKVNG (SEQ ID NO:109);
(k) VHMPLGFLGP (SEQ ID NO:110);
(l) QARAK (SEQ ID NO:111);
(m) VHMPLGFLGPPMAKK (SEQ ID NO:112);
(n) KKAAP (SEQ ID NO:113); and
(o) PMAKK (SEQ ID NO:114), wherein X is any amino acid.

In one aspect, the protease cleavable linker comprises the protease recognition sequence PMAKK (SEQ ID NO:114).

In one aspect, the second antigen binding moiety is capable of binding to FolR1 and comprises a heavy chain variable region comprising:
a) a heavy chain complementarity determining region (CDR H) 1 amino acid sequence of NAWMS (SEQ ID NO:54);
b) a CDR H2 amino acid sequence of RIKSKTDGGTTDYAAPVKG (SEQ ID NO:55); and
c) a CDR H3 amino acid sequence of PWEWSWYDY (SEQ ID NO:56); and a light chain variable region comprising:
d) a light chain (CDR L)1 amino acid sequence of GSSTGAVTTSNYAN (SEQ ID NO:20);
e) a CDR L2 amino acid sequence of GTNKRAP (SEQ ID NO:21); and
f) a CDR L3 amino acid sequence of ALWYSNLWV (SEQ ID NO:22).

In one aspect, the second antigen binding moiety is capable of binding to TYRP1 and comprises a heavy chain variable region comprising:
a) a heavy chain complementarity determining region (CDR H) 1 amino acid sequence of DYFLH (SEQ ID NO:24);
b) a CDR H2 amino acid sequence of WINPDNGNTVYAQKFQG (SEQ ID NO:25); and
c) a CDR H3 amino acid sequence of RDYTYEKAALDY (SEQ ID NO:26); and a light chain variable region comprising:
d) a light chain (CDR L)1 amino acid sequence of RASGNIYNYLA (SEQ ID NO:28);
e) a CDR L2 amino acid sequence of DAKTLAD (SEQ ID NO:29); and
f) a CDR L3 amino acid sequence of QHFWSLPFT (SEQ ID NO:30).

In another aspect, provides is an idiotype-specific polypeptide for reversibly concealing an anti-CD3 antigen binding site of a molecule, wherein the idiotype-specific polypeptide comprises a heavy chain variable region comprising:
(a) a heavy chain complementarity determining region (CDR H) 1 amino acid sequence of DYSMN (SEQ ID NO:58);
(b) a CDR H2 amino acid sequence selected from the group consisting of

(SEQ ID NO: 59)
WINTETGEPRYTDDFKG,
(SEQ ID NO: 84)
WINTETGEPRYTDDFTG
and
(SEQ ID NO: 86)
WINTETGEPRYTQGFKG;

(c) a CDR H3 amino acid sequence of EGDYDVFDY (SEQ ID NO:60); and a light chain variable region comprising:
(d) a light chain (CDR L)1 amino acid sequence selected from the group consisting of

(SEQ ID NO: 62)
RASKSVSTSSYSYMH
and
(SEQ ID NO: 82)
KSSKSVSTSSYSYMH;

(e) a CDR L2 amino acid sequence of YVSYLES (SEQ ID NO:63); and
(f) a CDR L3 amino acid sequence selected from the group consisting of

(SEQ ID NO: 64)
QHSREFPYT
and
(SEQ ID NO: 88)
QQSREFPYT.

In one aspect, the idiotype-specific polypeptide comprises a heavy chain variable region comprising:
(a) a heavy chain complementarity determining region (CDR H) 1 amino acid sequence of DYSMN (SEQ ID NO:58);
(b) a CDR H2 amino acid sequence of WINTETGEPRYTDDFKG (SEQ ID NO:59);
(c) a CDR H3 amino acid sequence of EGDYDVFDY (SEQ ID NO:60); and a light chain variable region comprising:
(d) a light chain (CDR L)1 amino acid sequence of RASKSVSTSSYSYMH (SEQ ID NO:62);
(e) a CDR L2 amino acid sequence of YVSYLES (SEQ ID NO:63); and
(f) a CDR L3 amino acid sequence of QHSREFPYT (SEQ ID NO:64).

In one aspect, the idiotype-specific polypeptide comprises a heavy chain variable region comprising:
(a) a heavy chain complementarity determining region (CDR H) 1 amino acid sequence of SYGVS (SEQ ID NO:58);
(b) a CDR H2 amino acid sequence of IIWGDGSTNYHSALIS (SEQ ID NO:59);
(c) a CDR H3 amino acid sequence of GITTVVDDYYAMDY (SEQ ID NO:60); and a light chain variable region comprising:
(d) a light chain (CDR L)1 amino acid sequence of KSSKSVSTSSYSYMH (SEQ ID NO:82);
(e) a CDR L2 amino acid sequence of AATFLAD (SEQ ID NO:63); and
(f) a CDR L3 amino acid sequence of QHYYSTPYT (SEQ ID NO:64).

In one aspect, the idiotype-specific polypeptide comprises a heavy chain variable region comprising:
(a) a heavy chain complementarity determining region (CDR H) 1 amino acid sequence of SYGVS (SEQ ID NO:58);
(b) a CDR H2 amino acid sequence of WINTETGEPRYTDDFTG (SEQ ID NO:84);
(c) a CDR H3 amino acid sequence of GITTVVDDYYAMDY (SEQ ID NO:60); and a light chain variable region comprising:
(d) a light chain (CDR L)1 amino acid sequence of KSSKSVSTSSYSYMH (SEQ ID NO: 82);
(e) a CDR L2 amino acid sequence of AATFLAD (SEQ ID NO:63); and
(f) a CDR L3 amino acid sequence of QHYYSTPYT (SEQ ID NO:64).

In one aspect, the idiotype-specific polypeptide comprises a heavy chain variable region comprising:
(a) a heavy chain complementarity determining region (CDR H) 1 amino acid sequence of SYGVS (SEQ ID NO:58);
(b) a CDR H2 amino acid sequence of WINTETGEPRYTQGFKG (SEQ ID NO:86);
(c) a CDR H3 amino acid sequence of GITTVVDDYYAMDY (SEQ ID NO:60); and a light chain variable region comprising:
(d) a light chain (CDR L)1 amino acid sequence of KSSKSVSTSSYSYMH (SEQ ID NO: 82);
(e) a CDR L2 amino acid sequence of AATFLAD (SEQ ID NO:63); and
(f) a CDR L3 amino acid sequence of QHYYSTPYT (SEQ ID NO:64).

In one aspect, the idiotype-specific polypeptide is an anti-idiotype scFv.

In one aspect, the idiotype-specific polypeptide is covalently attached to the molecule through a linker.

In one aspect, the linker is a peptide linker.

In one aspect, the linker is a protease-cleavable linker.

In one aspect, the peptide linker comprises at least one protease recognition site.

In one aspect, the protease recognition sequence is selected from the group consisting of:
(a) RQARVVNG (SEQ ID NO:100);
(b) VHMPLGFLGPGRSRGSFP (SEQ ID NO:101);
(c) RQARVVNGXXXXXVPLSLYSG (SEQ ID NO:102);
(d) RQARVVNGVPLSLYSG (SEQ ID NO:103);
(e) PLGLWSQ (SEQ ID NO:104);
(f) VHMPLGFLGPRQARVVNG (SEQ ID NO:105);
(g) FVGGTG (SEQ ID NO:106);
(h) KKAAPVNG (SEQ ID NO:107);
(i) PMAKKVNG (SEQ ID NO:108);
(j) QARAKVNG (SEQ ID NO:109);
(k) VHMPLGFLGP (SEQ ID NO:110);
(l) QARAK (SEQ ID NO:111);
(m) VHMPLGFLGPPMAKK (SEQ ID NO:112);
(n) KKAAP (SEQ ID NO:113); and
(o) PMAKK (SEQ ID NO:114), wherein X is any amino acid.

In one aspect, the protease cleavable linker comprises the protease recognition sequence

(SEQ ID NO: 114)
PMAKK.

In one aspect, the ideotype-specific polypeptide is part of a T-cell activating bispecific molecule.

In another aspect, provided is a pharmaceutical composition comprising the protease-activatable T cell activating bispecific molecule as hereinbefore described or the the idiotype-specific polypeptide as hereinbefore described and a pharmaceutically acceptable carrier.

In another aspect, provided is an isolated polynucleotide encoding the protease-activatable T cell activating bispecific antigen binding molecule as hereinbefore described or the idiotype-specific polypeptide as hereinbefore described.

In one aspect, provided is a a vector, particularly an expression vector, comprising the polynucleotide as hereinbefore described.

In one aspect, provided is a host cell comprising the polynucleotide as hereinbefore described or the vector as hereinbefore described.

In another aspect, provided is a method of producing a protease-activatable T cell activating bispecific molecule, comprising the steps of a) culturing the host cell as hereinbefore described under conditions suitable for the expression of the protease-activatable T cell activating bispecific molecule and b) recovering the protease-activatable T cell activating bispecific molecule.

In another aspect, provided is a protease-activatable T cell activating bispecific molecule as hereinbefore described, the idiotype-specific polypeptide as hereinbefore described or the pharmaceutical composition as hereinbefore described for use as a medicament.

In one aspect, the medicament is for treating or delaying progression of cancer, treating or delaying progression of an immune related disease, or enhancing or stimulating an immune response or function in an individual.

In another aspect, provided is the use of the protease-activatable T cell activating bispecific molecule as hereinbefore described or the idiotype-specific polypeptide as hereinbefore described for the manufacture of a medicament for the treatment of a disease.

In one aspect, the disease is a cancer.

In another aspect, provided is a method of treating a disease in an individual, comprising administering to said individual a therapeutically effective amount of a composition comprising the protease-activatable T cell activating bispecific molecule as hereinbefore described.

In one aspect, the method is for treating or delaying progression of cancer, treating or delaying progression of an immune related disease, or enhancing or stimulating an immune response or function in an individual.

### SHORT DESCRIPTION OF THE FIGURES

FIGS. 1A-Z. Exemplary configurations of the (multispecific) antibodies of the invention. (FIG. 1A, FIG. 1D) Illustration of the “1+1 CrossMab” molecule. (FIG. 1B, FIG. 1E) Illustration of the “2+1 IgG Crossfab” molecule with alternative order of Crossfab and Fab components (“inverted”). (FIG. 1C, FIG. 1F) Illustration of the “2+1 IgG Crossfab” molecule. (FIG. 1G, FIG. 1K) Illustration of the “1+1 IgG Crossfab” molecule with alternative order of Crossfab and Fab components (“inverted”). (FIG. 1H, FIG. 1L) Illustration of the “1+1 IgG Crossfab” molecule. (FIG. 1I, FIG. 1M) Illustration of the “2+1 IgG Crossfab” molecule with two CrossFabs. (FIG. 1J, FIG. 1N) Illustration of the “2+1 IgG Crossfab” molecule with two CrossFabs and alternative order of Crossfab and Fab components (“inverted”). (FIG. 1O, FIG. 1S) Illustration of the “Fab-Crossfab” molecule. (FIG. 1P, FIG. 1T) Illustration of the “Crossfab-Fab” molecule. (FIG. 1Q, FIG. 1U) Illustration of the “(Fab)₂-Crossfab” molecule. (FIG. 1R, FIG. 1V) Illustration of the “Crossfab-(Fab)₂” molecule. (FIG. 1W, FIG. 1Y) Illustration of the “Fab-(Crossfab)₂” molecule. (FIG. 1X, Z) Illustration of the “(Crossfab)₂-Fab” molecule. Black dot: optional modification in the Fc domain promoting heterodimerization. ++, −−: amino acids of opposite charges optionally introduced in the CH1 and CL domains. Crossfab molecules are depicted as comprising an exchange of VH and VL regions, but may—in aspects wherein no charge modifications are introduced in CH1 and CL domains—alternatively comprise an exchange of the CH1 and CL domains.

FIGS. 2A-E. (FIG. 2A) Schematic illustration of the T-cell bispecific antibody (TCB) molecules used in the Examples. All tested TCB antibody molecules were produced as “2+1 IgG CrossFab, inverted” with charge modifications (VH/VL exchange in CD3 binder, charge modifications in target cell antigen binders, EE=147E, 213E; RK=123R, 124K). (FIGS. 2B-2E) Components for the assembly of the TCB: light chain of anti-TYRP1 Fab molecule with charge modifications in CH1 and CL (FIG. 2B), light chain of anti-CD3 crossover Fab molecule (FIG. 2C), heavy chain with knob and PG LALA mutations in Fc region (FIG. 2D), heavy chain with hole and PG LALA mutations in Fc region (FIG. 2E).

FIG. 3. Schematic illustration of the surface plasmon resonance (SPR) setup used in Example 3. Anti-PG antibody coupled to a C1 sensorchip. Human and cynomolgus CD3 (fused to an Fc region) are passed over the surface to analyze the interaction of the anti-CD3 antibody in the TCB with CD3.

FIGS. 4A-B. The TCBs containing optimized anti-CD3 antibodies were tested in a Jurkat NFAT reporter assay with CHO-K1 TYRP1 clone 76 as target cells. Comparison was done to a TCB containing CD3_{orig}. Activation of Jurkat NFAT reporter cells was determined by measuring luminescence after 4 hours (FIG. 4A) and 24 hours (FIG. 4B) upon treatment.

FIGS. 5A-B. Tumor cell killing of the melanoma cell line M150543 with PBMCs from a healthy donor was assessed when treated with TCBs either containing the optimized anti-CD3 antibodies or the parental binder CD3_{orig}. Tumor cell killing was measured by quantification of LDH release after 24 hours (FIG. 5A) and 48 hours (FIG. 5B).

FIGS. 6A-D. CD25 and CD69 upregulation on CD8 T cells (FIG. 6A, FIG. 6B) and on CD4 T cells (FIG. 6C, FIG. 6D) was analyzed for PBMCs from a healthy donor treated with TCBs either containing the optimized anti-CD3 antibodies or the parental binder CD3_{orig}, in presence of the M150543 melanoma cell line as target cells. Analysis was done by flow cytometry after 48 hours. FIGS. 7A-B. CD25 expression on CD8 (FIG. 7A) and on CD4 T cells (FIG. 7B) was analyzed for PBMCs from a healthy donor treated with TCBs either containing the optimized anti-CD3 antibodies or the parental binder CD3_{orig}, in absence of tumor target cells. Analysis was done by flow cytometry after 48 hours.

FIGS. 8A-D. (FIG. 8A) Schematic illustration of the monovalent IgG molecules generated in Example 19. The monovalent IgG molecules were produced as human IgG₁ with a VH/VL exchange in the CD3 binder. (FIGS. 8B-8E) Components for the assembly of the monovalent IgG: light chain of anti-CD3 crossover Fab molecule (FIG. 8B), heavy chain with knob and PG LALA mutations in Fc region (FIG. 8C), heavy chain with hole and PG LALA mutations in Fc region (FIG. 8D).

FIGS. 9A-C. (FIG. 9A): Classical 2+1 TCB molecule with a CD3 Fab fused via a (G4S)2 linker (L1) to VH of inner FOLR1 Fab. Heterodimerization by knob-into-hole technology, PGLALA mutation in Fc. (FIG. 9B): FOLR1 proTCB in which a CD3 anti-idiotypic scFv (VH-VL orientation) is fused to the CD3 VH. The linker (L2; 33 aa in total) contains a specific protease cleavable sequence. (G4S)4 linker (L3) between VH and VL of the scFv. (FIG. 9C): Same proTCB as in B but no protease clevage site in linker between scFv and CD3 Fab. The light chain in the molecules is identical in each Fab (common light chain).

FIGS. 10A-E. (FIG. 10A): Jurkat NFAT activation mediated by TYRP1 TCB containing different CD3 binders. Jurkat NFAT activation mediated by TYRP1 TCB with different CD3 binders is shown. TYRP1 TCB (used at EC90 concentration determined in previous assay) were incubated with TYRP1 positive target cells (CHO-huTYRP1 clone 76) and Jurkat NFAT effector cells (E:T 2.5:1) for 22 h at 37° C. The dotted line shows the Jurkat NFAT activation incubated with target cells but without any TCB. DP47 non-targeting TCB was used as negative control. Each dot represents the mean of triplicates. Standard deviation is indicated by error bars.

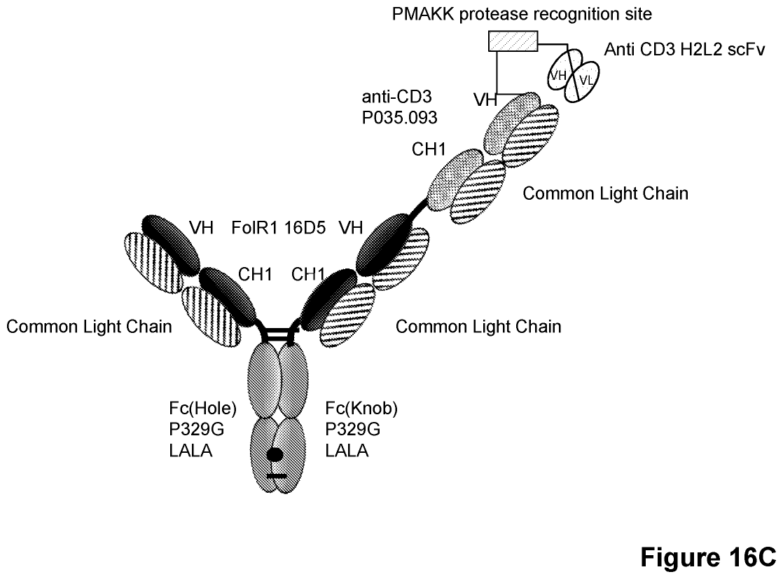

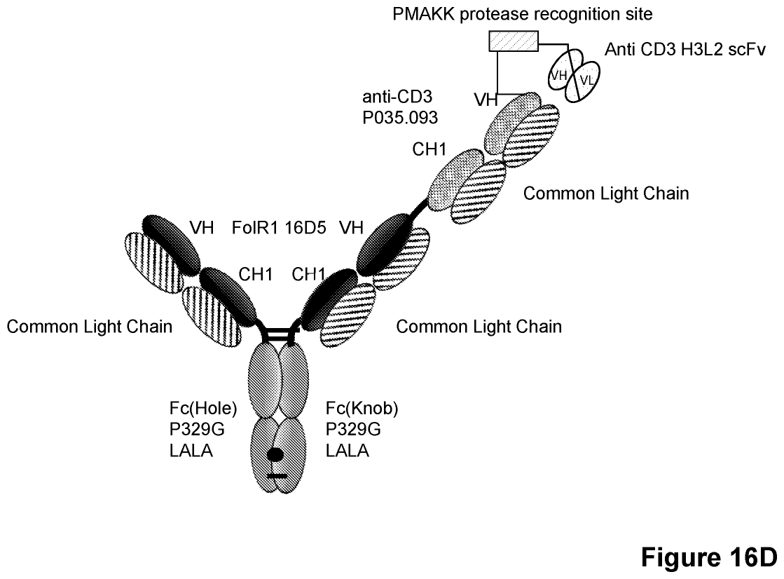

(FIG. 10B): Blocking capacity of anti-idiotypic 4.24.72 IgG measured by reduction of Jurkat NFAT activation mediated by TYRP1 TCB. Jurkat NFAT activation mediated by TYRP1 TCB with different CD3 binders is shown. TYRP1 TCB (used at EC90 concentration determined in previous assay) was incubated with TYRP1 positive target cells (CHO-huTYRP1 clone 76) and Jurkat NFAT effector cells (E:T 2.5:1) for 22 h at 37° C. Dose-dependent blocking of CD3 binder by anti-idiotypic (anti-ID) 4.24.72 IgG is shown. Each dot represents the mean of triplicates. Standard deviation is indicated by error bars. The dotted line shows the Jurkat NFAT activation incubated with target cells but without any TCB. DP47 non-targeting TCB was used as negative control. For the calculation of EC-50 values the nonlinear fit “log(agonist) vs. response—Variable slope (four parameters)” was calculated (GraphPad Prism6).

(FIG. 10C): Blocking capacity of anti-ID 4.32.63 IgG measured by reduction of Jurkat NFAT activation mediated by TYRP1 TCB. Jurkat NFAT activation mediated by TYRP1 TCB with different CD3 binders is shown. TYRP1 TCB (used at EC90 concentration determined in previous assay) was incubated with TYRP1 positive target cells (CHO-huTYRP1 clone 76) and Jurkat NFAT effector cells (E:T 2.5:1) for 22 h at 37° C. Dose-dependent blocking of CD3 binder by anti-idiotypic 4.32.63 IgG is shown. Each dot represents the mean of triplicates. Standard deviation is indicated by error bars. The dotted line shows the Jurkat NFAT activation incubated with target cells but without any TCB. DP47 non-targeting TCB was used as negative control. For the calculation of EC-50 values the nonlinear fit “log(agonist) vs. response—Variable slope (four parameters)” was calculated (GraphPad Prism6).

(FIG. 10D): Blocking capacity of anti-ID 4.15.64 IgG measured by reduction of Jurkat NFAT activation mediated by TYRP1 TCB. Jurkat NFAT activation mediated by TYRP1 TCB with different CD3 binders is shown. TYRP1 TCB (used at EC90 concentration determined in previous assay) was incubated with TYRP1 positive target cells (CHO-huTYRP1 clone 76) and Jurkat NFAT effector cells (E:T 2.5:1) for 22 h at 37° C. Dose-dependent blocking of CD3 binder by anti-idiotypic 4.15.64 IgG is shown. Each dot represents the mean of triplicates. Standard deviation is indicated by error bars. The dotted line shows the Jurkat NFAT activation incubated with target cells but without any TCB. DP47 non-targeting TCB was used as negative control. For the calculation of EC-50 values the nonlinear fit “log(agonist) vs. response—Variable slope (four parameters)” was calculated (GraphPad Prism6).

(FIG. 10E): Blocking capacity of anti-ID 4.21 IgG measured by reduction of Jurkat NFAT activation mediated by TYRP1 TCB. Jurkat NFAT activation mediated by TYRP1 TCB with different CD3 binders is shown. TYRP1 TCB (used at EC90 concentration determined in previous assay) was incubated with TYRP1 positive target cells (CHO-huTYRP1 clone 76) and Jurkat NFAT effector cells (E:T 2.5:1) for 22 h at 37° C. Dose-dependent blocking of CD3 binder by anti-idiotypic 4.21 IgG is shown. Each dot represents the mean of triplicates. Standard deviation is indicated by error bars. The dotted line shows the Jurkat NFAT activation incubated with target cells but without any TCB. DP47 non-targeting TCB was used as negative control. For the calculation of EC-50 values the nonlinear fit “log(agonist) vs. response—Variable slope (four parameters)” was calculated (GraphPad Prism6).

FIGS. 11A-D. (FIG. 11A): Jurkat NFAT activation mediated by FOLR1 TCB or FOLR1 pro-TCB with different CD3 binders is shown. FOLR1 (pro-)TCBs were incubated with huFOLR1 coated beads and Jurkat NFAT effector cells for 5-6 h at 37° C. FOLR1 pro-TCBs with anti-ID mask 4.24.72 do not mediate Jurkat NFAT activation in the indicated concentration range. FOLR1 TCBs however mediate dose-dependent Jurkat NFAT activation. Each dot represents the mean of triplicates. Standard deviation is indicated by error bars. The dotted line shows the Jurkat NFAT activation incubated with target cells but without any TCB.

(FIG. 11B): Dose-dependent target cell killing (HeLa cells with very high FOLR1 expression) was measured after 48 h of incubation of huPBMCs, TCB and FOLR1 positive target cells (E:T=10:1, effectors are human PBMCs). FOLR1 TCB and activated FOLR1 pro-TCB induce dose-dependent target cell killing with an EC50 around 0.29 pM. The masked FOLR1 pro-TCB (CD3 P035.093, mask 4.24.72 scFv) containing a non-cleavable linker shows a reduction of target cell lysis of about 239 fold when comparing EC50 values. Each dot represents the mean of triplicates. Standard deviation is indicated by error bars. The dotted line shows spontaneous release of target cells incubated with huPBMCs but without any TCB. For the calculation of EC-50 values the nonlinear fit “log(agonist) vs. response—Variable slope (four parameters)” was calculated (GraphPad Prism6).

(FIG. 11C): Dose-dependent T cell activation was analyzed for CD8 T cells by quantification of CD69. Median fluorescence intensity for CD69 was blotted for CD8 positive T cells. Target cells (HeLa cells with very high FOLR1 expression) were incubated with huPBMCs and TCBs for 48 h at 37° C. (E:T=10:1, effectors are human PBMCs). FOLR1 TCB and activated FOLR1 pro-TCB induce dose-dependent T cell activation. The masked FOLR1 pro-TCB (CD3 P035.093, mask 4.24.72 scFv) containing a non-cleavable linker shows no T cell activation (CD69 for CD8 T cells) in the indicated concentration range. Each dot represents the mean of triplicates. Standard deviation is indicated by error bars. For the calculation of EC-50 values the nonlinear fit “log(agonist) vs. response—Variable slope (four parameters)” was calculated (GraphPad Prism6).

(FIG. 11D): Dose-dependent T cell activation was measured for CD8 T cells by quantification of CD69. Percentage of CD69 positive CD8 T cells is shown. Target cells (HeLa cells with very high FOLR1 expression) were incubated with huPBMCs and TCBs for 48 h at 37° C. (E:T=10:1, effectors are human PBMCs). FOLR1 TCB and activated FOLR1 pro-TCB induce dose-dependent T cell activation. The masked FOLR1 pro-TCB (CD3 P035.093, mask 4.24.72 scFv) containing a non-cleavable linker shows reduced T cell activation (CD69 for CD8 T cells) in the indicated concentration range. However starting at 5 nM some CD69 positive CD8 T cells could be detected increasing to around 30% at the highest concentration used in here. Each dot represents the mean of triplicates. Standard deviation is indicated by error bars. For the calculation of EC-50 values the nonlinear fit “log(agonist) vs. response—Variable slope (four parameters)” was calculated (GraphPad Prism6).

FIGS. 12A-B. (FIG. 12A): Dose-dependent target cell killing (Hela high FOLR1 expression, Ovcar-3 and Skov-3 medium FOLR1 expression and HT-29 with low FOLR1 expression) was measured after 48 h of incubation of huPBMCs to analyze masking-efficiency of anti-ID 4.24.72 in pro-TCB format with CD3 P035.093. TCB and FOLR1 positive target cells (E:T=10:1, effectors are human PBMCs). FOLR1 TCB induces dose-dependent target cell killing on all cell lines (Hela, Skov-3, Ovcar-3) whereas the masked FOLR1 pro-TCB shows reduced target cell killing.

(FIG. 12B): Dose-dependent target cell killing (Skov-3 medium FOLR1 expression and HT-29 with low FOLR1 expression) was measured after 48 h of incubation of huPBMCs (E:T=10:1). FOLR1 TCB induces dose-dependent target cell killing on both cell lines (Skov-3, HT-29) whereas the masked FOLR1 pro-TCB shows reduced target cell killing. Each dot represents the mean of triplicates. Standard deviation is indicated by error bars. The dotted line shows spontaneous release of target cells incubated with huPBMCs but without any TCB. For the calculation of EC-50 values the nonlinear fit “log(agonist) vs. response—Variable slope (four parameters)” was calculated (GraphPad Prism6).

FIG. 13: Format of the one-armed IgGs of the humanization variants of the anti-idiotypic mask 4.24.72. Heterodimerization is achieved by using the knobs-into-holes technology.

FIGS. 14A-B. Jurkat NFAT activation mediated by TYRP1 TCB with different CD3 binders is shown. TYRP1 TCB (used at EC90 concentration determined in previous assay) was incubated with TYRP1 positive target cells (M150543) and Jurkat NFAT effector cells (E:T 2.5:1) for 5 h at 37° C. Dose-dependent blocking of CD3 binder by anti-idiotypic (anti-ID) 4.24.72 IgGs (parental and humanization variants) is shown for CD3 CH2527 (FIG. 14A) and CD3 P035.093 (FIG. 14B). Each dot represents the mean of triplicates. Standard deviation is indicated by error bars. For the calculation of EC-50 values the nonlinear fit “log(agonist) vs. response—Variable slope (four parameters)” was calculated (GraphPad Prism6).

FIGS. 15A-C. (FIG. 15A): Dose-dependent target cell killing (Ovcar-3 medium FOLR1 expression) was measured after 48 h of incubation of huPBMCs to analyze masking-efficiency of humanization variants of anti-ID 4.24.72 in pro-TCB format with CD3 P035.093. TCB and FOLR1 positive target cells (E:T=10:1, effectors are human PBMCs). FOLR1 TCB induces dose-dependent target cell killing on Ovcar-3 cells whereas the masked FOLR1 pro-TCB shows reduced target cell killing.

(FIG. 15B) and (FIG. 15C): Dose-dependent T cell activation was measured for CD8 T cells by quantification of CD69. Percentage of CD69 positive CD8 T cells (FIG. 15B) and Median fluorescence intensity FIG. 15C) are shown. Target cells (Ovcar-3 cells with medium FOLR1 expression) were incubated with huPBMCs and TCBs for 48 h at 37° C. (E:T=10:1, effectors are human PBMCs). FOLR1 TCB induces dose-dependent T cell activation. The masked FOLR1 pro-TCB (CD3 P035.093, humanization variants of mask 4.24.72 scFv) containing a non-cleavable linker show reduced T cell activation (CD69 for CD8 T cells) in the indicated concentration range. Regarding T cell activation no differences for masking-efficiency could be detected for the humanization variants. Each dot represents the mean of triplicates. Standard deviation is indicated by error bars. For the calculation of EC-50 values the nonlinear fit “log(agonist) vs. response—Variable slope (four parameters)” was calculated (GraphPad Prism6).

FIGS. 16A-D. depict schematics of different protease activatable FolR1 TCB molecules with humanized masking moieties.

(FIG. 16A): anti-CD3 P035.093 mask scFv H1L1 Matriptase site anti-FolR1 16D5 with common light chain P329G LALA 2+1 Fc(Hole) Fc (Knob), SEQ ID Nos 95, 66, 67.

(FIG. 16B): anti-CD3 P035.093 mask scFv H1L2 Matriptase site anti-FolR1 16D5 with common light chain P329G LALA 2+1 Fc(Hole) Fc (Knob), SEQ ID Nos 96, 66, 67.

(FIG. 16C): anti-CD3 P035.093 mask scFv H2L2 Matriptase site anti-FolR1 16D5 with common light chain P329G LALA 2+1 Fc (Hole) Fc (Knob), SEQ ID Nos 97, 66, 67.

(FIG. 16D): anti-CD3 P035.093 mask scFv H3L2 Matriptase site anti-FolR1 16D5 with common light chain P329G LALA 2+1 Fc(Hole) Fc (Knob) SEQ ID Nos 98, 66, 67.

### DETAILED DESCRIPTION

### Definitions

Terms are used herein as generally used in the art, unless otherwise defined in the following.

As used herein, the term “antigen binding molecule” refers in its broadest sense to a molecule that specifically binds an antigenic determinant. Examples of antigen binding molecules are immunoglobulins and derivatives, e.g., fragments, thereof.

The term “bispecific” means that the antigen binding molecule is able to specifically bind to at least two distinct antigenic determinants. Typically, a bispecific antigen binding molecule comprises two antigen binding sites, each of which is specific for a different antigenic determinant. In certain embodiments the bispecific antigen binding molecule is capable of simultaneously binding two antigenic determinants, particularly two antigenic determinants expressed on two distinct cells.

The term “valent” as used herein denotes the presence of a specified number of antigen binding sites in an antigen binding molecule. As such, the term “monovalent binding to an antigen” denotes the presence of one (and not more than one) antigen binding site specific for the antigen in the antigen binding molecule.

An “antigen binding site” refers to the site, i.e. one or more amino acid residues, of an antigen binding molecule which provides interaction with the antigen. For example, the antigen binding site of an antibody comprises amino acid residues from the complementarity determining regions (CDRs). A native immunoglobulin molecule typically has two antigen binding sites, a Fab molecule typically has a single antigen binding site.

As used herein, the term “antigen binding moiety” refers to a polypeptide molecule that specifically binds to an antigenic determinant. In one embodiment, an antigen binding moiety is able to direct the entity to which it is attached (e.g., a second antigen binding moiety) to a target site, for example to a specific type of tumor cell or tumor stroma bearing the antigenic determinant. In another embodiment an antigen binding moiety is able to activate signaling through its target antigen, for example a T cell receptor complex antigen. Antigen binding moieties include antibodies and fragments thereof as further defined herein. Particular antigen binding moieties include an antigen binding domain of an antibody, comprising an antibody heavy chain variable region and an antibody light chain variable region. In certain embodiments, the antigen binding moieties may comprise antibody constant regions as further defined herein and known in the art. Useful heavy chain constant regions include any of the five isotypes: α, δ, ε, γ, or μ. Useful light chain constant regions include any of the two isotypes: κ and λ.

As used herein, the term “antigenic determinant” is synonymous with “antigen” and “epitope,” and refers to a site (e.g., a contiguous stretch of amino acids or a conformational configuration made up of different regions of non-contiguous amino acids) on a polypeptide macromolecule to which an antigen binding moiety binds, forming an antigen binding moiety-antigen complex. Useful antigenic determinants can be found, for example, on the surfaces of tumor cells, on the surfaces of virus-infected cells, on the surfaces of other diseased cells, on the surface of immune cells, free in blood serum, and/or in the extracellular matrix (ECM). The proteins referred to as antigens herein (e.g., FolR1, HER1, HER2, CD3, Mesothelin) can be any native form of the proteins from any vertebrate source, including mammals such as primates (e.g., humans) and rodents (e.g., mice and rats), unless otherwise indicated. In a particular embodiment the antigen is a human protein. Where reference is made to a specific protein herein, the term encompasses the “full-length”, unprocessed protein as well as any form of the protein that results from processing in the cell. The term also encompasses naturally occurring variants of the protein, e.g., splice variants or allelic variants. Exemplary human proteins useful as antigens include, but are not limited to: FolR1, HER1 and CD3, particularly the epsilon subunit of CD3 (see UniProt no. P07766 (version 130), NCBI RefSeq no. NP_000724.1, SEQ ID NO: 54 for the human sequence; or UniProt no. Q95LI5 (version 49), NCBI GenBank no. BAB71849.1 for the cynomolgus [Macaca fascicularis] sequence). In certain embodiments the protease-activatable T cell activating bispecific molecule of the invention binds to an epitope of CD3 or a target cell antigen that is conserved among the CD3 or target antigen from different species. In certain embodiments the protease-activatable T cell activating bispecific molecule of the invention binds to CD3 and FolR1, but does not bind to FolR2 or FolR3. In certain embodiments the protease-activatable T cell activating bispecific molecule of the invention binds to CD3 and HER1. In certain embodiments the protease-activatable T cell activating bispecific molecule of the invention binds to CD3 and Mesothelin. In certain embodiments the protease-activatable T cell activating bispecific molecule of the invention binds to CD3 and HER2. By “specific binding” is meant that the binding is selective for the antigen and can be discriminated from unwanted or non-specific interactions. The ability of an antigen binding moiety to bind to a specific antigenic determinant can be measured either through an enzyme-linked immunosorbent assay (ELISA) or other techniques familiar to one of skill in the art, e.g., surface plasmon resonance (SPR) technique (analyzed on a BIAcore instrument) (Liljeblad et al., Glyco J 17, 323-329 (2000)), and traditional binding assays (Heeley, Endocr Res 28, 217-229 (2002)). In one embodiment, the extent of binding of an antigen binding moiety to an unrelated protein is less than about 10% of the binding of the antigen binding moiety to the antigen as measured, e.g., by SPR. In certain embodiments, an antigen binding moiety that binds to the antigen, or an antigen binding molecule comprising that antigen binding moiety, has a dissociation constant (K_{D}) of ≤1 μM, ≤100 nM, ≤10 nM, ≤1 nM, ≤0.1 nM, ≤0.01 nM, or ≤0.001 nM (e.g., 10⁻⁸M or less, e.g., from 10⁻⁸ M to 10⁻¹³ M, e.g., from 10⁻⁹ M to 10⁻¹³ M).

“Affinity” refers to the strength of the sum total of non-covalent interactions between a single binding site of a molecule (e.g., a receptor) and its binding partner (e.g., a ligand). Unless indicated otherwise, as used herein, “binding affinity” refers to intrinsic binding affinity which reflects a 1:1 interaction between members of a binding pair (e.g., an antigen binding moiety and an antigen, or a receptor and its ligand). The affinity of a molecule X for its partner Y can generally be represented by the dissociation constant (K_{D}), which is the ratio of dissociation and association rate constants (k_{off} and kₒₙ, respectively). Thus, equivalent affinities may comprise different rate constants, as long as the ratio of the rate constants remains the same. Affinity can be measured by well-established methods known in the art, including those described herein. A particular method for measuring affinity is Surface Plasmon Resonance (SPR).

“Reduced binding”, for example reduced binding to an Fc receptor, refers to a decrease in affinity for the respective interaction, as measured for example by SPR. For clarity the term includes also reduction of the affinity to zero (or below the detection limit of the analytic method), i.e. complete abolishment of the interaction. Conversely, “increased binding” refers to an increase in binding affinity for the respective interaction.

“T cell activation” as used herein refers to one or more cellular response of a T lymphocyte, particularly a cytotoxic T lymphocyte, selected from: proliferation, differentiation, cytokine secretion, cytotoxic effector molecule release, cytotoxic activity, and expression of activation markers. The protease-activatable T cell activating bispecific molecules of the invention are capable of inducing T cell activation. Suitable assays to measure T cell activation are known in the art described herein.

A “target cell antigen” as used herein refers to an antigenic determinant presented on the surface of a target cell, for example a cell in a tumor such as a cancer cell or a cell of the tumor stroma.

As used herein, the terms “first” and “second” with respect to antigen binding moieties etc., are used for convenience of distinguishing when there is more than one of each type of moiety. Use of these terms is not intended to confer a specific order or orientation of the protease-activatable T cell activating bispecific molecule unless explicitly so stated.

A “Fab molecule” refers to a protein consisting of the VH and CH1 domain of the heavy chain (the “Fab heavy chain”) and the VL and CL domain of the light chain (the “Fab light chain”) of an immunoglobulin.

By “fused” is meant that the components (e.g., a Fab molecule and an Fc domain subunit) are linked by peptide bonds, either directly or via one or more peptide linkers.

As used herein, the term “single-chain” refers to a molecule comprising amino acid monomers linearly linked by peptide bonds. In certain embodiments, one of the antigen binding moieties is a single-chain Fab molecule, i.e. a Fab molecule wherein the Fab light chain and the Fab heavy chain are connected by a peptide linker to form a single peptide chain. In a particular such embodiment, the C-terminus of the Fab light chain is connected to the N-terminus of the Fab heavy chain in the single-chain Fab molecule.

By a “crossover” Fab molecule (also termed “Crossfab”) is meant a Fab molecule wherein either the variable regions or the constant regions of the Fab heavy and light chain are exchanged, i.e. the crossover Fab molecule comprises a peptide chain composed of the light chain variable region and the heavy chain constant region, and a peptide chain composed of the heavy chain variable region and the light chain constant region. For clarity, in a crossover Fab molecule wherein the variable regions of the Fab light chain and the Fab heavy chain are exchanged, the peptide chain comprising the heavy chain constant region is referred to herein as the “heavy chain” of the crossover Fab molecule. Conversely, in a crossover Fab molecule wherein the constant regions of the Fab light chain and the Fab heavy chain are exchanged, the peptide chain comprising the heavy chain variable region is referred to herein as the “heavy chain” of the crossover Fab molecule.

In contrast thereto, by a “conventional” Fab molecule is meant a Fab molecule in its natural format, i.e. comprising a heavy chain composed of the heavy chain variable and constant regions (VH-CH1), and a light chain composed of the light chain variable and constant regions (VL-CL). The term “immunoglobulin molecule” refers to a protein having the structure of a naturally occurring antibody. For example, immunoglobulins of the IgG class are heterotetrameric glycoproteins of about 150,000 daltons, composed of two light chains and two heavy chains that are disulfide-bonded. From N- to C-terminus, each heavy chain has a variable region (VH), also called a variable heavy domain or a heavy chain variable domain, followed by three constant domains (CH1, CH2, and CH3), also called a heavy chain constant region. Similarly, from N- to C-terminus, each light chain has a variable region (VL), also called a variable light domain or a light chain variable domain, followed by a constant light (CL) domain, also called a light chain constant region. The heavy chain of an immunoglobulin may be assigned to one of five types, called α (IgA), δ (IgD), ε (IgE), γ (IgG), or μ (IgM), some of which may be further divided into subtypes, e.g., γ₁ (IgG₁), γ₂ (IgG₂), γ₃ (IgG₃), γ₄ (IgG₄), α₁ (IgA₁) and α₂ (IgA₂). The light chain of an immunoglobulin may be assigned to one of two types, called kappa (κ) and lambda (λ), based on the amino acid sequence of its constant domain. An immunoglobulin essentially consists of two Fab molecules and an Fc domain, linked via the immunoglobulin hinge region.

The term “antibody” herein is used in the broadest sense and encompasses various antibody structures, including but not limited to monoclonal antibodies, polyclonal antibodies, and antibody fragments so long as they exhibit the desired antigen-binding activity.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂, diabodies, linear antibodies, single-chain antibody molecules (e.g., scFv), and single-domain antibodies. For a review of certain antibody fragments, see Hudson et al., Nat Med 9, 129-134 (2003). For a review of scFv fragments, see e.g., Pluckthun, in The Pharmacology of Monoclonal Antibodies, vol. 113, Rosenburg and Moore eds., Springer-Verlag, New York, pp. 269-315 (1994); see also WO 93/16185; and U.S. Pat. Nos. 5,571,894 and 5,587,458. For discussion of Fab and F(ab′)₂ fragments comprising salvage receptor binding epitope residues and having increased in vivo half-life, see U.S. Pat. No. 5,869,046. Diabodies are antibody fragments with two antigen-binding sites that may be bivalent or bispecific. See, for example, EP 404,097; WO 1993/01161; Hudson et al., Nat Med 9, 129-134 (2003); and Hollinger et al., Proc Natl Acad Sci USA 90, 6444-6448 (1993). Triabodies and tetrabodies are also described in Hudson et al., Nat Med 9, 129-134 (2003). Single-domain antibodies are antibody fragments comprising all or a portion of the heavy chain variable domain or all or a portion of the light chain variable domain of an antibody. In certain embodiments, a single-domain antibody is a human single-domain antibody (Domantis, Inc., Waltham, Mass.; see e.g., U.S. Pat. No. 6,248,516 B1). Antibody fragments can be made by various techniques, including but not limited to proteolytic digestion of an intact antibody as well as production by recombinant host cells (e.g., E. coli or phage), as described herein.

The term “antigen binding domain” refers to the part of an antibody that comprises the area which specifically binds to and is complementary to part or all of an antigen. An antigen binding domain may be provided by, for example, one or more antibody variable domains (also called antibody variable regions). Particularly, an antigen binding domain comprises an antibody light chain variable region (VL) and an antibody heavy chain variable region (VH).

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three hypervariable regions (HVRs). See, e.g., Kindt et al., Kuby Immunology, 6^{th} ed., W.H. Freeman and Co., page 91 (2007). A single VH or VL domain may be sufficient to confer antigen-binding specificity.

The term “hypervariable region” or “HVR”, as used herein, refers to each of the regions of an antibody variable domain which are hypervariable in sequence and/or form structurally defined loops (“hypervariable loops”). Generally, native four-chain antibodies comprise six HVRs; three in the VH (H1, H2, H3), and three in the VL (L1, L2, L3). HVRs generally comprise amino acid residues from the hypervariable loops and/or from the complementarity determining regions (CDRs), the latter being of highest sequence variability and/or involved in antigen recognition. With the exception of CDR1 in VH, CDRs generally comprise the amino acid residues that form the hypervariable loops. Hypervariable regions (HVRs) are also referred to as “complementarity determining regions” (CDRs), and these terms are used herein interchangeably in reference to portions of the variable region that form the antigen binding regions. This particular region has been described by Kabat et al., U.S. Dept. of Health and Human Services, Sequences of Proteins of Immunological Interest (1983) and by Chothia et al., J Mol Biol 196:901-917 (1987), where the definitions include overlapping or subsets of amino acid residues when compared against each other. Nevertheless, application of either definition to refer to a CDR of an antibody or variants thereof is intended to be within the scope of the term as defined and used herein. The appropriate amino acid residues which encompass the CDRs as defined by each of the above cited references are set forth below in Table 1 as a comparison. The exact residue numbers which encompass a particular CDR will vary depending on the sequence and size of the CDR. Those skilled in the art can routinely determine which residues comprise a particular CDR given the variable region amino acid sequence of the antibody.

TABLE 1
CDR Definitions¹

| CDR | Kabat | Chothia | AbM² |
|---|---|---|---|
| V_{H} CDR1 | 31-35 | 26-32 | 26-35 |
| V_{H} CDR2 | 50-65 | 52-58 | 50-58 |
| V_{H} CDR3 | 95-102 | 95-102 | 95-102 |
| V_{L} CDR1 | 24-34 | 26-32 | 24-34 |
| V_{L} CDR2 | 50-56 | 50-52 | 50-56 |
| V_{L} CDR3 | 89-97 | 91-96 | 89-97 |

¹Numbering of all CDR definitions in Table 1 is according to the numbering conventions set forth by Kabat et al. (see below).
²“AbM” with a lowercase “b” as used in Table 1 refers to the CDRs as defined by Oxford Molecular's “AbM” antibody modeling software.

Kabat et al. also defined a numbering system for variable region sequences that is applicable to any antibody. One of ordinary skill in the art can unambiguously assign this system of “Kabat numbering” to any variable region sequence, without reliance on any experimental data beyond the sequence itself. As used herein, “Kabat numbering” refers to the numbering system set forth by Kabat et al., U.S. Dept. of Health and Human Services, “Sequence of Proteins of Immunological Interest” (1983). Unless otherwise specified, references to the numbering of specific amino acid residue positions in an antibody variable region are according to the Kabat numbering system.

The polypeptide sequences of the sequence listing are not numbered according to the Kabat numbering system. However, it is well within the ordinary skill of one in the art to convert the numbering of the sequences of the Sequence Listing to Kabat numbering.

“Framework” or “FR” refers to variable domain residues other than hypervariable region (HVR) residues. The FR of a variable domain generally consists of four FR domains: FR1, FR2, FR3, and FR4. Accordingly, the HVR and FR sequences generally appear in the following sequence in VH (or VL): FR1-H1(L1)-FR2-H2(L2)-FR3-H3(L3)-FR4.

The “class” of an antibody or immunoglobulin refers to the type of constant domain or constant region possessed by its heavy chain. There are five major classes of antibodies: IgA, IgD, IgE, IgG, and IgM, and several of these may be further divided into subclasses (isotypes), e.g., IgG₁, IgG₂, IgG₃, IgG₄, IgA₁, and IgA₂. The heavy chain constant domains that correspond to the different classes of immunoglobulins are called α, δ, ε, γ, and μ, respectively.

The term “Fc region” herein is used to define a C-terminal region of an immunoglobulin heavy chain that contains at least a portion of the constant region. The term includes native sequence Fc regions and variant Fc regions. In one aspect, a human IgG heavy chain Fc region extends from Cys226, or from Pro230, to the carboxyl-terminus of the heavy chain. However, antibodies produced by host cells may undergo post-translational cleavage of one or more, particularly one or two, amino acids from the C-terminus of the heavy chain. Therefore an antibody produced by a host cell by expression of a specific nucleic acid molecule encoding a full-length heavy chain may include the full-length heavy chain, or it may include a cleaved variant of the full-length heavy chain. This may be the case where the final two C-terminal amino acids of the heavy chain are glycine (G446) and lysine (K447, EU numbering system). Therefore, the C-terminal lysine (Lys447), or the C-terminal glycine (Gly446) and lysine (Lys447), of the Fc region may or may not be present. Amino acid sequences of heavy chains including an Fc region are denoted herein without C-terminal glycine-lysine dipeptide if not indicated otherwise. In one aspect, a heavy chain including an Fc region as specified herein, comprised in an antibody according to the invention, comprises an additional C-terminal glycine-lysine dipeptide (G446 and K447, EU numbering system). In one aspect, a heavy chain including an Fc region as specified herein, comprised in an antibody according to the invention, comprises an additional C-terminal glycine residue (G446, numbering according to EU index). Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, Md., 1991. A “subunit” of an Fc domain as used herein refers to one of the two polypeptides forming the dimeric Fc domain, i.e. a polypeptide comprising C-terminal constant regions of an immunoglobulin heavy chain, capable of stable self-association. For example, a subunit of an IgG Fc domain comprises an IgG CH2 and an IgG CH3 constant domain.

By “fused” is meant that the components (e.g. a Fab molecule and an Fc domain subunit) are linked by peptide bonds, either directly or via one or more peptide linkers.

A “modification promoting the association of the first and the second subunit of the Fc domain” is a manipulation of the peptide backbone or the post-translational modifications of an Fc domain subunit that reduces or prevents the association of a polypeptide comprising the Fc domain subunit with an identical polypeptide to form a homodimer. A modification promoting association as used herein particularly includes separate modifications made to each of the two Fc domain subunits desired to associate (i.e. the first and the second subunit of the Fc domain), wherein the modifications are complementary to each other so as to promote association of the two Fc domain subunits. For example, a modification promoting association may alter the structure or charge of one or both of the Fc domain subunits so as to make their association sterically or electrostatically favorable, respectively. Thus, (hetero)dimerization occurs between a polypeptide comprising the first Fc domain subunit and a polypeptide comprising the second Fc domain subunit, which might be non-identical in the sense that further components fused to each of the subunits (e.g., antigen binding moieties) are not the same. In some embodiments the modification promoting association comprises an amino acid mutation in the Fc domain, specifically an amino acid substitution. In a particular embodiment, the modification promoting association comprises a separate amino acid mutation, specifically an amino acid substitution, in each of the two subunits of the Fc domain.

The term “effector functions” refers to those biological activities attributable to the Fc region of an antibody, which vary with the antibody isotype. Examples of antibody effector functions include: C1q binding and complement dependent cytotoxicity (CDC), Fc receptor binding, antibody-dependent cell-mediated cytotoxicity (ADCC), antibody-dependent cellular phagocytosis (ADCP), cytokine secretion, immune complex-mediated antigen uptake by antigen presenting cells, down regulation of cell surface receptors (e.g., B cell receptor), and B cell activation.

As used herein, the terms “engineer, engineered, engineering”, are considered to include any manipulation of the peptide backbone or the post-translational modifications of a naturally occurring or recombinant polypeptide or fragment thereof. Engineering includes modifications of the amino acid sequence, of the glycosylation pattern, or of the side chain group of individual amino acids, as well as combinations of these approaches.

The term “amino acid mutation” as used herein is meant to encompass amino acid substitutions, deletions, insertions, and modifications. Any combination of substitution, deletion, insertion, and modification can be made to arrive at the final construct, provided that the final construct possesses the desired characteristics, e.g., reduced binding to an Fc receptor, or increased association with another peptide. Amino acid sequence deletions and insertions include amino- and/or carboxy-terminal deletions and insertions of amino acids. Particular amino acid mutations are amino acid substitutions. For the purpose of altering e.g., the binding characteristics of an Fc region, non-conservative amino acid substitutions, i.e. replacing one amino acid with another amino acid having different structural and/or chemical properties, are particularly preferred. Amino acid substitutions include replacement by non-naturally occurring amino acids or by naturally occurring amino acid derivatives of the twenty standard amino acids (e.g., 4-hydroxyproline, 3-methylhistidine, omithine, homoserine, 5-hydroxylysine). Amino acid mutations can be generated using genetic or chemical methods well known in the art. Genetic methods may include site-directed mutagenesis, PCR, gene synthesis and the like. It is contemplated that methods of altering the side chain group of an amino acid by methods other than genetic engineering, such as chemical modification, may also be useful. Various designations may be used herein to indicate the same amino acid mutation. For example, a substitution from proline at position 329 of the Fc domain to glycine can be indicated as 329G, G329, G329, P329G, or Pro329Gly.

As used herein, term “polypeptide” refers to a molecule composed of monomers (amino acids) linearly linked by amide bonds (also known as peptide bonds). The term “polypeptide” refers to any chain of two or more amino acids, and does not refer to a specific length of the product. Thus, peptides, dipeptides, tripeptides, oligopeptides, “protein,” “amino acid chain,” or any other term used to refer to a chain of two or more amino acids, are included within the definition of “polypeptide,” and the term “polypeptide” may be used instead of, or interchangeably with any of these terms. The term “polypeptide” is also intended to refer to the products of post-expression modifications of the polypeptide, including without limitation glycosylation, acetylation, phosphorylation, amidation, derivatization by known protecting/blocking groups, proteolytic cleavage, or modification by non-naturally occurring amino acids. A polypeptide may be derived from a natural biological source or produced by recombinant technology, but is not necessarily translated from a designated nucleic acid sequence. It may be generated in any manner, including by chemical synthesis. A polypeptide of the invention may be of a size of about 3 or more, 5 or more, 10 or more, 20 or more, 25 or more, 50 or more, 75 or more, 100 or more, 200 or more, 500 or more, 1,000 or more, or 2,000 or more amino acids. Polypeptides may have a defined three-dimensional structure, although they do not necessarily have such structure. Polypeptides with a defined three-dimensional structure are referred to as folded, and polypeptides which do not possess a defined three-dimensional structure, but rather can adopt a large number of different conformations, and are referred to as unfolded.

By an “isolated” polypeptide or a variant, or derivative thereof is intended a polypeptide that is not in its natural milieu. No particular level of purification is required. For example, an isolated polypeptide can be removed from its native or natural environment. Recombinantly produced polypeptides and proteins expressed in host cells are considered isolated for the purpose of the invention, as are native or recombinant polypeptides which have been separated, fractionated, or partially or substantially purified by any suitable technique.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or may be compiled from the source code. The ALIGN-2 program should be compiled for use on a UNIX operating system, including digital UNIX V4.0D. All sequence comparison parameters are set by the ALIGN-2 program and do not vary. In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100 times the fraction X/Y

where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

The term “polynucleotide” refers to an isolated nucleic acid molecule or construct, e.g., messenger RNA (mRNA), virally-derived RNA, or plasmid DNA (pDNA). A polynucleotide may comprise a conventional phosphodiester bond or a non-conventional bond (e.g., an amide bond, such as found in peptide nucleic acids (PNA). The term “nucleic acid molecule” refers to any one or more nucleic acid segments, e.g., DNA or RNA fragments, present in a polynucleotide.

By “isolated” nucleic acid molecule or polynucleotide is intended a nucleic acid molecule, DNA or RNA, which has been removed from its native environment. For example, a recombinant polynucleotide encoding a polypeptide contained in a vector is considered isolated for the purposes of the present invention. Further examples of an isolated polynucleotide include recombinant polynucleotides maintained in heterologous host cells or purified (partially or substantially) polynucleotides in solution. An isolated polynucleotide includes a polynucleotide molecule contained in cells that ordinarily contain the polynucleotide molecule, but the polynucleotide molecule is present extrachromosomally or at a chromosomal location that is different from its natural chromosomal location. Isolated RNA molecules include in vivo or in vitro RNA transcripts of the present invention, as well as positive and negative strand forms, and double-stranded forms. Isolated polynucleotides or nucleic acids according to the present invention further include such molecules produced synthetically. In addition, a polynucleotide or a nucleic acid may be or may include a regulatory element such as a promoter, ribosome binding site, or a transcription terminator.

By a nucleic acid or polynucleotide having a nucleotide sequence at least, for example, 95% “identical” to a reference nucleotide sequence of the present invention, it is intended that the nucleotide sequence of the polynucleotide is identical to the reference sequence except that the polynucleotide sequence may include up to five point mutations per each 100 nucleotides of the reference nucleotide sequence. In other words, to obtain a polynucleotide having a nucleotide sequence at least 95% identical to a reference nucleotide sequence, up to 5% of the nucleotides in the reference sequence may be deleted or substituted with another nucleotide, or a number of nucleotides up to 5% of the total nucleotides in the reference sequence may be inserted into the reference sequence. These alterations of the reference sequence may occur at the 5′ or 3′ terminal positions of the reference nucleotide sequence or anywhere between those terminal positions, interspersed either individually among residues in the reference sequence or in one or more contiguous groups within the reference sequence. As a practical matter, whether any particular polynucleotide sequence is at least 80%, 85%, 90%, 95%, 96%, 97%, 98% or 99% identical to a nucleotide sequence of the present invention can be determined conventionally using known computer programs, such as the ones discussed above for polypeptides (e.g., ALIGN-2).

The term “expression cassette” refers to a polynucleotide generated recombinantly or synthetically, with a series of specified nucleic acid elements that permit transcription of a particular nucleic acid in a target cell. The recombinant expression cassette can be incorporated into a plasmid, chromosome, mitochondrial DNA, plastid DNA, virus, or nucleic acid fragment. Typically, the recombinant expression cassette portion of an expression vector includes, among other sequences, a nucleic acid sequence to be transcribed and a promoter. In certain embodiments, the expression cassette of the invention comprises polynucleotide sequences that encode bispecific antigen binding molecules of the invention or fragments thereof.

The term “vector” or “expression vector” is synonymous with “expression construct” and refers to a DNA molecule that is used to introduce and direct the expression of a specific gene to which it is operably associated in a target cell. The term includes the vector as a self-replicating nucleic acid structure as well as the vector incorporated into the genome of a host cell into which it has been introduced. The expression vector of the present invention comprises an expression cassette. Expression vectors allow transcription of large amounts of stable mRNA. Once the expression vector is inside the target cell, the ribonucleic acid molecule or protein that is encoded by the gene is produced by the cellular transcription and/or translation machinery. In one embodiment, the expression vector of the invention comprises an expression cassette that comprises polynucleotide sequences that encode bispecific antigen binding molecules of the invention or fragments thereof.

The terms “host cell”, “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein. A host cell is any type of cellular system that can be used to generate the bispecific antigen binding molecules of the present invention. Host cells include cultured cells, e.g., mammalian cultured cells, such as CHO cells, BHK cells, NS0 cells, SP2/0 cells, YO myeloma cells, P3X63 mouse myeloma cells, PER cells, PER.C6 cells or hybridoma cells, yeast cells, insect cells, and plant cells, to name only a few, but also cells comprised within a transgenic animal, transgenic plant or cultured plant or animal tissue.

An “activating Fc receptor” is an Fc receptor that following engagement by an Fc domain of an antibody elicits signaling events that stimulate the receptor-bearing cell to perform effector functions. Human activating Fc receptors include FcγRIIIa (CD16a), FcγRI (CD64), FcγRIIa (CD32), and FcαRI (CD89).

Antibody-dependent cell-mediated cytotoxicity (ADCC) is an immune mechanism leading to the lysis of antibody-coated target cells by immune effector cells. The target cells are cells to which antibodies or derivatives thereof comprising an Fc region specifically bind, generally via the protein part that is N-terminal to the Fc region. As used herein, the term “reduced ADCC” is defined as either a reduction in the number of target cells that are lysed in a given time, at a given concentration of antibody in the medium surrounding the target cells, by the mechanism of ADCC defined above, and/or an increase in the concentration of antibody in the medium surrounding the target cells, required to achieve the lysis of a given number of target cells in a given time, by the mechanism of ADCC. The reduction in ADCC is relative to the ADCC mediated by the same antibody produced by the same type of host cells, using the same standard production, purification, formulation and storage methods (which are known to those skilled in the art), but that has not been engineered. For example the reduction in ADCC mediated by an antibody comprising in its Fc domain an amino acid substitution that reduces ADCC, is relative to the ADCC mediated by the same antibody without this amino acid substitution in the Fc domain. Suitable assays to measure ADCC are well known in the art (see e.g., PCT publication no. WO 2006/082515 or PCT publication no. WO 2012/130831).

An “effective amount” of an agent refers to the amount that is necessary to result in a physiological change in the cell or tissue to which it is administered.

A “therapeutically effective amount” of an agent, e.g., a pharmaceutical composition, refers to an amount effective, at dosages and for periods of time necessary, to achieve the desired therapeutic or prophylactic result. A therapeutically effective amount of an agent for example eliminates, decreases, delays, minimizes or prevents adverse effects of a disease.

An “individual” or “subject” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). Particularly, the individual or subject is a human.

The term “pharmaceutical composition” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical composition, other than an active ingredient, which is nontoxic to a subject. A pharmaceutically acceptable carrier includes, but is not limited to, a buffer, excipient, stabilizer, or preservative.

As used herein, “treatment” (and grammatical variations thereof such as “treat” or “treating”) refers to clinical intervention in an attempt to alter the natural course of a disease in the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, protease-activatable T cell activating bispecific molecules of the invention are used to delay development of a disease or to slow the progression of a disease. The term “package insert” is used to refer to instructions customarily included in commercial packages of therapeutic products, that contain information about the indications, usage, dosage, administration, combination therapy, contraindications and/or warnings concerning the use of such therapeutic products.

An “idiotype-specific polypeptide” as used herein refers to a polypeptide that recognizes the idiotype of an antigen-binding moiety, e.g., an antigen-binding moiety specific for CD3. The idiotype-specific polypeptide is capable of specifically binding to the variable region of the antigen-binding moiety and thereby reducing or preventing specific binding of the antigen-binding moiety to its cognate antigen. When associated with a molecule that comprises the antigen-binding moiety, the idiotype-specific polypeptide can function as a masking moiety of the molecule. Specifically disclosed herein are anti-idiotype antibodies or anti-idiotype-binding antibody fragments specific for the idiotype of anti-CD3 binding molecules.

“Protease” or “proteolytic enzyme” as used herein refers to any proteolytic enzyme that cleaves the linker at a recognition site and that is expressed by a target cell. Such proteases might be secreted by the target cell or remain associated with the target cell, e.g., on the target cell surface.

Examples of proteases include but are not limited to metalloproteinases, e.g., matrix metalloproteinase 1-28 and A Disintegrin And Metalloproteinase (ADAM) 2, 7-12, 15, 17-23, 28-30 and 33, serine proteases, e.g., urokinase-type plasminogen activator and Matriptase, cysteine protease, aspartic proteases, and members of the cathepsin family.

“Protease activatable” as used herein, with respect to the T cell activating bispecific molecule, refers to a T cell activating bispecific molecule having reduced or abrogated ability to activate T cells due to a masking moiety that reduces or abrogates the T cell activating bispecific molecule's ability to bind to CD3. Upon dissociation of the masking moiety by proteolytic cleavage, e.g., by proteolytic cleavage of a linker connecting the masking moiety to the T cell activating bispecific molecule, binding to CD3 is restored and the T cell activating bispecific molecule is thereby activated.

“Reversibly concealing” as used herein refers to the binding of a masking moiety or idiotype-specific polypeptide to an antigen-binding moiety or molecule such as to prevent the antigen-binding moiety or molecule from its antigen, e.g., CD3. This concealing is reversible in that the idiotype-specific polypeptide can be released from the antigen-binding moiety or molecule, e.g., by protease cleavage, and thereby freeing the antigen-binding moiety or molecule to bind to its antigen.

### DETAILED DESCRIPTION

In one aspect, the invention relates to a protease-activatable T cell activating bispecific molecule comprising
(a) a first antigen binding moiety capable of binding to CD3;
(b) a second antigen binding moiety capable of binding to a target cell antigen; and
(c) a masking moiety covalently attached to the T cell bispecific binding molecule through a protease-cleavable linker, wherein the masking moiety is capable of binding to the idiotype of the first or the second antigen binding moiety thereby reversibly concealing the first or second antigen binding moiety.

The first antigen binding moiety capable of binding to CD3 comprises an idiotype. In one embodiment, the masking moiety of the protease-activatable T cell activating bispecific molecule is covalently attached to the first antigen binding moiety. In one embodiment the masking moiety is covalently attached to the heavy chain variable region of the first antigen binding moiety. In one embodiment the masking moiety is covalently attached to the light chain variable region of the first antigen binding moiety. This covalent bond is separate from the specific binding, which is preferably non-covalent, of the masking moiety to the idiotype first antigen binding site. The idiotype of the first antigen binding moiety comprises its variable region. In one embodiment the masking moiety binds to amino acid residues that make contact with CD3 when the first antigen biding moiety is bound to CD3. In a preferred embodiment, the masking moiety is not the cognate antigen or fragments thereof of the first antigen binding moiety, i.e., the masking moiety is not a CD3 or fragments thereof. In one embodiment the masking moiety is an anti-idiotypic antibody or fragment thereof. In one embodiment, the masking moiety is an anti-idiotypic scFv. Exemplary embodiments of masking moieties which are anti-idiotypic scFv, and protease activatable T cell activating molecules comprising such masking moieties, are described in detail in the examples.

In one embodiment the protease-activatable T cell activating bispecific molecule comprises
(i) a first antigen binding moiety which is a Fab molecule capable of binding to CD3, and which comprises at least one heavy chain complementarity determining region (CDR) selected from the group consisting of SEQ ID NO: 2, SEQ ID NO: 4 and SEQ ID NO: 10 and at least one light chain CDR selected from the group of SEQ ID NO: 20, SEQ ID NO: 21, SEQ ID NO: 22;
(ii) a second antigen binding moiety which is a Fab molecule capable of binding to a target cell antigen.

In one embodiment the first antigen binding moiety comprises a heavy chain variable region comprising an amino acid sequence that is at least about 95%, 96%, 97%, 98%, 99% or 100% identical to an amino acid sequence of SEQ ID NO: 16 and a light chain variable region comprising an amino acid sequence that is at least about 95%, 96%, 97%, 98%, 99% or 100% identical to an amino acid sequence of SEQ ID NO: 23.

In one embodiment the first antigen binding moiety comprises the heavy chain variable region comprising an amino acid sequence of SEQ ID NO: 16 and the light chain variable region comprising an amino acid sequence of SEQ ID NO: 23.

In a specific embodiment the second antigen binding moiety is capable of binding to FolR1 and comprises at least one heavy chain complementarity determining region (CDR) selected from the group consisting of SEQ ID NO: 54, SEQ ID NO: 55 and SEQ ID NO: 56 and at least one light chain CDR selected from the group of SEQ ID NO: 20, SEQ ID NO: 21 and SEQ ID NO: 22.

In another specific embodiment, the second antigen binding moiety is capable of binding to FolR1 and comprises a heavy chain variable region comprising an amino acid sequence that is at least about 95%, 96%, 97%, 98%, 99% or 100% identical to the amino acid sequence of SEQ ID NO: 53 and a light chain variable region comprising an amino acid sequence that is at least about 95%, 96%, 97%, 98%, 99% or 100% identical to the amino acid sequence of SEQ ID NO: 23.

In another specific embodiment, the second antigen binding moiety is capable of binding to TYRP1 and comprises at least one heavy chain complementarity determining region (CDR) selected from the group consisting of SEQ ID NO: 24, SEQ ID NO: 25 and SEQ ID NO: 26 and at least one light chain CDR selected from the group of SEQ ID NO: 28, SEQ ID NO: 29 and SEQ ID NO: 30.

In another specific embodiment, the second antigen binding moiety is capable of binding to TYRP1 and comprises a heavy chain comprising an amino acid sequence that is at least about 95%, 96%, 97%, 98%, 99% or 100% identical to an amino acid sequence of SEQ ID NO: 27, and a light chain comprising an amino acid sequence that is at least about 95%, 96%, 97%, 98%, 99% or 100% identical to an amino acid sequence of SEQ ID NO: 31.

In one embodiment the present invention provides a protease-activatable T cell activating bispecific molecule comprising
(i) a first antigen binding moiety which is a Fab molecule capable of binding to CD3, comprising at least one heavy chain complementarity determining region (CDR) selected from the group consisting of SEQ ID NO: 2, SEQ ID NO: 4 and SEQ ID NO: 10 and at least one light chain CDR selected from the group of SEQ ID NO: 20, SEQ ID NO: 21, SEQ ID NO: 22;
(ii) a second antigen binding moiety which is a Fab molecule capable of binding to FolR1 comprising at least one heavy chain complementarity determining region (CDR) selected from the group consisting of SEQ ID NO: 54, SEQ ID NO: 55 and SEQ ID NO: 56 and at least one light chain CDR selected from the group of SEQ ID NO: 20, SEQ ID NO: 21 and SEQ ID NO: 22.

In one embodiment the present invention provides a protease-activatable T cell activating bispecific molecule comprising
(i) a first antigen binding moiety which is a Fab molecule capable of binding to CD3 comprising a heavy chain variable region comprising an amino acid sequence that is at least about 95%, 96%, 97%, 98%, 99% or 100% identical to the amino acid sequence of SEQ ID NO: 16 and a light chain variable region comprising an amino acid sequence that is at least about 95%, 96%, 97%, 98%, 99% or 100% identical to the amino acid sequence of SEQ ID NO: 23,
(ii) a second antigen binding moiety which is a Fab molecule capable of binding to FolR1 comprising heavy chain variable region comprising an amino acid sequence that is at least about 95%, 96%, 97%, 98%, 99% or 100% identical to the amino acid sequence of SEQ ID NO: 53 and a light chain variable region comprising an amino acid sequence that is at least about 95%, 96%, 97%, 98%, 99% or 100% identical to the amino acid sequence of SEQ ID NO: 23.

In one embodiment the present invention provides a protease-activatable T cell activating bispecific molecule comprising
(i) a first antigen binding moiety which is a Fab molecule capable of binding to CD3, comprising at least one heavy chain complementarity determining region (CDR) selected from the group consisting of SEQ ID NO: 2, SEQ ID NO: 4 and SEQ ID NO: 10 and at least one light chain CDR selected from the group of SEQ ID NO: 20, SEQ ID NO: 21, SEQ ID NO: 22;
(ii) a second antigen binding moiety which is a Fab molecule capable of binding to TYRP1 comprising at least one heavy chain complementarity determining region (CDR) selected from the group consisting of SEQ ID NO: 24, SEQ ID NO: 25 and SEQ ID NO: 26 and at least one light chain CDR selected from the group of SEQ ID NO: 28, SEQ ID NO: 29, SEQ ID NO: 30.

In one embodiment the present invention provides a protease-activatable T cell activating bispecific molecule comprising
(i) a first antigen binding moiety which is a Fab molecule capable of binding to CD3 comprising a heavy chain variable region comprising an amino acid sequence that is at least about 95%, 96%, 97%, 98%, 99% or 100% identical to the amino acid sequence of SEQ ID NO: 16 and a light chain variable region comprising an amino acid sequence that is at least about 95%, 96%, 97%, 98%, 99% or 100% identical to the amino acid sequence of SEQ ID NO: 23.
(ii) a second antigen binding moiety which is a Fab molecule capable of binding to TYRP1 comprising a heavy chain variable region comprising an amino acid sequence that is at least about 95%, 96%, 97%, 98%, 99% or 100% identical to the amino acid sequence of SEQ ID NO: 27 and a light chain variable region comprising an amino acid sequence that is at least about 95%, 96%, 97%, 98%, 99% or 100% identical to the amino acid sequence of SEQ ID NO: 31.

In one embodiment, the second antigen binding moiety is a conventional Fab molecule.

In a particular embodiment, the first antigen binding moiety is a crossover Fab molecule wherein the constant regions of the Fab light chain and the Fab heavy chain are exchanged, and the second antigen binding moiety is a conventional Fab molecule. In a further particular embodiment, the first and the second antigen binding moiety are fused to each other, optionally through a peptide linker.

In particular embodiments, the protease-activatable T cell activating bispecific molecule further comprises an Fc domain composed of a first and a second subunit capable of stable association.

In a further particular embodiment, not more than one antigen binding moiety capable of binding to CD3 is present in the protease-activatable T cell activating bispecific molecule (i.e. the protease-activatable T cell activating bispecific molecule provides monovalent binding to CD3).

Protease-Activatable T Cell Activating Bispecific Molecule Formats

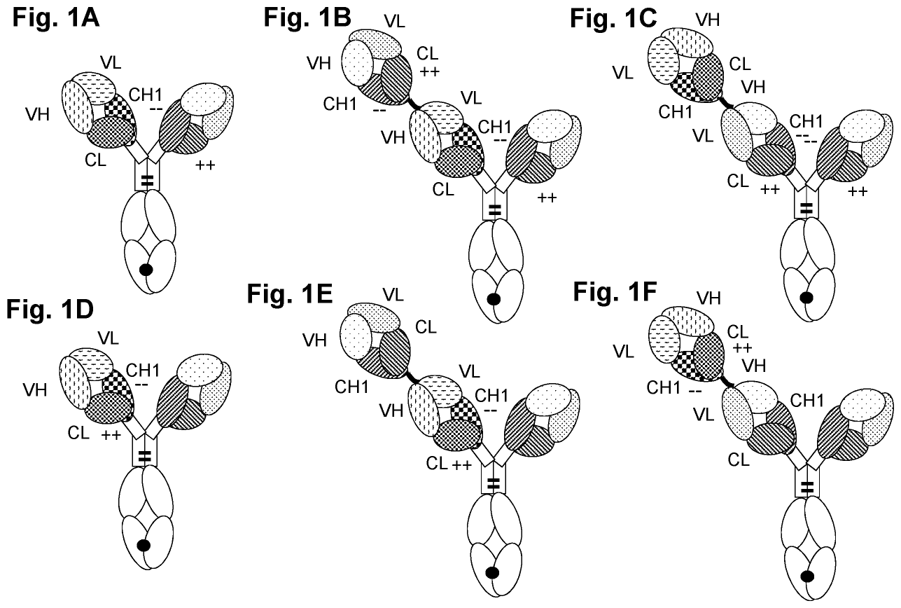

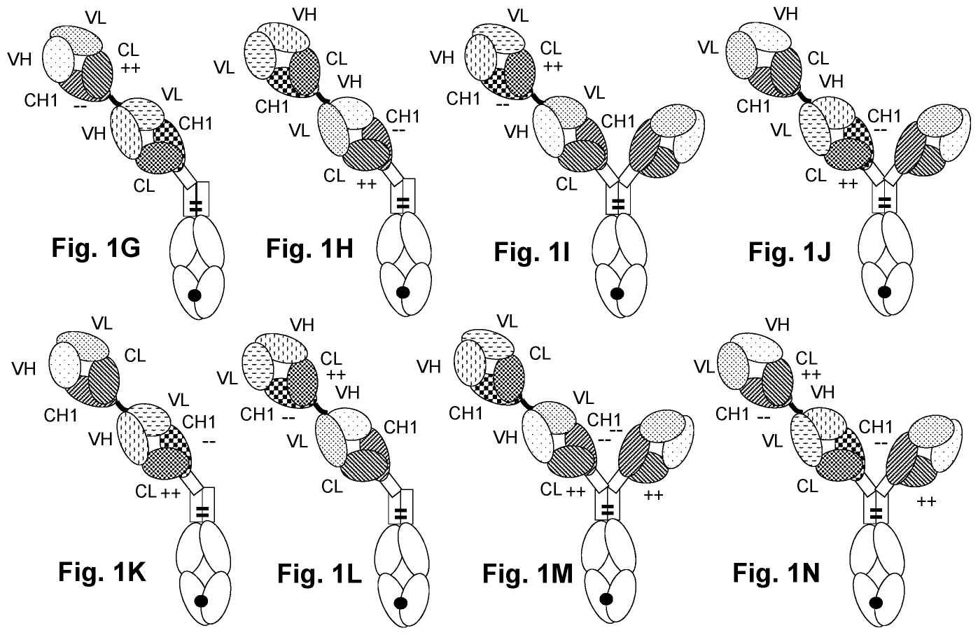

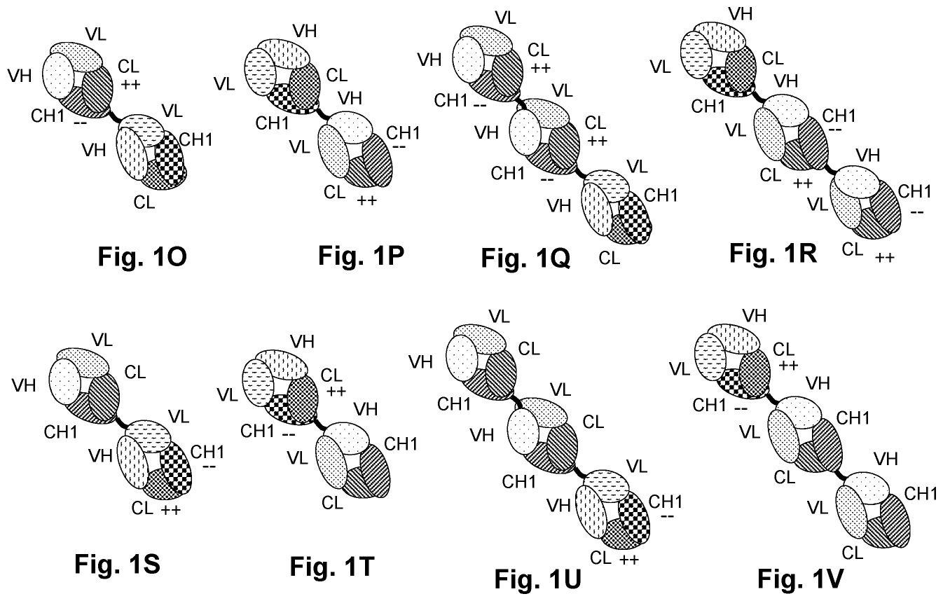

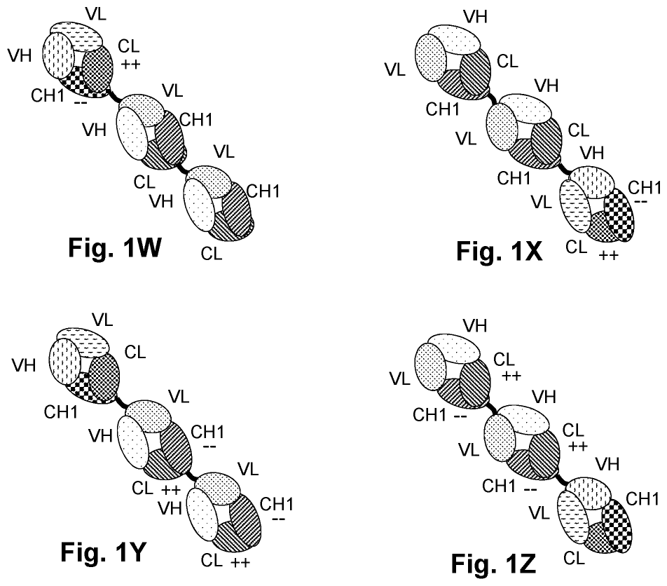

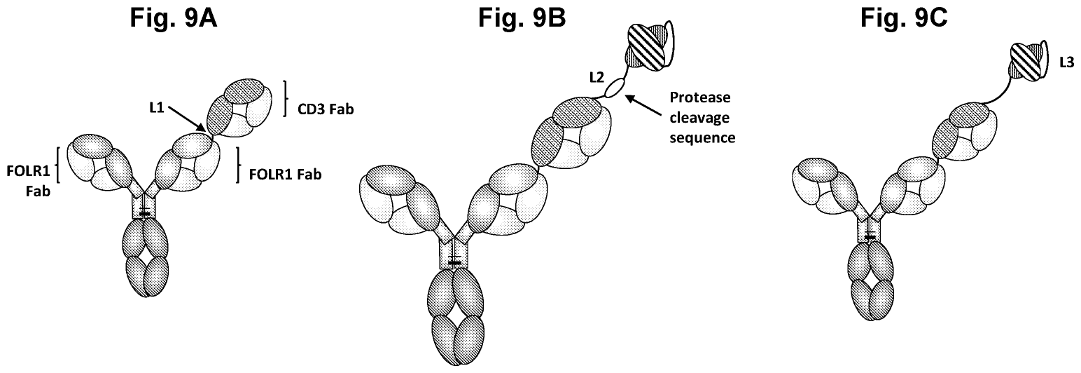

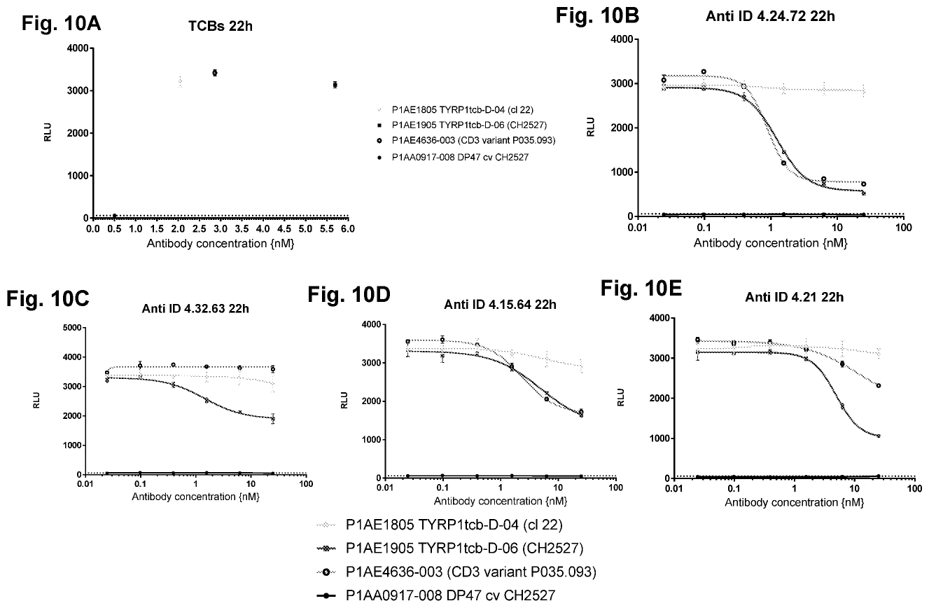

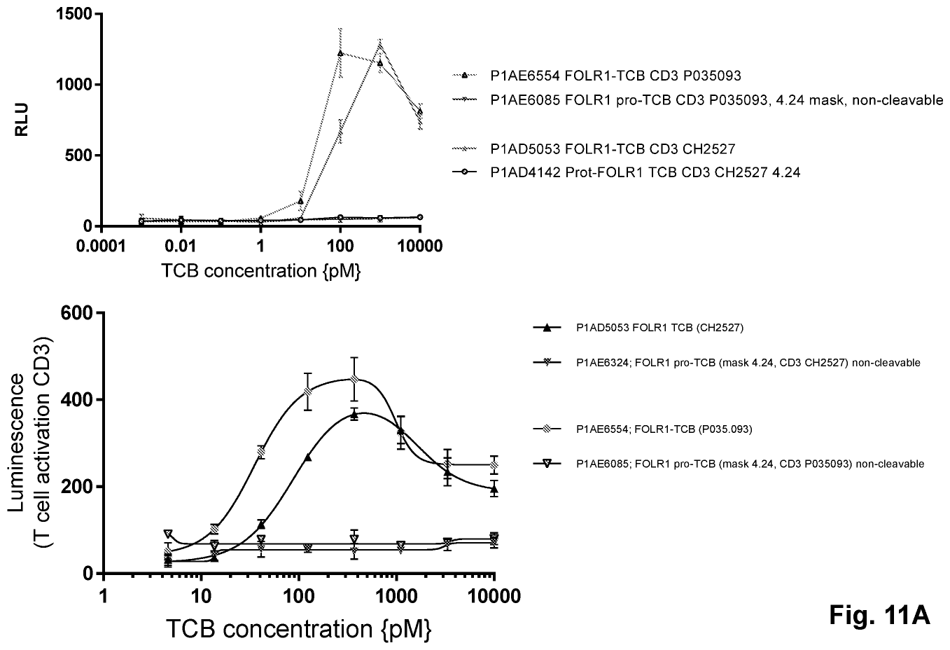

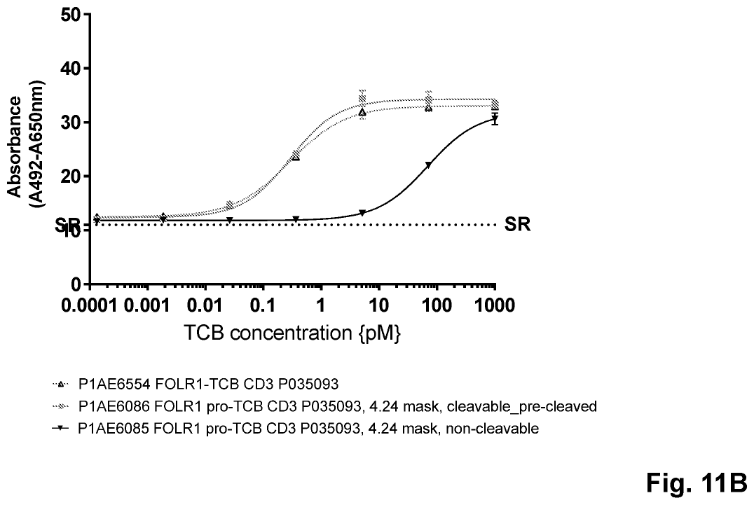

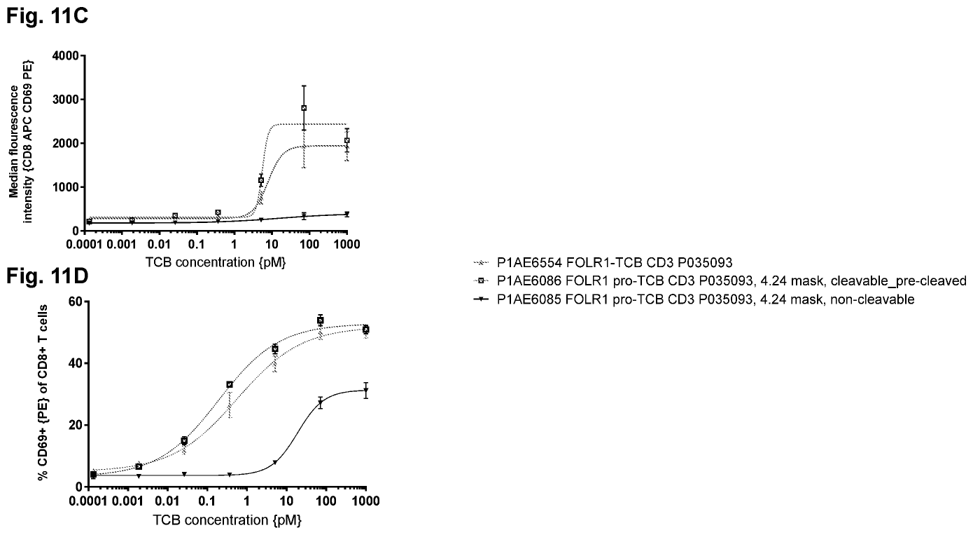

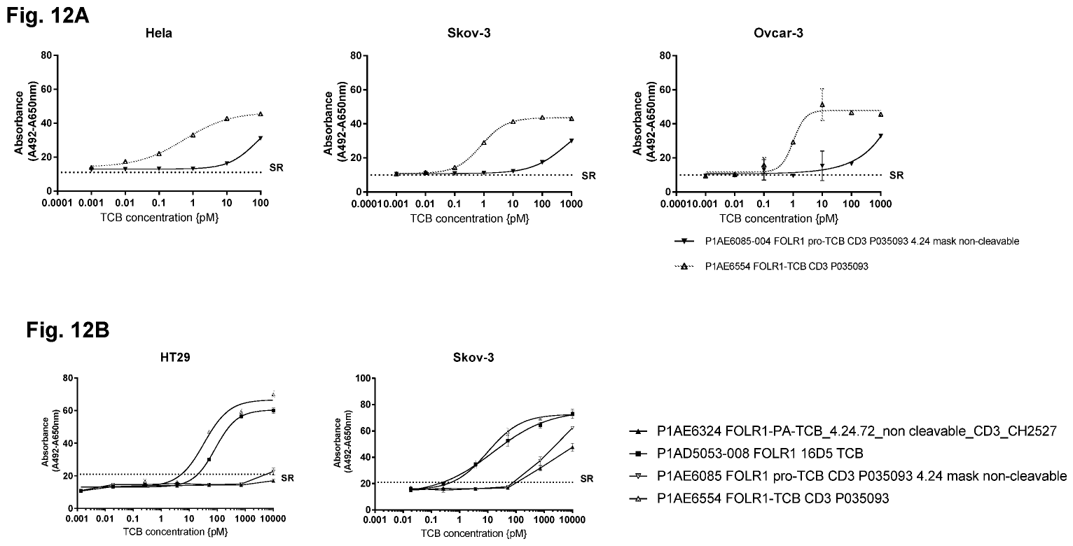

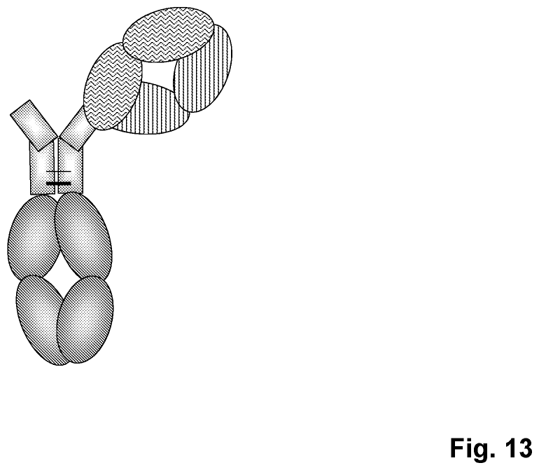

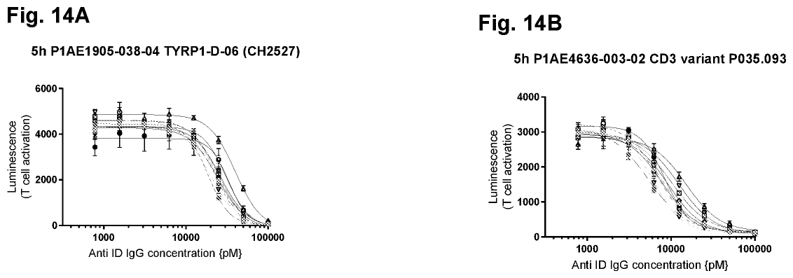

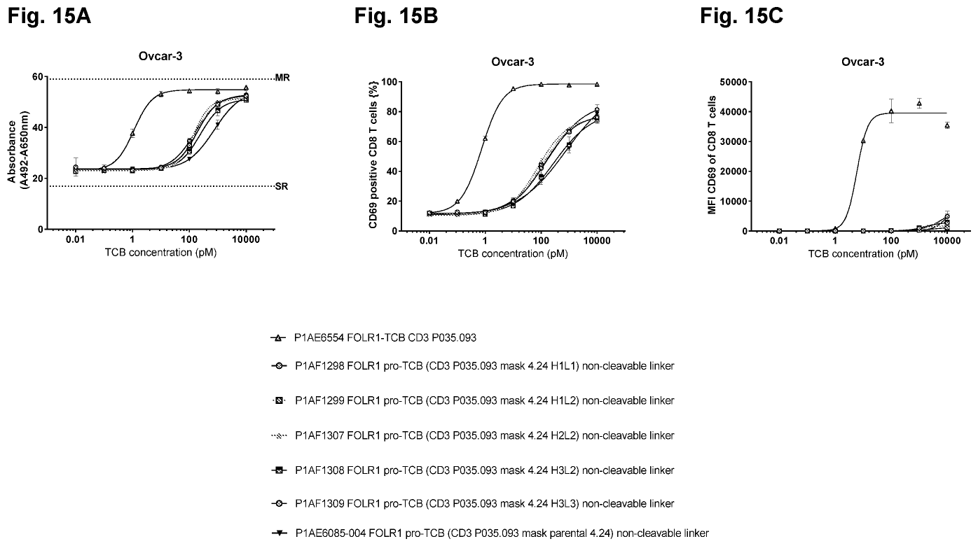

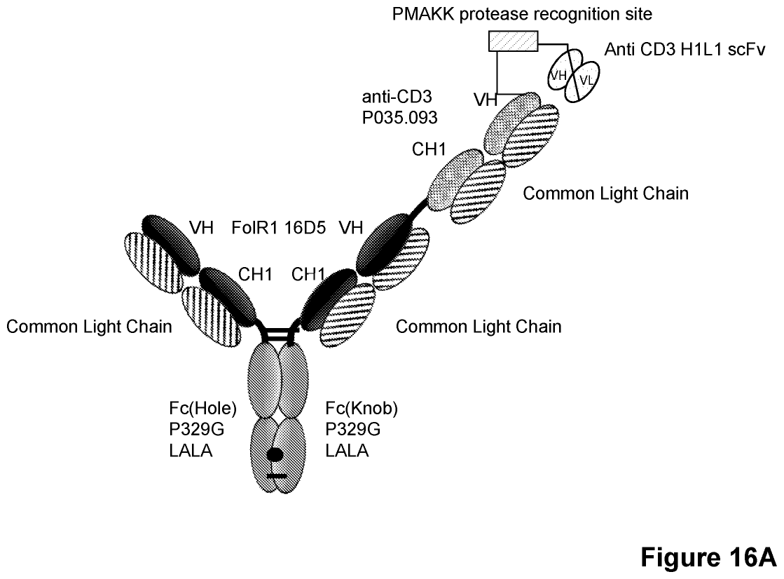

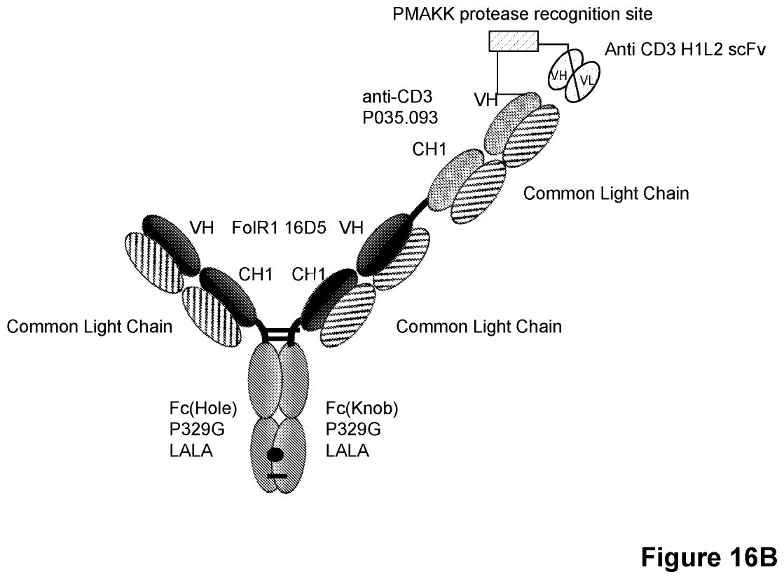

The components of the protease-activatable T cell activating bispecific molecule can be fused to each other in a variety of configurations. Exemplary configurations are depicted in FIGS. 1A-1Z, FIGS. 2A-2E, FIGS. 9A-9C and FIGS. 16A-16D.

In particular embodiments, the protease-activatable T cell activating bispecific molecule comprises an Fc domain composed of a first and a second subunit capable of stable association.

In some embodiments, the second antigen binding moiety is fused at the C-terminus of the Fab heavy chain to the N-terminus of the first or the second subunit of the Fc domain.

In one such embodiment, the first antigen binding moiety is fused at the C-terminus of the Fab heavy chain to the N-terminus of the Fab heavy chain of the second antigen binding moiety. In a specific such embodiment, the protease-activatable T cell activating bispecific molecule essentially consists of a first and a second antigen binding moiety, an Fc domain composed of a first and a second subunit, and optionally one or more peptide linkers, wherein the first antigen binding moiety is fused at the C-terminus of the Fab heavy chain to the N-terminus of the Fab heavy chain of the second antigen binding moiety, and the second antigen binding moiety is fused at the C-terminus of the Fab heavy chain to the N-terminus of the first or the second subunit of the Fc domain. Optionally, the Fab light chain of the first antigen binding moiety and the Fab light chain of the second antigen binding moiety may additionally be fused to each other. In another such embodiment, the first antigen binding moiety is fused at the C-terminus of the Fab heavy chain to the N-terminus of the first or second subunit of the Fc domain. In a specific such embodiment, the protease-activatable T cell activating bispecific molecule essentially consists of a first and a second antigen binding moiety, an Fc domain composed of a first and a second subunit, and optionally one or more peptide linkers, wherein the first and the second antigen binding moiety are each fused at the C-terminus of the Fab heavy chain to the N-terminus of one of the subunits of the Fc domain.

In other embodiments, the first antigen binding moiety is fused at the C-terminus of the Fab heavy chain to the N-terminus of the first or second subunit of the Fc domain.

In a particular such embodiment, the second antigen binding moiety is fused at the C-terminus of the Fab heavy chain to the N-terminus of the Fab heavy chain of the first antigen binding moiety.

In a specific such embodiment, the protease-activatable T cell activating bispecific molecule essentially consists of a first and a second antigen binding moiety, an Fc domain composed of a first and a second subunit, and optionally one or more peptide linkers, wherein the second antigen binding moiety is fused at the C-terminus of the Fab heavy chain to the N-terminus of the Fab heavy chain of the first antigen binding moiety, and the first antigen binding moiety is fused at the C-terminus of the Fab heavy chain to the N-terminus of the first or the second subunit of the Fc domain. Optionally, the Fab light chain of the first antigen binding moiety and the Fab light chain of the second antigen binding moiety may additionally be fused to each other. The antigen binding moieties may be fused to the Fc domain or to each other directly or through a peptide linker, comprising one or more amino acids, typically about 2-20 amino acids. Peptide linkers are known in the art and are described herein. Suitable, non-immunogenic peptide linkers include, for example, (G₄S)ₙ, (SG₄)ₙ, (G₄S)ₙ or G₄(SG₄)ₙ peptide linkers. “n” is generally a number between 1 and 10, typically between 2 and 4. A particularly suitable peptide linker for fusing the Fab light chains of the first and the second antigen binding moiety to each other is (G₄S)₂. An exemplary peptide linker suitable for connecting the Fab heavy chains of the first and the second antigen binding moiety is EPKSC(D)-(G₄S)₂ (SEQ ID NOs 105 and 106). Additionally, linkers may comprise (a portion of) an immunoglobulin hinge region. Particularly where an antigen binding moiety is fused to the N-terminus of an Fc domain subunit, it may be fused via an immunoglobulin hinge region or a portion thereof, with or without an additional peptide linker.

A protease-activatable T cell activating bispecific molecule with a single antigen binding moiety capable of binding to a target cell antigen is useful, particularly in cases where internalization of the target cell antigen is to be expected following binding of a high affinity antigen binding moiety. In such cases, the presence of more than one antigen binding moiety specific for the target cell antigen may enhance internalization of the target cell antigen, thereby reducing its availability.

In many other cases, however, it will be advantageous to have a protease-activatable T cell activating bispecific molecule comprising two or more antigen binding moieties specific for a target cell antigen (see examples in shown in FIGS. 1B, 1C, 1E, 1F, 1G, 1H, 1I, 1J, 1K, 1L, 1M, 1N, 1Q, 1R, 1U, iv), for example to optimize targeting to the target site or to allow crosslinking of target cell antigens.

Accordingly, in certain embodiments, the protease-activatable T cell activating bispecific molecule of the invention further comprises a third antigen binding moiety which is a Fab molecule capable of binding to a target cell antigen. In one embodiment, the third antigen binding moiety is a conventional Fab molecule. In one embodiment, the third antigen binding moiety is capable of binding to the same target cell antigen as the second antigen binding moiety. In a particular embodiment, the first antigen binding moiety is capable of binding to CD3, and the second and third antigen binding moieties are capable of binding to a target cell antigen. In a particular embodiment, the second and the third antigen binding moiety are identical (i.e. they comprise the same amino acid sequences).

In a particular embodiment, the first antigen binding moiety is capable of binding to CD3, and the second and third antigen binding moieties are capable of binding to FolR1, wherein the second and third antigen binding moieties comprise at least one heavy chain complementarity determining region (CDR) selected from the group consisting of SEQ ID NO: 54, SEQ ID NO: 55 and SEQ ID NO: 56 and at least one light chain CDR selected from the group of SEQ ID NO: 20, SEQ ID NO: 21 and SEQ ID NO: 22.

In a particular embodiment, the first antigen binding moiety is capable of binding to CD3, and comprises at least one heavy chain complementarity determining region (CDR) selected from the group consisting of SEQ ID NO: 2, SEQ ID NO: 4 and SEQ ID NO: 10 and at least one light chain CDR selected from the group of SEQ ID NO: 20, SEQ ID NO: 21, SEQ ID NO: 22; and the second and third antigen binding moieties are capable of binding to FolR1, wherein the second and third antigen binding moieties comprise at least one heavy chain complementarity determining region (CDR) selected from the group consisting of SEQ ID NO: 54, SEQ ID NO: 55 and SEQ ID NO: 56 and at least one light chain CDR selected from the group of SEQ ID NO: 20, SEQ ID NO: 21 and SEQ ID NO: 22.

In a particular embodiment, the first antigen binding moiety is capable of binding to CD3, and comprises at least one heavy chain complementarity determining region (CDR) selected from the group consisting of SEQ ID NO: 2, SEQ ID NO: 4 and SEQ ID NO: 10 and at least one light chain CDR selected from the group of SEQ ID NO: 20, SEQ ID NO: 21, SEQ ID NO: 22; and the second and third antigen binding moieties are capable of binding to FolR1, wherein the second and third antigen binding moieties comprise at least one heavy chain complementarity determining region (CDR) selected from the group consisting of SEQ ID NO: 54, SEQ ID NO: 55 and SEQ ID NO: 56 and at least one light chain CDR selected from the group of SEQ ID NO: 20, SEQ ID NO: 21 and SEQ ID NO: 22.

In a particular embodiment, the first antigen binding moiety is capable of binding to CD3, and comprises a heavy chain variable region comprising an amino acid sequence that is at least about 95%, 96%, 97%, 98%, 99% or 100% identical to the amino acid sequence of SEQ ID NO: 16 and a light chain variable region comprising an amino acid sequence that is at least about 95%, 96%, 97%, 98%, 99% or 100% identical to the amino acid sequence of SEQ ID NO: 23, and the second and third antigen binding moieties are capable of binding to FolR1, wherein the second and third antigen binding moieties comprise a heavy chain variable region comprising an amino acid sequence that is at least about 95%, 96%, 97%, 98%, 99% or 100% identical to the amino acid sequence of SEQ ID NO: 53 and a light chain variable region comprising an amino acid sequence that is at least about 95%, 96%, 97%, 98%, 99% or 100% identical to the amino acid sequence of SEQ ID NO: 23.

In one embodiment, the first antigen binding moiety is capable of binding to CD3, and the second and third antigen binding moieties are capable of binding to TYRP1, wherein the second and third antigen binding moieties comprise at least one heavy chain complementarity determining region (CDR) selected from the group consisting of SEQ ID NO: 2, SEQ ID NO: 4 and SEQ ID NO: 10 and at least one light chain CDR selected from the group of SEQ ID NO: 20, SEQ ID NO: 21 and SEQ ID NO: 22.

In one embodiment, the first antigen binding moiety is capable of binding to CD3, and comprises at least one heavy chain complementarity determining region (CDR) selected from the group consisting of SEQ ID NO: 2, SEQ ID NO: 4 and SEQ ID NO: 10 and at least one light chain CDR selected from the group of SEQ ID NO: 20, SEQ ID NO: 21, SEQ ID NO: 22; and the second and third antigen binding moieties are capable of binding to TYRP1, wherein the second and third antigen binding moieties comprise at least one heavy chain complementarity determining region (CDR) selected from the group consisting of SEQ ID NO: 24, SEQ ID NO: 25 and SEQ ID NO: 26 and at least one light chain CDR selected from the group of SEQ ID NO: 28, SEQ ID NO: 29 and SEQ ID NO: 30.

In one embodiment, the first antigen binding moiety is capable of binding to CD3, and comprises at least one heavy chain complementarity determining region (CDR) selected from the group consisting of SEQ ID NO: 2, SEQ ID NO: 4 and SEQ ID NO: 10 and at least one light chain CDR selected from the group of SEQ ID NO: 20, SEQ ID NO: 21 and SEQ ID NO: 22; and the second and third antigen binding moieties are capable of binding to TYRP1, wherein the second and third antigen binding moieties comprise at least one heavy chain complementarity determining region (CDR) selected from the group consisting of SEQ ID NO: 24, SEQ ID NO: 25 and SEQ ID NO: 26 and at least one light chain CDR selected from the group of SEQ ID NO: 28, SEQ ID NO: 29 and SEQ ID NO: 30.

In one embodiment, the first antigen binding moiety is capable of binding to CD3, and comprises a heavy chain variable region comprising an amino acid sequence that is at least about 95%, 96%, 97%, 98%, 99% or 100% identical to the amino acid sequence of SEQ ID NO: 16 and a light chain variable region comprising an amino acid sequence that is at least about 95%, 96%, 97%, 98%, 99% or 100% identical to the amino acid sequence of SEQ ID NO: 23 and the second and third antigen binding moieties are capable of binding to TYRP1, wherein the second and third antigen binding moieties comprise a heavy chain variable region comprising an amino acid sequence that is at least about 95%, 96%, 97%, 98%, 99% or 100% identical to the amino acid sequence of SEQ ID NO: 27 and a light chain variable region comprising an amino acid sequence that is at least about 95%, 96%, 97%, 98%, 99% or 100% identical to the amino acid sequence of SEQ ID NO: 31.

The second and the third antigen binding moiety may be fused to the Fc domain directly or through a peptide linker. In a particular embodiment the second and the third antigen binding moiety are each fused to the Fc domain through an immunoglobulin hinge region. In a specific embodiment, the immunoglobulin hinge region is a human IgG₁ hinge region. In one embodiment the second and the third antigen binding moiety and the Fc domain are part of an immunoglobulin molecule. In a particular embodiment the immunoglobulin molecule is an IgG class immunoglobulin. In an even more particular embodiment the immunoglobulin is an IgG₁ subclass immunoglobulin. In another embodiment the immunoglobulin is an IgG₄ subclass immunoglobulin. In a further particular embodiment the immunoglobulin is a human immunoglobulin. In other embodiments the immunoglobulin is a chimeric immunoglobulin or a humanized immunoglobulin. In one embodiment, the protease-activatable T cell activating bispecific molecule essentially consists of an immunoglobulin molecule capable of binding to a target cell antigen, and an antigen binding moiety capable of binding to CD3 wherein the antigen binding moiety is a Fab molecule, particularly a crossover Fab molecule, fused to the N-terminus of one of the immunoglobulin heavy chains, optionally via a peptide linker.

In a particular embodiment, the first and the third antigen binding moiety are each fused at the C-terminus of the Fab heavy chain to the N-terminus of one of the subunits of the Fc domain, and the second antigen binding moiety is fused at the C-terminus of the Fab heavy chain to the N-terminus of the Fab heavy chain of the first antigen binding moiety. In a specific such embodiment, the protease-activatable T cell activating bispecific molecule essentially consists of a first, a second and a third antigen binding moiety, an Fc domain composed of a first and a second subunit, and optionally one or more peptide linkers, wherein the second antigen binding moiety is fused at the C-terminus of the Fab heavy chain to the N-terminus of the Fab heavy chain of the first antigen binding moiety, and the first antigen binding moiety is fused at the C-terminus of the Fab heavy chain to the N-terminus of the first subunit of the Fc domain, and wherein the third antigen binding moiety is fused at the C-terminus of the Fab heavy chain to the N-terminus of the second subunit of the Fc domain. Optionally, the Fab light chain of the first antigen binding moiety and the Fab light chain of the second antigen binding moiety may additionally be fused to each other.

In one embodiment the present invention provides a protease-activatable T cell activating bispecific molecule comprising
(i) a first antigen binding moiety which is a Fab molecule capable of binding to CD3, comprising the heavy chain complementarity determining region (CDR) 1 of SEQ ID NO: 2, the heavy chain CDR 2 of SEQ ID NO: 4, the heavy chain CDR 3 of SEQ ID NO: 10, the light chain CDR 1 of SEQ ID NO: 20, the light chain CDR 2 of SEQ ID NO: 21 and the light chain CDR 3 of SEQ ID NO: 22, wherein the first antigen binding moiety is a crossover Fab molecule wherein either the variable or the constant regions, particularly the constant regions, of the Fab light chain and the Fab heavy chain are exchanged;
(ii) a second and a third antigen binding moiety each of which is a Fab molecule capable of binding to FolR1 comprising the heavy chain CDR 1 of SEQ ID NO: 54, the heavy chain CDR 2 of SEQ ID NO: 55, the heavy chain CDR 3 of SEQ ID NO: 56, the light chain CDR 1 of SEQ ID NO: 20, the light chain CDR 2 of SEQ ID NO: 21 and the light chain CDR3 of SEQ ID NO: 22.

In one embodiment the present invention provides a protease-activatable T cell activating bispecific molecule comprising
(i) a first antigen binding moiety which is a Fab molecule capable of binding to CD3 comprising a heavy chain variable region comprising an amino acid sequence that is at least about 95%, 96%, 97%, 98%, 99% or 100% identical to the amino acid sequence of SEQ ID NO: 16 and a light chain variable region comprising an amino acid sequence that is at least about 95%, 96%, 97%, 98%, 99% or 100% identical to the amino acid sequence of SEQ ID NO: 23, wherein the first antigen binding moiety is a crossover Fab molecule wherein either the variable or the constant regions, particularly the constant regions, of the Fab light chain and the Fab heavy chain are exchanged;
(ii) a second and a third antigen binding moiety each of which is a Fab molecule capable of binding to FolR1 comprising heavy chain variable region comprising an amino acid sequence that is at least about 95%, 96%, 97%, 98%, 99% or 100% identical to the amino acid sequence of SEQ ID NO: 53 and a light chain variable region comprising an amino acid sequence that is at least about 95%, 96%, 97%, 98%, 99% or 100% identical to the amino acid sequence of SEQ ID NO: 23.

In one embodiment the present invention provides a protease-activatable T cell activating bispecific molecule comprising
(i) a first antigen binding moiety which is a Fab molecule capable of binding to CD3, comprising the heavy chain complementarity determining region (CDR) 1 of SEQ ID NO: 2, the heavy chain CDR 2 of SEQ ID NO: 4, the heavy chain CDR 3 of SEQ ID NO: 10, the light chain CDR 1 of SEQ ID NO: 20, the light chain CDR 2 of SEQ ID NO: 21 and the light chain CDR 3 of SEQ ID NO: 22, wherein the first antigen binding moiety is a crossover Fab molecule wherein either the variable or the constant regions, particularly the constant regions, of the Fab light chain and the Fab heavy chain are exchanged;
(ii) a second and a third antigen binding moiety each of which is a Fab molecule capable of binding to TYRP1 comprising the heavy chain CDR 1 of SEQ ID NO: 24, the heavy chain CDR 2 of SEQ ID NO: 25, the heavy chain CDR 3 of SEQ ID NO: 26, the light chain CDR 1 of SEQ ID NO: 28, the light chain CDR 2 of SEQ ID NO: 29 and the light chain CDR3 of SEQ ID NO: 30.

The protease-activatable T cell activating bispecific molecule according to any of the ten above embodiments may further comprise (iii) an Fc domain composed of a first and a second subunit capable of stable association, wherein the second antigen binding moiety is fused at the C-terminus of the Fab heavy chain to the N-terminus of the Fab heavy chain of the first antigen binding moiety, and the first antigen binding moiety is fused at the C-terminus of the Fab heavy chain to the N-terminus of the first subunit of the Fc domain, and wherein the third antigen binding moiety is fused at the C-terminus of the Fab heavy chain to the N-terminus of the second subunit of the Fc domain.

In some of the protease-activatable T cell activating bispecific molecule of the invention, the Fab light chain of the first antigen binding moiety and the Fab light chain of the second antigen binding moiety are fused to each other, optionally via a linker peptide. Depending on the configuration of the first and the second antigen binding moiety, the Fab light chain of the first antigen binding moiety may be fused at its C-terminus to the N-terminus of the Fab light chain of the second antigen binding moiety, or the Fab light chain of the second antigen binding moiety may be fused at its C-terminus to the N-terminus of the Fab light chain of the first antigen binding moiety. Fusion of the Fab light chains of the first and the second antigen binding moiety further reduces mispairing of unmatched Fab heavy and light chains, and also reduces the number of plasmids needed for expression of some of the protease-activatable T cell activating bispecific molecule of the invention.

In certain embodiments the protease-activatable T cell activating bispecific molecule comprises a polypeptide wherein the Fab light chain variable region of the first antigen binding moiety shares a carboxy-terminal peptide bond with the Fab heavy chain constant region of the first antigen binding moiety (i.e. a the first antigen binding moiety comprises a crossover Fab heavy chain, wherein the heavy chain variable region is replaced by a light chain variable region), which in turn shares a carboxy-terminal peptide bond with an Fc domain subunit (VL₍₁₎-CH1₍₁₎-CH2-CH3(-CH4)), and a polypeptide wherein a the Fab heavy chain of the second antigen binding moiety shares a carboxy-terminal peptide bond with an Fc domain subunit (VH₍₂₎-CH1₍₂₎-CH2-CH3(-CH4)). In some embodiments the protease-activatable T cell activating bispecific molecule further comprises a polypeptide wherein the Fab heavy chain variable region of the first antigen binding moiety shares a carboxy-terminal peptide bond with the Fab light chain constant region of the first antigen binding moiety (VH₍₁₎-CL₍₁₎) and the Fab light chain polypeptide of the second antigen binding moiety (VL₍₂₎-CL₍₂₎). In certain embodiments the polypeptides are covalently linked, e.g., by a disulfide bond.

In alternative embodiments the protease-activatable T cell activating bispecific molecule comprises a polypeptide wherein the Fab heavy chain variable region of the first antigen binding moiety shares a carboxy-terminal peptide bond with the Fab light chain constant region of the first antigen binding moiety (i.e. the first antigen binding moiety comprises a crossover Fab heavy chain, wherein the heavy chain constant region is replaced by a light chain constant region), which in turn shares a carboxy-terminal peptide bond with an Fc domain subunit (VH₍₁₎-CL₍₁₎-CH2-CH3(-CH4)), and a polypeptide wherein the Fab heavy chain of the second antigen binding moiety shares a carboxy-terminal peptide bond with an Fc domain subunit (VH₍₂₎-CH1₍₂₎-CH2-CH3(-CH4)). In some embodiments the protease-activatable T cell activating bispecific molecule further comprises a polypeptide wherein the Fab light chain variable region of the first antigen binding moiety shares a carboxy-terminal peptide bond with the Fab heavy chain constant region of the first antigen binding moiety (VL₍₁₎-CH1₍₁₎) and the Fab light chain polypeptide of the second antigen binding moiety (VL₍₂₎-CL₍₂₎). In certain embodiments the polypeptides are covalently linked, e.g., by a disulfide bond.

In some embodiments, the protease-activatable T cell activating bispecific molecule comprises a polypeptide wherein the Fab light chain variable region of the first antigen binding moiety shares a carboxy-terminal peptide bond with the Fab heavy chain constant region of the first antigen binding moiety (i.e. the first antigen binding moiety comprises a crossover Fab heavy chain, wherein the heavy chain variable region is replaced by a light chain variable region), which in turn shares a carboxy-terminal peptide bond with the Fab heavy chain of the second antigen binding moiety, which in turn shares a carboxy-terminal peptide bond with an Fc domain subunit (VL₍₁₎-CH1₍₁₎-VH₍₂₎-CH1₍₂₎-CH2-CH3(-CH4)). In other embodiments, the protease-activatable T cell activating bispecific molecule comprises a polypeptide wherein the Fab heavy chain variable region of the first antigen binding moiety shares a carboxy-terminal peptide bond with the Fab light chain constant region of the first antigen binding moiety (i.e. the first antigen binding moiety comprises a crossover Fab heavy chain, wherein the heavy chain constant region is replaced by a light chain constant region), which in turn shares a carboxy-terminal peptide bond with the Fab heavy chain of the second antigen binding moiety, which in turn shares a carboxy-terminal peptide bond with an Fc domain subunit (VH₍₁₎-CL₍₁₎-VH₍₂₎-CH1₍₂₎-CH2-CH3(-CH4)). In still other embodiments, the protease-activatable T cell activating bispecific molecule comprises a polypeptide wherein the Fab heavy chain of the second antigen binding moiety shares a carboxy-terminal peptide bond with the Fab light chain variable region of the first antigen binding moiety which in turn shares a carboxy-terminal peptide bond with the Fab heavy chain constant region of the first antigen binding moiety (i.e. the first antigen binding moiety comprises a crossover Fab heavy chain, wherein the heavy chain variable region is replaced by a light chain variable region), which in turn shares a carboxy-terminal peptide bond with an Fc domain subunit (VH₍₂₎-CH1₍₂₎-VL₍₁₎-CH1₍₁₎-CH2-CH3(-CH4)). In other embodiments, the protease-activatable T cell activating bispecific molecule comprises a polypeptide wherein the Fab heavy chain of the second antigen binding moiety shares a carboxy-terminal peptide bond with the Fab heavy chain variable region of the first antigen binding moiety which in turn shares a carboxy-terminal peptide bond with the Fab light chain constant region of the first antigen binding moiety (i.e. the first antigen binding moiety comprises a crossover Fab heavy chain, wherein the heavy chain constant region is replaced by a light chain constant region), which in turn shares a carboxy-terminal peptide bond with an Fc domain subunit (VH₍₂₎-CH1₍₂₎-VH₍₁₎-CL₍₁₎-CH2-CH3 (-CH4)).

In some of these embodiments the protease-activatable T cell activating bispecific molecule further comprises a crossover Fab light chain polypeptide of the first antigen binding moiety, wherein the Fab heavy chain variable region of the first antigen binding moiety shares a carboxy-terminal peptide bond with the Fab light chain constant region of the first antigen binding moiety (VH₍₁₎-CL₍₁₎), and the Fab light chain polypeptide of the second antigen binding moiety (VL₍₂₎-CL₍₂₎). In others of these embodiments the protease-activatable T cell activating bispecific molecule further comprises a crossover Fab light chain polypeptide, wherein the Fab light chain variable region of the first antigen binding moiety shares a carboxy-terminal peptide bond with the Fab heavy chain constant region of the first antigen binding moiety (VL₍₁₎-CH1₍₁₎), and the Fab light chain polypeptide of the second antigen binding moiety (VL₍₂₎-CL₍₂₎). In still others of these embodiments the protease-activatable T cell activating bispecific molecule further comprises a polypeptide wherein the Fab light chain variable region of the first antigen binding moiety shares a carboxy-terminal peptide bond with the Fab heavy chain constant region of the first antigen binding moiety which in turn shares a carboxy-terminal peptide bond with the Fab light chain polypeptide of the second antigen binding moiety (VL₍₁₎-CH1₍₁₎-VL₍₂₎-CL₍₂₎), a polypeptide wherein the Fab heavy chain variable region of the first antigen binding moiety shares a carboxy-terminal peptide bond with the Fab light chain constant region of the first antigen binding moiety which in turn shares a carboxy-terminal peptide bond with the Fab light chain polypeptide of the second antigen binding moiety (VH₍₁₎-CL₍₁₎-VL₍₂₎-CL₍₂₎), a polypeptide wherein the Fab light chain polypeptide of the second antigen binding moiety shares a carboxy-terminal peptide bond with the Fab light chain variable region of the first antigen binding moiety which in turn shares a carboxy-terminal peptide bond with the Fab heavy chain constant region of the first antigen binding moiety (VL₍₂₎-CL₍₂₎-VL₍₁₎-CH1₍₁₎), or a polypeptide wherein the Fab light chain polypeptide of the second antigen binding moiety shares a carboxy-terminal peptide bond with the Fab heavy chain variable region of the first antigen binding moiety which in turn shares a carboxy-terminal peptide bond with the Fab light chain constant region of the first antigen binding moiety (VL₍₂₎-CL₍₂₎-VH₍₁₎-CL₍₁₎).

The protease-activatable T cell activating bispecific molecule according to these embodiments may further comprise (i) an Fc domain subunit polypeptide (CH2-CH3(-CH4)), or (ii) a polypeptide wherein the Fab heavy chain of a third antigen binding moiety shares a carboxy-terminal peptide bond with an Fc domain subunit (VH₍₃₎-CH1₍₃₎-CH2-CH3(-CH4)) and the Fab light chain polypeptide of a third antigen binding moiety (VL₍₃₎-CL₍₃₎). In certain embodiments the polypeptides are covalently linked, e.g., by a disulfide bond.

According to any of the above embodiments, components of the protease-activatable T cell activating bispecific molecule (e.g., antigen binding moiety, Fc domain) may be fused directly or through various linkers, particularly peptide linkers comprising one or more amino acids, typically about 2-20 amino acids, that are described herein or are known in the art. Suitable, non-immunogenic peptide linkers include, for example, (G₄S)ₙ, (SG₄)ₙ, (G₄S)ₙ or G₄(SG₄)ₙ peptide linkers, wherein n is generally a number between 1 and 10, typically between 2 and 4.

Fe Domain

The Fc domain of the protease-activatable T cell activating bispecific molecule consists of a pair of polypeptide chains comprising heavy chain domains of an immunoglobulin molecule. For example, the Fc domain of an immunoglobulin G (IgG) molecule is a dimer, each subunit of which comprises the CH2 and CH3 IgG heavy chain constant domains. The two subunits of the Fc domain are capable of stable association with each other. In one embodiment the protease-activatable T cell activating bispecific molecule of the invention comprises not more than one Fc domain.

In one embodiment according the invention the Fc domain of the protease-activatable T cell activating bispecific molecule is an IgG Fc domain. In a particular embodiment the Fc domain is an IgG₁ Fc domain. In another embodiment the Fc domain is an IgG₄ Fc domain. In a more specific embodiment, the Fc domain is an IgG₄ Fc domain comprising an amino acid substitution at position S228 (Kabat numbering), particularly the amino acid substitution S228P. This amino acid substitution reduces in vivo Fab arm exchange of IgG₄ antibodies (see Stubenrauch et al., Drug Metabolism and Disposition 38, 84-91 (2010)). In a further particular embodiment the Fc domain is human.

Fc Domain Modifications Promoting Heterodimerization

Protease-activatable T cell activating bispecific molecules according to the invention comprise different antigen binding moieties, fused to one or the other of the two subunits of the Fc domain, thus the two subunits of the Fc domain are typically comprised in two non-identical polypeptide chains. Recombinant co-expression of these polypeptides and subsequent dimerization leads to several possible combinations of the two polypeptides. To improve the yield and purity of protease-activatable T cell activating bispecific molecules in recombinant production, it will thus be advantageous to introduce in the Fc domain of the protease-activatable T cell activating bispecific molecule a modification promoting the association of the desired polypeptides. Accordingly, in particular embodiments the Fc domain of the protease-activatable T cell activating bispecific molecule according to the invention comprises a modification promoting the association of the first and the second subunit of the Fc domain. The site of most extensive protein-protein interaction between the two subunits of a human IgG Fc domain is in the CH3 domain of the Fc domain. Thus, in one embodiment said modification is in the CH3 domain of the Fc domain.

In a specific embodiment said modification is a so-called “knob-into-hole” modification, comprising a “knob” modification in one of the two subunits of the Fc domain and a “hole” modification in the other one of the two subunits of the Fc domain.

The knob-into-hole technology is described e.g., in U.S. Pat. Nos. 5,731,168; 7,695,936; Ridgway et al., Prot Eng 9, 617-621 (1996) and Carter, J Immunol Meth 248, 7-15 (2001). Generally, the method involves introducing a protuberance (“knob”) at the interface of a first polypeptide and a corresponding cavity (“hole”) in the interface of a second polypeptide, such that the protuberance can be positioned in the cavity so as to promote heterodimer formation and hinder homodimer formation. Protuberances are constructed by replacing small amino acid side chains from the interface of the first polypeptide with larger side chains (e.g., tyrosine or tryptophan). Compensatory cavities of identical or similar size to the protuberances are created in the interface of the second polypeptide by replacing large amino acid side chains with smaller ones (e.g., alanine or threonine).

Accordingly, in a particular embodiment, in the CH3 domain of the first subunit of the Fc domain of the protease-activatable T cell activating bispecific molecule an amino acid residue is replaced with an amino acid residue having a larger side chain volume, thereby generating a protuberance within the CH3 domain of the first subunit which is positionable in a cavity within the CH3 domain of the second subunit, and in the CH3 domain of the second subunit of the Fc domain an amino acid residue is replaced with an amino acid residue having a smaller side chain volume, thereby generating a cavity within the CH3 domain of the second subunit within which the protuberance within the CH3 domain of the first subunit is positionable.

The protuberance and cavity can be made by altering the nucleic acid encoding the polypeptides, e.g., by site-specific mutagenesis, or by peptide synthesis.

In a specific embodiment, in the CH3 domain of the first subunit of the Fc domain the threonine residue at position 366 is replaced with a tryptophan residue (T366W), and in the CH3 domain of the second subunit of the Fc domain the tyrosine residue at position 407 is replaced with a valine residue (Y407V). In one embodiment, in the second subunit of the Fc domain additionally the threonine residue at position 366 is replaced with a serine residue (T366S) and the leucine residue at position 368 is replaced with an alanine residue (L368A).

In yet a further embodiment, in the first subunit of the Fc domain additionally the serine residue at position 354 is replaced with a cysteine residue (S354C), and in the second subunit of the Fc domain additionally the tyrosine residue at position 349 is replaced by a cysteine residue (Y349C). Introduction of these two cysteine residues results in formation of a disulfide bridge between the two subunits of the Fc domain, further stabilizing the dimer (Carter, J Immunol Methods 248, 7-15 (2001)).

In a particular embodiment the antigen binding moiety capable of binding to CD3 is fused (optionally via the antigen binding moiety capable of binding to a target cell antigen) to the first subunit of the Fc domain (comprising the “knob” modification). Without wishing to be bound by theory, fusion of the antigen binding moiety capable of binding to CD3 to the knob-containing subunit of the Fc domain will (further) minimize the generation of antigen binding molecules comprising two antigen binding moieties capable of binding to CD3 (steric clash of two knob-containing polypeptides).

In an alternative embodiment a modification promoting association of the first and the second subunit of the Fc domain comprises a modification mediating electrostatic steering effects, e.g., as described in PCT publication WO 2009/089004. Generally, this method involves replacement of one or more amino acid residues at the interface of the two Fc domain subunits by charged amino acid residues so that homodimer formation becomes electrostatically unfavorable but heterodimerization electrostatically favorable.

Fc Domain Modifications Reducing Fc Receptor Binding and/or Effector Function

The Fc domain confers to the protease-activatable T cell activating bispecific molecule favorable pharmacokinetic properties, including a long serum half-life which contributes to good accumulation in the target tissue and a favorable tissue-blood distribution ratio. At the same time it may, however, lead to undesirable targeting of the protease-activatable T cell activating bispecific molecule to cells expressing Fc receptors rather than to the preferred antigen-bearing cells. Moreover, the co-activation of Fc receptor signaling pathways may lead to cytokine release which, in combination with the T cell activating properties and the long half-life of the antigen binding molecule, results in excessive activation of cytokine receptors and severe side effects upon systemic administration. Activation of (Fc receptor-bearing) immune cells other than T cells may even reduce efficacy of the protease-activatable T cell activating bispecific molecule due to the potential destruction of T cells e.g., by NK cells.

Accordingly, in particular embodiments the Fc domain of the protease-activatable T cell activating bispecific molecules according to the invention exhibits reduced binding affinity to an Fc receptor and/or reduced effector function, as compared to a native IgG₁ Fc domain. In one such embodiment the Fc domain (or the protease-activatable T cell activating bispecific molecule comprising said Fc domain) exhibits less than 50%, preferably less than 20%, more preferably less than 10% and most preferably less than 5% of the binding affinity to an Fc receptor, as compared to a native IgG₁ Fc domain (or a protease-activatable T cell activating bispecific molecule comprising a native IgG₁ Fc domain), and/or less than 50%, preferably less than 20%, more preferably less than 10% and most preferably less than 5% of the effector function, as compared to a native IgG₁ Fc domain domain (or a protease-activatable T cell activating bispecific molecule comprising a native IgG₁ Fc domain). In one embodiment, the Fc domain domain (or the protease-activatable T cell activating bispecific molecule comprising said Fc domain) does not substantially bind to an Fc receptor and/or induce effector function. In a particular embodiment the Fc receptor is an Fcγ receptor. In one embodiment the Fc receptor is a human Fc receptor. In one embodiment the Fc receptor is an activating Fc receptor. In a specific embodiment the Fc receptor is an activating human Fcγ receptor, more specifically human FcγRIIIa, FcγRI or FcγRIIa, most specifically human FcγRIIIa. In one embodiment the effector function is one or more selected from the group of CDC, ADCC, ADCP, and cytokine secretion. In a particular embodiment the effector function is ADCC. In one embodiment the Fc domain domain exhibits substantially similar binding affinity to neonatal Fc receptor (FcRn), as compared to a native IgG₁ Fc domain domain. Substantially similar binding to FcRn is achieved when the Fc domain (or the protease-activatable T cell activating bispecific molecule comprising said Fc domain) exhibits greater than about 70%, particularly greater than about 80%, more particularly greater than about 90% of the binding affinity of a native IgG₁ Fc domain (or the protease-activatable T cell activating bispecific molecule comprising a native IgG₁ Fc domain) to FcRn.

In certain embodiments the Fc domain is engineered to have reduced binding affinity to an Fc receptor and/or reduced effector function, as compared to a non-engineered Fc domain. In particular embodiments, the Fc domain of the protease-activatable T cell activating bispecific molecule comprises one or more amino acid mutation that reduces the binding affinity of the Fc domain to an Fc receptor and/or effector function. Typically, the same one or more amino acid mutation is present in each of the two subunits of the Fc domain. In one embodiment the amino acid mutation reduces the binding affinity of the Fc domain to an Fc receptor. In one embodiment the amino acid mutation reduces the binding affinity of the Fc domain to an Fc receptor by at least 2-fold, at least 5-fold, or at least 10-fold. In embodiments where there is more than one amino acid mutation that reduces the binding affinity of the Fc domain to the Fc receptor, the combination of these amino acid mutations may reduce the binding affinity of the Fc domain to an Fc receptor by at least 10-fold, at least 20-fold, or even at least 50-fold. In one embodiment the protease-activatable T cell activating bispecific molecule comprising an engineered Fc domain exhibits less than 20%, particularly less than 10%, more particularly less than 5% of the binding affinity to an Fc receptor as compared to a protease-activatable T cell activating bispecific molecule comprising a non-engineered Fc domain. In a particular embodiment the Fc receptor is an Fcγ receptor. In some embodiments the Fc receptor is a human Fc receptor. In some embodiments the Fc receptor is an activating Fc receptor. In a specific embodiment the Fc receptor is an activating human Fcγ receptor, more specifically human FcγRIIIa, FcγRI or FcγRIIa, most specifically human FcγRIIIa. Preferably, binding to each of these receptors is reduced. In some embodiments binding affinity to a complement component, specifically binding affinity to C1q, is also reduced. In one embodiment binding affinity to neonatal Fc receptor (FcRn) is not reduced. Substantially similar binding to FcRn, i.e. preservation of the binding affinity of the Fc domain to said receptor, is achieved when the Fc domain (or the protease-activatable T cell activating bispecific molecule comprising said Fc domain) exhibits greater than about 70% of the binding affinity of a non-engineered form of the Fc domain (or the protease-activatable T cell activating bispecific molecule comprising said non-engineered form of the Fc domain) to FcRn. The Fc domain, or protease-activatable T cell activating bispecific molecules of the invention comprising said Fc domain, may exhibit greater than about 80% and even greater than about 90% of such affinity. In certain embodiments the Fc domain of the protease-activatable T cell activating bispecific molecule is engineered to have reduced effector function, as compared to a non-engineered Fc domain. The reduced effector function can include, but is not limited to, one or more of the following: reduced complement dependent cytotoxicity (CDC), reduced antibody-dependent cell-mediated cytotoxicity (ADCC), reduced antibody-dependent cellular phagocytosis (ADCP), reduced cytokine secretion, reduced immune complex-mediated antigen uptake by antigen-presenting cells, reduced binding to NK cells, reduced binding to macrophages, reduced binding to monocytes, reduced binding to polymorphonuclear cells, reduced direct signaling inducing apoptosis, reduced crosslinking of target-bound antibodies, reduced dendritic cell maturation, or reduced T cell priming. In one embodiment the reduced effector function is one or more selected from the group of reduced CDC, reduced ADCC, reduced ADCP, and reduced cytokine secretion. In a particular embodiment the reduced effector function is reduced ADCC. In one embodiment the reduced ADCC is less than 20% of the ADCC induced by a non-engineered Fc domain (or a protease-activatable T cell activating bispecific molecule comprising a non-engineered Fc domain).

In one embodiment the amino acid mutation that reduces the binding affinity of the Fc domain to an Fc receptor and/or effector function is an amino acid substitution. In one embodiment the Fc domain comprises an amino acid substitution at a position selected from the group of E233, L234, L235, N297, P331 and P329. In a more specific embodiment the Fc domain comprises an amino acid substitution at a position selected from the group of L234, L235 and P329. In some embodiments the Fc domain comprises the amino acid substitutions L234A and L235A. In one such embodiment, the Fc domain is an IgG₁ Fc domain, particularly a human IgG₁ Fc domain. In one embodiment the Fc domain comprises an amino acid substitution at position P329. In a more specific embodiment the amino acid substitution is P329A or P329G, particularly P329G. In one embodiment the Fc domain comprises an amino acid substitution at position P329 and a further amino acid substitution at a position selected from E233, L234, L235, N297 and P331. In a more specific embodiment the further amino acid substitution is E233P, L234A, L235A, L235E, N297A, N297D or P331S. In particular embodiments the Fc domain comprises amino acid substitutions at positions P329, L234 and L235. In more particular embodiments the Fc domain comprises the amino acid mutations L234A, L235A and P329G (“P329G LALA”). In one such embodiment, the Fc domain is an IgG₁ Fc domain, particularly a human IgG₁ Fc domain. The “P329G LALA” combination of amino acid substitutions almost completely abolishes Fcγ receptor (as well as complement) binding of a human IgG₁ Fc domain, as described in PCT publication no. WO 2012/130831, incorporated herein by reference in its entirety. WO 2012/130831 also describes methods of preparing such mutant Fc domains and methods for determining its properties such as Fc receptor binding or effector functions.

IgG₄ antibodies exhibit reduced binding affinity to Fc receptors and reduced effector functions as compared to IgG₁ antibodies. Hence, in some embodiments the Fc domain of the protease-activatable T cell activating bispecific molecules of the invention is an IgG₄ Fc domain, particularly a human IgG₄ Fc domain. In one embodiment the IgG₄ Fc domain comprises amino acid substitutions at position 5228, specifically the amino acid substitution S228P. To further reduce its binding affinity to an Fc receptor and/or its effector function, in one embodiment the IgG₄ Fc domain comprises an amino acid substitution at position L235, specifically the amino acid substitution L235E. In another embodiment, the IgG₄ Fc domain comprises an amino acid substitution at position P329, specifically the amino acid substitution P329G. In a particular embodiment, the IgG₄ Fc domain comprises amino acid substitutions at positions S228, L235 and P329, specifically amino acid substitutions S228P, L235E and P329G. Such IgG₄ Fc domain mutants and their Fcγ receptor binding properties are described in PCT publication no. WO 2012/130831, incorporated herein by reference in its entirety.

In a particular embodiment the Fc domain exhibiting reduced binding affinity to an Fc receptor and/or reduced effector function, as compared to a native IgG₁ Fc domain, is a human IgG₁ Fc domain comprising the amino acid substitutions L234A, L235A and optionally P329G, or a human IgG₄ Fc domain comprising the amino acid substitutions S228P, L235E and optionally P329G.

In certain embodiments N-glycosylation of the Fc domain has been eliminated. In one such embodiment the Fc domain comprises an amino acid mutation at position N297, particularly an amino acid substitution replacing asparagine by alanine (N297A) or aspartic acid (N297D).

In addition to the Fc domains described hereinabove and in PCT publication no. WO 2012/130831, Fc domains with reduced Fc receptor binding and/or effector function also include those with substitution of one or more of Fc domain residues 238, 265, 269, 270, 297, 327 and 329 (U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581).

Mutant Fc domains can be prepared by amino acid deletion, substitution, insertion or modification using genetic or chemical methods well known in the art. Genetic methods may include site-specific mutagenesis of the encoding DNA sequence, PCR, gene synthesis, and the like. The correct nucleotide changes can be verified for example by sequencing.

Binding to Fc receptors can be easily determined e.g., by ELISA, or by Surface Plasmon Resonance (SPR) using standard instrumentation such as a BIAcore instrument (GE Healthcare), and Fc receptors such as may be obtained by recombinant expression. A suitable such binding assay is described herein. Alternatively, binding affinity of Fc domains or cell activating bispecific antigen binding molecules comprising an Fc domain for Fc receptors may be evaluated using cell lines known to express particular Fc receptors, such as human NK cells expressing FcγIIIa receptor.

Effector function of an Fc domain, or a protease-activatable T cell activating bispecific molecule comprising an Fc domain, can be measured by methods known in the art. A suitable assay for measuring ADCC is described herein. Other examples of in vitro assays to assess ADCC activity of a molecule of interest are described in U.S. Pat. No. 5,500,362; Hellstrom et al. Proc Natl Acad Sci USA 83, 7059-7063 (1986) and Hellstrom et al., Proc Natl Acad Sci USA 82, 1499-1502 (1985); U.S. Pat. No. 5,821,337; Bruggemann et al., J Exp Med 166, 1351-1361 (1987).

Alternatively, non-radioactive assays methods may be employed (see, for example, ACTI™ non-radioactive cytotoxicity assay for flow cytometry (CellTechnology, Inc. Mountain View, Calif.); and CytoTox 96® non-radioactive cytotoxicity assay (Promega, Madison, Wis.)). Useful effector cells for such assays include peripheral blood mononuclear cells (PBMC) and Natural Killer (NK) cells. Alternatively, or additionally, ADCC activity of the molecule of interest may be assessed in vivo, e.g., in a animal model such as that disclosed in Clynes et al., Proc Natl Acad Sci USA 95, 652-656 (1998).

In some embodiments, binding of the Fc domain to a complement component, specifically to C1q, is reduced. Accordingly, in some embodiments wherein the Fc domain is engineered to have reduced effector function, said reduced effector function includes reduced CDC. C1q binding assays may be carried out to determine whether the protease-activatable T cell activating bispecific molecule is able to bind C1q and hence has CDC activity. See e.g., C1q and C3c binding ELISA in WO 2006/029879 and WO 2005/100402. To assess complement activation, a CDC assay may be performed (see, for example, Gazzano-Santoro et al., J Immunol Methods 202, 163 (1996); Cragg et al., Blood 101, 1045-1052 (2003); and Cragg and Glennie, Blood 103, 2738-2743 (2004)).

Antigen Binding Moieties

The antigen binding molecule of the invention is bispecific, i.e. it comprises at least two antigen binding moieties capable of specific binding to two distinct antigenic determinants. According to the invention, the antigen binding moieties are Fab molecules (i.e. antigen binding domains composed of a heavy and a light chain, each comprising a variable and a constant region). In one embodiment said Fab molecules are human. In another embodiment said Fab molecules are humanized. In yet another embodiment said Fab molecules comprise human heavy and light chain constant regions.

At least one of the antigen binding moieties is a crossover Fab molecule. Such modification prevent mispairing of heavy and light chains from different Fab molecules, thereby improving the yield and purity of the protease-activatable T cell activating bispecific molecule of the invention in recombinant production. In a particular crossover Fab molecule useful for the protease-activatable T cell activating bispecific molecule of the invention, the constant regions of the Fab light chain and the Fab heavy chain are exchanged. In another crossover Fab molecule useful for the protease-activatable T cell activating bispecific molecule of the invention, the variable regions of the Fab light chain and the Fab heavy chain are exchanged.

In a particular embodiment according to the invention, the protease-activatable T cell activating bispecific molecule is capable of simultaneous binding to a target cell antigen, particularly a tumor cell antigen, and CD3. In one embodiment, the protease-activatable T cell activating bispecific molecule is capable of crosslinking a T cell and a target cell by simultaneous binding to a target cell antigen and CD3. In an even more particular embodiment, such simultaneous binding results in lysis of the target cell, particularly a tumor cell. In one embodiment, such simultaneous binding results in activation of the T cell. In other embodiments, such simultaneous binding results in a cellular response of a T lymphocyte, particularly a cytotoxic T lymphocyte, selected from the group of: proliferation, differentiation, cytokine secretion, cytotoxic effector molecule release, cytotoxic activity, and expression of activation markers. In one embodiment, binding of the protease-activatable T cell activating bispecific molecule to CD3 without simultaneous binding to the target cell antigen does not result in T cell activation.

In one embodiment, the protease-activatable T cell activating bispecific molecule is capable of re-directing cytotoxic activity of a T cell to a target cell. In a particular embodiment, said re-direction is independent of MHC-mediated peptide antigen presentation by the target cell and and/or specificity of the T cell.

Particularly, a T cell according to any of the embodiments of the invention is a cytotoxic T cell. In some embodiments the T cell is a CD4⁺ or a CD8⁺ T cell, particularly a CD8⁺ T cell.

CD3 Binding Moiety

The protease-activatable T cell activating bispecific molecule of the invention comprises at least one antigen binding moiety capable of binding to CD3 (also referred to herein as an “CD3 antigen binding moiety” or “first antigen binding moiety”). In a particular embodiment, the protease-activatable T cell activating bispecific molecule comprises not more than one antigen binding moiety capable of binding to CD3. In one embodiment the protease-activatable T cell activating bispecific molecule provides monovalent binding to CD3. The CD3 antigen binding is a crossover Fab molecule, i.e. a Fab molecule wherein either the variable or the constant regions of the Fab heavy and light chains are exchanged. In embodiments where there is more than one antigen binding moiety capable of binding to a target cell antigen comprised in the protease-activatable T cell activating bispecific molecule, the antigen binding moiety capable of binding to CD3 preferably is a crossover Fab molecule and the antigen binding moieties capable of binding to a target cell antigen are conventional Fab molecules.

In a particular embodiment CD3 is human CD3 or cynomolgus CD3, most particularly human CD3. In a particular embodiment the CD3 antigen binding moiety is cross-reactive for (i.e. specifically binds to) human and cynomolgus CD3. In some embodiments, the first antigen binding moiety is capable of binding to the epsilon subunit of CD3.

The CD3 antigen binding moiety comprises at least one heavy chain complementarity determining region (CDR) selected from the group consisting of SEQ ID NO: 2, SEQ ID NO: 4 and SEQ ID NO: 10 and at least one light chain CDR selected from the group of SEQ ID NO: 20, SEQ ID NO: 21, SEQ ID NO: 22.

In one embodiment the CD3 antigen binding moiety comprises the heavy chain CDR1 of SEQ ID NO: 2, the heavy chain CDR2 of SEQ ID NO: 4, the heavy chain CDR3 of SEQ ID NO: 10, the light chain CDR1 of SEQ ID NO: 20, the light chain CDR2 of SEQ ID NO: 21, and the light chain CDR3 of SEQ ID NO: 22.

In one embodiment the CD3 antigen binding moiety comprises a heavy chain variable region sequence that is at least about 95%, 96%, 97%, 98%, 99% or 100% identical to the amino acid sequence of SEQ ID NO: 16, and a light chain variable region sequence that is at least about 95%, 96%, 97%, 98%, 99% or 100% identical to the amino acid sequence of SEQ ID NO: 23. In one embodiment the CD3 antigen binding moiety comprises the heavy chain variable region sequence of SEQ ID NO: 16 and the light chain variable region sequence of SEQ ID NO: 23.

Target Cell Antigen Binding Moiety

The protease-activatable T cell activating bispecific molecule of the invention comprises at least one antigen binding moiety capable of binding to a target cell antigen (also referred to herein as an “target cell antigen binding moiety” or “second” or “third” antigen binding moiety). In certain embodiments, the protease-activatable T cell activating bispecific molecule comprises two antigen binding moieties capable of binding to a target cell antigen. In a particular such embodiment, each of these antigen binding moieties specifically binds to the same antigenic determinant. In an even more particular embodiment, all of these antigen binding moieties are identical. In one embodiment, the protease-activatable T cell activating bispecific molecule comprises an immunoglobulin molecule capable of binding to a target cell antigen. In one embodiment the protease-activatable T cell activating bispecific molecule comprises not more than two antigen binding moieties capable of binding to a target cell antigen.

In a preferred embodiment, the target cell antigen binding moiety is a Fab molecule, particularly a conventional Fab molecule that binds to a specific antigenic determinant and is able to direct the Protease-activatable T cell activating bispecific molecule to a target site, for example to a specific type of tumor cell that bears the antigenic determinant.

In certain embodiments the target cell antigen binding moiety specifically binds to a cell surface antigen. In a particular embodiment the target cell antigen binding moiety specifically binds to a Folate Receptor 1 (FolR1) on the surface of a target cell. In another specific such embodiment the target cell antigen binding moiety specifically binds to Tyrosinase Related Protein 1 (TYRP1), specifically, a human TYRP1.

In certain embodiments the target cell antigen binding moiety is directed to an antigen associated with a pathological condition, such as an antigen presented on a tumor cell or on a virus-infected cell. Suitable antigens are cell surface antigens, for example, but not limited to, cell surface receptors. In particular embodiments the antigen is a human antigen. In a specific embodiment the target cell antigen is selected from Folate Receptor 1 (FolR1) and Tyrosinase Related Protein 1 (TYRP1).

In particular embodiments the protease-activatable T cell activating bispecific molecule comprises at least one antigen binding moiety that is specific for FolR1. In one embodiment the FolR1 is a human FolR1. In one embodiment, the protease-activatable T cell activating bispecific molecule comprises at least one antigen binding moiety that is specific for human FolR1 and does not bind to human FolR2 or human FolR3. In one embodiment, the antigen binding moiety that is specific for FolR1 comprises at least one heavy chain complementarity determining region (CDR) selected from the group consisting of SEQ ID NO: 54, SEQ ID NO: 55 and SEQ ID NO: 56 and at least one light chain CDR selected from the group of SEQ ID NO: 20, SEQ ID NO: 21 and SEQ ID NO: 22.

In one embodiment, the antigen binding moiety that is specific for FolR1 comprises the heavy chain CDR1 of SEQ ID NO: 54, the heavy chain CDR2 of SEQ ID NO: 55, the heavy chain CDR3 of SEQ ID NO: 56, the light chain CDR1 of SEQ ID NO: 20, the light chain CDR2 of SEQ ID NO: 21, and the light chain CDR3 of SEQ ID NO: 22.

In a further embodiment, the antigen binding moiety that is specific for FolR1 comprises a heavy chain variable region sequence that is at least about 95%, 96%, 97%, 98%, 99% or 100% identical to SEQ ID NO: 53 and a light chain variable region sequence that is at least about 95%, 96%, 97%, 98%, 99% or 100% identical to SEQ ID NO: 23, or variants thereof that retain functionality.

In one embodiment, the antigen binding moiety that is specific for FolR1 comprises the heavy chain variable region comprising an amino acid sequence of SEQ ID NO: 53 and the light chain variable region comprising an amino acid sequence of SEQ ID NO: 23.

Masking Moiety

The protease-activatable T cell activating bispecific molecule of the invention comprises at least one masking moiety. Others have tried to mask binding of an antibody by capping the binding moiety with a fragment of the antigen recognized by the binding moiety (e.g., WO2013128194). This approach has several limitations. For example, using the antigen allows for less flexibility in reducing the affinity of the binding moiety. This is so because the affinity has to be high enough to be reliably masked by the antigen mask. Also, dissociated antigen could potentially bind to and interact with its cognate receptor(s) in vivo and cause undesirable signals to the cell expressing such receptor. In contrast, the approach described herein uses an anti-idiotype antibody or fragment thereof as a mask. Two countervailing considerations for designing an effective masking moiety are 1. effectiveness of the masking and 2. reversibility of the masking. If the affinity is too low, masking would be inefficient. However, if the affinity is too high, the masking process might not be readily reversible. It was not predictable whether a high affinity anti-idiotype mask or a low affinity anti-idiotype mask would work better. As described herein, higher affinity masking moieties performed overall better in masking the antigen binding side and, at the same time, could be effectively removed for activation of the molecule. In one embodiment, the anti-idiotype mask has a KD of 1-8 nM. In one embodiment, anti-idiotype mask has a KD of 2 nM at 37° C. In one specific embodiment, the masking moiety recognizes the idiotype of the first antigen binding moiety capable of binding to a CD3, e.g., a human CD3. In one specific embodiment, the masking moiety recognizes the idiotype of the second antigen binding moiety capable of binding to a target cell antigen.

In one embodiment, the masking moiety masks a CD3-binding moiety and comprises at least one of the heavy chain CDR1 of SEQ ID NO: 2, the heavy chain CDR2 of SEQ ID NO: 4, the heavy chain CDR3 of SEQ ID NO: 10, the light chain CDR1 of SEQ ID NO: 20, the light chain CDR2 of SEQ ID NO: 21, and the light chain CDR3 of SEQ ID NO: 22. In one embodiment, the masking moiety comprises the heavy chain CDR1 of SEQ ID NO: 2, the heavy chain CDR2 of SEQ ID NO: 4, the heavy chain CDR3 of SEQ ID NO: 10, the light chain CDR1 of SEQ ID NO: 20, the light chain CDR2 of SEQ ID NO: 21, and the light chain CDR3 of SEQ ID NO: 22.

In one embodiment, the masking moiety masks a CD3-binding moiety and comprises a polypeptide sequence that is at least about 95%, 96%, 97%, 98%, 99% or 100% identical to SEQ ID NO: 16. In one embodiment, the masking moiety masks a CD3-binding moiety and comprises the polypeptide sequence of SEQ ID NO: 23.

In one embodiment masking moiety comprising at least one of the heavy chain CDR1 of SEQ ID NO: 58, a heavy chain CDR2 selected from the group consisting of SEQ ID NO: 59, SEQ ID NO: 84 and SEQ ID NO: 86, a heavy chain CDR3 of SEQ ID NO: 60, a light chain CDR1 selected from the group consisting of SEQ ID NO: 62 and SEQ ID NO: 82, the light chain CDR2 of SEQ ID NO: 63, and a light chain CDR3 selected from the group consisting of SEQ ID NO: 64 and SEQ ID NO: 88.

In one embodiment the masking moiety comprising at least one of the heavy chain CDR1 of SEQ ID NO: 58, the heavy chain CDR2 of SEQ ID NO: 59, the heavy chain CDR3 of SEQ ID NO: 60, the light chain CDR1 of SEQ ID NO: 62, the light chain CDR2 of SEQ ID NO: 63, and the light chain CDR3 of SEQ ID NO: 64. In one embodiments the masking moiety comprising a heavy chain variable region sequence that is at least about 95%, 96%, 97%, 98%, 99% or 100% identical to SEQ ID NO: 57 and a light chain variable region sequence that is at least about 95%, 96%, 97%, 98%, 99% or 100% identical to SEQ ID NO: 61, or variants thereof that retain functionality.

In one embodiment the masking moiety comprising at least one of the heavy chain CDR1 of SEQ ID NO: 58, the heavy chain CDR2 of SEQ ID NO: 59, the heavy chain CDR3 of SEQ ID NO: 60, the light chain CDR1 of SEQ ID NO: 82, the light chain CDR2 of SEQ ID NO: 63, and the light chain CDR3 of SEQ ID NO: 64.

In one embodiment the masking moiety comprising at least one of the heavy chain CDR1 of SEQ ID NO: 58, the heavy chain CDR2 of SEQ ID NO: 84, the heavy chain CDR3 of SEQ ID NO: 60, the light chain CDR1 of SEQ ID NO: 82, the light chain CDR2 of SEQ ID NO: 63, and the light chain CDR3 of SEQ ID NO: 64.

In one embodiment the masking moiety comprising at least one of the heavy chain CDR1 of SEQ ID NO: 58, the heavy chain CDR2 of SEQ ID NO: 86, the heavy chain CDR3 of SEQ ID NO: 60, the light chain CDR1 of SEQ ID NO: 82, the light chain CDR2 of SEQ ID NO: 63, and the light chain CDR3 of SEQ ID NO: 64.

In one embodiment the masking moiety comprising at least one of the heavy chain CDR1 of SEQ ID NO: 59, the heavy chain CDR2 of SEQ ID NO: 86, the heavy chain CDR3 of SEQ ID NO: 60, the light chain CDR1 of SEQ ID NO: 62, the light chain CDR2 of SEQ ID NO: 63, and the light chain CDR3 of SEQ ID NO: 88.

In a preferred embodiment, the masking moiety is humanized. In a preferred embodiment, the idiotype-specific polypeptide for reversibly concealing an anti-CD3 antigen binding site of a molecule is humanized. Methods to humanize immunoglobulins are well known in the art and herein described.

In one embodiment provided is a idiotype-specific polypeptide for reversibly concealing an anti-CD3 antigen binding site of a molecule, wherein the idiotype-specific polypeptide comprises a heavy chain variable region sequence that is at least about 95%, 96%, 97%, 98%, 99% or 100% identical to an amino acid sequence selected from the group consisting of SEQ ID NO: 79, SEQ ID NO:83, SEQ ID NO:84, SEQ ID NO:85 and SEQ ID NO:89, and a light chain variable region sequence that is at least about 95%, 96%, 97%, 98%, 99% or 100% identical to an amino acid sequence selected from the group consisting of SEQ ID NO: 80, SEQ ID NO:81, SEQ ID NO:87 and SEQ ID NO: 90.

In a preferred embodiment provided is a idiotype-specific polypeptide for reversibly concealing an anti-CD3 antigen binding site of a molecule, wherein the idiotype-specific polypeptide comprises a heavy chain variable region sequence that is at least about 95%, 96%, 97%, 98%, 99% or 100% identical to an amino acid sequence selected from the group consisting of SEQ ID NO: 79, SEQ ID NO:83 and SEQ ID NO:85, and a light chain variable region sequence that is at least about 95%, 96%, 97%, 98%, 99% or 100% identical to an amino acid sequence selected from the group consisting of SEQ ID NO: 80 and SEQ ID NO:81.

In one embodiment, provided is a idiotype-specific polypeptide for reversibly concealing an anti-CD3 antigen binding site of a molecule, wherein the idiotype-specific polypeptide comprises a heavy chain variable region sequence that is at least about 95%, 96%, 97%, 98%, 99% or 100% identical to SEQ ID NO: 79 and a light chain variable region sequence that is at least about 95%, 96%, 97%, 98%, 99% or 100% identical to SEQ ID NO: 80, In a preferred embodiment, provided is a idiotype-specific polypeptide for reversibly concealing an anti-CD3 antigen binding site of a molecule, wherein the idiotype-specific polypeptide comprises the heavy chain variable region sequence of SEQ ID NO: 79 and the light chain variable region sequence of SEQ ID NO: 80,

In one embodiment, provided is a idiotype-specific polypeptide for reversibly concealing an anti-CD3 antigen binding site of a molecule, wherein the idiotype-specific polypeptide comprises a heavy chain variable region sequence that is at least about 95%, 96%, 97%, 98%, 99% or 100% identical to SEQ ID NO: 79 and a light chain variable region sequence that is at least about 95%, 96%, 97%, 98%, 99% or 100% identical to SEQ ID NO: 81, In a preferred embodiment, provided is a idiotype-specific polypeptide for reversibly concealing an anti-CD3 antigen binding site of a molecule, wherein the idiotype-specific polypeptide comprises the heavy chain variable region sequence of SEQ ID NO: 79 and the light chain variable region sequence of SEQ ID NO: 81,

In one embodiment, provided is a idiotype-specific polypeptide for reversibly concealing an anti-CD3 antigen binding site of a molecule, wherein the idiotype-specific polypeptide comprises a heavy chain variable region sequence that is at least about 95%, 96%, 97%, 98%, 99% or 100% identical to SEQ ID NO: 83 and a light chain variable region sequence that is at least about 95%, 96%, 97%, 98%, 99% or 100% identical to SEQ ID NO: 81, In a preferred embodiment, provided is a idiotype-specific polypeptide for reversibly concealing an anti-CD3 antigen binding site of a molecule, wherein the idiotype-specific polypeptide comprises the heavy chain variable region sequence of SEQ ID NO: 83 and the light chain variable region sequence of SEQ ID NO: 81,

In one embodiment, provided is a idiotype-specific polypeptide for reversibly concealing an anti-CD3 antigen binding site of a molecule, wherein the idiotype-specific polypeptide comprises a heavy chain variable region sequence that is at least about 95%, 96%, 97%, 98%, 99% or 100% identical to SEQ ID NO: 85 and a light chain variable region sequence that is at least about 95%, 96%, 97%, 98%, 99% or 100% identical to SEQ ID NO: 81, In a preferred embodiment, provided is a idiotype-specific polypeptide for reversibly concealing an anti-CD3 antigen binding site of a molecule, wherein the idiotype-specific polypeptide comprises the heavy chain variable region sequence of SEQ ID NO: 85 and the light chain variable region sequence of SEQ ID NO: 81,

In one embodiment, provided is a idiotype-specific polypeptide for reversibly concealing an anti-CD3 antigen binding site of a molecule, wherein the idiotype-specific polypeptide comprises a heavy chain variable region sequence that is at least about 95%, 96%, 97%, 98%, 99% or 100% identical to SEQ ID NO: 84 and a light chain variable region sequence that is at least about 95%, 96%, 97%, 98%, 99% or 100% identical to SEQ ID NO: 87, In one embodiment, provided is a idiotype-specific polypeptide for reversibly concealing an anti-CD3 antigen binding site of a molecule, wherein the idiotype-specific polypeptide comprises the heavy chain variable region sequence of SEQ ID NO: 84 and the light chain variable region sequence of SEQ ID NO: 87,

In one embodiment, provided is a idiotype-specific polypeptide for reversibly concealing an anti-CD3 antigen binding site of a molecule, wherein the idiotype-specific polypeptide comprises a heavy chain variable region sequence that is at least about 95%, 96%, 97%, 98%, 99% or 100% identical to SEQ ID NO: 89 and a light chain variable region sequence that is at least about 95%, 96%, 97%, 98%, 99% or 100% identical to SEQ ID NO: 90, In one embodiment, provided is a idiotype-specific polypeptide for reversibly concealing an anti-CD3 antigen binding site of a molecule, wherein the idiotype-specific polypeptide comprises the heavy chain variable region sequence of SEQ ID NO: 89 and the light chain variable region sequence of SEQ ID NO: 90, In one embodiments the masking moiety is an anti-idiotypic scFv comprising a polypeptide sequence that is at least about 95%, 96%, 97%, 98%, 99% or 100% identical to SEQ ID NO: 91.

In one embodiment, the anti-idiotypic scFv comprises the polypeptide sequence of SEQ ID NO: 91.

In one embodiments the masking moiety is an anti-idiotypic scFv comprising a polypeptide sequence that is at least about 95%, 96%, 97%, 98%, 99% or 100% identical to SEQ ID NO: 92.

In one embodiment, the anti-idiotypic scFv comprises the polypeptide sequence of SEQ ID NO: 92.

In one embodiments the masking moiety is an anti-idiotypic scFv comprising a polypeptide sequence that is at least about 95%, 96%, 97%, 98%, 99% or 100% identical to SEQ ID NO: 93.

In one embodiment, the anti-idiotypic scFv comprises the polypeptide sequence of SEQ ID NO: 93.

In one embodiments the masking moiety is an anti-idiotypic scFv comprising a polypeptide sequence that is at least about 95%, 96%, 97%, 98%, 99% or 100% identical to SEQ ID NO: 94.

In one embodiment, the anti-idiotypic scFv comprises the polypeptide sequence of SEQ ID NO: 94.

Protease-Activatable T Cell Activating Bispecific Molecules Capable of Binding to CD3 and FolR1

The first antigen binding moiety capable of binding to CD3 as described herein above, the second antigen binding moiety capable of binding to FolR1 as described herein above, the Fc domain as described herein above and the masking moiety as described herein above can be fused to each other in a variety of configurations. Exemplary configurations and sequences are disclosed herein below.

In one embodiment the protease-activatable T cell activating bispecific molecule comprises a polypeptide sequence that is at least about 95%, 96%, 97%, 98%, 99% or 100% identical to SEQ ID NO: 65, a polypeptide sequence that is at least about 95%, 96%, 97%, 98%, 99% or 100% identical to SEQ ID NO: 66 and a polypeptide sequence that is at least about 95%, 96%, 97%, 98%, 99% or 100% identical to SEQ ID NO: 67.

In one embodiment the protease-activatable T cell activating bispecific molecule comprises the polypeptide sequence of SEQ ID NO: 65, the polypeptide sequence of SEQ ID NO: 66 and the polypeptide sequence of SEQ ID NO: 67.

In one embodiment the protease-activatable T cell activating bispecific molecule comprises a polypeptide sequence that is at least about 95%, 96%, 97%, 98%, 99% or 100% identical to SEQ ID NO: 74, a polypeptide sequence that is at least about 95%, 96%, 97%, 98%, 99% or 100% identical to SEQ ID NO: 66 and a polypeptide sequence that is at least about 95%, 96%, 97%, 98%, 99% or 100% identical to SEQ ID NO: 67.

In one embodiment the protease-activatable T cell activating bispecific molecule comprises the polypeptide sequence of SEQ ID NO: 74, the polypeptide sequence of SEQ ID NO: 66 and the polypeptide sequence of SEQ ID NO: 67.

In one embodiment the protease-activatable T cell activating bispecific molecule comprises a polypeptide sequence that is at least about 95%, 96%, 97%, 98%, 99% or 100% identical to SEQ ID NO: 76, a polypeptide sequence that is at least about 95%, 96%, 97%, 98%, 99% or 100% identical to SEQ ID NO: 66 and a polypeptide sequence that is at least about 95%, 96%, 97%, 98%, 99% or 100% identical to SEQ ID NO: 67.

In one embodiment the protease-activatable T cell activating bispecific molecule comprises the polypeptide sequence of SEQ ID NO: 76, the polypeptide sequence of SEQ ID NO: 66 and the polypeptide sequence of SEQ ID NO: 67.

In one embodiment the protease-activatable T cell activating bispecific molecule comprises a polypeptide sequence that is at least about 95%, 96%, 97%, 98%, 99% or 100% identical to SEQ ID NO: 95, a polypeptide sequence that is at least about 95%, 96%, 97%, 98%, 99% or 100% identical to SEQ ID NO: 66 and a polypeptide sequence that is at least about 95%, 96%, 97%, 98%, 99% or 100% identical to SEQ ID NO: 67.

In one embodiment the protease-activatable T cell activating bispecific molecule comprises the polypeptide sequence of SEQ ID NO: 95, the polypeptide sequence of SEQ ID NO: 66 and the polypeptide sequence of SEQ ID NO: 67. In one embodiment the protease-activatable T cell activating bispecific molecule comprises one polypeptide of SEQ ID NO: 95, one polypeptide of SEQ ID NO: 66 and two polypeptide of SEQ ID NO: 67.

In one embodiment the protease-activatable T cell activating bispecific molecule comprises a polypeptide sequence that is at least about 95%, 96%, 97%, 98%, 99% or 100% identical to SEQ ID NO: 96, a polypeptide sequence that is at least about 95%, 96%, 97%, 98%, 99% or 100% identical to SEQ ID NO: 66 and a polypeptide sequence that is at least about 95%, 96%, 97%, 98%, 99% or 100% identical to SEQ ID NO: 67.

In one embodiment the protease-activatable T cell activating bispecific molecule comprises the polypeptide sequence of SEQ ID NO: 96, the polypeptide sequence of SEQ ID NO: 66 and the polypeptide sequence of SEQ ID NO: 67. In one embodiment the protease-activatable T cell activating bispecific molecule comprises one polypeptide of SEQ ID NO: 96, one polypeptide of SEQ ID NO: 66 and two polypeptide of SEQ ID NO: 67.

In one embodiment the protease-activatable T cell activating bispecific molecule comprises a polypeptide sequence that is at least about 95%, 96%, 97%, 98%, 99% or 100% identical to SEQ ID NO: 97, a polypeptide sequence that is at least about 95%, 96%, 97%, 98%, 99% or 100% identical to SEQ ID NO: 66 and a polypeptide sequence that is at least about 95%, 96%, 97%, 98%, 99% or 100% identical to SEQ ID NO: 67.

In one embodiment the protease-activatable T cell activating bispecific molecule comprises the polypeptide sequence of SEQ ID NO: 97, the polypeptide sequence of SEQ ID NO: 66 and the polypeptide sequence of SEQ ID NO: 67. In one embodiment the protease-activatable T cell activating bispecific molecule comprises one polypeptide of SEQ ID NO: 97, one polypeptide of SEQ ID NO: 66 and two polypeptide of SEQ ID NO: 67.

In one embodiment the protease-activatable T cell activating bispecific molecule comprises a polypeptide sequence that is at least about 95%, 96%, 97%, 98%, 99% or 100% identical to SEQ ID NO: 98, a polypeptide sequence that is at least about 95%, 96%, 97%, 98%, 99% or 100% identical to SEQ ID NO: 66 and a polypeptide sequence that is at least about 95%, 96%, 97%, 98%, 99% or 100% identical to SEQ ID NO: 67.

In one embodiment the protease-activatable T cell activating bispecific molecule comprises the polypeptide sequence of SEQ ID NO: 98, the polypeptide sequence of SEQ ID NO: 66 and the polypeptide sequence of SEQ ID NO: 67. In one embodiment the protease-activatable T cell activating bispecific molecule comprises one polypeptide of SEQ ID NO: 98, one polypeptide of SEQ ID NO: 66 and two polypeptide of SEQ ID NO: 67.

Linkers

In one aspect, the invention relates to an idiotype-specific polypeptide for reversibly concealing antigen binding of an antigen-binding of a molecule. In one embodiment, the invention relates to an idiotype-specific polypeptide for reversibly concealing an anti-CD3 antigen binding site of a molecule. Such idiotype-specific polypeptide for reversibly concealing an anti-CD3 antigen binding site must be capable of binding to the anti-CD3 antigen binding site's idiotype and thereby reducing or abrogating binding of the anti-CD3 antigen binding site to CD3. In one embodiment the idiotype-specific polypeptide is an anti-idiotype scFv. In one embodiment the idiotype-specific polypeptide is covalently attached to the molecule through a linker. In one embodiment the idiotype-specific polypeptide is covalently attached to the molecule through more than one linker. In one embodiment the idiotype-specific polypeptide is covalently attached to the molecule through two linkers. In one embodiment the linker is a peptide linker. In one embodiment the linker is a protease-cleavable linker.

In one embodiments the protease-activatable T cell activating bispecific molecule comprises a linker having a protease recognition site comprising a polypeptide sequence that is at least about 95%, 96%, 97%, 98%, 99% or 100% identical to SEQ ID NO: 68, 70, 75, 99, 115, 116, 117, 118, 119, 120, 121, 122, 123, 124, 125, 126 or 127. In one embodiment, the protease recognition site comprises the polypeptide sequence of SEQ ID NO: 100, 101, 102, 103, 104, 105, 106, 107, 108, 109, 110, 111, 112, 113 or 114. In a preferred embodiment, the protease recognition site comprises the polypeptide sequence of SEQ ID NO: 114.

In one embodiment the protease is selected from the group consisting of metalloproteinase, e.g., matrix metalloproteinase (MMP) 1-28 and A Disintegrin And Metalloproteinase (ADAM) 2, 7-12, 15, 17-23, 28-30 and 33, serine protease, e.g., urokinase-type plasminogen activator and Matriptase, cysteine protease, aspartic protease, and cathepsin protease. In one specific embodiment the protease is MMP9 or MMP2. In a further specific embodiment, the protease is Matriptase.

Polynucleotides

The invention further provides isolated polynucleotides encoding a protease-activatable T cell activating bispecific molecule as described herein or a fragment thereof. In some embodiments, said fragment is an antigen binding fragment.

The polynucleotides encoding protease-activatable T cell activating bispecific molecules of the invention may be expressed as a single polynucleotide that encodes the entire protease-activatable T cell activating bispecific molecule or as multiple (e.g., two or more) polynucleotides that are co-expressed. Polypeptides encoded by polynucleotides that are co-expressed may associate through, e.g., disulfide bonds or other means to form a functional protease-activatable T cell activating bispecific molecule. For example, the light chain portion of an antigen binding moiety may be encoded by a separate polynucleotide from the portion of the protease-activatable T cell activating bispecific molecule comprising the heavy chain portion of the antigen binding moiety, an Fc domain subunit and optionally (part of) another antigen binding moiety. When co-expressed, the heavy chain polypeptides will associate with the light chain polypeptides to form the antigen binding moiety. In another example, the portion of the protease-activatable T cell activating bispecific molecule comprising one of the two Fc domain subunits and optionally (part of) one or more antigen binding moieties could be encoded by a separate polynucleotide from the portion of the protease-activatable T cell activating bispecific molecule comprising the the other of the two Fc domain subunits and optionally (part of) an antigen binding moiety. When co-expressed, the Fc domain subunits will associate to form the Fc domain.

In some embodiments, the isolated polynucleotide encodes the entire protease-activatable T cell activating bispecific molecule according to the invention as described herein. In other embodiments, the isolated polynucleotide encodes a polypeptides comprised in the protease-activatable T cell activating bispecific molecule according to the invention as described herein.

In another embodiment, the present invention is directed to an isolated polynucleotide encoding a protease-activatable T cell activating bispecific molecule of the invention or a fragment thereof, wherein the polynucleotide comprises a sequence that encodes a variable region sequence. In another embodiment, the present invention is directed to an isolated polynucleotide encoding a protease-activatable T cell activating bispecific molecule or fragment thereof, wherein the polynucleotide comprises a sequence that encodes a polypeptide sequence as shown in SEQ ID NOs 65, 66, 67, 69, 74, 76, 91, 92, 93, 94, 95, 96, 97, 98, or a fragment thereof.

The polynucleotides encoding idiotype-specific polypeptides of the invention may be expressed as a single polynucleotide that encodes the entire idiotype-specific polypeptide or as multiple (e.g., two or more) polynucleotides that are co-expressed. Polypeptides encoded by polynucleotides that are co-expressed may associate through, e.g., disulfide bonds or other means to form a functional idiotype-specific polypeptide, e.g., a masking moiety. For example, in one embodiment the idiotype-specific polypeptide is an anti-idiotypic scFv (single chain variable fragment) wherein the light chain variable portion of the anti-idiotypic scFv may be encoded by a separate polynucleotide from the portion of the anti-idiotypic scFv comprising the heavy chain variable portion of the anti-idiotypic scFv. When co-expressed, the heavy chain polypeptides will associate with the light chain polypeptides to form the anti-idiotypic scFv. In some embodiments, the isolated polynucleotide encodes the idiotype-specific polypeptide according to the invention as described herein.

In certain embodiments the polynucleotide or nucleic acid is DNA. In other embodiments, a polynucleotide of the present invention is RNA, for example, in the form of messenger RNA (mRNA). RNA of the present invention may be single stranded or double stranded.

Recombinant Methods

protease-activatable T cell activating bispecific molecules of the invention may be obtained, for example, by solid-state peptide synthesis (e.g., Merrifield solid phase synthesis) or recombinant production. For recombinant production one or more polynucleotide encoding the protease-activatable T cell activating bispecific molecule (fragment), e.g., as described above, is isolated and inserted into one or more vectors for further cloning and/or expression in a host cell. Such polynucleotide may be readily isolated and sequenced using conventional procedures. In one embodiment a vector, preferably an expression vector, comprising one or more of the polynucleotides of the invention is provided. Methods which are well known to those skilled in the art can be used to construct expression vectors containing the coding sequence of a protease-activatable T cell activating bispecific molecule (fragment) along with appropriate transcriptional/translational control signals. These methods include in vitro recombinant DNA techniques, synthetic techniques and in vivo recombination/genetic recombination. See, for example, the techniques described in Maniatis et al., MOLECULAR CLONING: A LABORATORY MANUAL, Cold Spring Harbor Laboratory, N.Y. (1989); and Ausubel et al., CURRENT PROTOCOLS IN MOLECULAR BIOLOGY, Greene Publishing Associates and Wiley Interscience, N.Y (1989). The expression vector can be part of a plasmid, virus, or may be a nucleic acid fragment. The expression vector includes an expression cassette into which the polynucleotide encoding the protease-activatable T cell activating bispecific molecule (fragment) (i.e. the coding region) is cloned in operable association with a promoter and/or other transcription or translation control elements. As used herein, a “coding region” is a portion of nucleic acid which consists of codons translated into amino acids. Although a “stop codon” (TAG, TGA, or TAA) is not translated into an amino acid, it may be considered to be part of a coding region, if present, but any flanking sequences, for example promoters, ribosome binding sites, transcriptional terminators, introns, 5′ and 3′ untranslated regions, and the like, are not part of a coding region. Two or more coding regions can be present in a single polynucleotide construct, e.g., on a single vector, or in separate polynucleotide constructs, e.g., on separate (different) vectors. Furthermore, any vector may contain a single coding region, or may comprise two or more coding regions, e.g., a vector of the present invention may encode one or more polypeptides, which are post- or co-translationally separated into the final proteins via proteolytic cleavage. In addition, a vector, polynucleotide, or nucleic acid of the invention may encode heterologous coding regions, either fused or unfused to a polynucleotide encoding the protease-activatable T cell activating bispecific molecule (fragment) of the invention, or variant or derivative thereof. Heterologous coding regions include without limitation specialized elements or motifs, such as a secretory signal peptide or a heterologous functional domain. An operable association is when a coding region for a gene product, e.g., a polypeptide, is associated with one or more regulatory sequences in such a way as to place expression of the gene product under the influence or control of the regulatory sequence(s). Two DNA fragments (such as a polypeptide coding region and a promoter associated therewith) are “operably associated” if induction of promoter function results in the transcription of mRNA encoding the desired gene product and if the nature of the linkage between the two DNA fragments does not interfere with the ability of the expression regulatory sequences to direct the expression of the gene product or interfere with the ability of the DNA template to be transcribed. Thus, a promoter region would be operably associated with a nucleic acid encoding a polypeptide if the promoter was capable of effecting transcription of that nucleic acid. The promoter may be a cell-specific promoter that directs substantial transcription of the DNA only in predetermined cells. Other transcription control elements, besides a promoter, for example enhancers, operators, repressors, and transcription termination signals, can be operably associated with the polynucleotide to direct cell-specific transcription. Suitable promoters and other transcription control regions are disclosed herein. A variety of transcription control regions are known to those skilled in the art. These include, without limitation, transcription control regions, which function in vertebrate cells, such as, but not limited to, promoter and enhancer segments from cytomegaloviruses (e.g., the immediate early promoter, in conjunction with intron-A), simian virus 40 (e.g., the early promoter), and retroviruses (such as, e.g., Rous sarcoma virus). Other transcription control regions include those derived from vertebrate genes such as actin, heat shock protein, bovine growth hormone and rabbit â-globin, as well as other sequences capable of controlling gene expression in eukaryotic cells. Additional suitable transcription control regions include tissue-specific promoters and enhancers as well as inducible promoters (e.g., promoters inducible tetracyclins). Similarly, a variety of translation control elements are known to those of ordinary skill in the art. These include, but are not limited to ribosome binding sites, translation initiation and termination codons, and elements derived from viral systems (particularly an internal ribosome entry site, or IRES, also referred to as a CITE sequence). The expression cassette may also include other features such as an origin of replication, and/or chromosome integration elements such as retroviral long terminal repeats (LTRs), or adeno-associated viral (AAV) inverted terminal repeats (ITRs).

Polynucleotide and nucleic acid coding regions of the present invention may be associated with additional coding regions which encode secretory or signal peptides, which direct the secretion of a polypeptide encoded by a polynucleotide of the present invention. For example, if secretion of the protease-activatable T cell activating bispecific molecule is desired, DNA encoding a signal sequence may be placed upstream of the nucleic acid encoding a protease-activatable T cell activating bispecific molecule of the invention or a fragment thereof. According to the signal hypothesis, proteins secreted by mammalian cells have a signal peptide or secretory leader sequence which is cleaved from the mature protein once export of the growing protein chain across the rough endoplasmic reticulum has been initiated. Those of ordinary skill in the art are aware that polypeptides secreted by vertebrate cells generally have a signal peptide fused to the N-terminus of the polypeptide, which is cleaved from the translated polypeptide to produce a secreted or “mature” form of the polypeptide. In certain embodiments, the native signal peptide, e.g., an immunoglobulin heavy chain or light chain signal peptide is used, or a functional derivative of that sequence that retains the ability to direct the secretion of the polypeptide that is operably associated with it. Alternatively, a heterologous mammalian signal peptide, or a functional derivative thereof, may be used. For example, the wild-type leader sequence may be substituted with the leader sequence of human tissue plasminogen activator (TPA) or mouse β-glucuronidase.

DNA encoding a short protein sequence that could be used to facilitate later purification (e.g., a histidine tag) or assist in labeling the protease-activatable T cell activating bispecific molecule may be included within or at the ends of the protease-activatable T cell activating bispecific molecule (fragment) encoding polynucleotide.

In a further embodiment, a host cell comprising one or more polynucleotides of the invention is provided. In certain embodiments a host cell comprising one or more vectors of the invention is provided. The polynucleotides and vectors may incorporate any of the features, singly or in combination, described herein in relation to polynucleotides and vectors, respectively. In one such embodiment a host cell comprises (e.g., has been transformed or transfected with) a vector comprising a polynucleotide that encodes (part of) a protease-activatable T cell activating bispecific molecule of the invention. As used herein, the term “host cell” refers to any kind of cellular system which can be engineered to generate the protease-activatable T cell activating bispecific molecules of the invention or fragments thereof. Host cells suitable for replicating and for supporting expression of protease-activatable T cell activating bispecific molecules are well known in the art. Such cells may be transfected or transduced as appropriate with the particular expression vector and large quantities of vector containing cells can be grown for seeding large scale fermenters to obtain sufficient quantities of the protease-activatable T cell activating bispecific molecule for clinical applications. Suitable host cells include prokaryotic microorganisms, such as E. coli, or various eukaryotic cells, such as Chinese hamster ovary cells (CHO), insect cells, or the like. For example, polypeptides may be produced in bacteria in particular when glycosylation is not needed. After expression, the polypeptide may be isolated from the bacterial cell paste in a soluble fraction and can be further purified. In addition to prokaryotes, eukaryotic microbes such as filamentous fungi or yeast are suitable cloning or expression hosts for polypeptide-encoding vectors, including fungi and yeast strains whose glycosylation pathways have been “humanized”, resulting in the production of a polypeptide with a partially or fully human glycosylation pattern. See Gemgross, Nat Biotech 22, 1409-1414 (2004), and Li et al., Nat Biotech 24, 210-215 (2006). Suitable host cells for the expression of (glycosylated) polypeptides are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells. Plant cell cultures can also be utilized as hosts. See e.g., U.S. Pat. Nos. 5,959,177, 6,040,498, 6,420,548, 7,125,978, and 6,417,429 (describing PLANTIBODIES™ technology for producing antibodies in transgenic plants). Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293T cells as described, e.g., in Graham et al., J Gen Virol 36, 59 (1977)), baby hamster kidney cells (BHK), mouse sertoli cells (TM4 cells as described, e.g., in Mather, Biol Reprod 23, 243-251 (1980)), monkey kidney cells (CV1), African green monkey kidney cells (VERO-76), human cervical carcinoma cells (HELA), canine kidney cells (MDCK), buffalo rat liver cells (BRL 3A), human lung cells (W138), human liver cells (Hep G2), mouse mammary tumor cells (MMT 060562), TRI cells (as described, e.g., in Mather et al., Annals N.Y. Acad Sci 383, 44-68 (1982)), MRC 5 cells, and FS4 cells. Other useful mammalian host cell lines include Chinese hamster ovary (CHO) cells, including dhfr⁻ CHO cells (Urlaub et al., Proc Natl Acad Sci USA 77, 4216 (1980)); and myeloma cell lines such as YO, NS0, P3X63 and Sp2/0. For a review of certain mammalian host cell lines suitable for protein production, see, e.g., Yazaki and Wu, Methods in Molecular Biology, Vol. 248 (B. K. C. Lo, ed., Humana Press, Totowa, N.J.), pp. 255-268 (2003). Host cells include cultured cells, e.g., mammalian cultured cells, yeast cells, insect cells, bacterial cells and plant cells, to name only a few, but also cells comprised within a transgenic animal, transgenic plant or cultured plant or animal tissue. In one embodiment, the host cell is a eukaryotic cell, preferably a mammalian cell, such as a Chinese Hamster Ovary (CHO) cell, a human embryonic kidney (HEK) cell or a lymphoid cell (e.g., YO, NS0, Sp20 cell). Standard technologies are known in the art to express foreign genes in these systems. Cells expressing a polypeptide comprising either the heavy or the light chain of an antigen binding domain such as an antibody, may be engineered so as to also express the other of the antibody chains such that the expressed product is an antibody that has both a heavy and a light chain.

In one embodiment, a method of producing a protease-activatable T cell activating bispecific molecule according to the invention is provided, wherein the method comprises culturing a host cell comprising a polynucleotide encoding the protease-activatable T cell activating bispecific molecule, as provided herein, under conditions suitable for expression of the protease-activatable T cell activating bispecific molecule, and recovering the protease-activatable T cell activating bispecific molecule from the host cell (or host cell culture medium).

The components of the protease-activatable T cell activating bispecific molecule are genetically fused to each other. Protease-activatable T cell activating bispecific molecules can be designed such that its components are fused directly to each other or indirectly through a linker sequence. The composition and length of the linker may be determined in accordance with methods well known in the art and may be tested for efficacy. Examples of linker sequences between different components of protease-activatable T cell activating bispecific molecules are found in the sequences provided herein. Additional sequences may also be included to incorporate a cleavage site to separate the individual components of the fusion if desired, for example an endopeptidase recognition sequence.

In certain embodiments the one or more antigen binding moieties of the protease-activatable T cell activating bispecific molecules comprise at least an antibody variable region capable of binding an antigenic determinant. Variable regions can form part of and be derived from naturally or non-naturally occurring antibodies and fragments thereof. Methods to produce polyclonal antibodies and monoclonal antibodies are well known in the art (see e.g., Harlow and Lane, “Antibodies, a laboratory manual”, Cold Spring Harbor Laboratory, 1988). Non-naturally occurring antibodies can be constructed using solid phase-peptide synthesis, can be produced recombinantly (e.g., as described in U.S. Pat. No. 4,186,567) or can be obtained, for example, by screening combinatorial libraries comprising variable heavy chains and variable light chains (see e.g., U.S. Pat. No. 5,969,108 to McCafferty).

Any animal species of antibody, antibody fragment, antigen binding domain or variable region can be used in the protease-activatable T cell activating bispecific molecules of the invention. Non-limiting antibodies, antibody fragments, antigen binding domains or variable regions useful in the present invention can be of murine, primate, or human origin. If the protease-activatable T cell activating bispecific molecule is intended for human use, a chimeric form of antibody may be used wherein the constant regions of the antibody are from a human. A “humanized” or fully human form of the antibody can also be prepared in accordance with methods well known in the art (see e. g. U.S. Pat. No. 5,565,332 to Winter). Humanization may be achieved by various methods including, but not limited to (a) grafting the non-human (e.g., donor antibody) CDRs onto human (e.g., recipient antibody) framework and constant regions with or without retention of critical framework residues (e.g., those that are important for retaining good antigen binding affinity or antibody functions), (b) grafting only the non-human specificity-determining regions (SDRs or a-CDRs; the residues critical for the antibody-antigen interaction) onto human framework and constant regions, or (c) transplanting the entire non-human variable domains, but “cloaking” them with a human-like section by replacement of surface residues. Humanized antibodies and methods of making them are reviewed, e.g., in Almagro and Fransson, Front Biosci 13, 1619-1633 (2008), and are further described, e.g., in Riechmann et al., Nature 332, 323-329 (1988); Queen et al., Proc Natl Acad Sci USA 86, 10029-10033 (1989); U.S. Pat. Nos. 5,821,337, 7,527,791, 6,982,321, and 7,087,409; Jones et al., Nature 321, 522-525 (1986); Morrison et al., Proc Natl Acad Sci 81, 6851-6855 (1984); Morrison and Oi, Adv Immunol 44, 65-92 (1988); Verhoeyen et al., Science 239, 1534-1536 (1988); Padlan, Molec Immun 31(3), 169-217 (1994); Kashmiri et al., Methods 36, 25-34 (2005) (describing SDR (a-CDR) grafting); Padlan, Mol Immunol 28, 489-498 (1991) (describing “resurfacing”); Dall'Acqua et al., Methods 36, 43-60 (2005) (describing “FR shuffling”); and Osboum et al., Methods 36, 61-68 (2005) and Klimka et al., Br J Cancer 83, 252-260 (2000) (describing the “guided selection” approach to FR shuffling). Human antibodies and human variable regions can be produced using various techniques known in the art. Human antibodies are described generally in van Dijk and van de Winkel, Curr Opin Pharmacol 5, 368-74 (2001) and Lonberg, Curr Opin Immunol 20, 450-459 (2008). Human variable regions can form part of and be derived from human monoclonal antibodies made by the hybridoma method (see e.g., Monoclonal Antibody Production Techniques and Applications, pp. 51-63 (Marcel Dekker, Inc., New York, 1987)). Human antibodies and human variable regions may also be prepared by administering an immunogen to a transgenic animal that has been modified to produce intact human antibodies or intact antibodies with human variable regions in response to antigenic challenge (see e.g., Lonberg, Nat Biotech 23, 1117-1125 (2005). Human antibodies and human variable regions may also be generated by isolating Fv clone variable region sequences selected from human-derived phage display libraries (see e.g., Hoogenboom et al. in Methods in Molecular Biology 178, 1-37 (O'Brien et al., ed., Human Press, Totowa, N.J., 2001); and McCafferty et al., Nature 348, 552-554; Clackson et al., Nature 352, 624-628 (1991)). Phage typically display antibody fragments, either as single-chain Fv (scFv) fragments or as Fab fragments.

In certain embodiments, the antigen binding moieties useful in the present invention are engineered to have enhanced binding affinity according to, for example, the methods disclosed in U.S. Pat. Appl. Publ. No. 2004/0132066, the entire contents of which are hereby incorporated by reference. The ability of the protease-activatable T cell activating bispecific molecule of the invention to bind to a specific antigenic determinant can be measured either through an enzyme-linked immunosorbent assay (ELISA) or other techniques familiar to one of skill in the art, e.g., surface plasmon resonance technique (analyzed on a BIACORE T100 system) (Liljeblad, et al., Glyco J 17, 323-329 (2000)), and traditional binding assays (Heeley, Endocr Res 28, 217-229 (2002)). Competition assays may be used to identify an antibody, antibody fragment, antigen binding domain or variable domain that competes with a reference antibody for binding to a particular antigen, e.g., an antibody that competes with the V9 antibody for binding to CD3. In certain embodiments, such a competing antibody binds to the same epitope (e.g., a linear or a conformational epitope) that is bound by the reference antibody. Detailed exemplary methods for mapping an epitope to which an antibody binds are provided in Morris (1996) “Epitope Mapping Protocols,” in Methods in Molecular Biology vol. 66 (Humana Press, Totowa, N.J.). In an exemplary competition assay, immobilized antigen (e.g., CD3) is incubated in a solution comprising a first labeled antibody that binds to the antigen (e.g., V9 antibody, described in U.S. Pat. No. 6,054,297) and a second unlabeled antibody that is being tested for its ability to compete with the first antibody for binding to the antigen. The second antibody may be present in a hybridoma supernatant. As a control, immobilized antigen is incubated in a solution comprising the first labeled antibody but not the second unlabeled antibody. After incubation under conditions permissive for binding of the first antibody to the antigen, excess unbound antibody is removed, and the amount of label associated with immobilized antigen is measured. If the amount of label associated with immobilized antigen is substantially reduced in the test sample relative to the control sample, then that indicates that the second antibody is competing with the first antibody for binding to the antigen. See Harlow and Lane (1988) Antibodies: A Laboratory Manual ch. 14 (Cold Spring Harbor Laboratory, Cold Spring Harbor, N.Y.).

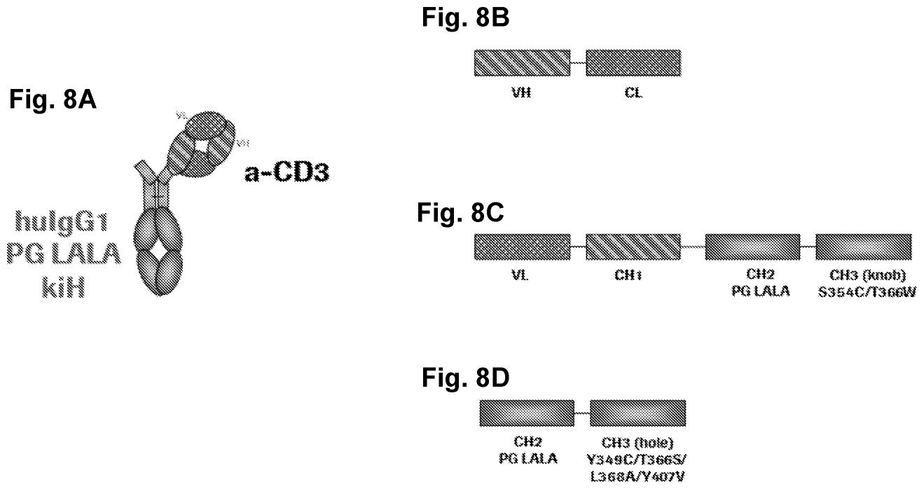

Protease-activatable T cell activating bispecific molecules prepared as described herein may be purified by art-known techniques such as high performance liquid chromatography, ion exchange chromatography, gel electrophoresis, affinity chromatography, size exclusion chromatography, and the like. The actual conditions used to purify a particular protein will depend, in part, on factors such as net charge, hydrophobicity, hydrophilicity etc., and will be apparent to those having skill in the art. For affinity chromatography purification an antibody, ligand, receptor or antigen can be used to which the protease-activatable T cell activating bispecific molecule binds. For example, for affinity chromatography purification of protease-activatable T cell activating bispecific molecules of the invention, a matrix with protein A or protein G may be used. Sequential Protein A or G affinity chromatography and size exclusion chromatography can be used to isolate a protease-activatable T cell activating bispecific molecule essentially as described in the Examples. The purity of the protease-activatable T cell activating bispecific molecule can be determined by any of a variety of well-known analytical methods including gel electrophoresis, high pressure liquid chromatography, and the like. For example, the heavy chain fusion proteins expressed as described in the Examples were shown to be intact and properly assembled as demonstrated by reducing SDS-PAGE (see, e.g., FIGS. 8-12). Three bands were resolved at approximately Mr 25,000, Mr 50,000 and Mr 75,000, corresponding to the predicted molecular weights of the protease-activatable T cell activating bispecific molecule light chain, heavy chain and heavy chain/light chain fusion protein.

Assays

protease-activatable T cell activating bispecific molecules provided herein may be identified, screened for, or characterized for their physical/chemical properties and/or biological activities by various assays known in the art.

Affinity Assays

The affinity of the protease-activatable T cell activating bispecific molecule for an Fc receptor or a target antigen can be determined in accordance with the methods set forth in the Examples by surface plasmon resonance (SPR), using standard instrumentation such as a BIAcore instrument (GE Healthcare), and receptors or target proteins such as may be obtained by recombinant expression. Alternatively, binding of protease-activatable T cell activating bispecific molecules for different receptors or target antigens may be evaluated using cell lines expressing the particular receptor or target antigen, for example by flow cytometry (FACS). A specific illustrative and exemplary embodiment for measuring binding affinity is described in the following and in the Examples below.

According to one embodiment, K_{D} is measured by surface plasmon resonance using a BIACORE® T100 machine (GE Healthcare) at 25° C.

To analyze the interaction between the Fc-portion and Fc receptors, His-tagged recombinant Fc-receptor is captured by an anti-Penta His antibody (Qiagen) immobilized on CMS chips and the bispecific constructs are used as analytes. Briefly, carboxymethylated dextran biosensor chips (CMS, GE Healthcare) are activated with N-ethyl-N′-(3-dimethylaminopropyl)-carbodiimide hydrochloride (EDC) and N-hydroxysuccinimide (NHS) according to the supplier's instructions. Anti Penta-His antibody is diluted with 10 mM sodium acetate, pH 5.0, to 40 μg/ml before injection at a flow rate of 5 μl/min to achieve approximately 6500 response units (RU) of coupled protein. Following the injection of the ligand, 1 M ethanolamine is injected to block unreacted groups. Subsequently the Fc-receptor is captured for 60 s at 4 or 10 nM. For kinetic measurements, four-fold serial dilutions of the bispecific construct (range between 500 nM and 4000 nM) are injected in HBS-EP (GE Healthcare, 10 mM HEPES, 150 mM NaCl, 3 mM EDTA, 0.05% Surfactant P20, pH 7.4) at 25° C. at a flow rate of 30 μl/min for 120 s.

To determine the affinity to the target antigen, bispecific constructs are captured by an anti-human Fab specific antibody (GE Healthcare) that is immobilized on an activated CMS-sensor chip surface as described for the anti Penta-His antibody. The final amount of coupled protein is is approximately 12000 R U. The bispecific constructs are captured for 90 s at 300 nM. The target antigens are passed through the flow cells for 180 s at a concentration range from 250 to 1000 nM with a flowrate of 30 μl/min. The dissociation is monitored for 180 s.

Bulk refractive index differences are corrected for by subtracting the response obtained on reference flow cell. The steady state response was used to derive the dissociation constant K_{D} by non-linear curve fitting of the Langmuir binding isotherm. Association rates (kₒₙ) and dissociation rates (k_{off}) are calculated using a simple one-to-one Langmuir binding model (BIACORE® T100 Evaluation Software version 1.1.1) by simultaneously fitting the association and dissociation sensorgrams. The equilibrium dissociation constant (K_{D}) is calculated as the ratio k_{off}/kₒₙ. See, e.g., Chen et al., J Mol Biol 293, 865-881 (1999).

Activity Assays

Biological activity of the protease-activatable T cell activating bispecific molecules of the invention can be measured by various assays as described in the Examples. Biological activities may for example include the induction of proliferation of T cells, the induction of signaling in T cells, the induction of expression of activation markers in T cells, the induction of cytokine secretion by T cells, the induction of lysis of target cells such as tumor cells, and the induction of tumor regression and/or the improvement of survival.

Compositions, Formulations, and Routes of Administration

In a further aspect, the invention provides pharmaceutical compositions comprising any of the protease-activatable T cell activating bispecific molecules provided herein, e.g., for use in any of the below therapeutic methods. In one embodiment, a pharmaceutical composition comprises any of the protease-activatable T cell activating bispecific molecules provided herein and a pharmaceutically acceptable carrier. In another embodiment, a pharmaceutical composition comprises any of the protease-activatable T cell activating bispecific molecules provided herein and at least one additional therapeutic agent, e.g., as described below.

Further provided is a method of producing a protease-activatable T cell activating bispecific molecule of the invention in a form suitable for administration in vivo, the method comprising (a) obtaining a protease-activatable T cell activating bispecific molecule according to the invention, and (b) formulating the protease-activatable T cell activating bispecific molecule with at least one pharmaceutically acceptable carrier, whereby a preparation of protease-activatable T cell activating bispecific molecule is formulated for administration in vivo.

Pharmaceutical compositions of the present invention comprise a therapeutically effective amount of one or more protease-activatable T cell activating bispecific molecule dissolved or dispersed in a pharmaceutically acceptable carrier. The phrases “pharmaceutical or pharmacologically acceptable” refers to molecular entities and compositions that are generally non-toxic to recipients at the dosages and concentrations employed, i.e. do not produce an adverse, allergic or other untoward reaction when administered to an animal, such as, for example, a human, as appropriate. The preparation of a pharmaceutical composition that contains at least one protease-activatable T cell activating bispecific molecule and optionally an additional active ingredient will be known to those of skill in the art in light of the present disclosure, as exemplified by Remington's Pharmaceutical Sciences, 18th Ed. Mack Printing Company, 1990, incorporated herein by reference. Moreover, for animal (e.g., human) administration, it will be understood that preparations should meet sterility, pyrogenicity, general safety and purity standards as required by FDA Office of Biological Standards or corresponding authorities in other countries. Preferred compositions are lyophilized formulations or aqueous solutions. As used herein, “pharmaceutically acceptable carrier” includes any and all solvents, buffers, dispersion media, coatings, surfactants, antioxidants, preservatives (e.g., antibacterial agents, antifungal agents), isotonic agents, absorption delaying agents, salts, preservatives, antioxidants, proteins, drugs, drug stabilizers, polymers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient, its use in the therapeutic or pharmaceutical compositions is contemplated.

The composition may comprise different types of carriers depending on whether it is to be administered in solid, liquid or aerosol form, and whether it need to be sterile for such routes of administration as injection. Protease-activatable T cell activating bispecific molecules of the present invention (and any additional therapeutic agent) can be administered intravenously, intradermally, intraarterially, intraperitoneally, intralesionally, intracranially, intraarticularly, intraprostatically, intrasplenically, intrarenally, intrapleurally, intratracheally, intranasally, intravitreally, intravaginally, intrarectally, intratumorally, intramuscularly, intraperitoneally, subcutaneously, subconjunctivally, intravesicularlly, mucosally, intrapericardially, intraumbilically, intraocularally, orally, topically, locally, by inhalation (e.g., aerosol inhalation), injection, infusion, continuous infusion, localized perfusion bathing target cells directly, via a catheter, via a lavage, in cremes, in lipid compositions (e.g., liposomes), or by other method or any combination of the forgoing as would be known to one of ordinary skill in the art (see, for example, Remington's Pharmaceutical Sciences, 18th Ed. Mack Printing Company, 1990, incorporated herein by reference). Parenteral administration, in particular intravenous injection, is most commonly used for administering polypeptide molecules such as the protease-activatable T cell activating bispecific molecules of the invention.

Parenteral compositions include those designed for administration by injection, e.g., subcutaneous, intradermal, intralesional, intravenous, intraarterial intramuscular, intrathecal or intraperitoneal injection. For injection, the protease-activatable T cell activating bispecific molecules of the invention may be formulated in aqueous solutions, preferably in physiologically compatible buffers such as Hanks' solution, Ringer's solution, or physiological saline buffer. The solution may contain formulatory agents such as suspending, stabilizing and/or dispersing agents. Alternatively, the protease-activatable T cell activating bispecific molecules may be in powder form for constitution with a suitable vehicle, e.g., sterile pyrogen-free water, before use. Sterile injectable solutions are prepared by incorporating the protease-activatable T cell activating bispecific molecules of the invention in the required amount in the appropriate solvent with various of the other ingredients enumerated below, as required. Sterility may be readily accomplished, e.g., by filtration through sterile filtration membranes. Generally, dispersions are prepared by incorporating the various sterilized active ingredients into a sterile vehicle which contains the basic dispersion medium and/or the other ingredients. In the case of sterile powders for the preparation of sterile injectable solutions, suspensions or emulsion, the preferred methods of preparation are vacuum-drying or freeze-drying techniques which yield a powder of the active ingredient plus any additional desired ingredient from a previously sterile-filtered liquid medium thereof. The liquid medium should be suitably buffered if necessary and the liquid diluent first rendered isotonic prior to injection with sufficient saline or glucose. The composition must be stable under the conditions of manufacture and storage, and preserved against the contaminating action of microorganisms, such as bacteria and fungi. It will be appreciated that endotoxin contamination should be kept minimally at a safe level, for example, less that 0.5 ng/mg protein. Suitable pharmaceutically acceptable carriers include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as polyethylene glycol (PEG). Aqueous injection suspensions may contain compounds which increase the viscosity of the suspension, such as sodium carboxymethyl cellulose, sorbitol, dextran, or the like. Optionally, the suspension may also contain suitable stabilizers or agents which increase the solubility of the compounds to allow for the preparation of highly concentrated solutions. Additionally, suspensions of the active compounds may be prepared as appropriate oily injection suspensions. Suitable lipophilic solvents or vehicles include fatty oils such as sesame oil, or synthetic fatty acid esters, such as ethyl cleats or triglycerides, or liposomes.

Active ingredients may be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences (18th Ed. Mack Printing Company, 1990). Sustained-release preparations may be prepared. Suitable examples of sustained-release preparations include semipermeable matrices of solid hydrophobic polymers containing the polypeptide, which matrices are in the form of shaped articles, e.g., films, or microcapsules. In particular embodiments, prolonged absorption of an injectable composition can be brought about by the use in the compositions of agents delaying absorption, such as, for example, aluminum monostearate, gelatin or combinations thereof.

In addition to the compositions described previously, the protease-activatable T cell activating bispecific molecules may also be formulated as a depot preparation. Such long acting formulations may be administered by implantation (for example subcutaneously or intramuscularly) or by intramuscular injection. Thus, for example, the protease-activatable T cell activating bispecific molecules may be formulated with suitable polymeric or hydrophobic materials (for example as an emulsion in an acceptable oil) or ion exchange resins, or as sparingly soluble derivatives, for example, as a sparingly soluble salt.

Pharmaceutical compositions comprising the protease-activatable T cell activating bispecific molecules of the invention may be manufactured by means of conventional mixing, dissolving, emulsifying, encapsulating, entrapping or lyophilizing processes. Pharmaceutical compositions may be formulated in conventional manner using one or more physiologically acceptable carriers, diluents, excipients or auxiliaries which facilitate processing of the proteins into preparations that can be used pharmaceutically. Proper formulation is dependent upon the route of administration chosen.

The protease-activatable T cell activating bispecific molecules may be formulated into a composition in a free acid or base, neutral or salt form. Pharmaceutically acceptable salts are salts that substantially retain the biological activity of the free acid or base. These include the acid addition salts, e.g., those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups can also be derived from inorganic bases such as for example, sodium, potassium, ammonium, calcium or ferric hydroxides; or such organic bases as isopropylamine, trimethylamine, histidine or procaine. Pharmaceutical salts tend to be more soluble in aqueous and other protic solvents than are the corresponding free base forms.

Therapeutic Methods and Compositions

Any of the protease-activatable T cell activating bispecific molecules provided herein may be used in therapeutic methods. Protease-activatable T cell activating bispecific molecules of the invention can be used as immunotherapeutic agents, for example in the treatment of cancers.

For use in therapeutic methods, protease-activatable T cell activating bispecific molecules of the invention would be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners.

In one aspect, protease-activatable T cell activating bispecific molecules of the invention for use as a medicament are provided. In further aspects, protease-activatable T cell activating bispecific molecules of the invention for use in treating a disease are provided. In certain embodiments, protease-activatable T cell activating bispecific molecules of the invention for use in a method of treatment are provided. In one embodiment, the invention provides a protease-activatable T cell activating bispecific molecule as described herein for use in the treatment of a disease in an individual in need thereof. In certain embodiments, the invention provides a protease-activatable T cell activating bispecific molecule for use in a method of treating an individual having a disease comprising administering to the individual a therapeutically effective amount of the protease-activatable T cell activating bispecific molecule. In certain embodiments the disease to be treated is a proliferative disorder. In a particular embodiment the disease is cancer. In certain embodiments the method further comprises administering to the individual a therapeutically effective amount of at least one additional therapeutic agent, e.g., an anti-cancer agent if the disease to be treated is cancer. In further embodiments, the invention provides a protease-activatable T cell activating bispecific molecule as described herein for use in inducing lysis of a target cell, particularly a tumor cell. In certain embodiments, the invention provides a protease-activatable T cell activating bispecific molecule for use in a method of inducing lysis of a target cell, particularly a tumor cell, in an individual comprising administering to the individual an effective amount of the protease-activatable T cell activating bispecific molecule to induce lysis of a target cell. An “individual” according to any of the above embodiments is a mammal, preferably a human.

In a further aspect, the invention provides for the use of a protease-activatable T cell activating bispecific molecule of the invention in the manufacture or preparation of a medicament. In one embodiment the medicament is for the treatment of a disease in an individual in need thereof. In a further embodiment, the medicament is for use in a method of treating a disease comprising administering to an individual having the disease a therapeutically effective amount of the medicament. In certain embodiments the disease to be treated is a proliferative disorder. In a particular embodiment the disease is cancer. In one embodiment, the method further comprises administering to the individual a therapeutically effective amount of at least one additional therapeutic agent, e.g., an anti-cancer agent if the disease to be treated is cancer. In a further embodiment, the medicament is for inducing lysis of a target cell, particularly a tumor cell. In still a further embodiment, the medicament is for use in a method of inducing lysis of a target cell, particularly a tumor cell, in an individual comprising administering to the individual an effective amount of the medicament to induce lysis of a target cell. An “individual” according to any of the above embodiments may be a mammal, preferably a human.

In a further aspect, the invention provides a method for treating a disease. In one embodiment, the method comprises administering to an individual having such disease a therapeutically effective amount of a protease-activatable T cell activating bispecific molecule of the invention. In one embodiment a composition is administered to said invididual, comprising the protease-activatable T cell activating bispecific molecule of the invention in a pharmaceutically acceptable form. In certain embodiments the disease to be treated is a proliferative disorder. In a particular embodiment the disease is cancer. In certain embodiments the method further comprises administering to the individual a therapeutically effective amount of at least one additional therapeutic agent, e.g., an anti-cancer agent if the disease to be treated is cancer. An “individual” according to any of the above embodiments may be a mammal, preferably a human. In a further aspect, the invention provides a method for inducing lysis of a target cell, particularly a tumor cell. In one embodiment the method comprises contacting a target cell with a protease-activatable T cell activating bispecific molecule of the invention in the presence of a T cell, particularly a cytotoxic T cell. In a further aspect, a method for inducing lysis of a target cell, particularly a tumor cell, in an individual is provided. In one such embodiment, the method comprises administering to the individual an effective amount of a protease-activatable T cell activating bispecific molecule to induce lysis of a target cell. In one embodiment, an “individual” is a human.

In certain embodiments the disease to be treated is a proliferative disorder, particularly cancer. Non-limiting examples of cancers include bladder cancer, brain cancer, head and neck cancer, pancreatic cancer, lung cancer, breast cancer, ovarian cancer, uterine cancer, cervical cancer, endometrial cancer, esophageal cancer, colon cancer, colorectal cancer, rectal cancer, gastric cancer, prostate cancer, blood cancer, skin cancer, squamous cell carcinoma, bone cancer, and kidney cancer. Other cell proliferation disorders that can be treated using a protease-activatable T cell activating bispecific molecule of the present invention include, but are not limited to neoplasms located in the: abdomen, bone, breast, digestive system, liver, pancreas, peritoneum, endocrine glands (adrenal, parathyroid, pituitary, testicles, ovary, thymus, thyroid), eye, head and neck, nervous system (central and peripheral), lymphatic system, pelvic, skin, soft tissue, spleen, thoracic region, and urogenital system. Also included are pre-cancerous conditions or lesions and cancer metastases. In certain embodiments the cancer is chosen from the group consisting of renal cell cancer, skin cancer, lung cancer, colorectal cancer, breast cancer, brain cancer, head and neck cancer. A skilled artisan readily recognizes that in many cases the protease-activatable T cell activating bispecific molecule may not provide a cure but may only provide partial benefit. In some embodiments, a physiological change having some benefit is also considered therapeutically beneficial. Thus, in some embodiments, an amount of protease-activatable T cell activating bispecific molecule that provides a physiological change is considered an “effective amount” or a “therapeutically effective amount”. The subject, patient, or individual in need of treatment is typically a mammal, more specifically a human.

In some embodiments, an effective amount of a protease-activatable T cell activating bispecific molecule of the invention is administered to a cell. In other embodiments, a therapeutically effective amount of a protease-activatable T cell activating bispecific molecule of the invention is administered to an individual for the treatment of disease.

For the prevention or treatment of disease, the appropriate dosage of a protease-activatable T cell activating bispecific molecule of the invention (when used alone or in combination with one or more other additional therapeutic agents) will depend on the type of disease to be treated, the route of administration, the body weight of the patient, the type of T cell activating bispecific antigen binding molecule, the severity and course of the disease, whether the T cell activating bispecific antigen binding molecule is administered for preventive or therapeutic purposes, previous or concurrent therapeutic interventions, the patient's clinical history and response to the protease-activatable T cell activating bispecific molecule, and the discretion of the attending physician. The practitioner responsible for administration will, in any event, determine the concentration of active ingredient(s) in a composition and appropriate dose(s) for the individual subject. Various dosing schedules including but not limited to single or multiple administrations over various time-points, bolus administration, and pulse infusion are contemplated herein.

The protease-activatable T cell activating bispecific molecule is suitably administered to the patient at one time or over a series of treatments. Depending on the type and severity of the disease, about 1 μg/kg to 15 mg/kg (e.g., 0.1 mg/kg-10 mg/kg) of protease-activatable T cell activating bispecific molecule can be an initial candidate dosage for administration to the patient, whether, for example, by one or more separate administrations, or by continuous infusion. One typical daily dosage might range from about 1 μg/kg to 100 mg/kg or more, depending on the factors mentioned above. For repeated administrations over several days or longer, depending on the condition, the treatment would generally be sustained until a desired suppression of disease symptoms occurs. One exemplary dosage of the T cell activating bispecific antigen binding molecule would be in the range from about 0.005 mg/kg to about 10 mg/kg. In other non-limiting examples, a dose may also comprise from about 1 microgram/kg body weight, about 5 microgram/kg body weight, about 10 microgram/kg body weight, about 50 microgram/kg body weight, about 100 microgram/kg body weight, about 200 microgram/kg body weight, about 350 microgram/kg body weight, about 500 microgram/kg body weight, about 1 milligram/kg body weight, about 5 milligram/kg body weight, about 10 milligram/kg body weight, about 50 milligram/kg body weight, about 100 milligram/kg body weight, about 200 milligram/kg body weight, about 350 milligram/kg body weight, about 500 milligram/kg body weight, to about 1000 mg/kg body weight or more per administration, and any range derivable therein. In non-limiting examples of a derivable range from the numbers listed herein, a range of about 5 mg/kg body weight to about 100 mg/kg body weight, about 5 microgram/kg body weight to about 500 milligram/kg body weight, etc., can be administered, based on the numbers described above. Thus, one or more doses of about 0.5 mg/kg, 2.0 mg/kg, 5.0 mg/kg or 10 mg/kg (or any combination thereof) may be administered to the patient. Such doses may be administered intermittently, e.g., every week or every three weeks (e.g., such that the patient receives from about two to about twenty, or e.g., about six doses of the protease-activatable T cell activating bispecific molecule). An initial higher loading dose, followed by one or more lower doses may be administered. However, other dosage regimens may be useful. The progress of this therapy is easily monitored by conventional techniques and assays.

The protease-activatable T cell activating bispecific molecule of the invention will generally be used in an amount effective to achieve the intended purpose. For use to treat or prevent a disease condition, the protease-activatable T cell activating bispecific molecules of the invention, or pharmaceutical compositions thereof, are administered or applied in a therapeutically effective amount. Determination of a therapeutically effective amount is well within the capabilities of those skilled in the art, especially in light of the detailed disclosure provided herein.

For systemic administration, a therapeutically effective dose can be estimated initially from in vitro assays, such as cell culture assays. A dose can then be formulated in animal models to achieve a circulating concentration range that includes the IC₅₀ as determined in cell culture. Such information can be used to more accurately determine useful doses in humans.

Initial dosages can also be estimated from in vivo data, e.g., animal models, using techniques that are well known in the art. One having ordinary skill in the art could readily optimize administration to humans based on animal data.

Dosage amount and interval may be adjusted individually to provide plasma levels of the protease-activatable T cell activating bispecific molecules which are sufficient to maintain therapeutic effect. Usual patient dosages for administration by injection range from about 0.1 to 50 mg/kg/day, typically from about 0.5 to 1 mg/kg/day. Therapeutically effective plasma levels may be achieved by administering multiple doses each day. Levels in plasma may be measured, for example, by HPLC.

In cases of local administration or selective uptake, the effective local concentration of the protease-activatable T cell activating bispecific molecules may not be related to plasma concentration. One having skill in the art will be able to optimize therapeutically effective local dosages without undue experimentation.

A therapeutically effective dose of the protease-activatable T cell activating bispecific molecules described herein will generally provide therapeutic benefit without causing substantial toxicity. Toxicity and therapeutic efficacy of a protease-activatable T cell activating bispecific molecule can be determined by standard pharmaceutical procedures in cell culture or experimental animals. Cell culture assays and animal studies can be used to determine the LD₅₀ (the dose lethal to 50% of a population) and the ED₅₀ (the dose therapeutically effective in 50% of a population). The dose ratio between toxic and therapeutic effects is the therapeutic index, which can be expressed as the ratio LD₅₀/ED₅₀. Protease-activatable T cell activating bispecific molecule that exhibit large therapeutic indices are preferred. In one embodiment, the protease-activatable T cell activating bispecific molecule according to the present invention exhibits a high therapeutic index. The data obtained from cell culture assays and animal studies can be used in formulating a range of dosages suitable for use in humans. The dosage lies preferably within a range of circulating concentrations that include the ED₅₀ with little or no toxicity. The dosage may vary within this range depending upon a variety of factors, e.g., the dosage form employed, the route of administration utilized, the condition of the subject, and the like. The exact formulation, route of administration and dosage can be chosen by the individual physician in view of the patient's condition (see, e.g., Fingl et al., 1975, in: The Pharmacological Basis of Therapeutics, Ch. 1, p. 1, incorporated herein by reference in its entirety).

The attending physician for patients treated with protease-activatable T cell activating bispecific molecules of the invention would know how and when to terminate, interrupt, or adjust administration due to toxicity, organ dysfunction, and the like. Conversely, the attending physician would also know to adjust treatment to higher levels if the clinical response were not adequate (precluding toxicity). The magnitude of an administered dose in the management of the disorder of interest will vary with the severity of the condition to be treated, with the route of administration, and the like. The severity of the condition may, for example, be evaluated, in part, by standard prognostic evaluation methods. Further, the dose and perhaps dose frequency will also vary according to the age, body weight, and response of the individual patient.

Other Agents and Treatments

The protease-activatable T cell activating bispecific molecules of the invention may be administered in combination with one or more other agents in therapy. For instance, a protease-activatable T cell activating bispecific molecule of the invention may be co-administered with at least one additional therapeutic agent. The term “therapeutic agent” encompasses any agent administered to treat a symptom or disease in an individual in need of such treatment. Such additional therapeutic agent may comprise any active ingredients suitable for the particular indication being treated, preferably those with complementary activities that do not adversely affect each other. In certain embodiments, an additional therapeutic agent is an immunomodulatory agent, a cytostatic agent, an inhibitor of cell adhesion, a cytotoxic agent, an activator of cell apoptosis, or an agent that increases the sensitivity of cells to apoptotic inducers. In a particular embodiment, the additional therapeutic agent is an anti-cancer agent, for example a microtubule disruptor, an antimetabolite, a topoisomerase inhibitor, a DNA intercalator, an alkylating agent, a hormonal therapy, a kinase inhibitor, a receptor antagonist, an activator of tumor cell apoptosis, or an antiangiogenic agent.

Such other agents are suitably present in combination in amounts that are effective for the purpose intended. The effective amount of such other agents depends on the amount of protease-activatable T cell activating bispecific molecule used, the type of disorder or treatment, and other factors discussed above. The protease-activatable T cell activating bispecific molecule are generally used in the same dosages and with administration routes as described herein, or about from 1 to 99% of the dosages described herein, or in any dosage and by any route that is empirically/clinically determined to be appropriate.

Such combination therapies noted above encompass combined administration (where two or more therapeutic agents are included in the same or separate compositions), and separate administration, in which case, administration of the protease-activatable T cell activating bispecific molecule of the invention can occur prior to, simultaneously, and/or following, administration of the additional therapeutic agent and/or adjuvant. Protease-activatable T cell activating bispecific molecules of the invention can also be used in combination with radiation therapy.

Articles of Manufacture

In another aspect of the invention, an article of manufacture containing materials useful for the treatment, prevention and/or diagnosis of the disorders described above is provided. The article of manufacture comprises a container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing the condition and may have a sterile access port (for example the container may be an intravenous solution bag or a vial having a stopper pierceable by a hypodermic injection needle). At least one active agent in the composition is a protease-activatable T cell activating bispecific molecule of the invention. The label or package insert indicates that the composition is used for treating the condition of choice. Moreover, the article of manufacture may comprise (a) a first container with a composition contained therein, wherein the composition comprises a protease-activatable T cell activating bispecific molecule of the invention; and (b) a second container with a composition contained therein, wherein the composition comprises a further cytotoxic or otherwise therapeutic agent. The article of manufacture in this embodiment of the invention may further comprise a package insert indicating that the compositions can be used to treat a particular condition. Alternatively, or additionally, the article of manufacture may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

### Exemplary Embodiments

1. A protease-activatable T cell activating bispecific molecule comprising
(a) a first antigen binding moiety capable of binding to CD3, wherein the first antigen binding moiety comprises
(a) a first antigen binding moiety capable of binding to CD3, wherein the first antigen binding moiety comprises
   (i) a heavy chain variable region (VH) comprising a heavy chain complementary determining region (HCDR) 1 of SEQ ID NO: 2, a HCDR 2 of SEQ ID NO: 4, and a HCDR 3 of SEQ ID NO: 10, and
   (ii) a light chain variable region (VL) comprising a light chain complementarity determining region (LCDR) 1 of SEQ ID NO: 20, a LCDR 2 of SEQ ID NO: 21 and a LCDR 3 of SEQ ID NO: 22;
(b) a second antigen binding moiety capable of binding to a target cell antigen; and
(c) a masking moiety covalently attached to the T cell bispecific binding molecule through a protease-cleavable linker, wherein the masking moiety is capable of binding to the idiotype of the first or the second antigen binding moiety thereby reversibly concealing the first antigen binding moiety.

2. The protease-activatable T cell activating bispecific molecule of embodiment 1, wherein the VH comprises an amino acid sequence that is at least about 95%, 96%, 97%, 98%, 99% or 100% identical to the amino acid sequence of SEQ ID NO: 16, and/or the VL comprises an amino acid sequence that is at least about 95%, 96%, 97%, 98%, 99% or 100% identical to the amino acid sequence of SEQ ID NO: 23.

3. The protease-activatable T cell activating bispecific molecule of embodiment 1 or 2, wherein the masking moiety is covalently attached to the first antigen binding moiety and reversibly conceals the first antigen binding moiety.

4. The protease-activatable T cell activating bispecific molecule of any one of embodiments 1-3, wherein the masking moiety is covalently attached to the heavy chain variable region of the first antigen binding moiety.

5. The protease-activatable T cell activating bispecific molecule of any one of embodiments 1-3, wherein the masking moiety is covalently attached to the light chain variable region of the first antigen binding moiety.

6. The protease-activatable T cell activating bispecific molecule of any one of embodiments 1-5, wherein the masking moiety is an scFv.

7. The protease-activatable T cell activating bispecific molecule of any one of embodiments 2-6, wherein the protease-activatable T cell activating bispecific molecule comprises a second masking moiety reversibly concealing the second antigen binding moiety.

8. The protease-activatable T cell activating bispecific molecule of any one of embodiments 1-7, wherein the protease is expressed by the target cell.

9. The protease-activatable T cell activating bispecific molecule of any one of embodiments 1-8, wherein the second antigen binding moiety is a crossover Fab molecule wherein either the variable or the constant regions of the Fab light chain and the Fab heavy chain are exchanged.

10. The protease-activatable T cell activating bispecific molecule of any one of embodiments 1-9, wherein the second antigen binding moiety is a crossover Fab molecule wherein the constant regions of the Fab light chain and the Fab heavy chain are exchanged.

11. The protease-activatable T cell activating bispecific molecule of any one of embodiments 1-10, wherein the first antigen binding moiety is a conventional Fab molecule.

12. The protease-activatable T cell activating bispecific molecule of any one of embodiments 1-11, comprising not more than one antigen binding moiety capable of binding to CD3.

13. The protease-activatable T cell activating bispecific molecule of any one of embodiments 1-12, comprising a third antigen binding moiety which is a Fab molecule capable of binding to a target cell antigen.

14. The protease-activatable T cell activating bispecific molecule of embodiment 13, wherein the third antigen binding moiety is identical to the second antigen binding moiety.

15. The protease-activatable T cell activating bispecific molecule of any one of embodiments 1-14, wherein the second antigen binding moiety is capable of binding to FolR1 or TYRP1.

16. The protease-activatable T cell activating bispecific molecule of any one of embodiments 1-14, wherein the second antigen binding moiety is capable of binding to FolR1.

17. The protease-activatable T cell activating bispecific molecule of any one of embodiments 1-14, wherein the second antigen binding moiety is capable of binding to TYRP1.

18. The protease-activatable T cell activating bispecific molecule of any one of embodiments 1-17, wherein the first and the second antigen binding moiety are fused to each other, optionally via a peptide linker.

19. The protease-activatable T cell activating bispecific molecule of any one of embodiments 1-18, wherein the second antigen binding moiety is fused at the C-terminus of the Fab heavy chain to the N-terminus of the Fab heavy chain of the first antigen binding moiety.

20. The protease-activatable T cell activating bispecific molecule of any one of embodiments 1-18, wherein the first antigen binding moiety is fused at the C-terminus of the Fab heavy chain to the N-terminus of the Fab heavy chain of the second antigen binding moiety.

21. The protease-activatable T cell activating bispecific molecule of any one of embodiments 1-20, wherein the Fab light chain of the first antigen binding moiety and the Fab light chain of the second antigen binding moiety are fused to each other, optionally via a peptide linker.

22. The protease-activatable T cell activating bispecific molecule of any one of embodiments 1-21, additionally comprising an Fc domain composed of a first and a second subunit capable of stable association.

23. The protease-activatable T cell activating bispecific molecule of embodiment 22, wherein the Fc domain is an IgG, specifically an IgG1 or IgG4, Fc domain.

24. The protease-activatable T cell activating bispecific molecule of embodiment 22 or 23, wherein the Fc domain is a human Fc domain.

25. The protease-activatable T cell activating bispecific molecule of any one of embodiments 22-24, wherein the Fc domain exhibits reduced binding affinity to an Fc receptor and/or reduced effector function, as compared to a native IgG1 Fc domain.

26. The protease-activatable T cell activating bispecific molecule of embodiment 25, wherein the Fc domain comprises one or more amino acid substitution that reduces binding to an Fc receptor and/or effector function.

27. The protease-activatable T cell activating bispecific molecule of embodiment 26, wherein said one or more amino acid substitution is at one or more position selected from the group of L234, L235, and P329 (Kabat numbering).

28. The protease-activatable T cell activating bispecific molecule of embodiment 27, wherein each subunit of the Fc domain comprises three amino acid substitutions that reduce binding to an activating Fc receptor and/or effector function wherein said amino acid substitutions are L234A, L235A and P329G.

29. The protease-activatable T cell activating bispecific molecule of any one of embodiments 25-28, wherein the Fc receptor is an Fcγ receptor.

30. The protease-activatable T cell activating bispecific molecule of any one of embodiments 25-28, wherein the effector function is antibody-dependent cell-mediated cytotoxicity (ADCC).

31. The protease-activatable T cell activating bispecific molecule of any one of embodiments 1-30, wherein the masking moiety comprises a heavy chain variable region comprising at least one of:
(a) a heavy chain complementarity determining region (CDR H) 1 amino acid sequence of DYSMN (SEQ ID NO:58);
(b) a CDR H2 amino acid sequence selected from the group consisting of

(SEQ ID NO: 59)
WINTETGEPRYTDDFKG,
(SEQ ID NO: 84)
WINTETGEPRYTDDFTG
and
(SEQ ID NO: 86)
WINTETGEPRYTQGFKG;

(c) a CDR H3 amino acid sequence of EGDYDVFDY (SEQ ID NO:60.

32. The protease-activatable T cell activating bispecific molecule of any one of embodiments 1-31, wherein the masking moiety comprises a light chain variable region comprising at least one of:
(d) a light chain (CDR L)1 amino acid sequence selected from the group consisting of RASKSVSTSSYSYMH (SEQ ID NO:62) and KSSKSVSTSSYSYMH (SEQ ID NO:82);
(e) a CDR L2 amino acid sequence of YVSYLES (SEQ ID NO:63); and
(f) a CDR L3 amino acid sequence selected from the group consisting of

(SEQ ID NO: 64)
QHSREFPYT
and
(SEQ ID NO: 88)
QQSREFPYT.

33. The protease-activatable T cell activating bispecific molecule of any one of embodiments 1-30, wherein the masking moiety comprises a heavy chain variable region comprising:
(a) a heavy chain complementarity determining region (CDR H) 1 amino acid sequence of DYSMN (SEQ ID NO:58);
(b) a CDR H2 amino acid sequence selected from the group consisting of

(SEQ ID NO: 59)
WINTETGEPRYTDDFKG,
(SEQ ID NO: 84)
WINTETGEPRYTDDFTG
and
(SEQ ID NO: 86)
WINTETGEPRYTQGFKG;

(c) a CDR H3 amino acid sequence of EGDYDVFDY (SEQ ID NO:60); and a light chain variable region comprising:
(d) a light chain (CDR L)1 amino acid sequence selected from the group consisting of

(SEQ ID NO: 62)
RASKSVSTSSYSYMH
and
(SEQ ID NO: 82)
KSSKSVSTSSYSYMH;

(e) a CDR L2 amino acid sequence of YVSYLES (SEQ ID NO:63); and
(f) a CDR L3 amino acid sequence selected from the group consisting of

(SEQ ID NO: 64)
QHSREFPYT
and
(SEQ ID NO: 88)
QQSREFPYT.

34. The protease-activatable T cell activating bispecific molecule of any one of embodiments 1-30, wherein the masking moiety comprises a heavy chain variable region comprising:
(a) a heavy chain complementarity determining region (CDR H) 1 amino acid sequence of DYSMN (SEQ ID NO:58);
(b) a CDR H2 amino acid sequence of WINTETGEPRYTDDFKG (SEQ ID NO:59);
(c) a CDR H3 amino acid sequence of EGDYDVFDY (SEQ ID NO:60); and a light chain variable region comprising:
(d) a light chain (CDR L)1 amino acid sequence of RASKSVSTSSYSYMH (SEQ ID NO:62);
(e) a CDR L2 amino acid sequence of YVSYLES (SEQ ID NO:63); and
(f) a CDR L3 amino acid sequence of QHSREFPYT (SEQ ID NO:64).

35. The protease-activatable T cell activating bispecific molecule of any one of embodiments 1-30, wherein the masking moiety comprises a heavy chain variable region comprising:
(a) a heavy chain complementarity determining region (CDR H) 1 amino acid sequence of SYGVS (SEQ ID NO:58);
(b) a CDR H2 amino acid sequence of IIWGDGSTNYHSALIS (SEQ ID NO:59);
(c) a CDR H3 amino acid sequence of GITTVVDDYYAMDY (SEQ ID NO:60); and a light chain variable region comprising:
(d) a light chain (CDR L)1 amino acid sequence of KSSKSVSTSSYSYMH (SEQ ID NO: 82);
(e) a CDR L2 amino acid sequence of AATFLAD (SEQ ID NO:63); and
(f) a CDR L3 amino acid sequence of QHSREFPYT (SEQ ID NO:64).

36. The protease-activatable T cell activating bispecific molecule of any one of embodiments 1-30, wherein the masking moiety comprises a heavy chain variable region comprising:
(a) a heavy chain complementarity determining region (CDR H) 1 amino acid sequence of SYGVS (SEQ ID NO:58);
(b) a CDR H2 amino acid sequence of WINTETGEPRYTDDFTG (SEQ ID NO:84);
(c) a CDR H3 amino acid sequence of GITTVVDDYYAMDY (SEQ ID NO:60); and a light chain variable region comprising:
(d) a light chain (CDR L)1 amino acid sequence of KSSKSVSTSSYSYMH (SEQ ID NO:82);
(e) a CDR L2 amino acid sequence of AATFLAD (SEQ ID NO:63); and
(f) a CDR L3 amino acid sequence of QHYYSTPYT (SEQ ID NO:64).

37. The protease-activatable T cell activating bispecific molecule of any one of embodiments 1-30, wherein the masking moiety comprises a heavy chain variable region comprising:
(a) a heavy chain complementarity determining region (CDR H) 1 amino acid sequence of SYGVS (SEQ ID NO:58);
(b) a CDR H2 amino acid sequence of WINTETGEPRYTQGFKG (SEQ ID NO:86);
(c) a CDR H3 amino acid sequence of GITTVVDDYYAMDY (SEQ ID NO:60); and a light chain variable region comprising:
(d) a light chain (CDR L)1 amino acid sequence of KSSKSVSTSSYSYMH (SEQ ID NO: 82);
(e) a CDR L2 amino acid sequence of AATFLAD (SEQ ID NO:63); and
(f) a CDR L3 amino acid sequence of QHYYSTPYT (SEQ ID NO:64).

38. The protease-activatable T cell activating bispecific molecule of any one of embodiments 1-37, wherein the masking moiety is humanized.

39. The protease-activatable T cell activating bispecific molecule of any one of embodiments 1-38, wherein the masking moiety is human.

40. The protease-activatable T cell activating bispecific molecule of any one of embodiments 1-39, wherein the protease cleavable linker comprises at least one protease recognition sequence.

41. The protease-activatable T cell activating bispecific molecule of embodiment 40, wherein the protease cleavable linker comprises a protease recognition sequence.

42. The protease-activatable T cell activating bispecific molecule of embodiment 41, wherein the protease recognition sequence is selected from the group consisting of:

(a)
(SEQ ID NO: 100)
RQARVVNG;
(b)
(SEQ ID NO: 101)
VHMPLGFLGPGRSRGSFP;
(c)
(SEQ ID NO: 102)
RQARVVNGXXXXXVPLSLYSG,
wherein X is any amino acid;
(d)
(SEQ ID NO: 103)
RQARVVNGVPLSLYSG;
(e)
(SEQ ID NO: 104)
PLGLWSQ;
(f)
(SEQ ID NO: 105)
VHMPLGFLGPRQARVVNG;
(g)
(SEQ ID NO: 106)
FVGGTG;
(h)
(SEQ ID NO: 107)
KKAAPVNG;
(i)
(SEQ ID NO: 108)
PMAKKVNG;
(j)
(SEQ ID NO: 109)
QARAKVNG;
(k)
(SEQ ID NO: 110)
VHMPLGFLGP;
(l)
(SEQ ID NO: 111)
QARAK;
(m)
(SEQ ID NO: 112)
VHMPLGFLGPPMAKK;
(n)
(SEQ ID NO: 113)
KKAAP;
and
(o)
(SEQ ID NO: 114)
PMAKK.

43. The protease-activatable T cell activating bispecific molecule of embodiment 40 or 41, wherein the protease cleavable linker comprises the protease recognition sequence PMAKK (SEQ ID NO:114).

44. The protease-activatable T cell activating bispecific molecule of embodiment 40 or 41, wherein the protease cleavable linker comprises the protease recognition sequence

(SEQ ID NO: 112)
VHMPLGFLGPPMAKK.

45. The protease-activatable T cell activating bispecific molecule of embodiment 40 or 41, wherein the protease cleavable linker comprises the protease recognition sequence VHMPLGFLGPRQARVVNG (SEQ ID NO:105).

46. The protease-activatable T cell activating bispecific molecule of embodiment 40 or 41, wherein the protease cleavable linker comprises the protease recognition sequence RQARVVNG (SEQ ID NO:100) or the protease recognition sequence VHMPLGFLGPRQARVVNG (SEQ ID NO:105).

47. The protease-activatable T cell activating bispecific molecule of any one of embodiments 1 to 46, wherein the protease is selected from the group consisting of metalloproteinase, serine protease, cysteine protease, aspartic proteases, and cathepsin protease.

48. The protease-activatable T cell activating bispecific molecule of embodiment 47, wherein the metalloproteinase is a matrix metalloproteinase (MMP), preferably MMP9 or MMP2.

49. The protease-activatable T cell activating bispecific molecule of embodiment 47, wherein the serine protease is Matriptase.

50. The protease-activatable T cell activating bispecific molecule of any one of embodiments 1 to 49, wherein the second antigen binding moiety is capable of binding to FolR1 and comprises at least one heavy chain complementarity determining region (CDR) selected from the group consisting of SEQ ID NO: 54, SEQ ID NO: 55 and SEQ ID NO: 56 and/or at least one light chain CDR selected from the group of SEQ ID NO: 20, SEQ ID NO: 21 and SEQ ID NO: 22.

51. The protease-activatable T cell activating bispecific molecule of any one of embodiments 1-49, wherein the second antigen binding moiety is capable of binding to FolR1 and comprises at least one heavy chain complementarity determining region (CDR) selected from the group consisting of SEQ ID NO: 54, SEQ ID NO: 55 and SEQ ID NO: 56 and at least one light chain CDR selected from the group of SEQ ID NO: 20, SEQ ID NO: 21 and SEQ ID NO: 22.

52. The protease-activatable T cell activating bispecific molecule of any one of embodiments 1-51, wherein the second antigen binding moiety is capable of binding to FolR1 and comprises a heavy chain variable region comprising:
a) a heavy chain complementarity determining region (CDR H) 1 amino acid sequence of NAWMS (SEQ ID NO:54);
b) a CDR H2 amino acid sequence of RIKSKTDGGTTDYAAPVKG (SEQ ID NO:55); and
c) a CDR H3 amino acid sequence of PWEWSWYDY (SEQ ID NO:56); and a light chain variable region comprising:
d) a light chain (CDR L)1 amino acid sequence of GSSTGAVTTSNYAN (SEQ ID NO:20);
e) a CDR L2 amino acid sequence of GTNKRAP (SEQ ID NO:21); and
f) a CDR L3 amino acid sequence of ALWYSNLWV (SEQ ID NO:22).

53. The protease-activatable T cell activating bispecific molecule of any one of embodiments 1-52, wherein the second antigen binding moiety comprises a heavy chain variable region comprising an amino acid sequence that is at least about 95%, 96%, 97%, 98%, 99% or 100% identical to the amino acid sequence of SEQ ID NO: 53 and a light chain variable region comprising an amino acid sequence that is at least about 95%, 96%, 97%, 98%, 99% or 100% identical to the amino acid sequence of SEQ ID NO: 23.

54. The protease-activatable T cell activating bispecific molecule of any one of embodiments 1-53, wherein the second antigen binding moiety is capable of binding to FolR1 and comprises a heavy chain variable region comprising the amino acid sequence of SEQ ID NO: 53 and a light chain variable region comprising the amino acid sequence of SEQ ID NO: 23.

55. The protease-activatable T cell activating bispecific molecule of any one of embodiments 1-49, wherein the second antigen binding moiety is capable of binding to TYRP1 and comprises at least one heavy chain complementarity determining region (CDR) selected from the group consisting of SEQ ID NO: 24, SEQ ID NO: 25 and SEQ ID NO: 26 and/or at least one light chain CDR selected from the group of SEQ ID NO: 28, SEQ ID NO: 29 and SEQ ID NO: 30.

56. The protease-activatable T cell activating bispecific molecule of any one of embodiments 1-49 and 55, wherein the second antigen binding moiety is capable of binding to TYRP1 and comprises at least one heavy chain complementarity determining region (CDR) selected from the group consisting of of SEQ ID NO: 24, SEQ ID NO: 25 and SEQ ID NO: 26 and at least one light chain CDR selected from the group of SEQ ID NO: 28, SEQ ID NO: 29 and SEQ ID NO: 30.

57. The protease-activatable T cell activating bispecific molecule of any one of embodiments 1-49 and 55-56, wherein the second antigen binding moiety is capable of binding to TYRP1 and comprises a heavy chain variable region comprising:
a) a heavy chain complementarity determining region (CDR H) 1 amino acid sequence of NAWMS (SEQ ID NO:24);
b) a CDR H2 amino acid sequence of RIKSKTDGGTTDYAAPVKG (SEQ ID NO:25); and
c) a CDR H3 amino acid sequence of PWEWSWYDY (SEQ ID NO:26); and a light chain variable region comprising:
d) a light chain (CDR L)1 amino acid sequence of GSSTGAVTTSNYAN (SEQ ID NO:28);
e) a CDR L2 amino acid sequence of GTNKRAP (SEQ ID NO:29); and
f) a CDR L3 amino acid sequence of ALWYSNLWV (SEQ ID NO:30).

58. The protease-activatable T cell activating bispecific molecule of any one of embodiments 1-49 and 55-57, wherein the second antigen binding moiety comprises a heavy chain variable region comprising an amino acid sequence that is at least about 95%, 96%, 97%, 98%, 99% or 100% identical to the amino acid sequence of SEQ ID NO: 27 and a light chain variable region comprising an amino acid sequence that is at least about 95%, 96%, 97%, 98%, 99% or 100% identical to the amino acid sequence of SEQ ID NO: 31.

59. The protease-activatable T cell activating bispecific molecule of any one of embodiments 1-53, wherein the second antigen binding moiety is capable of binding to TYRP1 and comprises a heavy chain variable region comprising the amino acid sequence of SEQ ID NO: 27 and a light chain variable region comprising the amino acid sequence of SEQ ID NO: 31.

60. The protease-activatable T cell activating bispecific molecule of any one of embodiments 1-54, comprising
a) at least one heavy chain comprising the amino acid sequence of SEQ ID NO:66;
b) at least one light chain comprising the amino acid sequence of SEQ ID NO:67.

61. The protease-activatable T cell activating bispecific molecule of any one of embodiments 1-50, comprising
(a) a first heavy chain comprising the amino acid sequence of SEQ ID NO:65;
(b) a second heavy chain comprising the amino acid sequence of SEQ ID NO:66; and
(c) a light chain comprising an amino acid sequence of SEQ ID NO:67.

62. The protease-activatable T cell activating bispecific molecule of any one of embodiments 1-54, comprising
(a) a first heavy chain comprising the amino acid sequence of SEQ ID NO:69;
(b) a second heavy chain comprising the amino acid sequence of SEQ ID NO:66; and
(c) a light chain comprising an amino acid sequence of SEQ ID NO:67.

63. The protease-activatable T cell activating bispecific molecule of any one of embodiments 1-54, comprising
(a) a first heavy chain comprising the amino acid sequence of SEQ ID NO:74;
(b) a second heavy chain comprising the amino acid sequence of SEQ ID NO:66; and
(c) a light chain comprising an amino acid sequence of SEQ ID NO:67.

64. The protease-activatable T cell activating bispecific molecule of any one of embodiments 1-54, comprising
(a) a first heavy chain comprising the amino acid sequence of SEQ ID NO:76;
(b) a second heavy chain comprising the amino acid sequence of SEQ ID NO:66; and
(c) a light chain comprising an amino acid sequence of SEQ ID NO:67.

65. The protease-activatable T cell activating bispecific molecule of any one of embodiments 1-54, comprising
(a) a first heavy chain comprising the amino acid sequence of SEQ ID NO:95;
(b) a second heavy chain comprising the amino acid sequence of SEQ ID NO:66; and
(c) a light chain comprising an amino acid sequence of SEQ ID NO:67.

66. The protease-activatable T cell activating bispecific molecule of any one of embodiments 1-54, comprising
(a) a first heavy chain comprising the amino acid sequence of SEQ ID NO:96;
(b) a second heavy chain comprising the amino acid sequence of SEQ ID NO:66; and
(c) a light chain comprising an amino acid sequence of SEQ ID NO:67.

67. The protease-activatable T cell activating bispecific molecule of any one of embodiments 1-54, comprising
(a) a first heavy chain comprising the amino acid sequence of SEQ ID NO:97;
(b) a second heavy chain comprising the amino acid sequence of SEQ ID NO:66; and
(c) a light chain comprising an amino acid sequence of SEQ ID NO:67.

68. The protease-activatable T cell activating bispecific molecule of any one of embodiments 1-54, comprising
(a) a first heavy chain comprising the amino acid sequence of SEQ ID NO:98;
(b) a second heavy chain comprising the amino acid sequence of SEQ ID NO:66; and
(c) a light chain comprising an amino acid sequence of SEQ ID NO:67.

69. The protease activatable T cell activating bispecific molecule of any one of embodiments 60-68, comprising
(c) two light chains comprising an amino acid sequence of SEQ ID NO:67.

70. The protease-activatable T cell activating bispecific molecule of any one of embodiments 1-69, wherein the masking moiety comprises a scFv comprising an amino acid sequence that is at least about 95%, 96%, 97%, 98%, 99% or 100% identical to the amino acid sequence of the amino acid sequence of SEQ ID NO:91.

71. The protease-activatable T cell activating bispecific molecule of any one of embodiments 1-69, wherein the masking moiety comprises a scFv comprising an amino acid sequence that is at least about 95%, 96%, 97%, 98%, 99% or 100% identical to the amino acid sequence of the amino acid sequence of SEQ ID NO:92.

72. The protease-activatable T cell activating bispecific molecule of any one of embodiments 1-69, wherein the masking moiety comprises a scFv comprising an amino acid sequence that is at least about 95%, 96%, 97%, 98%, 99% or 100% identical to the amino acid sequence of the amino acid sequence of SEQ ID NO:93.

73. The protease-activatable T cell activating bispecific molecule of any one of embodiments 1-69, wherein the masking moiety comprises a scFv comprising an amino acid sequence that is at least about 95%, 96%, 97%, 98%, 99% or 100% identical to the amino acid sequence of the amino acid sequence of SEQ ID NO:94.

74. The protease-activatable T cell activating bispecific molecule of any one of embodiments 70-73, wherein the binding affinity of the masking moiety to the first antigen binding moiety as measured by SPR is about the same or higher compared to the binding affinity of a masking moiety comprising the amino acid sequence selected from the group consisting of SEQ ID NO:91, SEQ ID NO:92, SEQ ID NO:93 and SEQ ID NO:94.

75. An idiotype-specific polypeptide capable of reversibly concealing an anti-CD3 antigen binding site of a molecule, wherein the idiotype-specific polypeptide comprises a heavy chain variable region sequence that is at least about 95%, 96%, 97%, 98%, 99% or 100% identical to an amino acid sequence selected from the group consisting of SEQ ID NO: 79, SEQ ID NO:83 and SEQ ID NO:85, and a light chain variable region sequence that is at least about 95%, 96%, 97%, 98%, 99% or 100% identical to an amino acid sequence selected from the group consisting of SEQ ID NO: 80 and SEQ ID NO:81,

76. The idiotype-specific polypeptide of claim 28, wherein the idiotype-specific polypeptide comprises a heavy chain variable region sequence that is at least about 95%, 96%, 97%, 98%, 99% or 100% identical to SEQ ID NO: 79 and a light chain variable region sequence that is at least about 95%, 96%, 97%, 98%, 99% or 100% identical to SEQ ID NO: 80,

77. The idiotype-specific polypeptide of claim 28, wherein the idiotype-specific polypeptide comprises a heavy chain variable region sequence that is at least about 95%, 96%, 97%, 98%, 99% or 100% identical to SEQ ID NO: 79 and a light chain variable region sequence that is at least about 95%, 96%, 97%, 98%, 99% or 100% identical to SEQ ID NO: 81,

78. The idiotype-specific polypeptide of claim 28, wherein the idiotype-specific polypeptide comprises a heavy chain variable region sequence that is at least about 95%, 96%, 97%, 98%, 99% or 100% identical to SEQ ID NO: 83 and a light chain variable region sequence that is at least about 95%, 96%, 97%, 98%, 99% or 100% identical to SEQ ID NO: 81,

79. The idiotype-specific polypeptide of claim 28, wherein the idiotype-specific polypeptide comprises a heavy chain variable region sequence that is at least about 95%, 96%, 97%, 98%, 99% or 100% identical to SEQ ID NO: 85 and a light chain variable region sequence that is at least about 95%, 96%, 97%, 98%, 99% or 100% identical to SEQ ID NO: 81,

80. The idiotype-specific polypeptide of embodiment 75, wherein the idiotype-specific polypeptide is an anti-idiotype scFv, an anti-idiotype Fab or an anti-idiotype scFab.

81. The idiotype-specific polypeptide of any one of embodiments 75-80, wherein the idiotype-specific polypeptide is an scFv.

82. The idiotype-specific polypeptide of any one of embodiments 75-80, wherein the idiotype-specific polypeptide is covalently attached to the molecule through a linker.

83. The idiotype-specific polypeptide of embodiment 82, wherein the linker is a peptide linker.

84. The idiotype-specific polypeptide of embodiment 82 or 83, wherein the linker is a protease-cleavable linker.

85. The idiotype-specific polypeptide of any one of embodiments 82-84, wherein the peptide linker comprises at least one protease recognition sequence.

86. The idiotype-specific polypeptide of embodiment 85 wherein the protease is selected from the group consisting of metalloproteinase, serine protease, cysteine protease, aspartic proteases, and cathepsin protease.

87. The idiotype-specific polypeptide of embodiment 91, wherein the metalloproteinase is a matrix metalloproteinase (MMP), preferably MMP9 or MMP2.

88. The idiotype-specific polypeptide of embodiment 86, wherein the serine protease is Matriptase.

89. The idiotype-specific polypeptide of any one of embodiments 85-88, wherein the protease recognition sequence is selected from the group consisting of:

(a)
(SEQ ID NO: 100)
RQARVVNG;
(b)
(SEQ ID NO: 101)
VHMPLGFLGPGRSRGSFP;
(c)
(SEQ ID NO: 102)
RQARVVNGXXXXXVPLSLYSG,
wherein X is any amino acid;
(d)
(SEQ ID NO: 103)
RQARVVNGVPLSLYSG;
(e)
(SEQ ID NO: 104)
PLGLWSQ;
(f)
(SEQ ID NO: 105)
VHMPLGFLGPRQARVVNG;
(g)
(SEQ ID NO: 106)
FVGGTG;
(h)
(SEQ ID NO: 107)
KKAAPVNG;
(i)
(SEQ ID NO: 108)
PMAKKVNG;
(j)
(SEQ ID NO: 109)
QARAKVNG;
(k)
(SEQ ID NO: 110)
VHMPLGFLGP;
(l)
(SEQ ID NO: 111)
QARAK;
(m)
(SEQ ID NO: 112)
VHMPLGFLGPPMAKK;
(n)
(SEQ ID NO: 113)
KKAAP;
and
(o)
(SEQ ID NO: 114)
PMAKK.

90. The idiotype-specific polypeptide of any one of embodiments 80-83, wherein the protease cleavable linker comprises the protease recognition sequence PMAKK (SEQ ID NO:114).

91. The idiotype-specific polypeptide of any one of embodiments 80-90, wherein the idiotype-specific polypeptide is part of a T-cell activating bispecific molecule.

92. The idiotype-specific polypeptide of embodiments 75-92 wherein the idiotype-specific polypeptide comprises a heavy chain variable region comprising the amino acid sequence of SEQ ID NO: 79 and a light chain variable region comprising the amino acid sequence of SEQ ID NO: 80.

93. The idiotype-specific polypeptide of embodiments 75-92 wherein the idiotype-specific polypeptide comprises a heavy chain variable region comprising the amino acid sequence of SEQ ID NO: 79 and a light chain variable region comprising the amino acid sequence of SEQ ID NO: 81.

94. The idiotype-specific polypeptide of embodiments 75-92 wherein the idiotype-specific polypeptide comprises a heavy chain variable region comprising the amino acid sequence of SEQ ID NO: 83 and a light chain variable region comprising the amino acid sequence of SEQ ID NO: 84.

95. The idiotype-specific polypeptide of embodiments 75-92 wherein the idiotype-specific polypeptide comprises a heavy chain variable region comprising the amino acid sequence of SEQ ID NO: 85 and a light chain variable region comprising the amino acid sequence of SEQ ID NO: 86.

96. The idiotype-specific polypeptide of embodiments 75-92 wherein the idiotype-specific polypeptide comprises a heavy chain variable region comprising the amino acid sequence of SEQ ID NO: 84 and a light chain variable region comprising the amino acid sequence of SEQ ID NO: 87.

97. The idiotype-specific polypeptide of embodiments 75-92 wherein the idiotype-specific polypeptide comprises a heavy chain variable region comprising the amino acid sequence of SEQ ID NO: 89 and a light chain variable region comprising the amino acid sequence of SEQ ID NO:90.

98. The idiotype-specific polypeptide of embodiments 75 to 97, wherein the anti-CD3 antigen binding site comprises a heavy chain variable region comprising the amino acid sequence of SEQ ID NO: 16 and a light chain variable region comprising the amino acid sequence of SEQ ID NO: 23.

99. The idiotype-specific polypeptide of embodiments 75 to 98, wherein the idiotype-specific polypeptide is humanized.

100. An isolated polynucleotide encoding the protease-activatable T cell activating bispecific antigen binding molecule of any one of embodiments 1-74 or the idiotype-specific polypeptide of any one of embodiments 75-99.

101. A polypeptide encoded by the polynucleotide of embodiment 100.

102. A vector, particularly an expression vector, comprising the polynucleotide of embodiment 100.

103. A host cell comprising the polynucleotide of embodiment 99 or the vector of embodiment 102.

104. A method of producing a protease-activatable T cell activating bispecific molecule, comprising the steps of a) culturing the host cell of embodiment 103 under conditions suitable for the expression of the protease-activatable T cell activating bispecific molecule and b) recovering the protease-activatable T cell activating bispecific molecule.

105. A protease-activatable T cell activating bispecific molecule produced by the method of embodiment 104.

106. A method of producing an idiotype-specific polypeptide, comprising the steps of a) culturing the host cell of embodiment 103 under conditions suitable for the expression of the idiotype-specific polypeptide and b) recovering the an idiotype-specific polypeptide.

107. An idiotype-specific polypeptide produced by the method of embodiment 106.

108. A pharmaceutical composition comprising the protease-activatable T cell activating bispecific molecule of any one of embodiments 1 to 74 and a pharmaceutically acceptable carrier.

109. A pharmaceutical composition comprising the idiotype-specific polypeptide of any one of embodiments 75 to 99 and a pharmaceutically acceptable carrier.

110. A protease-activatable T cell activating bispecific molecule of any one of embodiments 1 to 74, the idiotype-specific polypeptide of any one of embodiments 75 to 99 or the composition of embodiment 108 for use as a medicament.

111. The protease-activatable T cell activating bispecific molecule for use according to embodiment 110, wherein the medicament is for treating or delaying progression of cancer, treating or delaying progression of an immune related disease, or enhancing or stimulating an immune response or function in an individual.

112. The protease-activatable T cell activating bispecific molecule of any one of embodiments 1 to 74 or the idiotype-specific polypeptide of any one of embodiments 75 to 99 for use in the treatment of a disease in an individual in need thereof.

113. The protease-activatable T cell activating bispecific molecule or the idiotype-specific polypeptide for use in the treatment of a disease in an individual in need thereof of embodiment 112, wherein the disease is a cancer.

114. Use of the protease-activatable T cell activating bispecific molecule of any one of embodiments 1 to 74 or the idiotype-specific polypeptide of any one of embodiments 75 to 99 for the manufacture of a medicament for the treatment of a disease.

115. The use of embodiment 114, wherein the disease is a cancer.

116. A method of treating a disease in an individual, comprising administering to said individual a therapeutically effective amount of a composition comprising the protease-activatable T cell activating bispecific molecule of any one of embodiments 1 to 74 or composition of embodiment 108.

117. A method for inducing lysis of a target cell, comprising contacting a target cell with the protease-activatable T cell activating bispecific molecule of any one of embodiments 1 to 74 or composition of embodiment 108 in the presence of a T cell.

118. The method of embodiment 117 wherein the target cell is a cancer cell.

119. The method of embodiment 117 or 118, wherein the target cell expresses a protease capable of activating the protease-activatable T cell activating bispecific molecule.

120. A humanized anti-idiotype CD3 antibody or antigen-binding fragment thereof specific for an idiotype of an anti-CD3 antigen-binding molecule, wherein the anti-idiotype CD3 antibody or fragment thereof when bound to the anti-CD3 antigen-binding molecule specifically blocks binding of the anti-CD3 antigen-binding molecule to CD3.

121. The anti-idiotype CD3 antibody or antigen-binding fragment thereof of embodiment 120, wherein the anti-idiotype CD3 antibody or fragment thereof is reversibly associated with the anti-CD3 antigen-binding molecule through a peptide linker comprising a protease recognition site.

122. The anti-idiotype CD3 antibody or antigen-binding fragment thereof of embodiment 120 or 121, wherein the CD3 is a mouse, monkey or human CD3.

123. A method of reducing in vivo toxicity of a T cell activating bispecific molecule comprising attaching an idiotype-specific polypeptide of any one of embodiments 75 to 99 to the T cell activating bispecific molecule with a protease-cleavable linker to form a protease-activatable T cell activating bispecific molecule, wherein the in vivo toxicity of the protease-activatable T cell activating bispecific molecule is reduced compared to toxicity of the T cell activating bispecific molecule.

124. The invention as described hereinbefore.

EXEMPLARY SEQUENCES
CDR definition according to Kabat

| | | |
|---|---|---|
| CD3_{orig} | TYAMN | 1 |
| HCDR1 | | |
| CD3ₒₚₜ | SYAMN | 2 |
| HCDR1 | | |
| (P033.078) | | |
| (P035.093) | | |
| (P021.045) | | |
| CD3ₒₚₜ | NYAMN | 3 |
| HCDR1 | | |
| (P035.064) | | |
| (P004.042) | | |
| CD3_{orig} | RIRSKYNNYATYYADSVKG | 4 |
| HCDR2, | | |
| CD3ₒₚₜ | | |
| HCDR2 | | |
| (P035.093) | | |
| (P021.045) | | |
| CD3ₒₚₜ | RIRSKYNEYATYYADSVKG | 5 |
| HCDR2 | | |
| (P033.078) | | |
| CD3ₒₚₜ | RIRSKHNGYATYYADSVKG | 6 |
| HCDR2 | | |
| (P035.064) | | |
| CD3ₒₚₜ | RIRTKYNEYATYYADSVKG | 7 |
| HCDR2 | | |
| (P004.042) | | |
| CD3_{orig} | HGNFGNSYVSWFAY | 8 |
| HCDR3 | | |
| CD3ₒₚₜ | ASNFPSSFVSYFGY | 9 |
| HCDR3 | | |
| (P033.078) | | |
| CD3ₒₚₜ | ASNFPASYVSYFAY | 10 |
| HCDR3 | | |
| (P035.093) | | |
| CD3ₒₚₜ | ASNFPSSYVSYFGY | 11 |
| HCDR3 | | |
| (P035.064) | | |
| CD3ₒₚₜ | ASNFPSSYVSYFAY | 12 |
| HCDR3 | | |
| (P021.045) | | |
| CD3ₒₚₜ | ASNFPQSYVSYFGY | 13 |
| HCDR3 | | |
| (P004.042) | | |
| CD3_{orig} VH | EVQLLESGGGLVQPGGSLRLSCAASGFTFSTYAMNWVRQA | 14 |
| | PGKGLEWVSRIRSKYNNYATYYADSVKGRFTISRDDSKNT | |
| | LYLQMNSLRAEDTAVYYCVRHGNFGNSYVSWFAYWGQG | |
| | TLVTVSS | |
| CD3ₒₚₜ VH | EVQLLESGGGLVQPGGSLRLSCAASGFTFESYAMNWVRQA | 15 |
| (P033.078) | PGKGLEWVSRIRSKYNEYATYYADSVKGRFTI | |
| | SRDDSKNTLYLQMNSLRAEDTAVYYCVRASNFPSSFVSYF | |
| | GYWGQGTLVTVSS | |
| CD3ₒₚₜ VH | EVQLLESGGGLVQPGGSLRLSCAASGFTFSSYAMNWVRQA | 16 |
| (P035.093) | PGKGLEWVSRIRSKYNNYATYYADSVKGRFTISRDDSKNT | |
| | LYLQMNSLRAEDTAVYYCVRASNFPASYVSYFAYWGQGT | |
| | LVTVSS | |
| CD3ₒₚₜ VH | EVQLLESGGGLVQPGGSLRLSCAASGFDFDNYAMNWVRQ | 17 |
| (P035.064) | APGKGLEWVSRIRSKHNGYATYYADSVKGRFTISRDDSKN | |
| | TLYLQMNSLRAEDTAVYYCVRASNFPSSYVSYFGYWGQG | |
| | TLVTVSS | |
| CD3ₒₚₜ VH | EVQLLESGGGLVQPGGSLRLSCAASGFTFSSYAMNWVRQA | 18 |
| (P021.045) | PGKGLEWVSRIRSKYNNYATYYADSVKGRFTISRDDSKNT | |
| | LYLQMNSLRAEDTAVYYCVRASNFPSSYVSYFAYWGQGT | |
| | LVTVSS | |
| CD3ₒₚₜ VH | EVQLLESGGGLVQPGGSLRLSCAASGFQFDNYAMNWVRQ | 19 |
| (P004.042) | APGKGLEWVSRIRTKYNEYATYYADSVKGRFTISRDDSKN | |
| | TLYLQMNSLRAEDTAVYYCVRASNFPQSYVSYFGYWGQG | |
| | TLVTVSS | |
| CD3_{orig}/ | GSSTGAVTTSNYAN | 20 |
| CD3ₒₚₜ | | |
| LCDR1 | | |
| CD3_{orig}/ | GTNKRAP | 21 |
| CD3ₒₚₜ | | |
| LCDR2 | | |
| CD3_{orig}/ | ALWYSNLWV | 22 |
| CD3ₒₚₜ | | |
| LCDR3 | | |
| CD3_{orig}/ | QAVVTQEPSLTVSPGGTVTLTCGSSTGAVTTSNYANWVQE | 23 |
| CD3ₒₚₜ VL | KPGQAFRGLIGGTNKRAPGTPARFSGSLLGGKAALTLSGAQ | |
| | PEDEAEYYCALWYSNLWVFGGGTKLTVL | |
| TYRP1 | DYFLH | 24 |
| HCDR1 | | |
| TYRP1 | WINPDNGNTVYAQKFQG | 25 |
| HCDR2 | | |
| TYRP1 | RDYTYEKAALDY | 26 |
| HCDR3 | | |
| TYRP1 VH | QVQLVQSGAEVKKPGASVKVSCKASGFNIKDYFLHWVRQ | 27 |
| | APGQGLEWMGWINPDNGNTVYAQKFQGRVTMTADTSTST | |
| | VYMELSSLRSEDTAVYYCTRRDYTYEKAALDYWGQGTLV | |
| | TVSS | |
| TYRP1 | RASGNIYNYLA | 28 |
| LCDR1 | | |
| TYRP1 | DAKTLAD | 29 |
| LCDR2 | | |
| TYRP1 | QHFWSLPFT | 30 |
| LCDR3 | | |
| TYRP1 VL | DIQMTQSPSSLSASVGDRVTITCRASGNIYNYLAWYQQKPG | 31 |
| | KVPKLLIYDAKTLADGVPSRFSGSGSGTDFTLTISSLQPEDV | |
| | ATYYCQHFWSLPFTFGQGTKLEIK | |
| TYRP1 VH- | QVQLVQSGAEVKKPGASVKVSCKASGFNIKDYFLHWVRQ | 32 |
| CH1(EE)- | APGQGLEWMGWINPDNGNTVYAQKFQGRVTMTADTSTST | |
| CD3_{orig}/CD3ₒₚₜ | VYMELSSLRSEDTAVYYCTRRDYTYEKAALDYWGQGTLV | |
| VL-CH1- | TVSSASTKGPSVFPLAPSSKSTSGGTAALGCLVEDYFPEPVT | |
| Fc (knob, | VSWNSGALTSGVHTFPAVLQSSGLYSLSSVVTVPSSSLGTQ | |
| PGLALA) | TYICNVNHKPSNTKVDEKVEPKSCDGGGGSGGGGSQAVVT | |
| | QEPSLTVSPGGTVTLTCGSSTGAVTTSNYANWVQEKPGQA | |
| | FRGLIGGTNKRAPGTPARFSGSLLGGKAALTLSGAQPEDEA | |
| | EYYCALWYSNLWVFGGGTKLTVLSSASTKGPSVFPLAPSS | |
| | KSTSGGTAALGCLVKDYFPEPVTVSWNSGALTSGVHTFPA | |
| | VLQSSGLYSLSSVVTVPSSSLGTQTYICNVNHKPSNTKVDK | |
| | KVEPKSCDKTHTCPPCPAPEAAGGPSVFLFPPKPKDTLMISR | |
| | TPEVTCVVVDVSHEDPEVKFNWYVDGVEVHNAKTKPREE | |
| | QYNSTYRVVSVLTVLHQDWLNGKEYKCKVSNKALGAPIE | |
| | KTISKAKGQPREPQVYTLPPCRDELTKNQVSLWCLVKGFY | |
| | PSDIAVEWESNGQPENNYKTTPPVLDSDGSFFLYSKLTVDK | |
| | SRWQQGNVFSCSVMHEALHNHYTQKSLSLSP | |
| TYRP1 VH- | QVQLVQSGAEVKKPGASVKVSCKASGFNIKDYFLHWVRQ | 33 |
| CH1(EE)-Fc | APGQGLEWMGWINPDNGNTVYAQKFQGRVTMTADTSTST | |
| (hole, | VYMELSSLRSEDTAVYYCTRRDYTYEKAALDYWGQGTLV | |
| PGLALA) | TVSSASTKGPSVFPLAPSSKSTSGGTAALGCLVEDYFPEPVT | |
| | VSWNSGALTSGVHTFPAVLQSSGLYSLSSVVTVPSSSLGTQ | |
| | TYICNVNHKPSNTKVDEKVEPKSCDKTHTCPPCPAPEAAG | |
| | GPSVFLFPPKPKDTLMISRTPEVTCVVVDVSHEDPEVKFNW | |
| | YVDGVEVHNAKTKPREEQYNSTYRVVSVLTVLHQDWLNG | |
| | KEYKCKVSNKALGAPIEKTISKAKGQPREPQVCTLPPSRDE | |
| | LTKNQVSLSCAVKGFYPSDIAVEWESNGQPENNYKTTPPV | |
| | LDSDGSFFLVSKLTVDKSRWQQGNVFSCSVMHEALHNHY | |
| | TQKSLSLSP | |
| TYRP1 VL- | DIQMTQSPSSLSASVGDRVTITCRASGNIYNYLAWYQQKPG | 34 |
| CL(RK) | KVPKLLIYDAKTLADGVPSRFSGSGSGTDFTLTISSLQPEDV | |
| | ATYYCQHFWSLPFTFGQGTKLEIKRTVAAPSVFIFPPSDRKL | |
| | KSGTASVVCLLNNFYPREAKVQWKVDNALQSGNSQESVT | |
| | EQDSKDSTYSLSSTLTLSKADYEKHKVYACEVTHQGLSSPV | |
| | TKSFNRGEC | |
| CD3_{orig} VH- | EVQLLESGGGLVQPGGSLRLSCAASGFTFSTYAMNWVRQA | 35 |
| CL | PGKGLEWVSRIRSKYNNYATYYADSVKGRFTISRDDSKNT | |
| | LYLQMNSLRAEDTAVYYCVRHGNFGNSYVSWFAYWGQG | |
| | TLVTVSSASVAAPSVFIFPPSDEQLKSGTASVVCLLNNFYPR | |
| | EAKVQWKVDNALQSGNSQESVTEQDSKDSTYSLSSTLTLS | |
| | KADYEKHKVYACEVTHQGLSSPVTKSFNRGEC | |
| CD3ₒₚₜ | EVQLLESGGGLVQPGGSLRLSCAASGFTFESYAMNWVRQA | 36 |
| (P033.078) | PGKGLEWVSRIRSKYNEYATYYADSVKGRFTISRDDSKNT | |
| VH-CL | LYLQMNSLRAEDTAVYYCVRASNFPSSFVSYFGYWGQGTL | |
| | VTVSSASVAAPSVFIFPPSDEQLKSGTASVVCLLNNFYPREA | |
| | KVQWKVDNALQSGNSQESVTEQDSKDSTYSLSSTLTLSKA | |
| | DYEKHKVYACEVTHQGLSSPVTKSFNRGEC | |
| CD3ₒₚₜ | EVQLLESGGGLVQPGGSLRLSCAASGFTFSSYAMNWVRQA | 37 |
| (P035.093) | PGKGLEWVSRIRSKYNNYATYYADSVKGRFTISRDDSKNT | |
| VH-CL | LYLQMNSLRAEDTAVYYCVRASNFPASYVSYFAYWGQGT | |
| | LVTVSSASVAAPSVFIFPPSDEQLKSGTASVVCLLNNFYPRE | |
| | AKVQWKVDNALQSGNSQESVTEQDSKDSTYSLSSTLTLSK | |
| | ADYEKHKVYACEVTHQGLSSPVTKSFNRGEC | |
| CD3ₒₚₜ | EVQLLESGGGLVQPGGSLRLSCAASGFDFDNYAMNWVRQ | 38 |
| (P035.064) | APGKGLEWVSRIRSKHNGYATYYADSVKGRFTISRDDSKN | |
| VH-CL | TLYLQMNSLRAEDTAVYYCVRASNFPSSYVSYFGYWGQG | |
| | TLVTVSSASVAAPSVFIFPPSDEQLKSGTASVVCLLNNFYPR | |
| | EAKVQWKVDNALQSGNSQESVTEQDSKDSTYSLSSTLTLS | |
| | KADYEKHKVYACEVTHQGLSSPVTKSFNRGEC | |
| CD3ₒₚₜ | EVQLLESGGGLVQPGGSLRLSCAASGFTFSSYAMNWVRQA | 39 |
| (P021.045) | PGKGLEWVSRIRSKYNNYATYYADSVKGRFTISRDDSKNT | |
| VH-CL | LYLQMNSLRAEDTAVYYCVRASNFPSSYVSYFAYWGQGT | |
| | LVTVSSASVAAPSVFIFPPSDEQLKSGTASVVCLLNNFYPRE | |
| | AKVQWKVDNALQSGNSQESVTEQDSKDSTYSLSSTLTLSK | |
| | ADYEKHKVYACEVTHQGLSSPVTKSFNRGEC | |
| CD3ₒₚₜ | EVQLLESGGGLVQPGGSLRLSCAASGFQFDNYAMNWVRQ | 40 |
| (P004.042) | APGKGLEWVSRIRTKYNEYATYYADSVKGRFTISRDDSKN | |
| VH-CL | TLYLQMNSLRAEDTAVYYCVRASNFPQSYVSYFGYWGQG | |
| | TLVTVSSASVAAPSVFIFPPSDEQLKSGTASVVCLLNNFYPR | |
| | EAKVQWKVDNALQSGNSQESVTEQDSKDSTYSLSSTLTLS | |
| | KADYEKHKVYACEVTHQGLSSPVTKSFNRGEC | |
| Human CD3 | QDGNEEMGGITQTPYKVSISGTTVILTCPQYPGSEILWQHN | 41 |
| epsilon | DKNIGGDEDDKNIGSDEDHLSLKEFSELEQSGYYVCYPRGS | |
| stalk- | KPEDANFYLYLRARVSENCVDEQLYFQGGSPKSADKTHTC | |
| Fc(knob)- | PPCPAPELLGGPSVFLFPPKPKDTLMISRTPEVTCVVVDVSH | |
| Avi | EDPEVKFNWYVDGVEVHNAKTKPREEQYNSTYRVVSVLT | |
| | VLHQDWLNGKEYKCKVSNKALPAPIEKTISKAKGQPREPQ | |
| | VYTLPPCRDELTKNQVSLWCLVKGFYPSDIAVEWESNGQP | |
| | ENNYKTTPPVLDSDGSFFLYSKLTVDKSRWQQGNVFSCSV | |
| | MHEALHNHYTQKSLSLSPGKSGGLNDIFEAQKIEWHE | |
| Human CD3 | FKIPIEELEDRVFVNCNTSITWVEGTVGTLLSDITRLDLGKRI | 42 |
| delta stalk- | LDPRGIYRCNGTDIYKDKESTVQVHYRMCRSEQLYFQGDK | |
| Fc (hole)- | THTCPPCPAPELLGGPSVFLFPPKPKDTLMISRTPEVTCVVV | |
| Avi | DVSHEDPEVKFNWYVDGVEVHNAKTKPREEQYNSTYRVV | |
| | SVLTVLHQDWLNGKEYKCKVSNKALPAPIEKTISKAKGQP | |
| | REPQVCTLPPSRDELTKNQVSLSCAVKGFYPSDIAVEWESN | |
| | GQPENNYKTTPPVLDSDGSFFLVSKLTVDKSRWQQGNVFS | |
| | CSVMHEALHNHYTQKSLSLSPGKSGGLNDIFEAQKIEWHE | |
| Cynomolgus | QDGNEEMGSITQTPYQVSISGTTVILTCSQHLGSEAQWQHN | 43 |
| CD3 epsilon | GKNKEDSGDRLFLPEFSEMEQSGYYVCYPRGSNPEDASHH | |
| stalk-Fc | LYLKARVSENCVDEQLYFQGGSPKSADKTHTCPPCPAPELL | |
| (knob)-Avi | GGPSVFLFPPKPKDTLMISRTPEVTCVVVDVSHEDPEVKFN | |
| | WYVDGVEVHNAKTKPREEQYNSTYRVVSVLTVLHQDWL | |
| | NGKEYKCKVSNKALPAPIEKTISKAKGQPREPQVYTLPPCR | |
| | DELTKNQVSLWCLVKGFYPSDIAVEWESNGQPENNYKTTP | |
| | PVLDSDGSFFLYSKLTVDKSRWQQGNVFSCSVMHEALHNH | |
| | YTQKSLSLSPGKSGGLNDIFEAQKIEWHE | |
| Cynomolgus | FKIPVEELEDRVFVKCNTSVTWVEGTVGTLLTNNTRLDLG | 44 |
| CD3 delta | KRILDPRGIYRCNGTDIYKDKESAVQVHYRMSQNCVDEQL | |
| stalk-Fc | YFQGGSPKSADKTHTCPPCPAPELLGGPSVFLFPPKPKDTL | |
| (hole)-Avi | MISRTPEVTCVVVDVSHEDPEVKFNWYVDGVEVHNAKTK | |
| | PREEQYNSTYRVVSVLTVLHQDWLNGKEYKCKVSNKALP | |
| | APIEKTISKAKGQPREPQVCTLPPSRDELTKNQVSLSCAVKG | |
| | FYPSDIAVEWESNGQPENNYKTTPPVLDSDGSFFLVSKLTV | |
| | DKSRWQQGNVFSCSVMHEALHNHYTQKSLSLSPGKSGGL | |
| | NDIFEAQKIEWHE | |
| Human CD3 | QDGNEEMGGITQTPYKVSISGTTVILTCPQYPGSEILWQHN | 45 |
| | DKNIGGDEDDKNIGSDEDHLSLKEFSELEQSGYYVCYPRGS | |
| | KPEDANFYLYLRARVCENCMEMDVMSVATIVIVDICITGG | |
| | LLLLVYYWSKNRKAKAKPVTRGAGAGGRQRGQNKERPPP | |
| | VPNPDYEPIRKGQRDLYSGLNQRRI | |
| Cynomolgus | QDGNEEMGSITQTPYQVSISGTTVILTCSQHLGSEAQWQHN | 46 |
| CD3 | GKNKEDSGDRLFLPEFSEMEQSGYYVCYPRGSNPEDASHH | |
| | LYLKARVCENCMEMDVMAVATIVIVDICITLGLLLLVYYW | |
| | SKNRKAKAKPVTRGAGAGGRQRGQNKERPPPVPNPDYEPI | |
| | RKGQQDLYSGLNQRRI | |
| hIgG1 Fc | DKTHTCPPCPAPELLGGPSVFLFPPKPKDTLMISRTPEVTCV | 47 |
| region | VVDVSHEDPEVKFNWYVDGVEVHNAKTKPREEQYNSTYR | |
| | VVSVLTVLHQDWLNGKEYKCKVSNKALPAPIEKTISKAKG | |
| | QPREPQVYTLPPSRDELTKNQVSLTCLVKGFYPSDIAVEWE | |
| | SNGQPENNYKTTPPVLDSDGSFFLYSKLTVDKSRWQQGNV | |
| | FSCSVMHEALHNHYTQKSLSLSP | |
| linker | GGGGSGGGGS | 48 |
| linker | DGGGGSGGGGS | 49 |
| Human | RTVAAPSVFIFPPSDEQLKSGTASVVCLLNNFYPREAKVQW | 50 |
| kappa CL | KVDNALQSGNSQESVTEQDSKDSTYSLSSTLTLSKADYEK | |
| domain | HKVYACEVTHQGLSSPVTKSFNRGEC | |
| Human | QPKAAPSVTLFPPSSEELQANKATLVCLISDFYPGAVTVAW | 51 |
| lambda CL | KADSSPVKAGVETTTPSKQSNNKYAASSYLSLTPEQWKSH | |
| domain | RSYSCQVTHEGSTVEKTVAPTECS | |
| Human IgG1 | ASTKGPSVFPLAPSSKSTSGGTAALGCLVKDYFPEPVTVSW | 52 |
| heavy chain | NSGALTSGVHTFPAVLQSSGLYSLSSVVTVPSSSLGTQTYIC | |
| constant | NVNHKPSNTKVDKKVEPKSCDKTHTCPPCPAPELLGGPSVF | |
| region (CH1- | LFPPKPKDTLMISRTPEVTCVVVDVSHEDPEVKFNWYVDG | |
| CH2-CH3) | VEVHNAKTKPREEQYNSTYRVVSVLTVLHQDWLNGKEYK | |
| | CKVSNKALPAPIEKTISKAKGQPREPQVYTLPPSRDELTKN | |
| | QVSLTCLVKGFYPSDIAVEWESNGQPENNYKTTPPVLDSD | |
| | GSFFLYSKLTVDKSRWQQGNVFSCSVMHEALHNHYTQKS | |
| | LSLSP | |

Protease-Activatable T Cell Activating Bispecific Molecule with Improved Anti-CD3 (P035.093) Binder

CDR Definition According to Kabat

CD3 P035.093

| | | |
|---|---|---|
| CD3ₒₚₜ VH | EVQLLESGGGLVQPGGSLRLSCAASGFTFSSYAMNWVRQ | 16 |
| (P035.093) | APGKGLEWVSRIRSKYNNYATYYADSVKGRFTISRDDSK | |
| | NTLYLQMNSLRAEDTAVYYCVRASNFPASYVSYFAYWG | |
| | QGTLVTVSS | |
| CDRH1 | SYAMN | 2 |
| CDRH2 | RIRSKYNNYATYYADSVKG | 4 |
| CDRH3 | ASNFPASYVSYFAY | 10 |
| VL | QAVVTQEPSLTVSPGGTVTLTCGSSTGAVTTSNYANWVQ | 23 |
| | EKPGQAFRGLIGGTNKRAPGTPARFSGSLLGGKAALTLSG | |
| | AQPEDEAEYYCALWYSNLWVFGGGTKLTVL | |
| CDRL1 | GSSTGAVTTSNYAN | 20 |
| CDRL2 | GTNKRAP | 21 |
| CDRL3 | ALWYSNLWV | 23 |

FOLR1_16D5

| | | |
|---|---|---|
| VH | EVQLVESGGGLVKPGGSLRLSCAASGFTFSNAWMSWVR | 53 |
| | QAPGKGLEWVGRIKSKTDGGTTDYAAPVKGRFTISRDDS | |
| | KNTLYLQMNSLKTEDTAVYYCTTPWEWSWYDYWGQGT | |
| | LVTVSS | |
| CDRH1 | NAWMS | 54 |
| CDRH2 | RIKSKTDGGTTDYAAPVKG | 55 |
| CDRH3 | PWEWSWYDY | 56 |
| VL | QAVVTQEPSLTVSPGGTVTLTCGSSTGAVTTSNYANWVQ | 23 |
| | EKPGQAFRGLIGGTNKRAPGTPARFSGSLLGGKAALTLSG | |
| | AQPEDEAEYYCALWYSNLWVFGGGTKLTVL | |
| CDRL1 | GSSTGAVTTSNYAN | 20 |
| CDRL2 | GTNKRAP | 21 |
| CDRL3 | ALWYSNLWV | 22 |

ID_4.24.72

| | | |
|---|---|---|
| VH | QIQLVQSGPELKKPGETVKISCKASGYTVTDYSMNWVKQ | 57 |
| | APGKCLKWMGWINTETGEPRYTDDFKGRFAFSLETSAST | |
| | AYLQINNLKNEDSATYFCAREGDYDVFDYWGHGTTLKV | |
| | SS | |
| CDRH1 | DYSMN | 58 |
| CDRH2 | WINTETGEPRYTDDFKG | 59 |
| CDRH3 | EGDYDVFDY | 60 |
| VL | DIVLTQSPASLAVSLGQRATISCRASKSVSTSSYSYMHWY | 61 |
| | QQKPGQPPKLLIKYVSYLESGVPARFSGSGSGTDFTLNIHP | |
| | VEEEDAATYYCQHSREFPYTFGCGTKLEIK | |
| CDRL1 | RASKSVSTSSYSYMH | 62 |
| CDRL2 | YVSYLES | 63 |
| CDRL3 | QHSREFPYT | 64 |

FOLR1 proTCB with protease linker and anti-CD3 P035.093

| | | |
|---|---|---|
| K chain | QIQLVQSGPELKKPGETVKISCKASGYTVTDYSMNWVKQ | 65 |
| | APGKCLKWMGWINTETGEPRYTDDFKGRFAFSLETSAST | |
| | AYLQINNLKNEDSATYFCAREGDYDVFDYWGHGTTLKV | |
| | SSGGGGSGGGGSGGGGSGGGGSDIVLTQSPASLAVSLGQ | |
| | RATISCRASKSVSTSSYSYMHWYQQKPGQPPKLLIKYVSY | |
| | LESGVPARFSGSGSGTDFTLNIHPVEEEDAATYYCQHSRE | |
| | FPYTFGCGTKLEIKSGGGSGGGGSPMAKKGGGGSGGGGS | |
| | GGGGSGGSEVQLLESGGGLVQPGGSLRLSCAASGFTFSSY | |
| | AMNWVRQAPGKGLEWVSRIRSKYNNYATYYADSVKGR | |
| | FTISRDDSKNTLYLQMNSLRAEDTAVYYCVRASNFPASY | |
| | VSYFAYWGQGTLVTVSSASTKGPSVFPLAPSSKSTSGGTA | |
| | ALGCLVKDYFPEPVTVSWNSGALTSGVHTFPAVLQSSGL | |
| | YSLSSVVTVPSSSLGTQTYICNVNHKPSNTKVDKKVEPKS | |
| | CDGGGGSGGGGSEVQLVESGGGLVKPGGSLRLSCAASGF | |
| | TFSNAWMSWVRQAPGKGLEWVGRIKSKTDGGTTDYAAP | |
| | VKGRFTISRDDSKNTLYLQMNSLKTEDTAVYYCTTPWEW | |
| | SWYDYWGQGTLVTVSSASTKGPSVFPLAPSSKSTSGGTA | |
| | ALGCLVKDYFPEPVTVSWNSGALTSGVHTFPAVLQSSGL | |
| | YSLSSVVTVPSSSLGTQTYICNVNHKPSNTKVDKKVEPKS | |
| | CDKTHTCPPCPAPEAAGGPSVFLFPPKPKDTLMISRTPEVT | |
| | CVVVDVSHEDPEVKFNWYVDGVEVHNAKTKPREEQYNS | |
| | TYRVVSVLTVLHQDWLNGKEYKCKVSNKALGAPIEKTIS | |
| | KAKGQPREPQVYTLPPCRDELTKNQVSLWCLVKGFYPSD | |
| | IAVEWESNGQPENNYKTTPPVLDSDGSFFLYSKLTVDKSR | |
| | WQQGNVFSCSVMHEALHNHYTQKSLSLSP | |
| H chain | EVQLVESGGGLVKPGGSLRLSCAASGFTFSNAWMSWVR | 66 |
| | QAPGKGLEWVGRIKSKTDGGTTDYAAPVKGRFTISRDDS | |
| | KNTLYLQMNSLKTEDTAVYYCTTPWEWSWYDYWGQGT | |
| | LVTVSSASTKGPSVFPLAPSSKSTSGGTAALGCLVKDYFPE | |
| | PVTVSWNSGALTSGVHTFPAVLQSSGLYSLSSVVTVPSSS | |
| | LGTQTYICNVNHKPSNTKVDKKVEPKSCDKTHTCPPCPAP | |
| | EAAGGPSVFLFPPKPKDTLMISRTPEVTCVVVDVSHEDPE | |
| | VKFNWYVDGVEVHNAKTKPREEQYNSTYRVVSVLTVLH | |
| | QDWLNGKEYKCKVSNKALGAPIEKTISKAKGQPREPQVC | |
| | TLPPSRDELTKNQVSLSCAVKGFYPSDIAVEWESNGQPEN | |
| | NYKTTPPVLDSDGSFFLVSKLTVDKSRWQQGNVFSCSVM | |
| | HEALHNHYTQKSLSLSP | |
| L chain | QAVVTQEPSLTVSPGGTVTLTCGSSTGAVTTSNYANWVQ | 67 |
| | EKPGQAFRGLIGGTNKRAPGTPARFSGSLLGGKAALTLSG | |
| | AQPEDEAEYYCALWYSNLWVFGGGTKLTVLGQPKAAPS | |
| | VTLFPPSSEELQANKATLVCLISDFYPGAVTVAWKADSSP | |
| | VKAGVETTTPSKQSNNKYAASSYLSLTPEQWKSHRSYSC | |
| | QVTHEGSTVEKTVAPTECS | |
| Matriptase | SGGGSGGGGSPMAKKGGGGSGGGGSGGGGSGGS | 68 |
| linker | | |

FOLR1proTCB with protease linker CD3: P035.093

| | | |
|---|---|---|
| K chain | QIQLVQSGPELKKPGETVKISCKASGYTVTDYSMNWVKQ | 69 |
| | APGKCLKWMGWINTETGEPRYTDDFKGRFAFSLETSAST | |
| | AYLQINNLKNEDSATYFCAREGDYDVFDYWGHGTTLKV | |
| | SSGGGGSGGGGSGGGGSGGGGSDIVLTQSPASLAVSLGQ | |
| | RATISCRASKSVSTSSYSYMHWYQQKPGQPPKLLIKYVSY | |
| | LESGVPARFSGSGSGTDFTLNIHPVEEEDAATYYCQHSRE | |
| | FPYTFGCGTKLEIKGGGGSVHMPLGFLGPRQARVVNGGG | |
| | GGSGGGGSEVQLLESGGGLVQPGGSLRLSCAASGFTFSSY | |
| | AMNWVRQAPGKGLEWVSRIRSKYNNYATYYADSVKGR | |
| | FTISRDDSKNTLYLQMNSLRAEDTAVYYCVRASNFPASY | |
| | VSYFAYWGQGTLVTVSSASTKGPSVFPLAPSSKSTSGGTA | |
| | ALGCLVKDYFPEPVTVSWNSGALTSGVHTFPAVLQSSGL | |
| | YSLSSVVTVPSSSLGTQTYICNVNHKPSNTKVDKKVEPKS | |
| | CDGGGGSGGGGSEVQLVESGGGLVKPGGSLRLSCAASGF | |
| | TFSNAWMSWVRQAPGKGLEWVGRIKSKTDGGTTDYAAP | |
| | VKGRFTISRDDSKNTLYLQMNSLKTEDTAVYYCTTPWEW | |
| | SWYDYWGQGTLVTVSSASTKGPSVFPLAPSSKSTSGGTA | |
| | ALGCLVKDYFPEPVTVSWNSGALTSGVHTFPAVLQSSGL | |
| | YSLSSVVTVPSSSLGTQTYICNVNHKPSNTKVDKKVEPKS | |
| | CDKTHTCPPCPAPEAAGGPSVFLFPPKPKDTLMISRTPEVT | |
| | CVVVDVSHEDPEVKFNWYVDGVEVHNAKTKPREEQYNS | |
| | TYRVVSVLTVLHQDWLNGKEYKCKVSNKALGAPIEKTIS | |
| | KAKGQPREPQVYTLPPCRDELTKNQVSLWCLVKGFYPSD | |
| | IAVEWESNGQPENNYKTTPPVLDSDGSFFLYSKLTVDKSR | |
| | WQQGNVFSCSVMHEALHNHYTQKSLSLSP | |
| Linker | GGGGSVHMPLGFLGPRQARVVNGGGGGSGGGGS | 70 |
| H chain | As in FOLR1 proTCB with protease linker | 66 |
| | anti-CD3 P035.093 | |
| L chair | As in FOLR1 proTCB with protease linker | 67 |
| | anti-CD3 P035.093 | |

FOLR1proTCB with non-cleavable linker CD3: P035.093

| | | |
|---|---|---|
| K chain | QIQLVQSGPELKKPGETVKISCKASGYTVTDYSMNWVKQ | 71 |
| | APGKCLKWMGWINTETGEPRYTDDFKGRFAFSLETSAST | |
| | AYLQINNLKNEDSATYFCAREGDYDVFDYWGHGTTLKV | |
| | SSGGGGSGGGGSGGGGSGGGGSDIVLTQSPASLAVSLGQ | |
| | RATISCRASKSVSTSSYSYMHWYQQKPGQPPKLLIKYVSY | |
| | LESGVPARFSGSGSGTDFTLNIHPVEEEDAATYYCQHSRE | |
| | FPYTFGCGTKLEIKGGGGSGGGGSGGGGSGGGGGGGSGG | |
| | GGSGGGGSEVQLLESGGGLVQPGGSLRLSCAASGFTFSSY | |
| | AMNWVRQAPGKGLEWVSRIRSKYNNYATYYADSVKGR | |
| | FTISRDDSKNTLYLQMNSLRAEDTAVYYCVRASNFPASY | |
| | VSYFAYWGQGTLVTVSSASTKGPSVFPLAPSSKSTSGGTA | |
| | ALGCLVKDYFPEPVTVSWNSGALTSGVHTFPAVLQSSGL | |
| | YSLSSVVTVPSSSLGTQTYICNVNHKPSNTKVDKKVEPKS | |
| | CDGGGGSGGGGSEVQLVESGGGLVKPGGSLRLSCAASGF | |
| | TFSNAWMSWVRQAPGKGLEWVGRIKSKTDGGTTDYAAP | |
| | VKGRFTISRDDSKNTLYLQMNSLKTEDTAVYYCTTPWEW | |
| | SWYDYWGQGTLVTVSSASTKGPSVFPLAPSSKSTSGGTA | |
| | ALGCLVKDYFPEPVTVSWNSGALTSGVHTFPAVLQSSGL | |
| | YSLSSVVTVPSSSLGTQTYICNVNHKPSNTKVDKKVEPKS | |
| | CDKTHTCPPCPAPEAAGGPSVFLFPPKPKDTLMISRTPEVT | |
| | CVVVDVSHEDPEVKFNWYVDGVEVHNAKTKPREEQYNS | |
| | TYRVVSVLTVLHQDWLNGKEYKCKVSNKALGAPIEKTIS | |
| | KAKGQPREPQVYTLPPCRDELTKNQVSLWCLVKGFYPSD | |
| | IAVEWESNGQPENNYKTTPPVLDSDGSFFLYSKLTVDKSR | |
| | WQQGNVFSCSVMHEALHNHYTQKSLSLSP | |
| Linker | GGGGSGGGGSGGGGSGGGGGGGSGGGGSGGGGS | 72 |
| H chain | As in FOLR1 proTCB with protease linker | 66 |
| | anti-CD3 P035.093 | |
| L chain | As in FOLR1 proTCB with protease linker | 67 |
| | anti-CD3 P035.093 | |

FOLR1 TCB CD3: P035.093

| | | |
|---|---|---|
| K chain | EVQLLESGGGLVQPGGSLRLSCAASGFTFSSYAMNWVRQ | 73 |
| | APGKGLEWVSRIRSKYNNYATYYADSVKGRFTISRDDSK | |
| | NTLYLQMNSLRAEDTAVYYCVRASNFPASYVSYFAYWG | |
| | QGTLVTVSSASTKGPSVFPLAPSSKSTSGGTAALGCLVKD | |
| | YFPEPVTVSWNSGALTSGVHTFPAVLQSSGLYSLSSVVTV | |
| | PSSSLGTQTYICNVNHKPSNTKVDKKVEPKSCDGGGGSG | |
| | GGGSEVQLVESGGGLVKPGGSLRLSCAASGFTFSNAWMS | |
| | WVRQAPGKGLEWVGRIKSKTDGGTTDYAAPVKGRFTISR | |
| | DDSKNTLYLQMNSLKTEDTAVYYCTTPWEWSWYDYWG | |
| | QGTLVTVSSASTKGPSVFPLAPSSKSTSGGTAALGCLVKD | |
| | YFPEPVTVSWNSGALTSGVHTFPAVLQSSGLYSLSSVVTV | |
| | PSSSLGTQTYICNVNHKPSNTKVDKKVEPKSCDKTHTCPP | |
| | CPAPEAAGGPSVFLFPPKPKDTLMISRTPEVTCVVVDVSH | |
| | EDPEVKFNWYVDGVEVHNAKTKPREEQYNSTYRVVSVL | |
| | TVLHQDWLNGKEYKCKVSNKALGAPIEKTISKAKGQPRE | |
| | PQVYTLPPCRDELTKNQVSLWCLVKGFYPSDIAVEWESN | |
| | GQPENNYKTTPPVLDSDGSFFLYSKLTVDKSRWQQGNVF | |
| | SCSVMHEALHNHYTQKSLSLSP | |
| H chain | As in FOLR1 proTCB with protease linker | 66 |
| | anti-CD3 P035.093 | |
| L chain | As in FOLR1 proTCB with protease linker | 67 |
| | anti-CD3 P035.093 | |

FOLR1proTCB with matriptase linker CD3: CH2527

| | | |
|---|---|---|
| K chain | QIQLVQSGPELKKPGETVKISCKASGYTVTDYSMNWVKQ | 74 |
| | APGKCLKWMGWINTETGEPRYTDDFKGRFAFSLETSAST | |
| | AYLQINNLKNEDSATYFCAREGDYDVFDYWGHGTTLKV | |
| | SSGGGGSGGGGSGGGGSGGGGSDIVLTQSPASLAVSLGQ | |
| | RATISCRASKSVSTSSYSYMHWYQQKPGQPPKLLIKYVSY | |
| | LESGVPARFSGSGSGTDFTLNIHPVEEEDAATYYCQHSRE | |
| | FPYTFGCGTKLEIKGGGGSGGGGSRQARVVNGGGGGSGG | |
| | GGSGGGGSEVQLLESGGGLVQPGGSLRLSCAASGFTFSTY | |
| | AMNWVRQAPGKGLEWVSRIRSKYNNYATYYADSVKGR | |
| | FTISRDDSKNTLYLQMNSLRAEDTAVYYCVRHGNFGNSY | |
| | VSWFAYWGQGTLVTVSSASTKGPSVFPLAPSSKSTSGGTA | |
| | ALGCLVKDYFPEPVTVSWNSGALTSGVHTFPAVLQSSGL | |
| | YSLSSVVTVPSSSLGTQTYICNVNHKPSNTKVDKKVEPKS | |
| | CDGGGGSGGGGSEVQLVESGGGLVKPGGSLRLSCAASGF | |
| | TFSNAWMSWVRQAPGKGLEWVGRIKSKTDGGTTDYAAP | |
| | VKGRFTISRDDSKNTLYLQMNSLKTEDTAVYYCTTPWEW | |
| | SWYDYWGQGTLVTVSSASTKGPSVFPLAPSSKSTSGGTA | |
| | ALGCLVKDYFPEPVTVSWNSGALTSGVHTFPAVLQSSGL | |
| | YSLSSVVTVPSSSLGTQTYICNVNHKPSNTKVDKKVEPKS | |
| | CDKTHTCPPCPAPEAAGGPSVFLFPPKPKDTLMISRTPEVT | |
| | CVVVDVSHEDPEVKFNWYVDGVEVHNAKTKPREEQYNS | |
| | TYRVVSVLTVLHQDWLNGKEYKCKVSNKALGAPIEKTIS | |
| | KAKGQPREPQVYTLPPCRDELTKNQVSLWCLVKGFYPSD | |
| | IAVEWESNGQPENNYKTTPPVLDSDGSFFLYSKLTVDKSR | |
| | WQQGNVFSCSVMHEALHNHYTQKSLSLSP | |
| Linker | GGGGSGGGGSRQARVVNGGGGGSGGGGSGGGGS | 75 |
| H chain | As in FOLR1 proTCB with protease linker | 66 |
| | anti-CD3 P035.093 | |
| L chain | As in FOLR1 proTCB with protease linker | 67 |
| | anti-CD3 P035.093 | |

FOLR1proTCB with MMP2/9-matriptase linker CD3:CH2527

| | | |
|---|---|---|
| K chain | QIQLVQSGPELKKPGETVKISCKASGYTVTDYSMNWVKQ | 76 |
| | APGKCLKWMGWINTETGEPRYTDDFKGRFAFSLETSAST | |
| | AYLQINNLKNEDSATYFCAREGDYDVFDYWGHGTTLKV | |
| | SSGGGGSGGGGSGGGGSGGGGSDIVLTQSPASLAVSLGQ | |
| | RATISCRASKSVSTSSYSYMHWYQQKPGQPPKLLIKYVSY | |
| | LESGVPARFSGSGSGTDFTLNIHPVEEEDAATYYCQHSRE | |
| | FPYTFGCGTKLEIKGGGGSVHMPLGFLGPRQARVVNGGG | |
| | GGSGGGGSEVQLLESGGGLVQPGGSLRLSCAASGFTFSTY | |
| | AMNWVRQAPGKGLEWVSRIRSKYNNYATYYADSVKGR | |
| | FTISRDDSKNTLYLQMNSLRAEDTAVYYCVRHGNFGNSY | |
| | VSWFAYWGQGTLVTVSSASTKGPSVFPLAPSSKSTSGGTA | |
| | ALGCLVKDYFPEPVTVSWNSGALTSGVHTFPAVLQSSGL | |
| | YSLSSVVTVPSSSLGTQTYICNVNHKPSNTKVDKKVEPKS | |
| | CDGGGGSGGGGSEVQLVESGGGLVKPGGSLRLSCAASGF | |
| | TFSNAWMSWVRQAPGKGLEWVGRIKSKTDGGTTDYAAP | |
| | VKGRFTISRDDSKNTLYLQMNSLKTEDTAVYYCTTPWEW | |
| | SWYDYWGQGTLVTVSSASTKGPSVFPLAPSSKSTSGGTA | |
| | ALGCLVKDYFPEPVTVSWNSGALTSGVHTFPAVLQSSGL | |
| | YSLSSVVTVPSSSLGTQTYICNVNHKPSNTKVDKKVEPKS | |
| | CDKTHTCPPCPAPEAAGGPSVFLFPPKPKDTLMISRTPEVT | |
| | CVVVDVSHEDPEVKFNWYVDGVEVHNAKTKPREEQYNS | |
| | TYRVVSVLTVLHQDWLNGKEYKCKVSNKALGAPIEKTIS | |
| | KAKGQPREPQVYTLPPCRDELTKNQVSLWCLVKGFYPSD | |
| | IAVEWESNGQPENNYKTTPPVLDSDGSFFLYSKLTVDKSR | |
| | WQQGNVFSCSVMHEALHNHYTQKSLSLSP | |
| Linker | GGGGSVHMPLGFLGPRQARVVNGGGGGSGGGGS | 77 |
| H chain | As in FOLR1 proTCB with protease linker | 66 |
| | anti-CD3 P035.093 | |
| L chain | As in FOLR1 proTCB with protease linker | 67 |
| | anti-CD3 P035.093 | |

FOLR1 proTCB with non-cleavable linker CD3: CH2527

| | | |
|---|---|---|
| K chain | QIQLVQSGPELKKPGETVKISCKASGYTVTDYSMNWVKQ | 78 |
| | APGKCLKWMGWINTETGEPRYTDDFKGRFAFSLETSAST | |
| | AYLQINNLKNEDSATYFCAREGDYDVFDYWGHGTTLKV | |
| | SSGGGGSGGGGSGGGGSGGGGSDIVLTQSPASLAVSLGQ | |
| | RATISCRASKSVSTSSYSYMHWYQQKPGQPPKLLIKYVSY | |
| | LESGVPARFSGSGSGTDFTLNIHPVEEEDAATYYCQHSRE | |
| | FPYTFGCGTKLEIKGGGGSGGGGSGGGGSGGGGGGGSGG | |
| | GGSGGGGSEVQLLESGGGLVQPGGSLRLSCAASGFTFSTY | |
| | AMNWVRQAPGKGLEWVSRIRSKYNNYATYYADSVKGR | |
| | FTISRDDSKNTLYLQMNSLRAEDTAVYYCVRHGNFGNSY | |
| | VSWFAYWGQGTLVTVSSASTKGPSVFPLAPSSKSTSGGTA | |
| | ALGCLVKDYFPEPVTVSWNSGALTSGVHTFPAVLQSSGL | |
| | YSLSSVVTVPSSSLGTQTYICNVNHKPSNTKVDKKVEPKS | |
| | CDGGGGSGGGGSEVQLVESGGGLVKPGGSLRLSCAASGF | |
| | TFSNAWMSWVRQAPGKGLEWVGRIKSKTDGGTTDYAAP | |
| | VKGRFTISRDDSKNTLYLQMNSLKTEDTAVYYCTTPWEW | |
| | SWYDYWGQGTLVTVSSASTKGPSVFPLAPSSKSTSGGTA | |
| | ALGCLVKDYFPEPVTVSWNSGALTSGVHTFPAVLQSSGL | |
| | YSLSSVVTVPSSSLGTQTYICNVNHKPSNTKVDKKVEPKS | |
| | CDKTHTCPPCPAPEAAGGPSVFLFPPKPKDTLMISRTPEVT | |
| | CVVVDVSHEDPEVKFNWYVDGVEVHNAKTKPREEQYNS | |
| | TYRVVSVLTVLHQDWLNGKEYKCKVSNKALGAPIEKTIS | |
| | KAKGQPREPQVYTLPPCRDELTKNQVSLWCLVKGFYPSD | |
| | IAVEWESNGQPENNYKTTPPVLDSDGSFFLYSKLTVDKSR | |
| | WQQGNVFSCSVMHEALHNHYTQKSLSLSP | |
| H chain | As in FOLR1 proTCB with protease linker | 66 |
| | anti-CD3 P035.093 | |
| L chain | As in FOLR1 proTCB with protease linker | 67 |
| | anti-CD3 P035.093 | |

Masking Moiety Humanization Variants

Mask humanization variant H1L1

| | | |
|---|---|---|
| VH | QVQLVQSGSELKKPGASVKVSCKASGYTVTDYSMNWVR | 79 |
| | QAPGQGLEWMGWINTETGEPRYTDDFKGRFVFSLDTSVS | |
| | TAYLQISSLKAEDTAVYYCAREGDYDVFDYWGQGTLVT | |
| | VSS | |
| CDRH1 | DYSMN | 58 |
| CDRH2 | WINTETGEPRYTDDFKG | 59 |
| CDRH3 | EGDYDVFDY | 60 |
| VL | DIVMTQSPDSLAVSLGERATINCRASKSVSTSSYSYMHWY | 80 |
| | QQKPGQPPKLLIKYVSYLESGVPDRFSGSGSGTDFTLTISS | |
| | LQAEDVAVYYCQHSREFPYTFGQGTKLEIK | |
| CDRL1 | RASKSVSTSSYSYMH | 62 |
| CDRL2 | YVSYLES | 63 |
| CDRL3 | QHSREFPYT | 64 |

Mask humanization variant H1L2

| | | |
|---|---|---|
| VH | QVQLVQSGSELKKPGASVKVSCKASGYTVTDYSMNWVR | 79 |
| | QAPGQGLEWMGWINTETGEPRYTDDFKGRFVFSLDTSVS | |
| | TAYLQISSLKAEDTAVYYCAREGDYDVFDYWGQGTLVT | |
| | VSS | |
| CDRH1 | DYSMN | 58 |
| CDRH2 | WINTETGEPRYTDDFKG | 59 |
| CDRH3 | EGDYDVFDY | 60 |
| VL | DIVMTQSPDSLAVSLGERATINCKSSKSVSTSSYSYMHWY | 81 |
| | QQKPGQPPKLLIKYVSYLESGVPDRFSGSGSGTDFTLTISS | |
| | LQAEDVAVYYCQHSREFPYTFGQGTKLEIK | |
| CDRL1 | KSSKSVSTSSYSYMH | 82 |
| CDRL2 | YVSYLES | 63 |
| CDRL3 | QHSREFPYT | 64 |

Mask humanization variant H2L2

| | | |
|---|---|---|
| VH | QVQLVQSGSELKKPGASVKVSCKASGYTVTDYSMNWVR | 83 |
| | QAPGQGLEWMGWINTETGEPRYTDDFTGRFVFSLDTSVS | |
| | TAYLQISSLKAEDTAVYYCAREGDYDVFDYWGQGTLVT | |
| | VSS | |
| CDRH1 | DYSMN | 58 |
| CDRH2 | WINTETGEPRYTDDFTG | 84 |
| CDRH3 | EGDYDVFDY | 60 |
| VL | DIVMTQSPDSLAVSLGERATINCKSSKSVSTSSYSYMHWY | 8 |
| | QQKPGQPPKLLIKYVSYLESGVPDRFSGSGSGTDFTLTISS | |
| | LQAEDVAVYYCQHSREFPYTFGQGTKLEIK | |
| CDRL1 | KSSKSVSTSSYSYMH | 82 |
| CDRL2 | YVSYLES | 63 |
| CDRL3 | QHSREFPYT | 64 |

Mask humanization variant H3L2

| | | |
|---|---|---|
| VH | QVQLVQSGSELKKPGASVKVSCKASGYTVTDYSMNWVR | 85 |
| | QAPGQGLEWMGWINTETGEPRYTQGFKGRFVFSLDTSVS | |
| | TAYLQISSLKAEDTAVYYCAREGDYDVFDYWGQGTLVT | |
| | VSS | |
| CDRH1 | DYSMN | 58 |
| CDRH2 | WINTETGEPRYTQGFKG | 86 |
| CDRH3 | EGDYDVFDY | 60 |
| VL | DIVMTQSPDSLAVSLGERATINCKSSKSVSTSSYSYMHWY | 81 |
| | QQKPGQPPKLLIKYVSYLESGVPDRFSGSGSGTDFTLTISS | |
| | LQAEDVAVYYCQHSREFPYTFGQGTKLEIK | |
| CDRL1 | KSSKSVSTSSYSYMH | 32 |
| CDRL2 | YVSYLES | 63 |
| CDRL3 | QHSREFPYT | 64 |

Mask humanization variant H3L3

| | | |
|---|---|---|
| VH | QVQLVQSGSELKKPGASVKVSCKASGYTVTDYSMNWVR | 84 |
| | QAPGQGLEWMGWINTETGEPRYTQGFKGRFVFSLDTSVS | |
| | TAYLQISSLKAEDTAVYYCAREGDYDVFDYWGQGTLVT | |
| | VSS | |
| CDRH1 | DYSMN | 58 |
| CDRH2 | WINTETGEPRYTQGFKG | 86 |
| CDRH3 | EGDYDVFDY | 60 |
| VL | DIVMTQSPDSLAVSLGERATINCRASKSVSTSSYSYMHWY | 87 |
| | QQKPGQPPKLLIKYVSYLESGVPDRFSGSGSGTDFTLTISS | |
| | LQAEDVAVYYCQQSREFPYTFGQGTKLEIK | |
| CDRL1 | RASKSVSTSSYSYMH | 62 |
| CDRL2 | YVSYLES | 63 |
| CDRL3 | QQSREFPYT | 88 |

Mask humanization variant H7L5

| | | |
|---|---|---|
| VH | QVQLVQSGAEVKKPGASVKVSCKASGYTVTDYSMNWV | 89 |
| | RQAPGQGLEWMGWINTETGEPRYTDDFKGRVTMTRDTSI | |
| | STAYMELSRLRSDDTAVYYCAREGDYDVFDYWGQGTLV | |
| | TVSS | |
| CDRH1 | DYSMN | 58 |
| CDRH2 | WINTETGEPRYTDDFKG | 59 |
| CDRH3 | EGDYDVFDY | 60 |
| VL | DIVMTQSPDSLAVSLGERATINCRASKSVSTSSYSYMHWY | 90 |
| | QQKPGQPPKLLIYYVSYLESGVPDRFSGSGSGTDFTLTISS | |
| | LQAEDVAVYYCQHSREFPYTFGQGTKLEIK | |
| CDRL1 | RASKSVSTSSYSYMH | 62 |
| CDRL2 | YVSYLES | 63 |
| CDRL3 | QHSREFPYT | 64 |

Masking Moiety scFv

| | | |
|---|---|---|
| H1L1 scFv | QVQLVQSGSELKKPGASVKVSCKASGYTVTDYSMNWVR | 91 |
| | QAPGQCLEWMGWINTETGEPRYTDDFKGRFVFSLDTSVS | |
| | TAYLQISSLKAEDTAVYYCAREGDYDVFDYWGQGTLVT | |
| | VSSGGGGSGGGGSGGGGSGGGGSDIVMTQSPDSLAVSLG | |
| | ERATINCRASKSVSTSSYSYMHWYQQKPGQPPKLLIKYVS | |
| | YLESGVPDRFSGSGSGTDFTLTISSLQAEDVAVYYCQHSR | |
| | EFPYTFGCGTKLEIK | |
| H1L2 scFv | QVQLVQSGSELKKPGASVKVSCKASGYTVTDYSMNWVR | 92 |
| | QAPGQCLEWMGWINTETGEPRYTDDFKGRFVFSLDTSVS | |
| | TAYLQISSLKAEDTAVYYCAREGDYDVFDYWGQGTLVT | |
| | VSSGGGGSGGGGSGGGGSGGGGSDIVMTQSPDSLAVSLG | |
| | ERATINCKSSKSVSTSSYSYMHWYQQKPGQPPKLLIKYVS | |
| | YLESGVPDRFSGSGSGTDFTLTISSLQAEDVAVYYCQHSR | |
| | EFPYTFGCGTKLEIK | |
| H2L2 scFv | QVQLVQSGSELKKPGASVKVSCKASGYTVTDYSMNWVR | 93 |
| | QAPGQCLEWMGWINTETGEPRYTDDFTGRFVFSLDTSVS | |
| | TAYLQISSLKAEDTAVYYCAREGDYDVFDYWGQGTLVT | |
| | VSSGGGGSGGGGSGGGGSGGGGSDIVMTQSPDSLAVSLG | |
| | ERATINCKSSKSVSTSSYSYMHWYQQKPGQPPKLLIKYVS | |
| | YLESGVPDRFSGSGSGTDFTLTISSLQAEDVAVYYCQHSR | |
| | EFPYTFGCGTKLEIK | |
| H3L2 scFv | QVQLVQSGSELKKPGASVKVSCKASGYTVTDYSMNWVR | 94 |
| | QAPGQCLEWMGWINTETGEPRYTQGFKGRFVFSLDTSVS | |
| | TAYLQISSLKAEDTAVYYCAREGDYDVFDYWGQGTLVT | |
| | VSSGGGGSGGGGSGGGGSGGGGSDIVMTQSPDSLAVSLG | |
| | ERATINCKSSKSVSTSSYSYMHWYQQKPGQPPKLLIKYVS | |
| | YLESGVPDRFSGSGSGTDFTLTISSLQAEDVAVYYCQHSR | |
| | EFPYTFGCGTKLEIK | |

FolR1 Protease Activatable T Cell Activating Bispecific Molecules (proTCB) with Humanized Mask and PMAKK Protease Recognition Sequence

FolR1 pro TCB P035.093 H1L1

| | | |
|---|---|---|
| K chain | QVQLVQSGSELKKPGASVKVSCKASGYTVTDYSMNWVR | 95 |
| | QAPGQCLEWMGWINTETGEPRYTDDFKGRFVFSLDTSVS | |
| | TAYLQISSLKAEDTAVYYCAREGDYDVFDYWGQGTLVT | |
| | VSSGGGGSGGGGSGGGGSGGGGSDIVMTQSPDSLAVSLG | |
| | ERATINCRASKSVSTSSYSYMHWYQQKPGQPPKLLIKYVS | |
| | YLESGVPDRFSGSGSGTDFTLTISSLQAEDVAVYYCQHSR | |
| | EFPYTFGCGTKLEIKGGGGSGGGGSPMAKKGGGGSGGGG | |
| | SGGGGSGGSEVQLLESGGGLVQPGGSLRLSCAASGFTFSS | |
| | YAMNWVRQAPGKGLEWVSRIRSKYNNYATYYADSVKG | |
| | RFTISRDDSKNTLYLQMNSLRAEDTAVYYCVRASNFPAS | |
| | YVSYFAYWGQGTLVTVSSASTKGPSVFPLAPSSKSTSGGT | |
| | AALGCLVKDYFPEPVTVSWNSGALTSGVHTFPAVLQSSG | |
| | LYSLSSVVTVPSSSLGTQTYICNVNHKPSNTKVDKKVEPK | |
| | SCDGGGGSGGGGSEVQLVESGGGLVKPGGSLRLSCAASG | |
| | FTFSNAWMSWVRQAPGKGLEWVGRIKSKTDGGTTDYAA | |
| | PVKGRFTISRDDSKNTLYLQMNSLKTEDTAVYYCTTPWE | |
| | WSWYDYWGQGTLVTVSSASTKGPSVFPLAPSSKSTSGGT | |
| | AALGCLVKDYFPEPVTVSWNSGALTSGVHTFPAVLQSSG | |
| | LYSLSSVVTVPSSSLGTQTYICNVNHKPSNTKVDKKVEPK | |
| | SCDKTHTCPPCPAPEAAGGPSVFLFPPKPKDTLMISRTPEV | |
| | TCVVVDVSHEDPEVKFNWYVDGVEVHNAKTKPREEQYN | |
| | STYRVVSVLTVLHQDWLNGKEYKCKVSNKALGAPIEKTI | |
| | SKAKGQPREPQVYTLPPCRDELTKNQVSLWCLVKGFYPS | |
| | DIAVEWESNGQPENNYKTTPPVLDSDGSFFLYSKLTVDKS | |
| | RWQQGNVFSCSVMHEALHNHYTQKSLSLSP | |
| H chain | As in FOLR1 proTCB with protease linker | 66 |
| | anti-CD3 P035.093 | |
| L chain | As in FOLR1 proTCB with protease linker | 67 |
| | anti-CD3 P035.093 | |
| Matriptase | SGGGSGGGGSPMAKKGGGGSGGGGSGGGGSGGS | 68 |
| linker | | |

FolR1 pro TCB P035.093 H1L2

| | | |
|---|---|---|
| K chain | QVQLVQSGSELKKPGASVKVSCKASGYTVTDYSMNWVR | 96 |
| | QAPGQCLEWMGWINTETGEPRYTDDFKGRFVFSLDTSVS | |
| | TAYLQISSLKAEDTAVYYCAREGDYDVFDYWGQGTLVT | |
| | VSSGGGGSGGGGSGGGGSGGGGSDIVMTQSPDSLAVSLG | |
| | ERATINCKSSKSVSTSSYSYMHWYQQKPGQPPKLLIKYVS | |
| | YLESGVPDRFSGSGSGTDFTLTISSLQAEDVAVYYCQHSR | |
| | EFPYTFGCGTKLEIKGGGGSGGGGSPMAKKGGGGSGGGG | |
| | SGGGGSGGSEVQLLESGGGLVQPGGSLRLSCAASGFTFSS | |
| | YAMNWVRQAPGKGLEWVSRIRSKYNNYATYYADSVKG | |
| | RFTISRDDSKNTLYLQMNSLRAEDTAVYYCVRASNFPAS | |
| | YVSYFAYWGQGTLVTVSSASTKGPSVFPLAPSSKSTSGGT | |
| | AALGCLVKDYFPEPVTVSWNSGALTSGVHTFPAVLQSSG | |
| | LYSLSSVVTVPSSSLGTQTYICNVNHKPSNTKVDKKVEPK | |
| | SCDGGGGSGGGGSEVQLVESGGGLVKPGGSLRLSCAASG | |
| | FTFSNAWMSWVRQAPGKGLEWVGRIKSKTDGGTTDYAA | |
| | PVKGRFTISRDDSKNTLYLQMNSLKTEDTAVYYCTTPWE | |
| | WSWYDYWGQGTLVTVSSASTKGPSVFPLAPSSKSTSGGT | |
| | AALGCLVKDYFPEPVTVSWNSGALTSGVHTFPAVLQSSG | |
| | LYSLSSVVTVPSSSLGTQTYICNVNHKPSNTKVDKKVEPK | |
| | SCDKTHTCPPCPAPEAAGGPSVFLFPPKPKDTLMISRTPEV | |
| | TCVVVDVSHEDPEVKFNWYVDGVEVHNAKTKPREEQYN | |
| | STYRVVSVLTVLHQDWLNGKEYKCKVSNKALGAPIEKTI | |
| | SKAKGQPREPQVYTLPPCRDELTKNQVSLWCLVKGFYPS | |
| | DIAVEWESNGQPENNYKTTPPVLDSDGSFFLYSKLTVDKS | |
| | RWQQGNVFSCSVMHEALHNHYTQKSLSLSP | |
| H chain | As in FOLR1 proTCB with protease linker | 66 |
| | anti-CD3 P035.093 | |
| L chain | As in FOLR1 proTCB with protease linker | 67 |
| | anti-CD3 P035.093 | |
| Matriptase | SGGGSGGGGSPMAKKGGGGSGGGGSGGGGSGGS | 68 |
| linker | | |

FolR1 pro TCB P035.093 H2L2

| | | |
|---|---|---|
| K chain | QVQLVQSGSELKKPGASVKVSCKASGYTVTDYSMNWVR | 97 |
| | QAPGQCLEWMGWINTETGEPRYTDDFTGRFVFSLDTSVS | |
| | TAYLQISSLKAEDTAVYYCAREGDYDVFDYWGQGTLVT | |
| | VSSGGGGSGGGGSGGGGSGGGGSDIVMTQSPDSLAVSLG | |
| | ERATINCKSSKSVSTSSYSYMHWYQQKPGQPPKLLIKYVS | |
| | YLESGVPDRFSGSGSGTDFTLTISSLQAEDVAVYYCQHSR | |
| | EFPYTFGCGTKLEIKGGGGSGGGGSPMAKKGGGGSGGGG | |
| | SGGGGSGGSEVQLLESGGGLVQPGGSLRLSCAASGFTFSS | |
| | YAMNWVRQAPGKGLEWVSRIRSKYNNYATYYADSVKG | |
| | RFTISRDDSKNTLYLQMNSLRAEDTAVYYCVRASNFPAS | |
| | YVSYFAYWGQGTLVTVSSASTKGPSVFPLAPSSKSTSGGT | |
| | AALGCLVKDYFPEPVTVSWNSGALTSGVHTFPAVLQSSG | |
| | LYSLSSVVTVPSSSLGTQTYICNVNHKPSNTKVDKKVEPK | |
| | SCDGGGGSGGGGSEVQLVESGGGLVKPGGSLRLSCAASG | |
| | FTFSNAWMSWVRQAPGKGLEWVGRIKSKTDGGTTDYAA | |
| | PVKGRFTISRDDSKNTLYLQMNSLKTEDTAVYYCTTPWE | |
| | WSWYDYWGQGTLVTVSSASTKGPSVFPLAPSSKSTSGGT | |
| | AALGCLVKDYFPEPVTVSWNSGALTSGVHTFPAVLQSSG | |
| | LYSLSSVVTVPSSSLGTQTYICNVNHKPSNTKVDKKVEPK | |
| | SCDKTHTCPPCPAPEAAGGPSVFLFPPKPKDTLMISRTPEV | |
| | TCVVVDVSHEDPEVKFNWYVDGVEVHNAKTKPREEQYN | |
| | STYRVVSVLTVLHQDWLNGKEYKCKVSNKALGAPIEKTI | |
| | SKAKGQPREPQVYTLPPCRDELTKNQVSLWCLVKGFYPS | |
| | DIAVEWESNGQPENNYKTTPPVLDSDGSFFLYSKLTVDKS | |
| | RWQQGNVFSCSVMHEALHNHYTQKSLSLSP | |
| H chain | As in FOLR1 proTCB with protease linker | 66 |
| | anti-CD3 P035.093 | |
| L chain | As in FOLR1 proTCB with protease linker | 67 |
| | anti-CD3 P035.093 | |
| Matriptase | SGGGSGGGGSPMAKKGGGGSGGGGSGGGGSGGS | 68 |
| linker | | |

FolR1 pro TCB P035.093 H3L2

| | | |
|---|---|---|
| K chain | QVQLVQSGSELKKPGASVKVSCKASGYTVTDYSMNWVR | 98 |
| | QAPGQCLEWMGWINTETGEPRYTQGFKGRFVFSLDTSVS | |
| | TAYLQISSLKAEDTAVYYCAREGDYDVFDYWGQGTLVT | |
| | VSSGGGGSGGGGSGGGGSGGGGSDIVMTQSPDSLAVSLG | |
| | ERATINCKSSKSVSTSSYSYMHWYQQKPGQPPKLLIKYVS | |
| | YLESGVPDRFSGSGSGTDFTLTISSLQAEDVAVYYCQHSR | |
| | EFPYTFGCGTKLEIKGGGGSGGGGSPMAKKGGGGSGGGG | |
| | SGGGGSGGSEVQLLESGGGLVQPGGSLRLSCAASGFTFSS | |
| | YAMNWVRQAPGKGLEWVSRIRSKYNNYATYYADSVKG | |
| | RFTISRDDSKNTLYLQMNSLRAEDTAVYYCVRASNFPAS | |
| | YVSYFAYWGQGTLVTVSSASTKGPSVFPLAPSSKSTSGGT | |
| | AALGCLVKDYFPEPVTVSWNSGALTSGVHTFPAVLQSSG | |
| | LYSLSSVVTVPSSSLGTQTYICNVNHKPSNTKVDKKVEPK | |
| | SCDGGGGSGGGGSEVQLVESGGGLVKPGGSLRLSCAASG | |
| | FTFSNAWMSWVRQAPGKGLEWVGRIKSKTDGGTTDYAA | |
| | PVKGRFTISRDDSKNTLYLQMNSLKTEDTAVYYCTTPWE | |
| | WSWYDYWGQGTLVTVSSASTKGPSVFPLAPSSKSTSGGT | |
| | AALGCLVKDYFPEPVTVSWNSGALTSGVHTFPAVLQSSG | |
| | LYSLSSVVTVPSSSLGTQTYICNVNHKPSNTKVDKKVEPK | |
| | SCDKTHTCPPCPAPEAAGGPSVFLFPPKPKDTLMISRTPEV | |
| | TCVVVDVSHEDPEVKFNWYVDGVEVHNAKTKPREEQYN | |
| | STYRVVSVLTVLHQDWLNGKEYKCKVSNKALGAPIEKTI | |
| | SKAKGQPREPQVYTLPPCRDELTKNQVSLWCLVKGFYPS | |
| | DIAVEWESNGQPENNYKTTPPVLDSDGSFFLYSKLTVDKS | |
| | RWQQGNVFSCSVMHEALHNHYTQKSLSLSP | |
| H chain | As in FOLR1 proTCB with protease linker | 66 |
| | anti-CD3 P035.093 | |
| L chain | As in FOLR1 proTCB with protease linker | 67 |
| | anti-CD3 P035.093 | |
| Matriptase | SGGGSGGGGSPMAKKGGGGSGGGGSGGGGSGGS | 68 |
| linker | | |

Exemplary Linkers and Recognition Sequences

| | | |
|---|---|---|
| MMP | GGGGSGGGGSGPLGLWSQGGGGSGGGGSGGGGSGG | 99 |
| Protease | | |
| linker | | |
| Protease | RQARVVNG | 100 |
| recognition | | |
| site 1 | | |
| Protease | VHMPLGFLGPGRSRGSFP | 101 |
| recognition | | |
| site 2 | | |
| Protease | RQARVVNGXXXXXVPLSLYSG | 102 |
| recognition | | |
| site 3 | | |
| Protease | RQARVVNGVPLSLYSG | 103 |
| recognition | | |
| site 4 | | |
| Protease | PLGLWSQ | 104 |
| recognition | | |
| site 5 | | |
| Protease | VHMPLGFLGPRQARVVNG | 105 |
| recognition | | |
| site 6 | | |
| Protease | FVGGTG | 106 |
| recognition | | |
| site 7 | | |
| Protease | KKAAPVNG | 107 |
| recognition | | |
| site 8 | | |
| Protease | PMAKKVNG | 108 |
| recognition | | |
| site 9 | | |
| Protease | QARAKVNG | 109 |
| recognition | | |
| site 10 | | |
| Protease | VHMPLGFLGP | 110 |
| recognition | | |
| site 11 | | |
| Protease | QARAK | 111 |
| recognition | | |
| site 12 | | |
| Protease | VHMPLGFLGPPMAKK | 112 |
| recognition | | |
| site 13 | | |
| Protease | KKAAP | 113 |
| recognition | | |
| site 14 | | |
| Protease | PMAKK | 114 |
| recognition | | |
| site 15 | | |
| Combined | GGGGSVHMPLGFLGPRQARVVNGGGGGSGGGGS | 115 |
| MMP9 | | |
| MK062, 33 | | |
| AA for | | |
| CD3 | | |
| Cathepsin | GGGGSGGGGSGGGGSFVGGTGGGGSGGGGSGGS | 116 |
| S/B | | |
| KKAAPV | GGGGSGGGGSKKAAPVNGGGGGSGGGGSGGGGS | 117 |
| NG | | |
| PMAKKV | GGGGSGGGGSPMAKKVNGGGGGSGGGGSGGGGS | 118 |
| NG | | |
| QARAKV | GGGGSGGGGSQARAKVNGGGGGSGGGGSGGGGS | 119 |
| NG | | |
| MMP9 | GGGGSGGGGSVHMPLGFLGPGGGGSGGGGSGGS | 120 |
| QARAK | GGGGSGGGGSQARAKGGGGSGGGGSGGGGSGGS | 121 |
| MMP9- | GGGGSVHMPLGFLGPPMAKKGGGGSGGGGSGGS | 122 |
| PMAKK | | |
| KKAAP | GGGGSGGGGSKKAAPGGGGSGGGGSGGGGSGGS | 123 |
| PMAKK | GGGGSGGGGSPMAKKGGGGSGGGGSGGGGSGGS | 124 |
| Combined | GGGGSVHMPLGFLGPGRSRGSFPGGGGS | 125 |
| NF9/Mat5 | | |
| linker | | |
| Combined | GGGGSGGGGSRQARVVNGGGGGSVPLSLYSGGGGGSGG | 126 |
| MK062 | GGS | |
| MMP9 | | |
| Combined | GGGGSGGGGSRQARVVNGVPLSLYSGGGGGSGGGGS | 127 |
| MK062 | | |
| MMP9 | | |

### EXAMPLES

5 The following are examples of methods and compositions of the invention. It is understood that various other embodiments may be practiced, given the general description provided above.

### Example 1—Preparation of Optimized Anti-CD3 (Multispecific) Antibodies

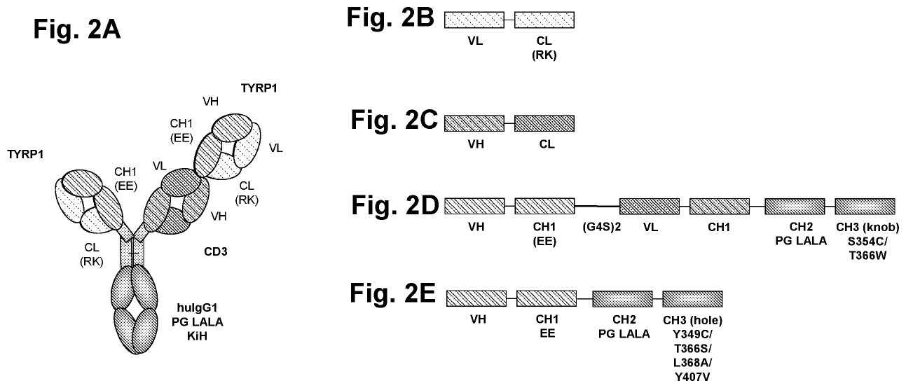

All optimized anti-CD3 antibodies (clones P033.078, P035.093, P035.064, P021.045, P004.042) were generated by phage display selection campaigns using libraries derived from a previously described (see e.g. WO 2014/131712, incorporated herein by reference) CD3 binder, termed “CD3_{orig}” herein and comprising the VH and VL sequences of SEQ ID NOs 14 and 23, respectively. In these libraries, positions N97 and N100 (Kabat numbering) located in the CDR3 region of the heavy chain were either silenced or removed. For direct comparison, all molecules were converted into T-cell bispecific antibody (TCB) format, as depicted in FIG. 2A, using an anti-TYRP1 antibody as exemplary target cell antigen binding moiety (SEQ ID NOs 24-31).

The variable region of heavy and light chain DNA sequences were subcloned in frame with either the constant heavy chain or the constant light chain pre-inserted into the respective recipient mammalian expression vectors as shown in FIGS. 2 B-E.

Sequences of the optimized anti-CD3 antibodies are given in the SEQ ID NOs indicated in Table 1.

TABLE 1
Sequences of optimized anti-CD3 antibodies generated in the present Examples.

| Clone | HCDR1 | HCDR2 | HCDR3 | VH | LCDR1 | LCDR2 | LCDR3 | VL |
|---|---|---|---|---|---|---|---|---|
| P033.078 | 2 | 5 | 9 | 15 | 20 | 21 | 22 | 23 |
| P035.093 | 2 | 4 | 10 | 16 | 20 | 21 | 22 | 23 |
| P035.064 | 3 | 6 | 11 | 17 | 20 | 21 | 22 | 23 |
| P021.045 | 2 | 4 | 12 | 18 | 20 | 21 | 22 | 23 |
| P004.042 | 3 | 7 | 13 | 19 | 20 | 21 | 22 | 23 |
| CD3_{orig} | 1 | 4 | 8 | 14 | 20 | 21 | 22 | 23 |

To improve correct pairing of the light chains with the corresponding heavy chains, mutations were introduced in the human CL (E123R, Q124K) and the human CH1 (K147E, K213E) of the TYRP1 binding Fab molecule.

For correct pairing of the heavy chains (formation of a heterodimeric molecule), knob-into-hole mutations were introduced in the constant region of the antibody heavy chains (T366W/S354C and T366S/L368A/Y407V/Y349C, respectively).

Furthermore, the P329G, L234A and L235A mutations were introduced in the constant region of the antibody heavy chains to abrogate binding to Fcγ receptors.

Full sequences of the prepared TCB molecules are given in SEQ ID NOs 32, 33, 34 and 36 (P033.078), SEQ ID NOs 32, 33, 34 and 37 (P035.093), SEQ ID NOs 32, 33, 34 and 38 (P035.064), SEQ ID NOs 32, 33, 34 and 39 (P021.045), SEQ ID NOs 32, 33, 34 and 40 (P004.042).

A corresponding molecule comprising CD3_{orig} as CD3 binder was also prepared.

The TCBs were prepared by Evitria (Switzerland) using their proprietary vector system with conventional (non-PCR based) cloning techniques and using suspension-adapted CHO K1 cells (originally received from ATCC and adapted to serum-free growth in suspension culture at Evitria). For the production, Evitria used its proprietary, animal-component free and serum-free media (eviGrow and eviMake2) and its proprietary transfection reagent (eviFect). The cells were transfected with the corresponding expression vectors in a 1:1:2:1 (“vector knob heavy chain”:“vector hole heavy chain”:“vector CD3 light chain”:“vector TYRP1 light chain”). Supernatant was harvested by centrifugation and subsequent filtration (0.2 μm filter) and, proteins were purified from the harvested supernatant by standard methods.

In brief, Fc containing proteins were purified from filtered cell culture supernatants by Protein A-affinity chromatography (equilibration buffer: 20 mM sodium citrate, 20 mM sodium phosphate, pH 7.5; elution buffer: 20 mM sodium citrate, pH 3.0). Elution was achieved at pH 3.0 followed by immediate pH neutralization of the sample. The protein was concentrated by centrifugation (Millipore Amicon® ULTRA-15, #UFC903096), and aggregated protein was separated from monomeric protein by size exclusion chromatography in 20 mM histidine, 140 mM sodium chloride, pH 6.0.

The concentrations of purified proteins were determined by measuring the absorption at 280 nm using the mass extinction coefficient calculated on the basis of the amino acid sequence according to Pace, et al., Protein Science, 1995, 4, 2411-1423. Purity and molecular weight of the proteins were analyzed by CE-SDS in the presence and absence of a reducing agent using a LabChipGXII (Perkin Elmer). Determination of the aggregate content was performed by HPLC chromatography at 25° C. using analytical size-exclusion column (TSKgel G3000 SW XL or UP-SW3000) equilibrated in running buffer (25 mM K2HPO₄, 125 mM NaCl, 200 mM L-arginine monohydrocloride, pH 6.7 or 200 mM KH₂PO₄, 250 mM KCl pH 6.2, respectively).

Results from the biochemical and biophysical analysis of the prepared TCB molecules are given in Table 2.

All TCB molecules could be produced in good quality.

TABLE 2
Biochemical and biophysical analysis
of anti-CD3 antibodies in TCB format.

| | | analytical size exclusion | | | CE-SDS |
|---|---|---|---|---|---|
| anti-CD3 | yield | chromatography [%] | | | (main peak) |
| antibody | [mg/l] | HMW | monomer | LMW | [%] |
| P033.078 | 32.8 | 0 | 100 | 0 | 100 |
| P035.093 | 26.8 | 0 | 100 | 0 | 100 |
| P035.064 | 46.4 | 0 | 100 | 0 | 100 |
| P021.045 | 25.9 | 0 | 100 | 0 | 100 |
| P004.042 | 28.7 | 0.4 | 99.6 | 0 | 100 |
| CD3_{orig} | 18.7 | 0 | 100 | 0 | 100 |

### Example 2—Determination of Thermal Stability of Optimized Anti-CD3 (Multispecific) Antibodies

Thermal stability of the anti-CD3 antibodies prepared in Example 1 (in TCB format) was monitored by Dynamic Light Scattering (DLS) and by monitoring of temperature dependent intrinsic protein fluorescence by applying a temperature ramp using an Optim 2 instrument (Avacta Analytical, UK).

10 μg of filtered protein sample with a protein concentration of 1 mg/ml was applied in duplicate to the Optim 2. The temperature was ramped from 25 to 85° C. at 0.1° C./min, with the ratio of fluorescence intensity at 350 nm/330 nm and scattering intensity at 266 nm being collected.

The results are shown in Table 3. The aggregation temperature (T_{agg}) and the midpoint of the observed temperature induced unfolding transition (Tₘ) of all the optimized CD3 binders produced in Example 1 is comparable or higher than for the previously described CD3 binder CD3_{orig}.

TABLE 3
Thermal stability of anti-CD3 antibodies in TCB format
as measured by dynamic light scattering and change of
temperature dependent intrinsic protein fluorescence.

| anti-CD3 | | |
|---|---|---|
| antibody | Tₘ [° C.] | T_{agg} [° C.] |
| P033.078 | 57 | 56 |
| P035.093 | 58.5 | 57 |
| P035.064 | 57.5 | 54 |
| P021.045 | 58.5 | 54 |
| P004.042 | 59 | 56 |
| CD3_{orig} | 57 | 54 |

### Example 3—Functional Characterization of Optimized Anti-CD3 (Multispecific) Antibodies 15 by Surface Plasmon Resonance (SPR)

All surface plasmon resonance (SPR) experiments were performed on a Biacore T200 at 25° C. with HBS-EP+ as running buffer (0.01 M HEPES pH 7.4, 0.15 M NaCl, 3 mM EDTA, 0.005% Surfactant P20; Biacore, Freiburg/Germany).

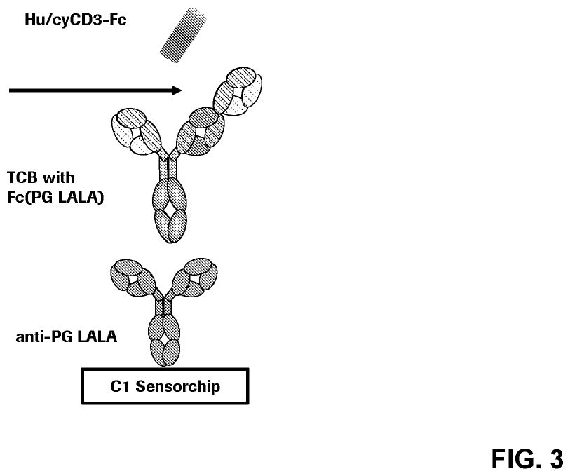

For affinity measurements, TCB molecules were captured on a C1 sensorchip (GE Healthcare) surface with immobilized anti-Fc(P329G) IgG (an antibody that specifically binds human IgG₁ Fc(P329G); “anti-PG antibody”—see WO 2017/072210, incorporated herein by reference). The experimental setup is schematically depicted in FIG. 3. Capture IgG was coupled to the sensorchip surface by direct immobilization of around 400 resonance units (RU) using the standard amine coupling kit (GE Healthcare Life Sciences).

To analyze the interaction to CD3, TCB molecules were captured for 80 s at 25 nM with a flow rate of 10 μl/min. Human and cynomolgus CD3ε stalk-Fc(knob)-Avi/CD3δ stalk-Fc(hole) (CD3ε/δ, see SEQ ID NOs 41 and 42 (human) and SEQ ID NOs 43 and 44 (cynomolgus)) were passed at a concentration of 0.122-125 nM with a flow rate of 30 μl/min through the flow cells for 300 s. The dissociation was monitored for 800 s.

Bulk refractive index differences were corrected for by subtracting the response obtained on the reference flow cell. Here, the antigens were flown over a surface with immobilized anti-PG antibody but on which HBS-EP has been injected instead of the TCB molecules.

Kinetic constants were derived using the Biacore T200 Evaluation Software (GE Healthcare Life Sciences), to fit rate equations for 1:1 Langmuir binding by numerical integration. The half-life (t_{1/2}) of the interaction was calculated using the formula t_{1/2}=ln 2/k_{off}.

In Table 4 all kinetic parameters of the binding of the optimized anti-CD3 antibodies compared to the previously described binder CD3_{orig} are listed. The optimized anti-CD3 antibodies (in TCB format) are binding to CD3ε/δ with K_{D} values in the in low nM range to high pM range, with K_{D}-values of 600 pM up to 1.54 nM for human CD3ε/δ and 200 pM to 700 pM for cynomolgus CD3ε/δ. Compared to CD3_{orig} the affinity of the binding to human CD3ε/δ of the optimized anti-CD3 antibodies is increased up to 7 to 10 fold as measured under same conditions by SPR.

The half-life of the monovalent binding to human CD3ε/δ is with 11.6 min for anti-CD3 antibody clone P033.078 up to 6-fold higher than the binding half-life of CD3_{orig}.

TABLE 4
Affinity of anti-CD3 antibodies (in TCB format)
to human and cynomolgus CD3ε/δ.

| | | Kinetic values at T = 25° C. | | | |
|---|---|---|---|---|---|
| | anti-CD3 | kₒₙ | k_{off} | K_{D} | t_{1/2} |
| antigen | antibody | [1/Ms] | [1/s] | [M] | [min] |
| Human | P033.078 | 1.66E+06 | 9.96E−04 | 6.00E−10 | 11.6 |
| CD3ε/δ | P035.093 | 3.75E+06 | 1.53E−03 | 4.10E−10 | 7.55 |
| | P035.064 | 1.83E+06 | 1.15E−03 | 6.30E−10 | 10 |
| | P021.045 | 3.10E+06 | 1.33E−03 | 4.30E−10 | 8.69 |
| | P004.042 | 1.92E+06 | 2.95E−03 | 1.54E−09 | 3.92 |
| | CD3_{orig} | 5.17E+05 | 3.38E−03 | 6.54E−09 | 3.42 |
| Cynomolgus | P033.078 | 2.20E+06 | 8.02E−04 | 3.70E−10 | 14.4 |
| CD3ε/δ | P035.093 | 4.89E+06 | 1.04E−03 | 2.10E−10 | 11.1 |
| | P035.064 | 2.44E+06 | 9.21E−04 | 3.80E−10 | 12.5 |
| | P021.045 | 4.88E+06 | 9.67E−04 | 2.00E−10 | 11.9 |
| | P004.042 | 3.85E+06 | 2.72E−03 | 7.10E−10 | 4.25 |
| | CD3_{orig} | 1.14E+06 | 2.52E−03 | 2.21E−09 | 4.58 |

### Example 4—Characterization of Optimized Anti-CD3 (Multispecific) Antibodies by Surface Plasmon Resonance (SPR) after Stress

In order to assess the effect of the deamidation site removal and its effect on the stability of the antibodies, the optimized anti-CD3 antibodies (in TCB format) were incubated for 14 days at 37° C., pH 7.4 and at 40° C., pH 6 and further analyzed by SPR for their binding capability to human CD3ε/δ. Samples stored at −80° C. pH 6 were used as reference. The reference samples and the samples stressed at 40° C. were in 20 mM His, 140 mM NaCl, pH 6.0, and the samples stressed at 37° C. in PBS, pH 7.4, all at a concentration of 1.0 mg/ml. After the stress period (14 days) samples in PBS were dialyzed back to 20 mM His, 140 mM NaCl, pH 6.0 for further analysis.

All SPR experiments were performed on a Biacore T200 instrument (GE Healthcare) at 25° C. with HBS-P+ (10 mM HEPES, 150 mM NaCl pH 7.4, 0.05% Surfactant P20) as running and dilution buffer. Biotinylated human CD3ε/δ (see Example 3, SEQ ID NOs 41 and 42) as well as biotinylated anti-huIgG (Capture Select, Thermo Scientific, #7103262100) were immobilized on a Series S Sensor Chip SA (GE Healthcare, #29104992), resulting in surface densities of at least 1000 resonance units (RU). Anti-CD3 antibodies with a concentration of 2 μg/ml were injected for 30 s at a flow rate of 5 μl/min, and dissociation was monitored for 120 s. The surface was regenerated by injecting 10 mM glycine pH 1.5 for 60 s. Bulk refractive index differences were corrected by subtracting blank injections and by subtracting the response obtained from a blank control flow cell. For evaluation, the binding response 5 seconds after injection end was taken. To normalize the binding signal, the CD3 binding was divided by the anti-huIgG response (the signal (RU) obtained upon capture of the CD3 antibody on the immobilized anti-huIgG antibody). The relative binding activity was calculated by referencing each temperature stressed sample to the corresponding, non-stressed sample.

As shown in Table 5, all anti-CD3 antibodies prepared in Example 1 show an improved binding upon stress to CD3ε/δ, as compared to CD3_{orig}.

TABLE 5
Binding activity of anti-CD3 antibodies (in
TCB format) to human CD3ε/δ after
incubation at pH 6/40° C. or pH 7.4/37° C. for 2 weeks.

binding activity [%]

| anti-CD3 | 2 weeks at | 2 weeks at |
|---|---|---|
| antibody | pH 6.0/40° C. | pH 7.4/37° C. |
| P033.078 | 99 | 99 |
| P035.093 | 97 | 95 |
| P035.064 | 97 | 96 |
| P021.045 | 99 | 95 |
| P004.042 | 98 | 98 |
| CD3_{orig} | 95 | 65 |

### Example 5—Jurkat NFAT Reporter Cell Assay with Optimized Anti-CD3 (Multispecific) Antibodies

The (TYRP1-targeted) TCBs containing the optimized anti-CD3 antibodies were tested in the Jurkat NFAT reporter cell assay in the presence of CHO-K1 TYRP1 clone 76 (cells were generated by stable transduction of CHO-K1 cells) as target cells. Jurkat NFAT reporter cells (Promega) were cultured in RPMI 1640 (Gibco) containing 10% FBS, 2 g/l glucose (Sigma), 2 g/l NaHCO₃(Sigma), 25 mM HEPES (Gibco), 1% GlutaMax (Gibco), 1×NEAA (Sigma), 1% SoPyr (Sigma) (Jurkat NFAT medium) at 0.1-0.5 mio cells/ml. CHO-K1 TYRP1 clone 76 cells were cultured in DMEM/F12+GlutaMAX (1×) (Gibco) containing 10% FBS and 6 μg/ml Puromycin (Invivogen). The assay was performed in Jurkat NFAT medium.

CHO-K1 TYRP1 clone 76 cells were detached using Trypsin (Gibco). The cells were counted and viability was checked. The target cells were re-suspended in assay medium and 10 000 cells were seeded per well in a white flat bottom 384 well plate. Then the TCBs were added at the indicated concentrations. Jurkat NFAT reporter cells were counted, viability was checked and 20 000 cells were seeded per well, corresponding to an effector-to-target (E:T) ratio of 2:1. Also, 2% end-volume of GloSensor cAMP Reagent (E1291, Promega) was added to each well. After the indicated incubation time, luminescence was measured using a Tecan Spark10M device.

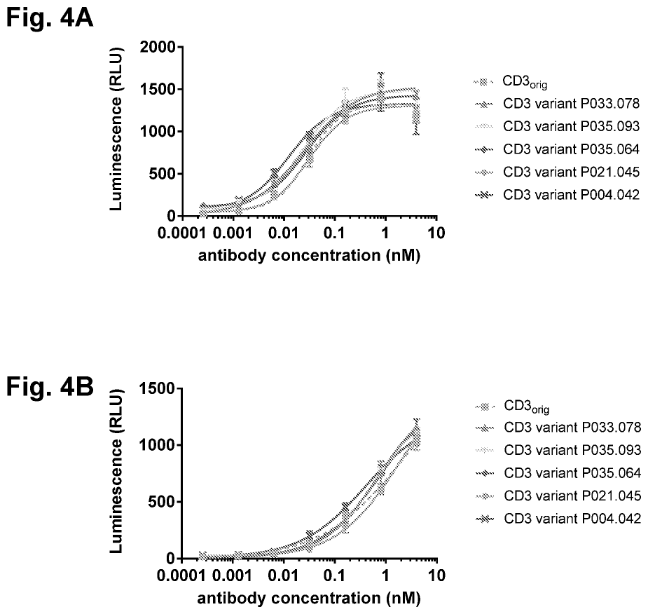

As shown in FIGS. 4A-B, the TCBs containing the optimized anti-CD3 antibodies had a similar functional activity on Jurkat NFAT reporter cells as the TCBs containing the parental binder CD3_{orig}. The tested TCBs induced CD3 activation in a concentration dependent manner.

### Example 6—Tumor Cell Killing of Primary Melanoma Cells with Optimized Anti-CD3 (Multispecific) Antibodies

The optimized anti-CD3 antibodies in (TYRP1-targeted) TCB format were tested in a tumor cell killing assay with freshly isolated human PBMCs, co-incubated with the human melanoma cell line M150543 (primary melanoma cell line, obtained from the dermatology cell bank of the University of Zurich). Tumor cell lysis was determined by quantification of LDH released into cell supernatants by apoptotic or necrotic cells after 24 h and 48 h. Activation of CD4 and CD8 T cells was analyzed by upregulation of CD69 and CD25 on both cell subsets after 48 h.

On the day before assay start, target cells (M150543) were detached using Trypsin (Gibco), washed once with PBS and re-suspended at a density of 0.3 mio cells/ml in growth medium (RPMI 1640 (Gibco) containing 10% FBS, 1% GlutaMax (Gibco) and 1% SoPyr (Sigma)). 100 μl of the cell suspension (containing 30 000 cells) were seeded into a 96 well flat bottom plate. The cells were incubated overnight at 37° C. in the incubator.

The next day, PBMCs were isolated from blood of a healthy donor and viability was checked. Medium was removed from plated target cells and 100 μl of assay medium (RPMI 1640 (Gibco) containing 2% FBS and 1% GlutaMax (Gibco)) were added to the wells. Antibodies were diluted in assay medium at indicated concentrations and 50 μl per well were added to the target cells. Assay medium was added to control wells. Isolated PBMCs were re-suspended at a density of 6 mio cells/ml, 50 μl were added per well resulting in 300 000 cells/well (E:T 10:1). For determination of spontaneous LDH release (minimal lysis=0%), PBMCs and target cells only were co-incubated. For determination of maximal LDH release (maximal lysis=100%), only assay medium was added to target cells. Control wells with PBMCs plus TCBs in absence of target cells were used to test the specificity of the TCBs. To determine if CD8 and CD4 T cells get activated in absence of tumor cells expressing the target, expression of CD25 was analyzed after 48 hours.

Few hours before the first LDH measurement, 50 μl of assay medium containing 4% Triton X-100 (Bio-Rad) was added to the wells containing target cells only (resulting in a final concentration of 1% Triton X-100 per well) for maximal LDH release. The assay was incubated in total for 48 h at 37° C. in the incubator. The first LDH measurement was performed 24 h after assay start. For this, the Cytotoxicity Detection Kit (LDH) (Roche/Sigma, #11644793001) was adjusted to room temperature before measurement. The assay plate was centrifuged for 4 min at 420×g and 50 μl of supernatant per well was transferred to a 96 well flat bottom plate for analysis. Then a reaction mixture of 1.25 μl of LDH Catalyst and 56.25 μl of LDH Substrate per well was prepared. 50 μl of the LDH reaction mixture was subsequently added to each well and absorbance was immediately measured using a TECAN Infinite F50 instrument. The measurement was repeated 48 h after assay start.

Afterwards PBMCs were harvested and analyzed by measuring CD25 and CD69 upregulation for activation. In detail, 100 μl of FACS buffer was added to each well and cells were transferred to a 96 well U bottom plate for FACS staining. The plate was centrifuged for 4 min at 400×g, supernatant was removed and cells were washed with 150 μl FACS buffer per well. The plate was again centrifuged for 4 min at 400×g and supernatant was removed. Subsequently 30 μl per well of the antibody mix containing CD4 APC (clone RPA-T4, BioLegend), CD8 FITC (clone SK1, BioLegend), CD25 BV421 (clone BC96, BioLegend) and CD69 PE (clone FN50, BioLegend) was added to the cells. The cells were incubated for 30 min in the fridge. Afterwards the cells were washed twice with FACS buffer and re-suspended in 100 μl FACS buffer containing 1% PFA per well. Before the measurement, cells were resuspended in 150 μl FACS buffer. The analysis was performed using a BD LSR Fortessa device.

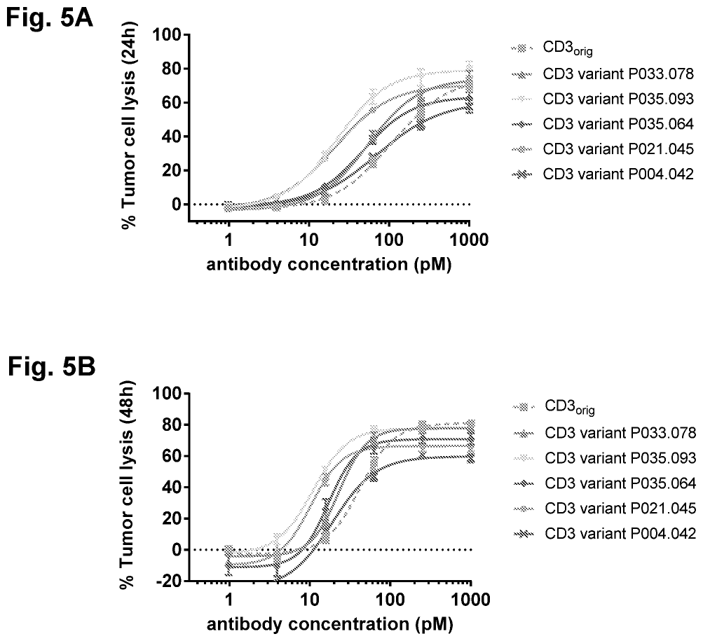

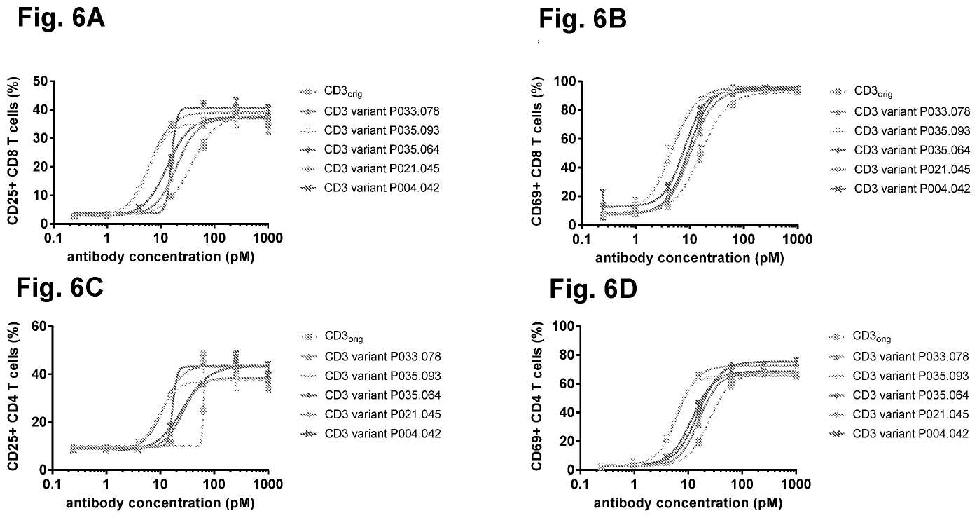

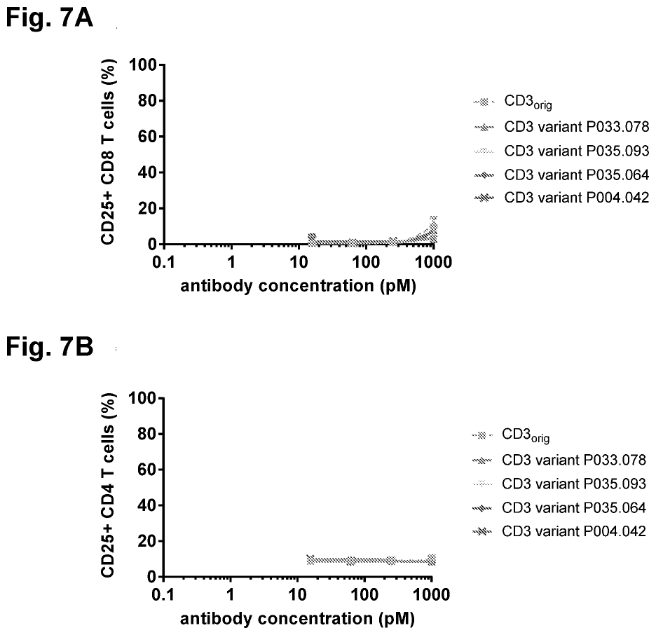

Treatment with TCBs containing the anti-CD3 antibody clone P035.093 and clone P021.045 led to highest tumor cell killing, the clone P033.078 and clone P035.064 resulted in a medium degree of tumor cell killing, followed by clone P004.042 inducing similar tumor cell killing compared to TCBs containing the parental binder CD3_{orig} (FIGS. 5A-B). Activation of T cells is highest when treated with TCBs containing the anti-CD3 antibody clone P035.093 and clone P021.045, whereas the TCBs containing the other anti-CD3 antibody clones led to similar T cell activation as to the TCBs containing the parental binder CD3_{orig} (FIGS. 6A-D).

As shown in FIGS. 7A-B, the tested TCBs did not induce CD25 upregulation on CD8 and CD4 T cells in absence of tumor target cells. This result shows that the tested CD3 binders depend on 5 crosslinking for example via binding to a tumor cell to induce T cell activation and are not able to induce T cell activation in a monovalent format.

### Example 7—Preparation of Optimized Anti-CD3 Antibodies

The optimized anti-CD3 antibodies clones P033.078, P035.093, and P004.042 were converted into monovalent human IgG₁ format, with crossed VH and VL domains on the CD3 binding moeity as depicted in FIG. 8A.

The variable region of heavy and light chain DNA sequences were subcloned in frame with either the constant heavy chain or the constant light chain pre-inserted into the respective recipient mammalian expression vectors as shown in FIGS. 8B-D.

For correct pairing of the heavy chains (formation of a heterodimeric molecule), knob-into-hole mutations were introduced in the constant region of the antibody heavy chains (T366W/S354C and T366S/L368A/Y407V/Y349C, respectively).

Furthermore, the P329G, L234A and L235A mutations were introduced in the constant region of the antibody heavy chains to abrogate binding to Fcγ receptors.

Corresponding molecules comprising CD3_{orig} as CD3 binder were also prepared.

The monovalent IgG molecules were prepared at Evitria (Switzerland), purified and analysed as described for the TCB molecules in Example 1. For transfection of the cells, the corresponding expression vectors were applied in a 1:1:1 ratio (“vector knob heavy chain”:“vector hole heavy chain”:“vector light chain”).

Results from the biochemical and biophysical analysis of the prepared monovalent IgG molecules are given in Table 6.

All monovalent IgG molecules could be produced in good quality.

TABLE 6
Biochemical and biophysical analysis of anti-
CD3 antibodies in monovalent IgG format.

| | | analytical size exclusion | | | CE-SDS |
|---|---|---|---|---|---|
| anti CD3 | yield | chromatography [%] | | | (main peak) |
| antibody | [mg/l] | HMW | monomer | LMW | [%] |
| P033.078 | 1560 | 0 | 98.9 | 1.1 | 94.6 |
| P035.093 | 2250 | 0 | 98.2 | 1.8 | 92.1 |
| P004.042 | 3360 | 0 | 100 | 0 | 84.5 |
| CD3_{orig} | 1447.5 | 0.9 | 99.1 | 0 | 90.5 |

### Example 8—Determination of Thermal Stability of Optimized Anti-CD3 Antibodies

Thermal stability of the anti-CD3 antibodies in monovalent IgG format (prepared in Example 19) was monitored by Dynamic Light Scattering (DLS) and by monitoring of temperature dependent intrinsic protein fluorescence as described in Example 2.

The results are shown in Table 7. The aggregation temperature (T_{agg}) and the midpoint of the observed temperature induced unfolding transition (Tₘ) of all the optimized CD3 binders in monovalent IgG format is comparable or higher than for the previously described CD3 binder CD3_{orig}.

TABLE 7
Thermal stability of anti-CD3 antibodies in monovalent IgG
format as measured by dynamic light scattering and change
of temperature dependent intrinsic protein fluorescence.

| anti CD3 | | |
|---|---|---|
| antibody | Tₘ [° C.] | T_{agg} [° C.] |
| P033.078 | 57.0 | 55.5 |
| P035.093 | 58.0 | 55.5 |
| P004.042 | 58.5 | 56.0 |
| CD3_{orig} | 55 | 53.0 |

### Example 9—Functional Characterization of Optimized Anti-CD3 Antibodies by Surface Plasmon Resonance (SPR)

SPR experiments were performed as described in Example 3, with the monovalent IgG molecules prepared in Example 7.

To analyze the interaction to CD3, IgG molecules were captured for 240 s at 50 nM with a flow rate of 5 μl/min. Human and cynomolgus CD3ε stalk-Fc(knob)-Avi/CD3δ-stalk-Fc(hole) were passed at a concentration of 0.061-250 nM with a flow rate of 30 μl/min through the flow cells for 300 s. The dissociation was monitored for 800 s.

In Table 8 all kinetic parameters of the binding of the optimized anti-CD3 antibodies compared to the previously described binder CD3_{orig} are listed. The optimized anti-CD3 antibodies (in monovalent IgG format) are binding to CD3ε/δ with K_{D} values in the in low nM range to high pM range, with K_{D}-values of 770 pM up to 1.36 nM for human CD3ε/δ and 200 pM to 400 pM for cynomolgus CD3ε/δ. Compared to CD3_{orig} the affinity of the binding to human CD3ε/δ of the optimized anti-CD3 antibodies is increased up to 3.5 to 15-fold as measured under same conditions by SPR.

The half-life of the monovalent binding to human CD3ε/δ is with 8.69 min for anti-CD3 antibody clone P033.078 more than 2-fold higher than the binding half-life of CD3_{orig}.

TABLE 8
Affinity of anti-CD3 antibodies (in monovalent IgG
format) to human and cynomolgus CD3ε/δ. Data
obtained from triplicate measurements.

| | | Kinetic values at T = 25° C. | | | |
|---|---|---|---|---|---|
| | anti CD3 | kₒₙ | k_{off} | K_{D} | t_{1/2} |
| antigen | antibody | [1/Ms] | [1/s] | [M] | [min] |
| Human | P033.078 | 1.73E+06 | 1.33E−03 | 7.71E−10 | 8.69 |
| CD3ε/δ | P035.093 | 3.08E+06 | 1.40E−03 | 4.56E−10 | 8.25 |
| | P004.042* | 6.28E+06 | 8.52E−03 | 1.36E−09 | 1.36 |
| | CD3_{orig} | 5.87E+05 | 2.90E−03 | 4.94E−09 | 3.98 |
| Cynomolgus | P033.078 | 2.60E+06 | 1.04E−03 | 4.03E−10 | 11.11 |
| CD3ε/δ | P035.093 | 4.38E+06 | 9.81E−04 | 2.24E−10 | 11.78 |
| | P004.042* | 1.85E+07 | 8.09E−03 | 4.39E−10 | 1.43 |
| | CD3_{orig} | 1.20E+06 | 2.45E−03 | 2.03E−09 | 4.72 |

*kinetic and affinity values may not be fully reliable, due to bad fit quality

### Example 10—Generation of Anti-Idiotypic Masks

Production and Evaluation of Anti-Idiotypic Masks as Chimeric IgGs

The chimeric IgGs described herein were prepared by Evitria using their proprietary vector system with conventional (non-PCR based) cloning techniques and using suspension-adapted CHO K1 cells (originally received from ATCC and adapted to serum-free growth in suspension culture at Evitria). For the production, Evitria used its proprietary, animal-component free and serum-free media (eviGrow and eviMake2) and its proprietary transfection reagent (eviFect). Supernatant was harvested by centrifugation and subsequent filtration (0.2 μm filter) and purified by standard methods.

Characterization of Anti-Idiotypic Masks—Binding to Different CD3 mAbs

SPR experiments were performed on a Biacore T200 with HBS-EP+ as running buffer (0.01 M HEPES pH 7.4, 0.15 M NaCl, 0.005% Surfactant P20 (BR-1006-69, GE Healthcare)). Three anti-idiotypic antibodies were directly immobilized by amine coupling on a CMS chip (GE Healthcare). A three-fold dilution series of the different T cell bispecifics (TCBs) was passed 20 over the ligand at 30 μl/min for 180 sec to record the association phase. The dissociation phase was monitored for 600 s and triggered by switching from the sample solution to HBS-EP+. The chip surface was regenerated after every cycle using one injection of 10 mM glycine pH 2.1 for 60 sec, followed by two injections of 30 sec. Bulk refractive index differences were corrected for by subtracting the response obtained on the reference flow cell 1. The affinity constants were derived from the kinetic rate constants by fitting to a 1:1 Langmuir binding using the Biaeval 5 software (GE Healthcare). The measure was performed with a single dilution series.

TABLE 9
Binding affinities of different masks to different CD3 binders.
SPR analysis was evaluated with masks as IgGs (immobilized on
CM5 chip) and TCBs with different CD3 binding Fabs as analytes.

| Anti- | | | | |
|---|---|---|---|---|
| idiotypic | CD3 | ka | kd | KD |
| IgG | binder | [1/Ms] | [1/s] | [nM] |
| 4.15.64 | P035.093 | 1.44E+04 | 1.74E−04 | 12.0 |
| | CH2527 | 1.69E+04 | 4.43E−04 | 26.2 |
| 4.24.72 | P035.093 | 1.48E+05 | 3.09E−04 | 2.1 |
| | CH2527 | 1.41E+05 | 6.75E−04 | 4.7 |
| 4.32.63 | P035.093 | no binding | | |
| | CH2527 | 2.92E+04 | 2.99E−04 | 10.0 |

Characterization of Anti-Idiotypic Masks—Developability

As one of the anti-idiotypic masks (4.15.64) exhibits an N-glycosylation site in the CDRL1 (NYS) this molecule was not taken into consideration any more, but only 4.24.72 and 4.32.63 masks were further more evaluated as they could be used for blocking of different CD3 binders

Binding of the anti-idiotypic antibodies 4.32.63 and 4.24.72 after 14 d incubation in either 20 mM His/HCl, 140 mM NaCl pH 6.0 at 40° C. or 1×PBS pH 7.4 at 37° C. was investigated by surface plasmon resonance using a Biacore T200 instrument (GE Healthcare). Briefly, monomeric FolR1-Fc (on flow cell 2) and an anti-PGLALA antibody (on flow cell 4) were immobilized on series S sensor chip CMS (CE Healthcare) using standard amine coupling chemistry, resulting of surface densities above 10000 resonance units (RU). Flow cells 1 and 3 were used as mock controls. FolR1 TCB-D-16D5 containing the CD3-CH2527 binding domain was injected only on the FolR1-Fc surface at a concentration of 10 μg/ml for 120 s at a flow rate of 5 μl/min, resulting a surface density above 1000 RU. Subsequently, the anti-idiotypic antibodies were injected onto all flow cells at a concentration of 1 μg/ml for 60 s and 120 s at a flow rate of 5 μl/min. The dissociation was monitored for 60 s. The FolR1-Fc surface was regenerated by injecting 10 mM Glycine pH 1.7 for 60 s, the anti-PGLALA surface by injecting 10 mM NaOH for 60 s. Bulk refractive index differences were corrected by subtracting the response obtained from flow cell 1 and 3 (mock surfaces).

To normalize the binding signals of the anti-idiotypic antibodies, the binding response of the FOLR1 TCB-D-16D5 surface was divided by the binding response of the anti-PGLALA surface. The relative active concentration was obtained by dividing the normalized response of the stressed samples by the normalized response of the unstressed reference sample for each molecule.

TABLE 10
Comparison of molecule stability of parental chimeric anti-idiotypic
masks (thermal stability and molecule integrity/activity after stress
conditions e.g incubation for 14 days in different buffers).

| | 4.32.63 | 4.24.72 |
|---|---|---|
| Thermal stability [Tagg ° C.] | 67.7 | 61.7 |

Remaining molecule integrity and activity after stress [%]:
CE-SDS; non-reducing

| | | |
|---|---|---|
| 40° C. pH6.0 | 96 | 97 |
| 37° C. pH7.4 | 94 | 94 |

CE-SDS; reducing

| | | |
|---|---|---|
| 40° C. pH6.0 | 98 | 99 |
| 37° C. pH7.4 | 98 | 98 |

Monomer content [%]

| | | |
|---|---|---|
| 40° C. pH6.0 | 99 | 98 |
| 37° C. pH7.4 | 99 | 98 |

Rel. active concentration [%]

| | | |
|---|---|---|
| 40° C. pH6.0 | 73 | 96 |
| 37° C. pH7.4 | 96 | 92 |

For the 4.32.63 mask a significant decrease in relative active concentration (73% remaining target binding) was observed after incubation for 14 days at 40° C. at pH6.0, whereas 4.24.72 is stable with 96% remaining target binding activity under these conditions.

### Example 11—Screening of Anti-Idiotypic Clones Against CD3 Binder P035.093

Binding and blocking capacity of anti-idiotypic (anti-ID) clones (4.24.72, 4.32.63, 4.21 and 4.15.64) against CD3 binder was tested using Jurkat NFAT activation assay with TYRP1 TCB (different CD3 binders). Blocking of anti-ID IgG can be seen in a reduction of Jurkat NFAT activation as a result of the blocked CD3 binder (FIGS. 10A-10E).

TYRP1 targeting T cell bispecific antibody (TCB) simultaneously binds to TYRP1 on target cell and CD3epsilon on T cell (Jurkat NFAT) thereby inducing T cell activation. T cell activation correlates with luminescence as the Jurkat NFAT cells express luciferase upon activation via CD3epsilon (CD3ε). Jurkat-NFAT reporter cell line (Promega) is a human acute lymphatic leukemia reporter cell line with a NFAT promoter, expressing human CD3ε. If TCB binds tumor target and CD3 (crosslinkage) binds CD3c Luciferase expression can be measured in Luminescence after addition of One-Glo substrate (Promega).

Jurkat NFAT assay medium: RPMI1640, 2 g/l Glucose, 2 g/l NaHCO₃, 10% FCS, 25 mM HEPES, 2 mM L-Glutamin, 1×NEAA, 1× Sodium-pyruvate

Jurkat NFAT cultivation medium: RPMI1640, 2 g/l Glucose, 2 g/l NaHCO₃, 10% FCS, 25 mM HEPES, 2 mM L-Glutamin, 1×NEAA, 1× Sodium-pyruvate; freshly added Hygromycine B 200 μg/ml.

TYRP1 positive target cells (CHO-huTYRP1 cl 76) and effector cells (Jurkat NFAT) were harvested, counted and checked for viability. The TCB was diluted in Jurkat assay medium (final concentration: EC90 concentration determined in previous assay, 50 μl/well). TCB, target cells (20.000/well in 50 μl/well) and Jurkat NFAT effector cells with cAMP (2% end volume) (50.000 cells/well in 50 μl/well) were mixed and added to a 96-well white walled flat bottom plate (Greiner BioOne). The E:T ratio was therefore 2.5:1. Anti-idiotypic IgGs were diluted in Jurkat assay medium before a dilution row was prepared and 50 μl per well were added. Cells were incubated for 22 h at 37° C. in a humidified incubator before they were taken out of the incubator for about 10 min to adapt to room temperature prior to Luminescence read out in Tecan Spark using 0.5 sec/well as detection time. The TYRP1 TCBs (different CD3 binders) induce Jurkat NFAT activation whereas a non-targeted TCB (CD3 CH2527) does not (FIG. 10A). When the anti-ID IgG binds to the CD3 binder it can block Jurkat NFAT activation as shown for the anti-ID 4.24.72 IgG that blocks all CD3 binders used in here except CD3 clone 22 and CD3 P033.005 (FIG. 10B). The anti-ID 4.32.63 IgG blocks only CD3 binder CH2527 (FIG. 10C). The anti-ID 4.15.64 IgG blocks all CD3 binders used in here except CD3 clone 22 (FIG. 10D). The anti-ID 4.21 IgG blocks all CD3 binders used in here except CD3 clone 22, whereas the strongest 5 blocking was observed for CD3 CH2527 (FIG. 10E). All together the anti-ID 4.24.72 showed the best blocking capacity in this assay set up and was therefore converted in pro-TCB format using CD3 P035.093.

### Example 12—Masking-Efficiency of Anti-ID 4.24.72 in FOLR1 Pro-TCB Format

Comparison of FOLR1 proTCBs with Different CD3 Binders

To compare masking of different CD3 binders with the anti-idiotypic mask 4.24.72, FOLR1 proTCB and the respective FOLR1 TCB molecules were produced. The proTCB molecules contained either a non-cleavable GS linker between mask and CD3 Fab or a linker sequence cleavable by MMP2/9 and matriptase.

15 All molecules were produced in sufficient amounts and at good quality. As expected, the proTCB molecules usually showed a lower yield as compared to the parental TCBs.

TABLE 11
Production and characterization of FOLR1 TCBs and FOLR1
proTCB containing different CD3 binding units.

| | Yield | Product peak | Main peak | Endotoxin |
|---|---|---|---|---|
| Description | [mg/L] | SEC [%] | CE-SDS [%] | [EU/ml] |
| FOLR1proTCB_CH2527_MT | 0.6 | 93.6 | 94.4 | <0.59 |
| FOLR1proTCB_CH2527_MMP-MT | 4.3 | 100.0 | 95.4 | <0.17 |
| FOLR1proTCB_CH2527_non-cleavable linker | 8.3 | 96.9 | 97.5 | <0.11 |
| FOLR1 TCB_CH2527 | 12.9 | 97.3 | 100.0 | <0.04 |
| FOLR1proTCB_P035.093_MMP-MT | 6.9 | 94.0 | 91.3 | 0.19 |
| FOLR1proTCB_P035.093_non-cleavable linker | 3.1 | 97.6 | 100.0 | n.d. |
| FOLR1 TCB_P035.093 | 5.6 | 96.0 | 98.6 | <0.14 |
| FOLR1proTCB_P021.045_MMP-MT | 3.7 | 93.1 | 94.2 | <0.1 |
| FOLR1proTCB_P021.045_non-cleavable linker | 1.0 | 95.8 | 100.0 | n.t. |
| FOLR1 TCB P021.045 | 4.4 | 94.1 | 100.0 | n.t. |
| FOLR1proTCB_P035.093_new MT | 1.9 | 98.4 | 97.4 | <0.45 |

MMP: cleavage site for hu MMP2/9;
MT: cleavage site for humatriptase

FOLR1 TCB and FOLR1 pro-TCBs were tested with Jurkat NFAT activation assay to see if anti-ID mask 4.24.72 blocks CD3 binders in pro-TCB format (anti-ID disulfide stabilized scFv N-terminally fused to CD3 binder). Jurkat NFAT assay was performed with huFOLR1 coated beads instead of target cells. 2×30 μl Streptavidin Dynabeads were diluted in 5 ml DPBS each. The beads were centrifuged at 400rcf for 4 min and supernatant was aspirated. Beads were coated with 20 μg of biotinylated FolR1 antigen in 1 ml for 1 h at 4° C., slowly rotating. After incubation, the bead-ag conjugates were washed with 5 ml DPBS each and resuspended in 4 ml assay medium. Effector cells (Jurkat NFAT) were harvested, counted and checked for viability. The TCBs were diluted in Jurkat assay medium. TCBs (10 μl/well), coated beads (10 μl/well) and Jurkat NFAT effector cells with cAMP (2% end volume) (20.000 cells/well in 20 μl/well) were mixed and added to a 384-well white walled flat bottom plate (Falcon/Corning). Plate was incubated for 5-6 h at 37° C. in a humidified incubator before they were taken out of the incubator for Luminescence read out in Tecan Spark using 0.5 sec/well as detection time.

FOLR1 pro-TCBs with anti-ID mask 4.24.72 do not mediate Jurkat NFAT activation in the indicated concentration range whereas FOLR1 TCBs do mediate dose-dependent Jurkat NFAT activation (FIG. 11A) meaning that the anti-ID 4.24.72 also works in pro-TCB format in terms of blocking.

The next step was to test the FOLR1 pro-TCB with the cleavable linker to test masking-efficiency in killing (more sensitive than Jurkat NFAT) and release of mask upon linker cleavage. T-cell killing mediated by FOLR1 (pro-) TCBs was assessed using HeLa (FolR1+++) cells. Human PBMCs were used as effector cells with an E:T ratio of 10:1. Human Peripheral blood mononuclear cells (PBMCs) were isolated from buffy coats obtained from healthy human donors. Buffy coat was diluted 1:1 with sterile PBS and layered over Histopaque gradient (Sigma, #H8889). After centrifugation (450×g, 30 minutes, w/o break, room temperature) the PBMC-containing interphase was transferred in a new falcon tube subsequently filled with 50 ml of PBS. The mixture was centrifuged (400×g, 10 minutes, room temperature), the supernatant discarded and the PBMC pellet resuspended in 2 ml ACK buffer for Erythrocytes lysis. After incubation at 37° C. for about 2-3 minutes the tubes were filled with sterile PBS to 50 ml and centrifuged at 350×g for 10 minutes. This washing step was repeated once prior to resuspension of PBMCs in RPMI1640 medium containing 10% FCS, 1× GlutaMax and 10% DMSO. PBMCs were slowly frozen in CoolCell® Cell Freezing Containers (BioCision) at −80° C. and then transferred to liquid nitrogen. One day before assay start adherent target cells were harvested with Trypsin/EDTA, counted, checked for viability and resuspended in assay medium (RPMI1640, 2% FCS, 1× GlutaMax). About 24 h before assay start PBMCs were thawed in advanced RPMI1640 medium (+2% FCS, 1× GlutaMax). PBMCs were centrifuged at 350 g for 7 min and resuspended in fresh medium (advanced RPMI1640, 2% FCS, 1× GlutaMax). PBMCs were kept for a maximum of 24 hours before they were used for an assay. Target cells were plated at a density of 20 000 cells/well using 96-well flat-bottom plates. The molecules were diluted in assay medium (RPMI1640, 2% FCS, 1× GlutaMax) and added at the indicated concentrations in triplicates. Plates were incubated at 37° C. for about 20 h in a humidified incubator. PBMCS were harvested and centrifuged at 350 g for 7 min before they were resuspended in assay medium (RPMI1640, 2% FCS, 1× GlutaMax). 0.2 mio PBMCs in 100 μl/well (E:T 10:1, based on the number of seeded target cells) were added before plates were incubated at 37° C. for 48 h. Target cell killing was assessed after 48 h of incubation at 37° C., 5% CO2 by quantification of LDH release into cell supernatants by apoptotic/necrotic cells (LDH detection kit, Roche Applied Science, #11 644 793 001). Standard response refers to target cells co-incubated with effector cells without any TCB.

FOLR1 TCB induces dose-dependent HeLa cell killing with an EC50 value ˜0.29 pM. The potency of the activated pro-TCB (pre-incubated with recombinant matriptase for linker cleavage) was comparable to the FOLR1 TCB. The pro-TCB containing a non-cleavable linker mediated reduced target cell killing (EC50 about 239 fold increased) (FIG. 11C). In addition to target cell killing the T-cell activation was assessed after 48 h of incubation at 37° C., 5% CO2 by quantification of CD69 on CD8 positive T cells. Regarding MFI for CD69 on CD8 positive T cells the potency of FOLR1 TCB and pre-activated FOR1 pro-TCB is comparable and no CD8 T cell activation can be detected for the masked pro-TCB (non-clevable). Regarding percentage of CD69 positive CD8 T cells, the masked pro-TCB shows an increase in CD69 positive CD8 T cells >5 nM increasing to around 30% at the highest concentration used in here. Masking efficiency of anti-ID 4.24.72 was compared for different cell lines with different FOLR1 expression levels.

Dose-dependent target cell killing (Hela high FOLR1 expression, Ovcar-3 and Skov-3 medium FOLR1 expression and HT-29 with low FOLR1 expression) was measured after 48 h of incubation of huPBMCs to analyze masking-efficiency of anti-ID 4.24.72 in pro-TCB format with CD3 P035.093. TCB and FOLR1 positive target cells (E:T=10:1, effectors are human 5 PBMCs). FOLR1 TCB induces dose-dependent target cell killing on all cell lines (Hela, Skov-3, Ovcar-3, HT-29) whereas the masked FOLR1 pro-TCB shows reduced target cell killing (FIGS. 12A and 12B). Masking-efficiency seems to be dependent on FOLR1 expression level. Target cell killing induced by FOLR1 pro-TCB (non-cleavable) seems to be reduced the most for cells with lower FOLR1 expression level. Comparison of FOLR1 TCB with CD3 binder CH2527 and 10 FOLR1 TCB with CD3 binder P035.93 shows slightly higher potency for the TCB with CD3 P035.093 (FIG. 12B). Masking of both CD3 binders with anti-ID 4.24.72 is possible.

### Example 13—Humanization of Mask 4.24.72

As shown in Example 12, the FOLR1 proTCB was efficiently blocked with the mask 4.24.72, as shown in in a Jurkat NFAT T-cell activation assay. After linker cleavage, this proTCB molecule was fully active in a target cell killing assay. Therefore, and as this mask can be used with different CD3 binders this anti-idiotypic antibody was chosen for humanization. Ten different variable heavy chains and eight different variable light chains were designed and produced as monomeric one-armed IgGs (FIG. 13). Heterodimerization of the molecules was enabled by applying the knob-into-holes technology. The one-armed IgGs were transiently produced in 2 ml small scale in Expi293F cells (transfection was performed according to the manufacturer's recommendation). Initial binding to CD3 IgG (P035.093) and blocking of T-cell activation in a Jurkat NFAT reporter assay (described below) was evaluated directly using the production supernatant containing the one-armed molecules.

Screening of Humanization Variants for their Blocking of CD3 P035.093—Jukat NFAT Activation Assay

The humanization variants (IgG format) were screened for their blocking capacity of CD3 binder P035.093 (and CH2527) using Jurkat NFAT assay described above. The TCB was used at EC90 concentrations (determined in previous assay) and the anti-ID IgGs were titrated. The parental 4.24.72 IgG was used as a control. The parental 4.24.72 blocked the CD3 CH2527 and P035.09. The humanization variants also block CD3 CH2527 and P035.093 whereas they all seem to block P035.093 slightly better compared to CD3 CH2527 (FIGS. 14A and 14B). Taken together they are all masking CD3 P035.093. Based on the results six variants were selected and produced and purified as IgGs and proTCBs as described above (with non-cleavable linker in case of the proTCB format) for comparison with the parental clone.

Developability Anti-CD3 P035.093 4.24.72 Anti-Idiotypic Antibody and Corresponding Humanized Variants

Humanization variants of mask 4.24.72 contain potential sequence hot spots which might cause instability of the molecules. Therefore, they were analyzed for thermal stability and remaining target binding after 14 d stress conditions (40° C. at pH6.0 or 37° C. at pH7.4).

Binding of the anti-idiotypic antibody 4.24.72 and its humanized variants H1L1, H1L2, H2L2, H3L2, H3L3 and H7L5 after 14 d incubation in either 20 mM His/HCl, 140 mM NaCl pH 6.0 at 40° C. or 1×PBS pH 7.4 at 37° C., was investigated by surface plasmon resonance using a Biacore T200 instrument (GE Healthcare). Briefly, a biotinylated anti-human CD3 IgG (anti-CD3 P035.093) and a biotinylated anti-human IgG (ThermoScientific) were immobilized on a series s CAP chip according to the manufacturer's instruction using the Biotin CAPture Kit (GE Healthcare). The antibodies were immobilized on flow cell 2 and 3 by injecting 5 μg/ml each for 120 s at a flow rate of 5 μl/min, leading to surface densities above 1000 resonance units (RU). Flow cell 1 was kept as mock surface. Subsequently, the anti-idiotypic antibodies were injected onto all flow cells at a concentration of 1 μg/ml for 30 s. Dissociation was monitored for 30 s, the flow rate was set to 5 μl/min. The CAP chip was regenerated by injecting a mix of NaOH and Guadinium-hydrochloride for 120 s, provided in the biotin capture kit. Bulk refractive index differences were corrected by subtracting the response obtained from flow cell 1 (mock surface). To normalize the binding signals of the anti-idiotypic antibodies, the binding response of anti-human CD3 IgG surface was divided by the binding response of the anti-human IgG surface. The relative active concentration was obtained by dividing the normalized response of the stressed samples by the normalized response of the unstressed reference sample for each molecule.

TABLE 12
Comparison of selected variants of the humanized anti-idiotypic mask
4.24.72 with respect to aggregation temperature, and stability/activity
after stress test. Aggregation temperature above 58° C. and relative
retention time of less than 0.35 min on HIC column are seen as uncritical
values. Apart from samples H3L3 and H7L5 with only 87% and 80%
relative active concentration, all other molecules are stable under the
tested conditions.

| | H1L1 | H1L2 | H2L2 | H3L2 | H3L3 | H7L5 |
|---|---|---|---|---|---|---|
| Thermal | 61.0 | 60.2 | 59.3 | 60.0 | 64.2 | 61.0 |
| stability | | | | | | |
| Tagg ° C. | | | | | | |

Remaining molecule integrity and activity after stress [%]:
CE-SDS; non-reducing

| | | | | | | |
|---|---|---|---|---|---|---|
| 40° C. | 97 | 96 | 97 | 97 | 97 | 97 |
| pH6.0 | | | | | | |
| 37° C. | 95 | 95 | 95 | 95 | 94 | 97 |
| pH7.4 | | | | | | |

CE-SDS; reducing

| | | | | | | |
|---|---|---|---|---|---|---|
| 40° C. | 99 | 99 | 99 | 99 | 99 | 99 |
| pH6.0 | | | | | | |
| 37° C. | 98 | 98 | 98 | 98 | 98 | 98 |
| pH7.4 | | | | | | |

Monomer (SEC)

| | | | | | | |
|---|---|---|---|---|---|---|
| 40° C. | 99 | 99 | 99 | 99 | 99 | 99 |
| pH6.0 | | | | | | |
| 37° C. | 98 | 99 | 99 | 98 | 98 | 98 |
| pH7.4 | | | | | | |

Rel. active concentration

| | | | | | | |
|---|---|---|---|---|---|---|
| 40° C. | 99 | 97 | 98 | 97 | 97 | 98 |
| pH6.0 | | | | | | |
| 37° C. | 93 | 94 | 93 | 94 | 87 | 80 |
| pH7.4 | | | | | | |

Binding Kinetics of Anti-Idiotypic Antibodies to Anti-CD3 P035-093 Using SPR

Binding of the parental anti-CD3 P035.093 anti-idiotypic antibody 4.24.72 compared to the humanized variants H1L1, 4.24.72 H1L2, H2L2 and 4.24.72 H3L2 was investigated by surface plasmon resonance using a Biacore T200 instrument (GE Healthcare). Briefly, FOLR1-Fc was immobilized on a series s sensor chip C1 using standard amine coupling chemistry according to the manufacturer's instructions. Final surface densities were obtained between 700 and 1000 RU. Subsequently, FOLR1 CD3 TCB P035.093 was injected on the second flow cell for 30 s. The first flow cell was kept as mock surface. The anti-idiotypic antibodies were injected on both flow cells for 120 s at concentrations from 1.2 to 100 nM (1:3 dilution series). Dissociation was monitored for 300 s, the flow rate was set to 30 μl/min. The surface was regenerated by injecting 10 mM Glycine pH 2.0 for 60 s, followed by injecting 5 mM NaOH for 60 s at a flow rate of 5 μl/min. Bulk refractive index differences were corrected by subtracting the response obtained from flow cell one (mock surface) as well as by substracting buffer injections (double referencing). The derived curves were fitted to a 1:1 Langmuir binding model using the BIAevaluation software (GE Healthcare). The obtained fitting results showed Rmax values between 1 and 4 RU. All experiments were performed at 37° C. using HBS-N (10 mM HEPES, 150 mM NaCl pH 7.4, 0.05% Surfactant P-20).

Results (n=5):

TABLE 13
Binding affinities of CD3 P035.093 anti-idiotypic parental chimeric
antibody 4.24.72 and humanization variants thereof.

| Concept | KD | Stdev | t1/2 | |
|---|---|---|---|---|
| ID | (nM) | (nM) | diss (s) | Stdev (s) |
| 4.24.72 | 1.5 | 0.4 | 446 | 140 |
| H1L1 | 1.8 | 0.6 | 371 | 66 |
| H1L2 | 1.2 | 0.5 | 396 | 198 |
| H2L2 | 1.8 | 1.0 | 372 | 156 |
| H3L2 | 1.1 | 0.5 | 460 | 222 |

### Example 14—Target Cell Killing Mediated by FOLR1 Pro-TCBs (CD3 P035.93 and Humanization Variants as Mask)

Target cell killing was performed to test masking-efficiency of humanization variants of 4.24.72 anti-ID mask in pro-TCB format. FOLR1 positive target cells (Ovcar-3 with medium FOLR1 expression level) were incubated with huPBMCs and TCBs as described above. FOLR1 TCB was used as a positive control (FIGS. 15A-15C). All FOLR1 pro-TCBs (different humanization variants as mask and non-cleavable linker) show reduced target cell killing compared to FOLR1 TCB. Masking-efficiency is comparable for all humanization variants in this assay set up (FIG. 7). Additionally T cell activation was analyzed. FOLR1 TCB induces dose-dependent T cell activation (CD69 increase for CD8 positive T cells). The masked FOLR1 pro-TCB (CD3 P035.093, humanization variants of mask 4.24.72 scFv) containing a non-cleavable linker show reduced T cell activation (CD69 for CD8 T cells) in the indicated concentration range and no differences for masking-efficiency could be detected for the humanization variants.

### Example 15—Characterization of Optimized Anti-CD3 Antibodies by Surface Plasmon Resonance (SPR) after Stress

The experiment was performed as described in Example 4, using the monovalent IgG molecules prepared in Example 7. As shown in Table 14, all the optimized anti-CD3 antibodies show an improved binding upon stress to CD3ε/δ, as compared to CD3_{orig}.

TABLE 14
Binding activity of anti-CD3 antibodies (in monovalent
IgG format) to human CD3ε/δ after incubation
at pH 6/40° C. or pH 7.4/37° C. for 2 weeks.

binding activity [%]

| anti-CD3 | 2 weeks at | 2 weeks at |
|---|---|---|
| antibody | pH 6.0/40° C. | pH 7.4/37° C. |
| CD3_{orig} | 97 | 60 |
| P033.078 | 98 | 95 |
| P035.093 | 100 | 93 |
| P004.042 | 101 | 98 |

Although the foregoing invention has been described in some detail by way of illustration and example for purposes of clarity of understanding, the descriptions and examples should not be construed as limiting the scope of the invention. The disclosures of all patent and scientific literature cited herein are expressly incorporated in their entirety by reference.

1. A protease-activatable T cell activating bispecific molecule comprising
(a) a first antigen binding moiety capable of binding to CD3, wherein the first antigen binding moiety comprises
(i) a heavy chain variable region (VH) comprising a heavy chain complementary determining region (HCDR) 1 of SEQ ID NO: 2, a HCDR 2 of SEQ ID NO: 4, and a HCDR 3 of SEQ ID NO: 10, and
(ii) a light chain variable region (VL) comprising a light chain complementarity determining region (LCDR) 1 of SEQ ID NO: 20, a LCDR 2 of SEQ ID NO: 21 and a LCDR 3 of SEQ ID NO: 22;
(b) a second antigen binding moiety capable of binding to a target cell antigen; and
(c) a masking moiety covalently attached to the T cell bispecific binding molecule through a protease-cleavable linker, wherein the masking moiety is capable of binding to the idiotype of the first antigen binding moiety thereby reversibly concealing the first antigen binding moiety.

2. The protease-activatable T cell activating bispecific molecule of claim 1, wherein the VH comprises an amino acid sequence that is at least about 95%, 96%, 97%, 98%, 99% or 100% identical to the amino acid sequence of SEQ ID NO: 16, and/or the VL comprises an amino acid 20 sequence that is at least about 95%, 96%, 97%, 98%, 99% or 100% identical to the amino acid sequence of SEQ ID NO: 23.

3. The protease-activatable T cell activating bispecific molecule of claim 1 or 2, wherein the masking moiety is covalently attached to the first antigen binding moiety and reversibly conceals the first antigen binding moiety.

4. The protease-activatable T cell activating bispecific molecule of claim 1, wherein the masking moiety is covalently attached to the heavy chain variable region of the first antigen binding moiety.

5. The protease-activatable T cell activating bispecific molecule of claim 1, wherein the masking moiety is an scFv.

6. The protease-activatable T cell activating bispecific molecule of claim 1, wherein the second antigen binding moiety is a crossover Fab molecule wherein either the variable or the constant regions of the Fab light chain and the Fab heavy chain are exchanged.

7. The protease-activatable T cell activating bispecific molecule of claim 1, wherein the first antigen binding moiety is a conventional Fab molecule.

8. The protease-activatable T cell activating bispecific molecule of claim 1, comprising not more than one antigen binding moiety capable of binding to CD3.

9. The protease-activatable T cell activating bispecific molecule of claim 1, comprising a third antigen binding moiety which is a Fab molecule capable of binding to a target cell antigen.

10. The protease-activatable T cell activating bispecific molecule of claim 9, wherein the third antigen binding moiety is identical to the second antigen binding moiety.

11. The protease-activatable T cell activating bispecific molecule of claim 1, wherein the second antigen binding moiety is capable of binding to a target cell antigen selected from the group consisting of FolR1 and TYRP1.

12. The protease-activatable T cell activating bispecific molecule of claim 1, wherein the first and the second antigen binding moieties are fused to each other.

13. The protease-activatable T cell activating bispecific molecule of claim 12, wherein the first and second antigen binding moieties are fused to each other via a peptide linker.

14. The protease-activatable T cell activating bispecific molecule of claim 1, wherein the second antigen binding moiety is fused at the C-terminus of the Fab heavy chain to the N-terminus of the Fab heavy chain of the first antigen binding moiety.

15. The protease-activatable T cell activating bispecific molecule of claim 1, wherein the first antigen binding moiety is fused at the C-terminus of the Fab heavy chain to the N-terminus of the Fab heavy chain of the second antigen binding moiety.

16. The protease-activatable T cell activating bispecific molecule of claim 1, additionally comprising an Fc domain composed of a first and a second subunit capable of stable association.

17. The protease-activatable T cell activating bispecific molecule of claim 16, wherein the Fc domain is an IgG

18. The protease-activatable T cell activating bispecific molecule of claim 17, wherein the IgG is an IgG1 or IgG4.

19. The protease-activatable T cell activating bispecific molecule of claim 17 or 18 wherein the Fc domain exhibits reduced binding affinity to an Fc receptor and/or reduced effector function, as compared to a native IgG1 Fc domain.

20. The protease-activatable T cell activating bispecific molecule of claim 1, wherein the masking moiety comprises a heavy chain variable region comprising:
(a) a heavy chain complementarity determining region (CDR H) 1 amino acid sequence of DYSMN (SEQ ID NO:58);
(b) a CDR H2 amino acid sequence selected from the group consisting of
(SEQ ID NO: 59)
WINTETGEPRYTDDFKG,
(SEQ ID NO: 84)
WINTETGEPRYTDDFTG
and
(SEQ ID NO: 86)
WINTETGEPRYTQGFKG;
(c) a CDR H3 amino acid sequence of EGDYDVFDY (SEQ ID NO:60); and a light chain variable region comprising:
(d) a light chain (CDR L)1 amino acid sequence selected from the group consisting of RASKSVSTSSYSYMH (SEQ ID NO:62) and KSSKSVSTSSYSYMH (SEQ ID NO:82);
(e) a CDR L2 amino acid sequence of YVSYLES (SEQ ID NO:63); and
(f) a CDR L3 amino acid sequence selected from the group consisting of
(SEQ ID NO: 64)
QHSREFPYT
and
(SEQ ID NO: 88)
QQSREFPYT.

21. The protease-activatable T cell activating bispecific molecule of claim 1, wherein the masking moiety comprises a heavy chain variable region comprising:
(a) a heavy chain complementarity determining region (CDR H) 1 amino acid sequence of DYSMN (SEQ ID NO:58);
(b) a CDR H2 amino acid sequence of WINTETGEPRYTDDFKG (SEQ ID NO:59);
5 (c) a CDR H3 amino acid sequence of EGDYDVFDY (SEQ ID NO:60); and a light chain variable region comprising:
(d) a light chain (CDR L)1 amino acid sequence of RASKSVSTSSYSYMH (SEQ ID NO:62);
(e) a CDR L2 amino acid sequence of YVSYLES (SEQ ID NO:63); and
(f) a CDR L3 amino acid sequence of QHSREFPYT (SEQ ID NO:64).

22. The protease-activatable T cell activating bispecific molecule of claim 1, wherein the masking moiety comprises a heavy chain variable region comprising:
(a) a heavy chain complementarity determining region (CDR H) 1 amino acid sequence of SYGVS (SEQ ID NO:58);
(b) a CDR H2 amino acid sequence of IIWGDGSTNYHSALIS (SEQ ID NO:59);
(c) a CDR H3 amino acid sequence of GITTVVDDYYAMDY (SEQ ID NO:60); and a light chain variable region comprising:
(d) a light chain (CDR L)1 amino acid sequence of KSSKSVSTSSYSYMH (SEQ ID NO:82);
(e) a CDR L2 amino acid sequence of AATFLAD (SEQ ID NO:63); and
(f) a CDR L3 amino acid sequence of QHYYSTPYT (SEQ ID NO:64).

23. The protease-activatable T cell activating bispecific molecule of claim 1, wherein the masking moiety comprises a heavy chain variable region comprising:
(a) a heavy chain complementarity determining region (CDR H) 1 amino acid sequence of SYGVS (SEQ ID NO:58);
(b) a CDR H2 amino acid sequence of WINTETGEPRYTDDFTG (SEQ ID NO:84);
(c) a CDR H3 amino acid sequence of GITTVVDDYYAMDY (SEQ ID NO:60); and a light chain variable region comprising:
(d) a light chain (CDR L)1 amino acid sequence of KSSKSVSTSSYSYMH (SEQ ID NO:82);
(e) a CDR L2 amino acid sequence of AATFLAD (SEQ ID NO:63); and
(f) a CDR L3 amino acid sequence of QHYYSTPYT (SEQ ID NO:64).

24. The protease-activatable T cell activating bispecific molecule of claim 1, wherein the masking moiety comprises a heavy chain variable region comprising:
(a) a heavy chain complementarity determining region (CDR H) 1 amino acid sequence of SYGVS (SEQ ID NO:58);
(b) a CDR H2 amino acid sequence of WINTETGEPRYTQGFKG (SEQ ID NO:86);
(c) a CDR H3 amino acid sequence of GITTVVDDYYAMDY (SEQ ID NO:60); and a light chain variable region comprising:
(d) a light chain (CDR L)1 amino acid sequence of KSSKSVSTSSYSYMH (SEQ ID NO:82);
(e) a CDR L2 amino acid sequence of AATFLAD (SEQ ID NO:63); and
(f) a CDR L3 amino acid sequence of QHYYSTPYT (SEQ ID NO:64).

25. The protease-activatable T cell activating bispecific molecule of claim 1, wherein the protease cleavable linker comprises at least one protease recognition sequence.

26. The protease-activatable T cell activating bispecific molecule of claim 1, wherein the protease recognition sequence is selected from the group consisting of:
(a)
(SEQ ID NO: 100)
RQARVVNG;
(b)
(SEQ ID NO: 101)
VHMPLGFLGPGRSRGSFP;
(c)
(SEQ ID NO: 102)
RQARVVNGXXXXXVPLSLYSG, wherein X is any amino
acid;
(d)
(SEQ ID NO: 103)
RQARVVNGVPLSLYSG;
(e)
(SEQ ID NO: 104)
PLGLWSQ;
(f)
(SEQ ID NO: 105)
VHMPLGFLGPRQARVVNG;
(g)
(SEQ ID NO: 106)
FVGGTG;
(h)
(SEQ ID NO: 107)
KKAAPVNG;
(i)
(SEQ ID NO: 108)
PMAKKVNG;
(j)
(SEQ ID NO: 109)
QARAKVNG;
(k)
(SEQ ID NO: 110)
VHMPLGFLGP;
(l)
(SEQ ID NO: 111)
QARAK;
(m)
(SEQ ID NO: 112)
VHMPLGFLGPPMAKK;
(n)
(SEQ ID NO: 113)
KKAAP;
and
(o)
(SEQ ID NO: 114)
PMAKK.

27. The protease-activatable T cell activating bispecific molecule of claim 25 or 26, wherein the protease cleavable linker comprises the protease recognition sequence PMAKK (SEQ ID NO:114).

28. The protease-activatable T cell activating bispecific molecule of claim 1, wherein the second antigen binding moiety is capable of binding to FolR1 and comprises a heavy chain variable region comprising:
a) a heavy chain complementarity determining region (CDR H) 1 amino acid sequence of NAWMS (SEQ ID NO:54);
b) a CDR H2 amino acid sequence of RIKSKTDGGTTDYAAPVKG (SEQ ID NO:55); and
c) a CDR H3 amino acid sequence of PWEWSWYDY (SEQ ID NO:56); and a light chain variable region comprising:
d) a light chain (CDR L)1 amino acid sequence of GSSTGAVTTSNYAN (SEQ ID NO:20);
e) a CDR L2 amino acid sequence of GTNKRAP (SEQ ID NO:21); and
f) a CDR L3 amino acid sequence of ALWYSNLWV (SEQ ID NO:22).

29. The protease-activatable T cell activating bispecific molecule of claim 1, wherein the second antigen binding moiety is capable of binding to TYRP1 and comprises a heavy chain variable region comprising:
a) a heavy chain complementarity determining region (CDR H) 1 amino acid sequence of DYFLH (SEQ ID NO:24);
b) a CDR H2 amino acid sequence of WINPDNGNTVYAQKFQG (SEQ ID NO:25); and
c) a CDR H3 amino acid sequence of RDYTYEKAALDY (SEQ ID NO:26); and a light chain variable region comprising:
d) a light chain (CDR L)1 amino acid sequence of RASGNIYNYLA (SEQ ID NO:28);
e) a CDR L2 amino acid sequence of DAKTLAD (SEQ ID NO:29); and
f) a CDR L3 amino acid sequence of QHFWSLPFT (SEQ ID NO:30).

30. A protease-activatable T cell activating bispecific molecule comprising
(a) a first antigen binding moiety comprising a conventional Fab molecule capable of binding to CD3, wherein the first antigen binding moiety comprises
(i) a heavy chain variable region (VH) comprising a heavy chain complementary determining region (HCDR) 1 of SEQ ID NO: 2, a HCDR 2 of SEQ ID NO: 4, and a HCDR 3 of SEQ ID NO: 10, and
(ii) a light chain variable region (VL) comprising a light chain complementarity determining region (LCDR) 1 of SEQ ID NO: 20, a LCDR 2 of SEQ ID NO: 21 and a LCDR 3 of SEQ ID NO: 22;
(b) a second antigen binding moiety capable of binding to a target cell antigen, the moiety comprising a crossover Fab molecule wherein either the variable or the constant regions of the Fab light chain and the Fab heavy chain are exchanged;
(c) a masking moiety comprising an scFv covalently attached to the T cell bispecific binding molecule through a protease-cleavable linker, wherein the masking moiety is capable of binding to the idiotype of the first antigen binding moiety thereby reversibly concealing the first antigen binding moiety;
wherein the protease-cleavable linker comprises at least one protease recognition sequence selected from the group consisting of:
(i)
(SEQ ID NO: 100)
RQARVVNG;
(ii)
(SEQ ID NO: 101)
VHMPLGFLGPGRSRGSFP;
(iii)
(SEQ ID NO: 102)
RQARVVNGXXXXXVPLSLYSG, wherein X is any amino
acid;
(iv)
(SEQ ID NO: 103)
RQARVVNGVPLSLYSG;
(v)
(SEQ ID NO: 104)
PLGLWSQ;
(vi)
(SEQ ID NO: 105)
VHMPLGFLGPRQARVVNG;
(vii)
(SEQ ID NO: 106)
FVGGTG;
(viii)
(SEQ ID NO: 107)
KKAAPVNG;
(ix)
(SEQ ID NO: 108)
PMAKKVNG;
(x)
(SEQ ID NO: 109)
QARAKVNG;
(xi)
(SEQ ID NO: 110)
VHMPLGFLGP;
(xii)
(SEQ ID NO: 111)
QARAK;
(xiii)
(SEQ ID NO: 112)
VHMPLGFLGPPMAKK;
(xiv)
(SEQ ID NO: 113)
KKAAP;
and
(xv)
(SEQ ID NO: 114)
PMAKK.
wherein the masking moiety comprises a heavy chain variable region comprising:
(1) a heavy chain complementarity determining region (CDR H) 1 amino acid sequence of DYSMN (SEQ ID NO:58);
(2) a CDR H2 amino acid sequence selected from the group consisting of
(SEQ ID NO: 59)
WINTETGEPRYTDDFKG,
(SEQ ID NO: 84)
WINTETGEPRYTDDFTG
and
(SEQ ID NO: 86)
WINTETGEPRYTQGFKG;
(3) a CDR H3 amino acid sequence of EGDYDVFDY (SEQ ID NO:60); and a light chain variable region comprising:
(4) a light chain (CDR L)1 amino acid sequence selected from the group consisting of RASKSVSTSSYSYMH (SEQ ID NO:62) and KSSKSVSTSSYSYMH (SEQ ID NO:82);
(5) a CDR L2 amino acid sequence of YVSYLES (SEQ ID NO:63); and
(6) a CDR L3 amino acid sequence selected from the group consisting of
(SEQ ID NO: 64)
QHSREFPYT
and
(SEQ ID NO: 88)
QQSREFPYT;
(d) a third antigen binding moiety which is a Fab molecule capable of binding to a target cell antigen, wherein the third antigen binding moiety is identical to the second antigen binding moiety;
(e) an Fc domain composed of a first and a second subunit capable of stable association, wherein the Fc domain comprises an IgG;
wherein the second and third antigen binding moieties are capable of binding a target cell antigen of FolR1 and comprises a heavy chain variable region comprising:
I) a heavy chain complementarity determining region (CDR H) 1 amino acid sequence of NAWMS (SEQ ID NO:54);
II) a CDR H2 amino acid sequence of RIKSKTDGGTTDYAAPVKG (SEQ ID NO:55); and
III) a CDR H3 amino acid sequence of PWEWSWYDY (SEQ ID NO:56); and a light chain variable region comprising:
IV) a light chain (CDR L)1 amino acid sequence of GSSTGAVTTSNYAN (SEQ ID NO:20);
V) a CDR L2 amino acid sequence of GTNKRAP (SEQ ID NO:21); and
VI) a CDR L3 amino acid sequence of ALWYSNLWV (SEQ ID NO:22).
or TYRP1 comprising a heavy chain variable region comprising:
VII) a heavy chain complementarity determining region (CDR H) 1 amino acid sequence of DYFLH (SEQ ID NO:24);
VIII) a CDR H2 amino acid sequence of WINPDNGNTVYAQKFQG (SEQ ID NO:25); and
IX) a CDR H3 amino acid sequence of RDYTYEKAALDY (SEQ ID NO:26); and a light chain variable region comprising:
X) a light chain (CDR L)1 amino acid sequence of RASGNIYNYLA (SEQ ID NO:28);
XI) a CDR L2 amino acid sequence of DAKTLAD (SEQ ID NO:29); and
XII) a CDR L3 amino acid sequence of QHFWSLPFT (SEQ ID NO:30).
wherein the first and the second antigen binding moieties are fused to each other via a peptide linker;
wherein the second antigen binding moiety is fused at the C-terminus of the Fab heavy chain to the N-terminus of the Fab heavy chain of the first antigen binding moiety or the first antigen binding moiety is fused at the C-terminus of the Fab heavy chain to the N-terminus of the Fab heavy chain of the second antigen binding moiety;

31. The protease-activatable T cell activating bispecific molecule of claim 30, wherein the IgG of the Fc domain is an IgG1 or IgG4.

32. The protease-activatable T cell activating bispecific molecule of claim 31, wherein the Fc domain exhibits reduced binding affinity to an Fc receptor and/or reduced effector function, as compared to a native IgG1 Fc domain.

33. A idiotype-specific polypeptide capable of reversibly concealing an anti-CD3 antigen binding site of a molecule, wherein the idiotype-specific polypeptide comprises a heavy chain variable region sequence that is at least about 95%, 96%, 97%, 98%, 99% or 100% identical to an amino acid sequence selected from the group consisting of SEQ ID NO: 79, SEQ ID NO:83 and SEQ ID NO:85, and a light chain variable region sequence that is at least about 95%, 96%, 97%, 98%, 99% or 100% identical to an amino acid sequence selected from the group consisting of SEQ ID NO: 80 and SEQ ID NO:81;

34. The idiotype-specific polypeptide of claim 33, wherein the idiotype-specific polypeptide comprises
a) a heavy chain variable region sequence that is at least about 95%, 96%, 97%, 98%, 99% or 100% identical to SEQ ID NO: 79 and a light chain variable region sequence that is at least about 95%, 96%, 97%, 98%, 99% or 100% identical to SEQ ID NO: 80;
b) a heavy chain variable region sequence that is at least about 95%, 96%, 97%, 98%, 99% or 100% identical to SEQ ID NO: 79 and a light chain variable region sequence that is at least about 95%, 96%, 97%, 98%, 99% or 100% identical to SEQ ID NO: 81;
c) a heavy chain variable region sequence that is at least about 95%, 96%, 97%, 98%, 99% or 100% identical to SEQ ID NO: 83 and a light chain variable region sequence that is at least about 95%, 96%, 97%, 98%, 99% or 100% identical to SEQ ID NO: 81; or
d) a heavy chain variable region sequence that is at least about 95%, 96%, 97%, 98%, 99% or 100% identical to SEQ ID NO: 85 and a light chain variable region sequence that is at 20 least about 95%, 96%, 97%, 98%, 99% or 100% identical to SEQ ID NO: 81.

35. The idiotype-specific polypeptide of claim 33, wherein the idiotype-specific polypeptide is an scFv.

36. The idiotype-specific polypeptide of claim 33, wherein the idiotype-specific polypeptide is covalently attached to the molecule through a linker.

37. The idiotype-specific polypeptide of claim 36, wherein the linker is a peptide linker.

38. The idiotype-specific polypeptide of claim 36, wherein the linker is a protease-cleavable linker.

39. The idiotype-specific polypeptide of claim 38, wherein the peptide linker comprises at least one protease recognition site.

40. The idiotype-specific polypeptide of claim 39, wherein the protease recognition sequence is selected from the group consisting of:
(a)
(SEQ ID NO: 100)
RQARVVNG;
(b)
(SEQ ID NO: 101)
VHMPLGFLGPGRSRGSFP;
(c)
(SEQ ID NO: 102)
RQARVVNGXXXXXVPLSLYSG, wherein X is any amino
acid;
(d)
(SEQ ID NO: 103)
RQARVVNGVPLSLYSG;
(e)
(SEQ ID NO: 104)
PLGLWSQ;
(f)
(SEQ ID NO: 105)
VHMPLGFLGPRQARVVNG;
(g)
(SEQ ID NO: 106)
FVGGTG;
(h)
(SEQ ID NO: 107)
KKAAPVNG;
(i)
(SEQ ID NO: 108)
PMAKKVNG;
(j)
(SEQ ID NO: 109)
QARAKVNG;
(k)
(SEQ ID NO: 110)
VHMPLGFLGP;
(l)
(SEQ ID NO: 111)
QARAK;
(m)
(SEQ ID NO: 112)
VHMPLGFLGPPMAKK;
(n)
(SEQ ID NO: 113)
KKAAP;
and
(o)
(SEQ ID NO: 114)
PMAKK.

41. The idiotype-specific polypeptide of claim 40, wherein the protease cleavable linker comprises the protease recognition sequence PMAKK (SEQ ID NO:114).

42. The idiotype-specific polypeptide of claim 33, wherein the idiotype-specific polypeptide is part of a T-cell activating bispecific molecule.

43. A idiotype-specific polypeptide comprising an scFv capable of reversibly concealing an anti-CD3 antigen binding site of a molecule, wherein the idiotype-specific polypeptide comprises a heavy chain variable region sequence that is at least about 95%, 96%, 97%, 98%, 99% or 100% identical to an amino acid sequence selected from the group consisting of SEQ ID NO: 79, SEQ ID NO:83 and SEQ ID NO:85, and a light chain variable region sequence that is at least about 95%, 96%, 97%, 98%, 99% or 100% identical to an amino acid sequence selected from the group consisting of SEQ ID NO: 80 and SEQ ID NO:81;
wherein the idiotype-specific polypeptide is covalently attached to the molecule through a protease cleavable linker, the protease cleavable linker comprising at least one protease recognition site, selected from the group consisting of:
(a)
(SEQ ID NO: 100)
RQARVVNG;
(b)
(SEQ ID NO: 101)
VHMPLGFLGPGRSRGSFP;
(c)
(SEQ ID NO: 102)
RQARVVNGXXXXXVPLSLYSG, wherein X is any amino
acid;
(d)
(SEQ ID NO: 103)
RQARVVNGVPLSLYSG;
(e)
(SEQ ID NO: 104)
PLGLWSQ;
(f)
(SEQ ID NO: 105)
VHMPLGFLGPRQARVVNG;
(g)
(SEQ ID NO: 106)
FVGGTG;
(h)
(SEQ ID NO: 107)
KKAAPVNG;
(i)
(SEQ ID NO: 108)
PMAKKVNG;
(j)
(SEQ ID NO: 109)
QARAKVNG;
(k)
(SEQ ID NO: 110)
VHMPLGFLGP;
(l)
(SEQ ID NO: 111)
QARAK;
(m)
(SEQ ID NO: 112)
VHMPLGFLGPPMAKK;
(n)
(SEQ ID NO: 113)
KKAAP;
and
(o)
(SEQ ID NO: 114)
PMAKK.
wherein the idiotype-specific polypeptide is part of a T-cell activating bispecific molecule.

44. A pharmaceutical composition comprising the protease-activatable T cell activating bispecific molecule of claim 1 or the idiotype-specific polypeptide of claim 33 and a pharmaceutically acceptable carrier.

45. An isolated polynucleotide encoding the protease-activatable T cell activating bispecific antigen binding molecule of claim 1 or idiotype-specific polypeptide of claim 33.

46. A vector comprising the polynucleotide of claim 45.

47. The vector or claim 46, which is an expression vector.

48. A host cell comprising the polynucleotide of claim 45 or the vector of claim 47.

49. A method of producing a protease-activatable T cell activating bispecific molecule, comprising the steps of a) culturing the host cell of claim 48 under conditions suitable for the expression of the protease-activatable T cell activating bispecific molecule and b) recovering the protease-activatable T cell activating bispecific molecule.

50. A method of treating a disease in an individual, comprising administering to said individual a therapeutically effective amount of a composition comprising the protease-activatable T cell activating bispecific molecule of claim 1.

51. The method of claim 50, wherein treating the disease is treating or delaying progression of cancer, treating or delaying progression of an immune related disease, or enhancing or stimulating an immune response or function in an individual.
